(19) United States
(12) Patent Application Publication
(10) Pub. No.: US 2026/0355331 A1
(43) Pub. Date: Nov. 12, 2026

(54) CEREBLON DEGRADER CONJUGATES, AND USES THEREOF

(71) Applicant: GENENTECH, INC., South San Francisco, CA (US)

(72) Inventor: Thomas Harden PILLOW, South San Francisco, CA (US)

(73) Assignee: GENENTECH, INC., South San Francisco, CA (US)

(21) Appl. No.: 19/140,957

(22) PCT Filed: Dec. 22, 2023
(86) PCT No.: PCT/US2023/085698

§ 371 Date: Jun. 18, 2025

Related U.S. Application Data
(60) Provisional application No. 63/435,142, filed on Dec. 23, 2022.
(60) Provisional application No. 63/525,282, filed on Jul. 6, 2023.

(51) Int. Cl. A61K 47/68; A61P 35/00; C07D 401/14; C07D 471/16; C07D 487/04; C07D 495/14

(52) U.S. Cl. CPC A61K 47/6803; A61K 47/6851; A61K 47/6889; A61P 35/00; C07D 401/14; C07D 471/16; C07D 487/04; C07D 495/14

(57) ABSTRACT

Provided here are cereblon degrader antibody conjugates (cDACs) comprising a cereblon degrader moiety covalently attached to an antibody. The cDAC targets proteins for intracellular degradation and may be useful to treat diseases and disorders.

### CROSS REFERENCE TO RELATED APPLICATIONS

This application claims the benefit of priority to U.S. Provisional Application No. 63/435,142, filed Dec. 23, 2022, and U.S. Provisional Application No. 63/525,282, filed on Jul. 6, 2023, the content of each of which is incorporated by reference in its entirety.

### FIELD

The disclosure relates generally to cereblon degrader antibody conjugates (cDACs) compositions, intermediates for their manufacture, and methods of their use. The cDACs are useful for facilitating intracellular degradation of target proteins.

### BACKGROUND

Cereblon is a 442-amino acid multifunctional protein located in the cytoplasm, nucleus and peripheral membrane of the human brain and other tissues (Wada et al., Biochem. & Biophys. Res. Comm. 477:388-94 (2016)). Cereblon ensures normal metabolic function and normal physiological function of ion channels, which are important to maintaining cell growth and proliferation. Cereblon is also involved in the occurrence of many diseases, such as cancer (Shi et al, (2017) J. Immunol. Res. Article ID 9130608). Cereblon interacts with the DNA damage-binding protein-1 (DDB1), Cullin 4 (Cul4A and Cul4B), and regulator of Cullins 1 (RoC1) to form the functional E3 ubiquitin ligase complex, which is known as the CRL4/CRBN E3 ubiquitin ligase complex. Cereblon's role as part of this complex includes a number of targeting proteins for proteolysis (degradation) via a ubiquitin-proteasome pathway (Chang et al, (2011) Int. J. Biochem. Mol. Biol. 2(3):287-94). This complex ubiquitinates a number of other proteins. Cereblon is also implicated in the development of cerebral tissues and because of its expression in the hippocampus among other areas, is associated with memory and learning processes (Higgins, et al, (2004) Neurol. 63(10):1927-31).

Cereblon is a target for immunomodulatory drugs (IMiDs) which adjust immune responses and contain a glutarimide functional group (Kazantsev, A. et al, (2022) Expert Opinion on Therapeutic Patents, 32:2, 171-190; Kronke et al., (2015) Nature 523:183-8; Hagner et al., (2016) Blood 126(6):779-89). The IMiD class includes thalidomide and analogues: lenalidomide, pomalidomide, iberdomide, and apremilast. Thalidomide is approved by the FDA for treatment of multiple myeloma. Lenalidomide (REVLIMID®) and pomalidomide (POMALYST®), are approved by the FDA for treatment of multiple myeloma and other diseases. Cytokine modulation and T cell co-stimulation by IMiDs results in interleukin-2 production in T cells (Schafer et al., (2003) J. Pharmacol. & Exper. Ther. 305:1222-32). IMiDs have pleiotropic effects on a wide range of immune cells including natural killer (NK) cell activation and B cell and monocyte inhibition (Corral et al., (1999) J. Immunol. 163:380-6). Approved drugs, thalidomide and derivatives lenalidomide and pomalidomide have been repurposed as immunomodulatory drugs (IMiDs) for blood cancers (Ito T, et al (2020) Proc Jpn Acad Ser B Phys Biol Sci. 96(6):189-203). Structural studies have shown that IMiDs such as thalidomide, lenalidomide and pomalidomide bind in a shallow hydrophobic pocket on the surface of cereblon, and that the binding is mediated by the glutarimide ring.

As the binding protein for IMiDs, cereblon is responsible for the multiple effects of IMiDs like thalidomide and its analogs (P. Ottis, et al (2017) ACS Chem. Biol. 12 (4): 892-898; Shi Q, et al (2017) J Immunol Res. 2017:9130608; Sperling A S, et al (2019) Blood 134(2):160-170). Cereblon expression can affect cell metabolism and can be a causative effect of a disease even in the absence of IMiDs. Cereblon orthologs are highly conserved from plants to humans, which underscores its physiological importance (Zhihua H, et al (2011) Annu Rev Plant Biol. 62(1):299-334).

The ATP-dependent ubiquitin-proteasome system (UPS) is the main pathway for intracellular protein degradation. The UPS system, which includes ubiquitin (Ub), proteasomes, catalytic enzymes, and specific substrates, plays an important role in various biological processes. Ubiquitination occurs through a cascade of enzymatic events, particularly in the synergistic action of Ub-activating enzyme (E1), ubiquitin-conjugating enzyme (E2), and ubiquitin-ligase enzyme (E3). Once the substrate protein is polyubiquitinated, it will be recognized and degraded by the proteasome and UPS can digest the substrate protein into small peptides. Specific recognition of substrate proteins is the apparent function of E3, so E3 plays an important role in determining the specificity of Ub-mediated protein degradation (B. E. Smith, et al, (2019), Nat. Commun. 10 (1):131; K. M. Sakamoto, (2010) Pediatr. Res. 67 (5):505-508; M. Scheepstra, et al, (2019), Comput. Struct. Biotechnol. J. 17:160-176; P. Ottis, et al, (2017) ACS Chem. Biol. 12 (4):892-898).

Proteolysis-targeting chimeras (PROTACs) are heterobifunctional degrader constructs capable of targeted degradation of aberrantly acting proteins using the cell's ubiquitin-proteasome machinery. The main mechanism of PROTACs technology is to use UPS to degrade proteins of interest (POI) such as targeted proteins that are themselves disease mediators by bringing in proximity the E3 ubiquitin ligase with a POI that is to be targeted for degradation leading to degradation of the targeted protein (Lu et al, (2015) Cell Cancer 22(6):755-63; Wang, C. et al (2021) Eur J Med Chem. 225:113749). The E3 ligase ligand of PROTAC can hijack the E3 ligase and label the POI with ubiquitin. In this process, PROTAC itself is not degraded but is recycled to promote ubiquitination and degradation of other target proteins (M. L. Drummond, et al (2019), J. Chem. Inf Model. 59(4):1634-1644; S. An, et al (2018), EBioMedicine 36:553-562; W. Farnaby, et al, (2019), Nat. Chem. Biol. 15(7):672-680; M. S. Gadd, et al, (2017), Nat. Chem. Biol. 13(5):514-521; R. P. Nowak, et al, (2018) Nat. Chem. Biol. 14(7):706-714). This catalytic, event-driven modality operates in contrast to the function of conventional inhibitors, in which sequential target binding is necessary to stimulate the desired effect. For standard occupancy-driven typical small-molecule drugs, binding affinity is necessary for their efficacy. In contrast, PROTACs induce degradation of POI by UPS, an event-driven modality that can be used to overcome common drawbacks of traditional occupancy-driven small-molecule drugs (K. M. Sakomoto, et al, (2001), Proc. Natl. Acad. Sci. USA 98(15):8554-8559; P. Martin-Acosta, et al, (2021), Eur. J. Med. Chem. 210:112993; S. Zeng, et al, (2021) Eur. J. Med. Chem. 210:112981; M. Toure, et al, (2016) Angew Chem. Int. Ed. Engl. 55(6):1966-1973). “Molecular glue” are degrader constructs which mediate proximity-induced protein degradation interacting with the ligase (more frequently) or the target POI by inducing or stabilizing the protein-protein interaction between the E3 ubiquitin ligase and the POI to form ternary complexes that induce ubiquitination and degradation of target protein POIs (den Besten, W. et al (2020) Nature Chemical Biology 16:1158). Molecular glues can degrade otherwise unligandable proteins by orchestrating direct interactions between target and ligase (Mayor-Ruiz, C. et al (2020) Nature Chemical Biology 16:1199-1207; Dong G, et al (2021) J Med Chem. 64(15):10606-10620). The molecular glue degrader may target nuclear receptor GSPT1 (Huber, A. D., et al (2022) ACS Med. Chem. Lett. 13:1311-1320).

Although both molecular glue and PROTACs are bifunctional protein degraders, they have different mechanisms of action and structural requirements (den Besten, W., et al (2020) Nat Chem Biol 16:1157-1158). However, cereblon ligands can be components of both PROTAC and molecular glue degraders that recruit targeted POIs to CRL4/CRBN E3 ubiquitin ligase for degradation of the POI (Lu et al, (2015) Cell Cancer 22(6):755-63; Wang, C. et al (2021) Eur J Med Chem. 225:113749). Certain glutarimide compounds such as thalidomide, lenalidomide, and pomalidomide function as a molecular glue to enhance or induce interactions between E3 ligase and the target protein and thereby trigger ubiquitination and degradation (Dong G, et al (2021) J Med Chem. 64(15):10606-10620).

One protein of interest is bromodomain-containing protein 4 (BRD4). Certain small-molecule BRD4 inhibitors interfere with protein-protein interactions and have been the subject of antitumor drug development. Limitations include reversible binding of BRD4 inhibitors (e.g. JQ1, OTX015) requiring large systemic drug concentrations and sustained exposure to ensure adequate functional inhibition (J. Shi, et al (2018) Mol. Pharm. 15 (9):4139-4147).

Target protein ligands have been employed in PROTACs with pomalidomide through a PEG linker to various BRD4 target-protein ligands which induced significant degradation of BRD4 in BL (Burkitt's lymphoma) cells, with a DC50 value below 1 nM (J. Lu, et al (2015) Chem. Biol. 22(6):755-763). PROTACs with other BRD4 and BET target protein ligands showed significant effects on c-MYC, AML (acute myeloid leukemia) cells, and downstream cell proliferation and apoptosis induction in BL cells. (E. W. Georg, et al, (2015) Science 348 (6241):1376-1381). Such results demonstrate that cereblon-based PROTACs with BET provide a better and more efficient strategy in targeting BRD4 than traditional small-molecule inhibitors (L. Bai, et al, (2017) Canc. Res. 77(9): 2476-2487; C. Qin, et al, (2018) J. Med. Chem. 61(15):6685-6704; J. Zhang, et al (2020) Bioorg. Chem. 99:103817). Degradation of BET proteins was correlated with linker design in PROTACs (T. A. Bemis, et al (2021), Chem. Commun. 57(8):1026-1029).

Limitations or challenges exist for the design, preparation and use of compositions of antibodies covalently attached through linkers to drugs, payloads, and other biologically active moieties. Linkers can be classified into cleavable and non-cleavable linkers according to their chemical properties (Beck A, et al, (2017) Nat Rev Drug Discov. 16(6):315-37; Tsuchikama K, et al, (2018) Protein Cell. 9:33-46). Non-cleavable linkers consist of stable bonds resistant to proteolytic degradation, so that cleavage occurs only after lysosome internalization and complete degradation of the antibody. These linkers have higher stability than cleavable ones, but can suffer from lower membrane permeability. Conversely, cleavage of cleavable linkers can depend on external pH (acid-labile linkers), specific lysosomal proteases (protease-cleavable linkers) or glutathione reduction of disulfide linkers (Shen B-Q, et al, (2012) Nat Biotechnol; Bargh J D, et al, (2019) Chem Soc Rev. 48:4361-74). Thus, some linkers may be labile in the blood stream, releasing unacceptable amounts of the drug prior to internalization in a target cell (Khot, A. et al, (2015) Bioanalysis 7(13):1633-1648) while other linkers may provide stability in the bloodstream, but intracellular release effectiveness may be negatively impacted. Further, linkers that provide for desired intracellular release may have poor stability in the bloodstream. In addition, the amount of drug moiety loaded on the antibody, quantified as the drug to antibody ratio (DAR), the amount of aggregate that is formed in the conjugation reaction, and the yield of final purified conjugate that can be obtained are other parameters that need to be addressed and are often interrelated.

Accordingly, there is a continuing need for improvements in the design of antibody conjugates including linkers and attachment chemistry to provide for optimized safety and efficacy. Additionally, there is a need for enhanced and targeted delivery of cereblon ligand containing PROTACs and molecular glues to cells that contain the protein target. The combination of tumor-associated protein degradation and cereblon immunomodulatory activity may have enhanced therapeutic benefit in patients suffering from a variety of hyperproliferative disorders such as cancer.

### SUMMARY

The disclosure is generally directed to a conjugate composition, referred to as a cereblon degrader antibody conjugate or “cDAC”, where a cereblon degrader moiety is covalently attached to an antibody by an antibody linker. In some embodiments, the cereblon degrader moiety comprises a target protein ligand covalently attached to a cereblon-binding, E3 ubiquitin ligase ligand by a degrader linker. In other embodiments, the cereblon degrader moiety is a molecular glue. In some embodiments, the cereblon degrader moiety of the disclosed cDAC is targeted to the appropriate target cell and released as a cereblon degrader compound, thereby carrying out its function to stimulate/induce ubiquitination of a target protein and effect its degradation by the ubiquitin-proteasome system (UPS). The cDAC may have enhanced therapeutic benefit in patients suffering from a variety of hyperproliferative disorders such as cancer.

One aspect of the disclosure is a cereblon degrader antibody conjugate (cDAC) comprising a cereblon degrader moiety covalently attached to an antibody by a linker (e.g., an antibody linker) wherein the cereblon degrader moiety is a target protein ligand covalently attached to a cereblon-binding, E3 ubiquitin ligase ligand by a degrader linker, or molecular glue, and the antibody is a thiol-containing antibody.

Another aspect of the disclosure is a cDAC having a structure of Formula I:

Ab-[L₁-cD]ₚ I

or a pharmaceutically acceptable salt thereof,
wherein:
Ab is an antibody;
cD is a cereblon degrader moiety;
L₁ is a linker attached to the Ab and the cD; and
p is an integer from 1 to 14.

Another aspect of the disclosure is a cereblon degrader-linker intermediate having a structure of Formula II:

X-L₃-cD II
wherein:
X is a thiol-reactive group;
L₃ is a linker selected from:
   (i) a protease-cleavable, non-peptide linker having the formula:

-Str-PM-IM-
wherein Str is a stretcher unit covalently attached to X,
PM is a peptidomimetic unit, and
IM is an immolator unit covalently attached to cD;
   (ii) a disulfide linker selected from the formulae:

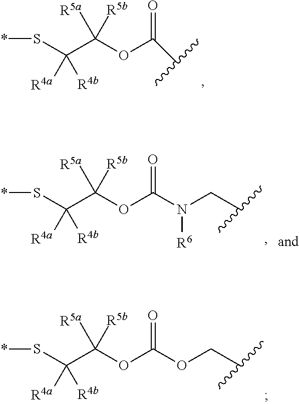

and
   (iii) a linker having the formula:

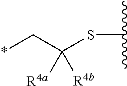

wherein * indicates the point of attachment to X,
R^{4a}, R^{4b}, R^{5a}, and R^{5a} are each independently selected from H and C₁-C₆ alkyl, or R^{4a} and R^{4b} together with the carbon atom to which they are bound form a three-, four-, or five-membered cycloalkyl or heterocyclyl, optionally substituted with F, Cl, and C₁-C₆ alkyl;
R⁶ is selected from H and C₁-C₆ alkyl,
the wavy line indicates the point of attachment to cD,
C₁-C₆ alkyl of R^{4a}, R^{4b}, R^{5a}, R^{5a} and R⁶ is independently and optionally substituted with one or more groups selected from F, Cl, —CN, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H; and
cD is a cereblon degrader moiety, wherein
(i) cD is a molecular glue (MG); or
(ii) cD is a cereblon degrader moiety having the formula:

TPL-L₂-E3UL
wherein:
TPL is a target protein ligand;
E3UL is a cereblon-binding, E3 ubiquitin ligase ligand; and
and L₂ is a degrader linker.

Another aspect of the disclosure is a cDAC prepared by conjugation of an antibody with a cereblon degrader intermediate of Formula II.

Another aspect of the disclosure is a process for preparing a cDAC comprising reacting a thiol-containing antibody with a cereblon degrader intermediate of Formula II.

Another aspect of the disclosure is a pharmaceutical composition comprising a therapeutically effective amount of the cDAC and one or more pharmaceutically acceptable diluent, vehicle, carrier or excipient.

Another aspect of the disclosure is a method for treating cancer comprising administering to a patient in need thereof a therapeutically effective amount of the cDAC.

Another aspect of the disclosure is use of the cDAC in the manufacture of a medicament for the treatment of cancer in a mammal.

Another aspect of the disclosure is use of the cDAC for the treatment of cancer in a mammal.

### BRIEF DESCRIPTION OF THE DRAWINGS

FIG. 1 shows an anti-proliferative effect of in vitro potency by a BRD4-cereblon degrader against KPL-4 and SK-BR-3 cells at 5 days. Cell viability as percent of control is plotted in a graph versus the concentration of cereblon degrader compound cD-5 (nM).

FIG. 2A shows anti-proliferative effects of in vitro potency by treatment after 5 days of HER2+ KPL-4 cells with anti-HER2 7C2 and anti-CD33 BRD4-cereblon degrader antibody conjugates cDAC-3, cDAC-4, cDAC-5, and cDAC-6 from Table 3. Cell viability as percent of control is plotted in a graph versus the concentration of cDAC (μg/mL).

FIG. 2B shows anti-proliferative effects of in vitro potency by treatment after 5 days of HER2+SK-BR-3 cells with anti-HER2 7C2 and anti-CD33 BRD4-cereblon degrader antibody conjugates cDAC-3, cDAC-4, cDAC-5, and cDAC-6. Cell viability as percent of control is plotted in a graph versus the concentration of cDAC (μg/mL).

FIG. 3A shows anti-proliferative effects of in vitro potency by treatment after 5 days of HER2−low/ER+ CAMA1 cells with anti-HER2 7C2 and anti-CD33 BRD4-cereblon degrader antibody conjugates cDAC-3, cDAC-4, cDAC-5, and cDAC-6. Cell viability as percent of control is plotted in a graph versus the concentration of cDAC (μg/mL).

FIG. 3B shows anti-proliferative effects of in vitro potency by treatment after 5 days of HER2−low/ER+ EFM19 cells with anti-HER2 7C2 and anti-CD33 BRD4-cereblon degrader antibody conjugates cDAC-3, cDAC-4, cDAC-5, and cDAC-6. Cell viability as percent of control is plotted in a graph versus the concentration of cDAC (μg/mL).

FIG. 4 shows anti-proliferative effects of in vitro potency by treatment after 7 days of various AML cell lines with anti-CD33 BRD4-cereblon degrader antibody conjugate cDAC-3. The AML cell lines were MV-4-11, EOL-1, Molm-13, Nomo-1, HL-60, and OCI-AML-2. Cell viability as percent of control is plotted in a graph versus the concentration of cDAC (μg/mL).

FIG. 5A shows anti-proliferative effects of in vitro potency by treatment after 5 days of EOL-1 AML cells with anti-HER2 7C2 and anti-CD33 BRD4-cereblon degrader antibody conjugates cDAC-3, cDAC-4, cDAC-5, and cDAC-6. Cell viability as percent of control is plotted in a graph versus the concentration of cDAC (μg/mL).

FIG. 5B shows anti-proliferative effects of in vitro potency by treatment after 5 days of HL-60 AML cells with anti-HER2 7C2 and anti-CD33 cereblon degrader antibody conjugates cDAC-3, cDAC-4, cDAC-5, and cDAC-6. Cell viability as percent of control is plotted in a graph versus the concentration of cDAC (μg/mL).

FIG. 6A shows anti-proliferative effects of in vitro potency by treatment after 3 days of Molm-13 AML cells with anti-HER2 7C2 and anti-CD33 BRD4-cereblon degrader antibody conjugates cDAC-3, cDAC-4, cDAC-5, and cDAC-6. Cell viability as percent of control is plotted in a graph versus the concentration of cDAC (μg/mL).

FIG. 6B shows anti-proliferative effects of in vitro potency by treatment after 3 days of MV-4-11 AML cells with anti-HER2 7C2 and anti-CD33 BRD4-cereblon degrader antibody conjugates cDAC-3, cDAC-4, cDAC-5, and cDAC-6. Cell viability as percent of control is plotted in a graph versus the concentration of cDAC (μg/mL).

FIG. 7 shows the in vivo efficacies of anti-CD33 BRD4-cereblon degrader antibody conjugates cDAC-3, cDAC-4, cDAC-5, and cDAC-6 at the following doses in reducing tumor volume over time (21 days) in a HL-60 xenograft mice model.
1) Vehicle (Histidine Buffer #8), 100 μL, IV once
2) cDAC-4, 3 mg/kg IV once
3) cDAC-3, 1 mg/kg IV once
4) cDAC-3, 3 mg/kg IV once
5) cDAC-3, 10 mg/kg IV once
6) cDAC-6, 3 mg/kg IV once
7) cDAC-5, 1 mg/kg IV once
8) cDAC-5, 3 mg/kg IV once

### DETAILED DESCRIPTION

Reference will now be made in detail to certain embodiments of the present disclosure, examples of which are illustrated in the accompanying structures and formulae. While the invention will be described in conjunction with the enumerated embodiments, it will be understood that they are not intended to limit the invention to those embodiments. On the contrary, the invention is intended to cover all alternatives, modifications, and equivalents, which may be included within the scope provided herein as defined by the claims.

One skilled in the art will recognize many methods and materials similar or equivalent to those described herein, which could be used in the practice of the present invention. The invention is in no way limited to the methods and materials described.

### DEFINITIONS

The term “antibody” is used in the broadest sense and specifically encompasses monoclonal antibodies (including full length monoclonal antibodies), polyclonal antibodies, multispecific antibodies (e.g., bispecific antibodies), and antibody fragments so long as they exhibit the desired biological activity. “Antibody fragment” and all grammatical variants thereof as used herein are defined as a portion of an intact antibody comprising the antigen binding site or variable region of the intact antibody, wherein the portion is free of the constant heavy chain domains (i.e., CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules; (2) single chain polypeptides containing only one light chain variable domain, or a fragment thereof that contains the three CDRs of the light chain variable domain, without an associated heavy chain moiety; (3) single chain polypeptides containing only one heavy chain variable region, or a fragment thereof containing the three CDRs of the heavy chain variable region, without an associated light chain moiety; (4) nanobodies comprising single Ig domains from non-human species or other specific single-domain binding modules; and (5) multispecific or multivalent structures formed from antibody fragments. In an antibody fragment comprising one or more heavy chains, the heavy chain(s) can contain any constant domain sequence (e.g., CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

“Antibody” refers to a polypeptide comprising an antigen binding region (including the complementarity determining region (CDRs)) from an immunoglobulin gene or fragments thereof. The term “antibody” specifically encompasses monoclonal antibodies (including full length monoclonal antibodies), polyclonal antibodies, multispecific antibodies (e.g., bispecific antibodies), and antibody fragments that exhibit the desired biological activity. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa) connected by disulfide bonds. Each chain is composed of structural domains, which are referred to as immunoglobulin domains. These domains are classified into different categories by size and function, e.g., variable domains or regions on the light and heavy chains (V_{L} and V_{H}, respectively) and constant domains or regions on the light and heavy chains (C_{L} and C_{H}, respectively). The N-terminus of each chain defines a variable region of about 100 to 110 or more amino acids, referred to as the paratope, primarily responsible for antigen recognition, i.e., the antigen binding domain. Light chains are classified as either kappa or lambda. Heavy chains are classified as gamma, mu, alpha, delta, or epsilon, which in turn define the immunoglobulin classes, IgG, IgM, IgA, IgD and IgE, respectively. IgG antibodies are large molecules of about 150 kDa composed of four peptide chains. IgG antibodies contain two identical class γ heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding domain. There are four IgG subclasses (IgG1, IgG2, IgG3, and IgG4) in humans, named in order of their abundance in serum (i.e., IgG1 is the most abundant).

Typically, the antigen binding domain of an antibody will be most critical in specificity and affinity of binding to cancer cells.

An antibody that targets a particular antigen includes a bispecific or multispecific antibody with at least one antigen binding region that targets the particular antigen. In some embodiments, the targeted monoclonal antibody is a bispecific antibody with at least one antigen binding region that targets tumor cells.

“Antibody construct” refers to an antibody or a fusion protein comprising (i) an antigen binding domain and (ii) an Fc domain.

In some embodiments, the binding agent is an antigen-binding antibody “fragment,” which is a construct that comprises at least an antigen-binding region of an antibody, alone or with other components that together constitute the antigen-binding construct. Many different types of antibody “fragments” are known in the art, including, for instance, (i) a Fab fragment, which is a monovalent fragment consisting of the V_{L}, V_{H}, C_{L}, and CH₁ domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a single chain Fv (scFv), which is a monovalent molecule consisting of the two domains of the Fv fragment (i.e., V_{L} and V_{H}) joined by a synthetic linker which enables the two domains to be synthesized as a single polypeptide chain.

The antibody or antibody fragments can be part of a larger construct, for example, a conjugate or fusion construct of the antibody fragment to additional regions. For instance, in some embodiments, the antibody fragment can be fused to an Fc region as described herein. In other embodiments, the antibody fragment (e.g., a Fab or scFv) can be part of a chimeric antigen receptor or chimeric T-cell receptor, for instance, by fusing to a transmembrane domain (optionally with an intervening linker or “stalk” (e.g., hinge region)) and optional intercellular signaling domain. For instance, the antibody fragment can be fused to the gamma and/or delta chains of a T-cell receptor, so as to provide a T-cell receptor like construct that binds TROP2. In yet another embodiment, the antibody fragment is part of a bispecific T-cell engager (BiTEs) comprising a CD1 or CD3 binding domain and linker.

“Epitope” means any antigenic determinant or epitopic determinant of an antigen to which an antigen binding domain binds (i.e., at the paratope of the antigen binding domain). Antigenic determinants usually consist of chemically active surface groupings of molecules, such as amino acids or sugar side chains, and usually have specific three dimensional structural characteristics, as well as specific charge characteristics.

The terms “Fc receptor” or “FcR” refer to a receptor that binds to the Fc region of an antibody. There are three main classes of Fc receptors: (1) FcγR which bind to IgG, (2) FcαR which binds to IgA, and (3) FcR which binds to IgE. The FcγR family includes several members, such as FcγI (CD64), FcγRIIA (CD32A), FcγRIIB (CD32B), FcγRIIIA (CD16A), and FcγRIIIB (CD16B). The Fcγ receptors differ in their affinity for IgG and also have different affinities for the IgG subclasses (e.g., IgG1, IgG2, IgG3, and IgG4).

“Amino acid” refers to any monomeric unit that can be incorporated into a peptide, polypeptide, or protein. Amino acids include naturally occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid). The amino acids can be glycosylated (e.g., N-linked glycans, O-linked glycans, phosphoglycans, C-linked glycans, or glypication) or deglycosylated. Amino acids may be referred to herein by either the commonly known three letter symbols or by the one-letter symbols recommended by the IUPAC-IUB Biochemical Nomenclature Commission.

Naturally occurring amino acids are those encoded by the genetic code, as well as those amino acids that are later modified, e.g., hydroxyproline, γ-carboxyglutamate, and O-phosphoserine. Naturally occurring α-amino acids include, but are not limited to, alanine (Ala), arginine (Arg), asparagine (Asn), aspartic acid (Asp), cysteine (Cys), glutamic acid (Glu), glutamine (Gln), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), valine (Val), and combinations thereof. Stereoisomers of naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Naturally occurring amino acids include those formed in proteins by post-translational modification, such as citrulline (Cit).

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally occurring amino acids. For example, “amino acid analogs” can be unnatural amino acids that have the same basic chemical structure as naturally occurring amino acids (i.e., a carbon that is bonded to a hydrogen, a carboxyl group, an amino group) but have modified side-chain groups or modified peptide backbones, e.g., homoserine, norleucine, methionine sulfoxide, and methionine methyl sulfonium. “Amino acid mimetics” refer to chemical compounds that have a structure that is different from the general chemical structure of an amino acid, but that functions in a manner similar to a naturally occurring amino acid.

An ‘amino acid side chain” refers to the group of an amino acid which defines the amino acid and distinguishes one amino acid from other amino acids. For example, the side chains for a group of representative amino acids are: glycine (—H), alanine (—CH₃), phenylalanine (—CH₂(C₆H₅)), lysine (—CH₂CH₂CH₂CH₂NH₂), arginine (—CH₂CH₂CH₂NHC(NH)NH₂), leucine —CH₂CH(CH₃)₂, and citrulline (—CH₂CH₂CH₂NHC(O)NH₂).

“Linker” refers to a functional group that covalently links two or more moieties in a compound or material. For example, the linking moiety can serve to covalently bond a drug moiety to an antibody construct in conjugate provided herein or between two or more ligand binding moieties.

“Linking moiety” refers to a functional group that covalently bonds two or more moieties in a compound. For example, the linking moiety can serve to covalently bond a drug moiety to an antibody in a conjugate. Useful bonds for connecting linking moieties to proteins and other materials include, but are not limited to, amides, amines, esters, carbamates, disulfides, ureas, thioethers, thiocarbamates, thiocarbonates, and thioureas.

“Divalent” refers to a chemical moiety that contains two points of attachment for linking two moieties; polyvalent linking moieties can have additional points of attachment for linking further functional groups. Divalent radicals may be denoted with the suffix “diyl”. For example, divalent linking moieties include divalent polymer moieties such as divalent poly(ethylene glycol), divalent cycloalkyl, divalent heterocycloalkyl, divalent aryl, and divalent heteroaryl group. A “divalent cycloalkyl, heterocycloalkyl, aryl, or heteroaryl group” refers to a cycloalkyl, heterocycloalkyl, aryl, or heteroaryl group having two points of attachment for covalently linking two moieties in a molecule or material. Cycloalkyl, heterocycloalkyl, aryl, or heteroaryl groups can be substituted or unsubstituted. Cycloalkyl, heterocycloalkyl, aryl, or heteroaryl groups can be substituted with one or more groups selected from halo, hydroxy, amino, alkylamino, amido, acyl, nitro, cyano, and alkoxy.

A wavy line (“ [symbol] ”) represents a point of attachment of the specified chemical moiety. If the specified chemical moiety has two wavy lines (“ [symbol] ”) present, it will be understood that the chemical moiety can be used bilaterally, i.e., as read from left to right or from right to left. In some embodiments, a specified moiety having two wavy lines (“ [symbol] ”) present is considered to be used as read from left to right.

“Alkyl” refers to a straight (linear) or branched, saturated, aliphatic radical having the number of carbon atoms indicated. Alkyl can include any number of carbons, for example from one to twelve. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl, 1-octyl, and the like.

Alkyl groups can be substituted or unsubstituted. Substituted alkyl groups can be substituted with one or more groups selected from halo, hydroxy, amino, oxo (═O), alkylamino, amido, acyl, nitro, cyano, and alkoxy. Substituted alkyl groups can be geminally substituted where a carbon atom of the alkyl forms a spiro, cycloalkyl group such as cyclopropyl, cyclobutyl, cyclopentyl, or cyclohexyl.

The term “alkyldiyl” refers to a divalent alkyl radical. Examples of alkyldiyl groups include, but are not limited to, methylene (—CH₂—), ethylene (—CH₂CH₂—), propylene (—CH₂CH₂CH₂—), and the like. An alkyldiyl group may also be referred to as an “alkylene” group.

Alkyldiyl groups can be substituted or unsubstituted. Substituted alkyldiyl groups can be substituted with one or more groups selected from halo, hydroxy, amino, oxo (═O), alkylamino, amido, acyl, nitro, cyano, and alkoxy. Substituted alkyldiyl groups can be geminally substituted where a carbon atom of the alkyl forms a spiro, cycloalkyl group such as cyclopropyl, cyclobutyl, cyclopentyl, or cyclohexyl.

“Alkenyl” refers to a straight (linear) or branched, unsaturated, aliphatic radical having the number of carbon atoms indicated and at least one carbon-carbon double bond, sp2. Alkenyl can include from two to about 12 or more carbons atoms. Alkenyl groups are radicals having “cis” and “trans” orientations, or alternatively, “E” and “Z” orientations. Examples include, but are not limited to, ethylenyl or vinyl (—CH═CH₂), allyl (—CH₂CH═CH₂), butenyl, pentenyl, and isomers thereof. Alkenyl groups can be substituted or unsubstituted. “Substituted alkenyl” groups can be substituted with one or more groups selected from halo, hydroxy, amino, oxo (═O), alkylamino, amido, acyl, nitro, cyano, and alkoxy.

The terms “alkenylene” or “alkenyldiyl” refer to a linear or branched-chain divalent hydrocarbon radical. Examples include, but are not limited to, ethylenylene or vinylene (—CH═CH—), allyl (—CH₂CH═CH—), and the like.

“Alkynyl” refers to a straight (linear) or branched, unsaturated, aliphatic radical having the number of carbon atoms indicated and at least one carbon-carbon triple bond, sp. Alkynyl can include from two to about 12 or more carbons atoms. For example, C₂-C₆ alkynyl includes, but is not limited to ethynyl (—C≡CH), propynyl (propargyl, —CH₂C≡CH), butynyl, pentynyl, hexynyl, and isomers thereof. Alkynyl groups can be substituted or unsubstituted. “Substituted alkynyl” groups can be substituted with one or more groups selected from halo, hydroxy, amino, oxo (═O), alkylamino, amido, acyl, nitro, cyano, and alkoxy.

The term “alkynylene” or “alkynyldiyl” refer to a divalent alkynyl radical.

The terms “carbocycle”, “carbocyclyl”, “carbocyclic ring” and “cycloalkyl” refer to a saturated or partially unsaturated, monocyclic, fused bicyclic, spiro, or bridged polycyclic ring assembly containing from 3 to 12 ring atoms, or the number of atoms indicated. Saturated monocyclic carbocyclic rings include, for example, cyclopropyl, cyclobutyl, cyclopentyl, cyclohexyl, and cyclooctyl. Saturated bicyclic and polycyclic carbocyclic rings include, for example, norbornane, [2.2.2] bicyclooctane, decahydronaphthalene and adamantane. Carbocyclic groups can also be partially unsaturated, having one or more double or triple bonds in the ring. Representative carbocyclic groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene.

The term “cycloalkyldiyl” refers to a divalent cycloalkyl radical.

“Aryl” refers to a monovalent aromatic hydrocarbon radical of 6-20 carbon atoms (C₆-C₂₀) derived by the removal of one hydrogen atom from a single carbon atom of a parent aromatic ring system. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl.

The terms “heterocycle”, “heterocyclyl”, and “heterocyclic ring” are used interchangeably herein and refer to a saturated or a partially unsaturated (i.e., having one or more double and/or triple bonds within the ring) carbocyclic radical of 3 to about 20 ring atoms in which at least one ring atom is a heteroatom selected from nitrogen, oxygen, phosphorus and sulfur, the remaining ring atoms being C, where one or more ring atoms is optionally substituted independently with one or more substituents described below. A heterocycle may be a monocycle having 3 to 7 ring members (2 to 6 carbon atoms and 1 to 4 heteroatoms selected from N, O, P, and S) or a bicycle having 7 to 10 ring members (4 to 9 carbon atoms and 1 to 6 heteroatoms selected from N, O, P, and S), for example: a bicyclo [4,5], [5,5], [5,6], or [6,6] system, or more rings. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. “Heterocyclyl” also includes radicals where heterocycle radicals are fused with a saturated, partially unsaturated ring, or aromatic carbocyclic or heterocyclic ring. Examples of heterocyclic rings include, but are not limited to, morpholin-4-yl, piperidin-1-yl, piperazinyl, piperazin-4-yl-2-one, piperazin-4-yl-3-one, pyrrolidin-1-yl, thiomorpholin-4-yl, S-dioxothiomorpholin-4-yl, azocan-1-yl, azetidin-1-yl, octahydropyrido[1,2-a]pyrazin-2-yl, [1,4]diazepan-1-yl, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, 3-azabicyco[3.1.0]hexanyl, 3-azabicyclo[4.1.0]heptanyl, azabicyclo[2.2.2]hexanyl, 3H-indolyl quinolizinyl and N-pyridyl ureas. Spiro heterocyclyl moieties are also included within the scope of this definition. Examples of spiro heterocyclyl moieties include azaspiro[2.5]octanyl and azaspiro[2.4]heptanyl. Examples of a heterocyclic group wherein 2 ring atoms are substituted with oxo (═O) moieties are pyrimidinonyl and 1,1-dioxo-thiomorpholinyl. The heterocycle groups herein are optionally substituted independently with one or more substituents described herein.

The term “heteroaryl” refers to a monovalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Heteroaryl groups are optionally substituted independently with one or more substituents described herein.

The heterocycle or heteroaryl groups may be carbon (carbon-linked), or nitrogen (nitrogen-linked) bonded where such is possible. By way of example and not limitation, carbon bonded heterocycles or heteroaryls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline.

By way of example and not limitation, nitrogen bonded heterocycles or heteroaryls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of an isoindole, or isoindoline, position 4 of a morpholine, and position 9 of a carbazole, or β-carboline.

The terms “halo” and “halogen,” by themselves or as part of another substituent, refer to a fluorine, chlorine, bromine, or iodine atom.

The term “carbonyl,” by itself or as part of another substituent, refers to C(═O) or —C(═O)—, i.e., a carbon atom double-bonded to oxygen and bound to two other groups in the moiety having the carbonyl.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds described herein may contain asymmetric or chiral centers, and therefore exist in different stereoisomeric forms. It is intended that all stereoisomeric forms of the compounds described herein, including but not limited to, diastereomers, enantiomers and atropisomers, as well as mixtures thereof such as racemic mixtures, form part of the present disclosure. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity. Enantiomers may be separated from a racemic mixture by a chiral separation method, such as supercritical fluid chromatography (SFC). Assignment of configuration at chiral centers in separated enantiomers may be tentative, and depicted in Table 1 structures for illustrative purposes, while stereochemistry is definitively established, such as from x-ray crystallographic data.

The terms “treat,” “treatment,” and “treating” refer to any indicia of success in the treatment or amelioration of an injury, pathology, condition (e.g., cancer), or symptom (e.g., cognitive impairment), including any objective or subjective parameter such as abatement; remission; diminishing of symptoms or making the symptom, injury, pathology, or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter, including, for example, the result of a physical examination.

The terms “cancer,” “neoplasm,” and “tumor” are used herein to refer to cells which exhibit autonomous, unregulated growth, such that the cells exhibit an aberrant growth phenotype characterized by a significant loss of control over cell proliferation. Cells of interest for detection, analysis, and/or treatment in the context of the present disclosure include cancer cells (e.g., cancer cells from an individual with cancer), malignant cancer cells, pre-metastatic cancer cells, metastatic cancer cells, and non-metastatic cancer cells. Cancers of virtually every tissue are known. The phrase “cancer burden” refers to the quantum of cancer cells or cancer volume in a subject. Reducing cancer burden accordingly refers to reducing the number of cancer cells or the cancer cell volume in a subject. The term “cancer cell” as used herein refers to any cell that is a cancer cell (e.g., from any of the cancers for which an individual can be treated, e.g., isolated from an individual having cancer) or is derived from a cancer cell, e.g., clone of a cancer cell. For example, a cancer cell can be from an established cancer cell line, can be a primary cell isolated from an individual with cancer, can be a progeny cell from a primary cell isolated from an individual with cancer, and the like. In some embodiments, the term can also refer to a portion of a cancer cell, such as a sub-cellular portion, a cell membrane portion, or a cell lysate of a cancer cell. Many types of cancers are known to those of skill in the art, including solid tumors such as carcinomas, sarcomas, glioblastomas, melanomas, lymphomas, and myelomas, and circulating cancers such as leukemias.

As used herein, the term “cancer” includes any form of cancer, including but not limited to, solid tumor cancers (e.g., skin, lung, prostate, breast, gastric, bladder, colon, ovarian, pancreas, kidney, liver, glioblastoma, medulloblastoma, leiomyosarcoma, head & neck squamous cell carcinomas, melanomas, and neuroendocrine) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors.

The phrases “effective amount” and “therapeutically effective amount” refer to a dose or amount of a therapeutic agent that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition (McGraw-Hill, 2006); and Remington: The Science and Practice of Pharmacy, 22^{nd} Edition, (Pharmaceutical Press, London, 2012)). In the case of cancer, the therapeutically effective amount of the therapeutic agent may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the therapeutic agent may prevent growth and/or kill existing cancer cells, it may be cytostatic and/or cytotoxic. For cancer therapy, efficacy can, for example, be measured by assessing the time to disease progression (TTP) and/or determining the response rate (RR)

“Recipient,” “individual,” “subject,” “host,” and “patient” are used interchangeably and refer to any mammalian subject for whom diagnosis, treatment, or therapy is desired (e.g., humans). “Mammal” for purposes of treatment refers to any animal classified as a mammal, including humans, domestic and farm animals, and zoo, sports, or pet animals, such as dogs, horses, cats, cows, sheep, goats, pigs, camels, etc. In certain embodiments, the mammal is human. A “patient” or “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the patient, individual, or subject is a human. In some embodiments, the patient may be a “cancer patient,” i.e. one who is suffering or at risk for suffering from one or more symptoms of cancer. A “patient population” refers to a group of cancer patients. Such populations can be used to demonstrate statistically significant efficacy and/or safety of a drug

As used herein, the term “administering” refers to parenteral, intravenous, intraperitoneal, intramuscular, intratumoral, intralesional, intranasal, or subcutaneous administration, oral administration, administration as a suppository, topical contact, intrathecal administration, or the implantation of a slow-release device, e.g., a mini-osmotic pump, to the subject.

The terms “residue,” “moiety” or “group” refers to a component that is covalently bound or linked to another component.

The term “covalently bound” or “covalently linked” refers to a chemical bond formed by sharing of one or more pairs of electrons.

The term “peptidomimetic” or PM as used herein means a non-peptide chemical moiety as part of a linker. Whereas peptides are short chains (two or more) of amino acid monomers linked by peptide (amide) bonds, a peptidomimetic chemical moiety includes non-amino acid chemical moieties. A peptidomimetic chemical moiety may also include one or more amino acid that are separated by one or more non-amino acid chemical units. A peptidomimetic chemical moiety does not contain in any portion of its chemical structure two or more adjacent amino acids that are linked by peptide bonds.

### Cereblon Degrader Antibody Conjugates

The cereblon degrader antibody conjugate (cDAC) provided herein comprises at least one (p) cereblon degrader moiety (cD) covalently attached to an antibody (Ab) by an antibody linker (L₁).

The cereblon degrader antibody conjugates (cDAC) induce target-specific degradation of tumor-associated proteins and bring specificity to minimize off-target toxicity effects. In embodiments, the cD forms a cereblon-based ternary complex between a target protein and the E3 ubiquitin ligase, cereblon.

Exemplary embodiments of cDAC have a structure of Formula I.

Ab-[L₁-cD]ₚ I
or a pharmaceutically acceptable salt thereof,
wherein:
Ab is the antibody;
L₁ is the antibody linker;
cD is the cereblon degrader moiety; and
p is an integer from 1 to 14.

In some embodiments, L₁ comprises an immolator moiety. In embodiments, L₁ is L₁ₐ-IM, wherein IM is an immolator moiety, and L₁ₐ is any remainder of the L₁ antibody linker. In embodiments, L₁ is an immolator moiety, IM.

In some embodiments, cD comprises a cereblon-binding, E3 ubiquitin ligase ligand, E3UL. In some embodiments, cD is E3UL-cDₐ, wherein E3UL is the cereblon-binding, E3 ubiquitin ligase ligand of the cereblon degrader moiety, and cDₐ is any remainder of the cD. In some embodiments, cDₐ is TPL-L₂-, wherein TPL is a target protein ligand and L₂ is a degrader linker. In some embodiments, TPL comprises a ligand that binds BRD4.

Exemplary embodiments of cDAC have a structure of Formula I′:

Ab-[L₁ₐ-IM-E3UL-CDₐ]ₚ I′

In some embodiments, the cereblon degrader moiety (cD) of the cDAC is covalently linked through an aminal group to the antibody linker (L₁).

In some embodiments, L₁ comprises an immolator moiety, IM, selected from:

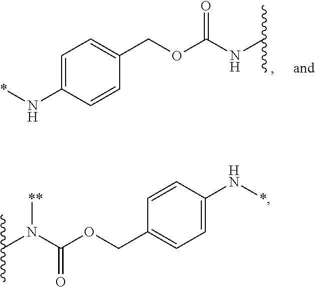

wherein
* indicates the point of attachment to L₁ₐ, ** indicates the point of attachment to cDₐ, and the wavy line indicates the point of attachment to E3UL.

Exemplary embodiments of cDAC have a structure of Formula I-A

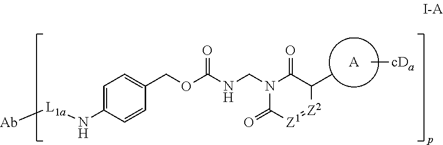

wherein
Ab is an antibody;
L₁ₐ is an antibody linker;
ring A is selected from C₆-C₂₀ aryl, C₃-C₂₀ carbocyclyl, C₃-C₂₀ heterocyclyl, and C₃-C₂₀ heteroaryl;
the dashed line [symbol] indicates an optional double bond;
Z¹ is selected from C(R¹)₂, CR¹, N, and NR^{1a}, and
Z² is selected from C(R²)₂, CR², N, and NR^{2a}, wherein
R¹ and R² are each independently selected from the group consisting of H, F, Cl, Br, I, —CN, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —NO₂, ═O, —OR^{a}, —OC(═O)R^{a}, —SR^{a}, —S(O)R^{a}, —S(O)₂R^{a}, —S(O)₂NR^{a}, and —S(O)₃H;
R^{1a} and R^{2a} are each independently selected from the group consisting of H, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —OR^{a}, —S(O)R^{a}, —S(O)₂R^{a}, —S(O)₂NR^{a}, and —S(O)₃H; or
(i) two geminal R¹ or two geminal R² form a 3-6 membered carbocyclyl or heterocyclyl spiro group, or
(ii) R¹ and R², R^{1a} and R², R¹ and R^{2a}, or R^{1a} and R^{2a} form a fused 5- or 6-membered aryl, carbocyclyl, heterocyclyl, or heteroaryl group, wherein
R^{a} and R^{b} are each independently selected from H, OH, C₁-C₆ alkyl, phenyl, and benzyl, wherein phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
wherein each alkyl, alkyldiyl, alkenyl, alkynyl, aryl, carbocyclyl, heterocyclyl, and heteroaryl is independently and optionally substituted with one or more groups selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H;
cDₐ is the remainder of a cereblon degrader moiety; and
p is an integer from 1 to 14.

In some embodiments, Z¹ is CR¹, and Z² is CR², and R¹ and R² are each H.

In some embodiments, ring A is C₃-C₂₀ heteroaryl.

In some embodiments, ring A is isoindoline substituted with ═O.

In some embodiments, the cDAC of Formula I has the structure of Formula I-A′

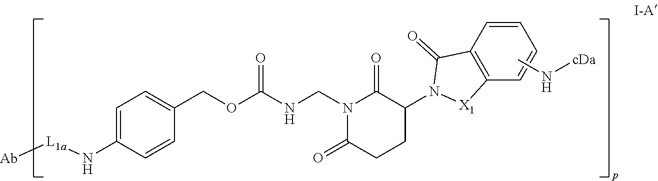

wherein X¹ is selected from CH₂ and C(═O).

In some embodiments, the antibody is a thiol-containing antibody.

In some embodiments, the thiol-containing antibody binds to a tumor-associated antigen or cell-surface receptor.

In some embodiments, the antibody is a cysteine-engineered antibody.

In some embodiments, the cysteine-engineered antibody comprising one or more cysteine mutation is selected from HC A118C, LC K149C, HC A140C, LC V205C, LC S121C, HC L174C, HC L177C, and HC Y373C.

In some embodiments, L₁ₐ is a protease-cleavable, non-peptide linker.

In some embodiments, cDₐ is TPL-L₂-, wherein TPL is a target protein ligand and L₂ is a degrader linker. In some embodiments, TPL comprises a ligand that binds BRD4.

In some embodiments, L₁ₐ has the structure of Formula L₁-A

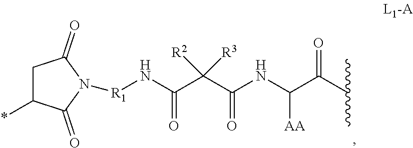

wherein
* indicates the point of attachment to a cysteine thiol of the Ab;
R¹ is selected from the group consisting of C₁-C₁₂ alkylene, C₁-C₁₂ alkylene-C(═O), C₁-C₁₂ alkylene-NH, (CH₂CH₂O)ᵣ, C₁-C₁₂ alkylene-NH, (CH₂CH₂O)ᵣ—C(═O), (CH₂CH₂O)ᵣ—C(═O), (CH₂CH₂O)ᵣ—CH₂, and C₁-C₁₂ alkylene-NHC(═O)CH₂CH(thiophen-3-yl), wherein
r is an integer ranging from 1 to 10, and
C₁-C₁₂ alkylene is optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H;
R² and R³ together form a C₃-C₇ cycloalkyl ring optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H; and
AA is a side chain of an amino acid selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, and citrulline.

In some embodiments, AA is selected from H, —CH₃, —CH₂(C₆H₅), —CH₂CH₂CH₂CH₂NH₂, —CH₂CH₂CH₂NHC(NH)NH₂, —CH₂CH(CH₃)₂, and —CH₂CH₂CH₂NHC(O)NH₂.

In some embodiments, R¹ is C₅ alkylene.

In some embodiments, R² and R³ together form a C₄ cycloalkyl ring.

In some embodiments, AA is —CH₃ or —CH₂CH₂CH₂NHC(O)NH₂.

In some embodiments,
R¹ is C₅ alkylene;
R² and R³ together form a C₄ cycloalkyl ring; and
AA is —CH₃ or —CH₂CH₂CH₂NHC(O)NH₂.

In some embodiments, cDₐ comprises (i) a target protein ligand covalently attached to a degrader linker, or (ii) a molecular glue moiety. In some embodiments, cDₐ is TPL-L₂-, wherein TPL is a target protein ligand and L₂ is a degrader linker. In some embodiments, TPL comprises a ligand that binds BRD4.

In some embodiments, p is 1, 2, 3, 4, 5, or 6.

Exemplary embodiments of cDAC have a structure of Formula I-B

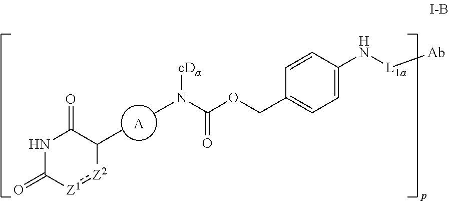

wherein
Ab is an antibody;
L₁ₐ is an antibody linker;
ring A is selected from C₆-C₂₀ aryl, C₃-C₂₀ carbocyclyl, C₂-C₂₀ heterocyclyl, and C₁-C₂₀ heteroaryl;
the dashed line [symbol] indicates an optional double bond;
Z¹ is selected from C(R¹)₂, CR¹, N, and NR^{1a}, and
Z² is selected from C(R²)₂, CR², N, and NR^{2a}, wherein
R¹ and R² are each independently selected from the group consisting of H, F, Cl, Br, I, —CN, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —NO₂, ═O, —OR^{a}, —OC(═O)R^{a}, —SR^{a}, —S(O)R^{a}, —S(O)₂R^{a}, —S(O)₂NR^{a}, and —S(O)₃H;
R^{1a} and R^{2a} are independently selected from the group consisting of H, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —ORₐ, —S(O)R^{a}, —S(O)₂R^{a}, —S(O)₂NR^{a}, and —S(O)₃H; or
(i) two geminal R¹ or two geminal R² form a 3-6 membered carbocyclyl or heterocyclyl spiro group, or
(ii) R¹ and R², R^{1a} and R², R¹ and R^{2a}, or R^{1a} and R^{2a} form a fused 5- or 6-membered aryl, carbocyclyl, heterocyclyl, or heteroaryl group, wherein
R^{a} and R^{b} are independently selected from H, OH, C1-C₆ alkyl, phenyl, and benzyl, where phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
wherein each alkyl, alkyldiyl, alkenyl, alkynyl, aryl, carbocyclyl, heterocyclyl, and heteroaryl is independently and optionally substituted with one or more groups selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H;
cDₐ is the remainder of a cereblon degrader moiety; and
p is an integer from 1 to 14.

In some embodiments, Z¹ is CR¹, and Z² is CR², and R¹ and R² are each H.

In some embodiments, ring A is C₃-C₂₀ heteroaryl.

In some embodiments, ring A is isoindoline substituted with ═O.

In some embodiments, cDₐ is TPL-L₂-, wherein TPL is a target protein ligand and L₂ is a degrader linker. In some embodiments, TPL comprises a ligand that binds BRD4.

In some embodiments, the cDAC of Formula I-B includes the structure of Formula I-B′

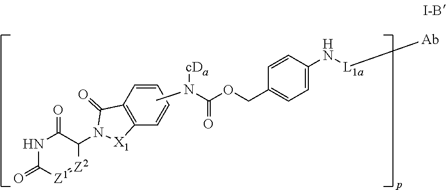

wherein X¹ is selected from CH₂ and C(═O).

In some embodiments, the antibody is a thiol-containing antibody.

In some embodiments, the thiol-containing antibody binds to a tumor-associated antigen or cell-surface receptor.

In some embodiments, the antibody is a cysteine-engineered antibody.

In some embodiments, the cysteine-engineered antibody has a cysteine mutation site selected from one or more of HC A118C, LC K149C, HC A140C, LC V205C, LC S121C, HC L174C, HC L177C, and HC Y373C.

In some embodiments, L₁ₐ is a protease-cleavable, non-peptide linker.

In some embodiments, L₁ₐ has the structure of Formula L₁-A

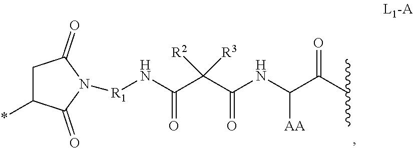

wherein
* indicates the point of attachment to a cysteine thiol of the Ab;
R¹ is selected from the group consisting of C₁-C₁₂ alkylene, C₁-C₁₂ alkylene-C(═O), C₁-C₁₂ alkylene-NH, (CH₂CH₂O)ᵣ, C₁-C₁₂ alkylene-NH, (CH₂CH₂O)ᵣ—C(═O), (CH₂CH₂O)ᵣ—C(═O), (CH₂CH₂O)ᵣ—CH₂, and C₁-C₁₂ alkylene-NHC(═O)CH₂CH(thiophen-3-yl), wherein
r is an integer ranging from 1 to 10, and
C₁-C₁₂ alkylene is optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H;
R² and R³ together form a C₃-C₇ cycloalkyl ring optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H; and
AA is a side chain of an amino acid selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, and citrulline.

In some embodiments, AA is selected from H, —CH₃, —CH₂(C₆H₅), —CH₂CH₂CH₂CH₂NH₂, —CH₂CH₂CH₂NHC(NH)NH₂, —CH₂CH(CH₃)₂, and —CH₂CH₂CH₂NHC(O)NH₂.

In some embodiments, R¹ is C₅ alkylene.

In some embodiments, R² and R³ together form a C₄ cycloalkyl ring.

In some embodiments, AA is —CH₃ or —CH₂CH₂CH₂NHC(O)NH₂.

In some embodiments:
R¹ is C₅ alkylene;
R² and R³ together form a C₄ cycloalkyl ring; and
AA is —CH₃ or —CH₂CH₂CH₂NHC(O)NH₂.

In some embodiments, cDa comprises (i) a target protein ligand covalently attached to a degrader linker, or (ii) a molecular glue moiety. In some embodiments, cDₐ is TPL-L₂-, wherein TPL is a target protein ligand and L₂ is a degrader linker. In some embodiments, TPL comprises a ligand that binds BRD4.

In some embodiments, p is 1, 2, 3, 4, 5, or 6.

In some embodiments, L₁ is an immolator moiety, IM.

Exemplary embodiments of cDAC have a structure of Formula I″:

Ab-[IM-E3UL-CDₐ]ₚ I″

In some embodiments, IM is:

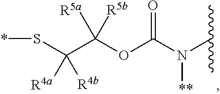

wherein
* indicates the point of attachment to Ab,
** indicates the point of attachment to cDₐ, and
the wavy line indicates the point of attachment to E3UL.

In such embodiments, R^{4a}, R^{4b}, R^{5a}, and R^{5a} are independently selected from H and C₁-C₆ alkyl, or R^{4a} and R^{4b} together with the carbon atom to which they are bound form a three-, four-, or five-membered cycloalkyl or heterocyclyl, optionally substituted with F, Cl, and C₁-C₆ alkyl, wherein C₁-C₆ alkyl is independently and optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H.

Exemplary embodiments of cDAC have a structure of Formula I-C

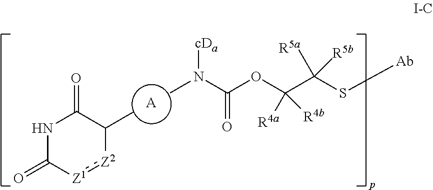

wherein
Ab is an antibody;
R^{4a}, R^{4b}, R^{5a}, and R^{5a} are each independently selected from H and C₁-C₆ alkyl, or R^{4a} and R^{4b} together with the carbon atom to which they are bound form a three-, four-, or five-membered cycloalkyl or heterocyclyl, optionally substituted with F, Cl, and C₁-C₆ alkyl, where C₁-C₆ alkyl is independently and optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H;
ring A is selected from C₆-C₂₀ aryl, C₃-C₂₀ carbocyclyl, C₂-C₂₀ heterocyclyl, and C₁-C₂₀ heteroaryl;
the dashed line [symbol] indicates an optional double bond;
Z¹ is selected from C(R¹)₂, CR¹, N, and NR^{1a}, and
Z² is selected from C(R²)₂, CR², N, and NR^{2a}, wherein
R¹ and R² are each independently selected from the group consisting of H, F, Cl, Br, I, —CN, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —NO₂, ═O, —OR^{a}, —OC(═O)R^{a}, —SR^{a}, —S(O)R^{a}, —S(O)₂R^{a}, —S(O)₂NR^{a}, and —S(O)₃H;
R^{1a} and R^{2a} are independently selected from the group consisting of H, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —OR^{a}, —S(O)R^{a}, —S(O)₂R^{a}, —S(O)₂NR^{a}, and —S(O)₃H; or
(i) two geminal R¹ or two geminal R² form a 3-6 membered carbocyclyl or heterocyclyl spiro group, or
(ii) R¹ and R², R^{1a} and R², R¹ and R^{2a}, or R^{1a} and R^{2a} form a fused 5- or 6-membered aryl, carbocyclyl, heterocyclyl, or heteroaryl group, wherein
R^{a} and R^{b} are independently selected from H, OH, C₁-C₆ alkyl, phenyl, and benzyl, where phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
wherein each alkyl, alkyldiyl, alkenyl, alkynyl, aryl, carbocyclyl, heterocyclyl, and heteroaryl is independently and optionally substituted with one or more groups selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H;
cDₐ is the remainder of a cereblon degrader moiety; and
p is an integer from 1 to 14.

In some embodiments, the sulfur is conjugated to a cysteine thiol of Ab to form a disulfide linkage.

In some embodiments, Z¹ is CR¹, and Z² is CR², and R¹ and R² are each H.

In some embodiments, ring A is C₃-C₂₀ heteroaryl.

In some embodiments, ring A is isoindoline substituted with ═O or oxo.

In some embodiments, cDₐ is TPL-L₂-, wherein TPL is a target protein ligand and L₂ is a degrader linker. In some embodiments, TPL comprises a ligand that binds BRD4.

In some embodiments, the cDAC of Formula I-C includes the structure of Formula I-C′

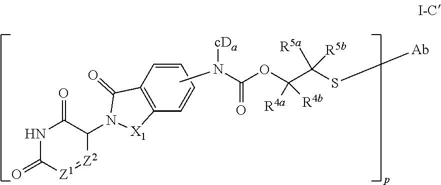

wherein
Ab is an antibody;
R^{4a}, R^{4b}, R^{5a}, and R^{5a} are each independently selected from H and C₁-C₆ alkyl, or R^{4a} and R^{4b} together with the carbon atom to which they are bound form a three-, four-, or five-membered cycloalkyl or heterocyclyl, optionally substituted with F, Cl, and C₁-C₆ alkyl, where C₁-C₆ alkyl is independently and optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H;
X¹ is selected from CH₂ and C(═O);
the dashed line [symbol] indicates an optional double bond;
Z¹ is selected from C(R¹)₂, CR¹, N, and NR^{1a}, and
Z² is selected from C(R²)₂, CR², N, and NR^{2a}, wherein
R¹ and R² are each independently selected from the group consisting of H, F, Cl, Br, I, —CN, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —NO₂, ═O, —OR^{a}, —OC(═O)R^{a}, —SR^{a}, —S(O)R^{a}, —S(O)₂R^{a}, —S(O)₂NR^{a}, and —S(O)₃H;
R^{1a} and R^{2a} are independently selected from the group consisting of H, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —ORₐ, —S(O)R^{a}, —S(O)₂R^{a}, —S(O)₂NR^{a}, and —S(O)₃H; or
(i) two geminal R¹ or two geminal R² form a 3-6 membered carbocyclyl or heterocyclyl spiro group, or
(ii) R¹ and R², R^{1a} and R², R¹ and R^{2a}, or R^{1a} and R^{2a} form a fused 5- or 6-membered aryl, carbocyclyl, heterocyclyl, or heteroaryl group, wherein
R^{a} and R^{b} are independently selected from H, OH, C₁-C₆ alkyl, phenyl, and benzyl, where phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
wherein each alkyl, alkyldiyl, alkenyl, alkynyl, aryl, carbocyclyl, heterocyclyl, and heteroaryl is independently and optionally substituted with one or more groups selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H;
cDₐ is the remainder of a cereblon degrader moiety; and
p is an integer from 1 to 14.

In some embodiments, the antibody is a thiol-containing antibody.

In some embodiments, the thiol-containing antibody binds to a tumor-associated antigen or cell-surface receptor.

In some embodiments, the antibody is a cysteine-engineered antibody.

In some embodiments, the cysteine-engineered antibody comprises one or more cysteine mutations selected from HC A118C, LC K149C, HC A140C, LC V205C, LC S121C, HC L174C, HC L177C, and HC Y373C.

In some embodiments, cDₐ comprises (i) a target protein ligand covalently attached to a degrader linker, or (ii) a molecular glue moiety. In some embodiments, cDₐ is TPL-L₂-, wherein TPL is a target protein ligand and L₂ is a degrader linker. In some embodiments, TPL comprises a ligand that binds BRD4.

In some embodiments, p is 1, 2, 3, 4, 5, or 6.

### Cereblon Degrader-Linker Intermediates

A cereblon degrader-linker intermediate (cDLI) is a reagent for the process of making a cereblon degrader antibody conjugate (cDAC) by conjugation with a thiol-containing antibody. The cereblon degrader-linker intermediate has a thiol-reactive functional group (X). The thiol-reactive functional group (X) is covalently attached to the cereblon degrader moiety (cD) by a linker (L₃).

In some embodiments, a cereblon degrader-linker intermediate has the structure of Formula II:

X-L₃-cD II
wherein:
X is a thiol-reactive group covalently attached to L₃;
L₃ is a linker covalently attached to X and cD; and
cD is a cereblon degrader moiety covalently attached to L₃.
cD may be a heterobifunctional bivalent cereblon degrader moiety or a molecular glue cereblon degrader moiety.

In some embodiments, L₃ comprises an immolator moiety. In embodiments, L₃ is L₃ₐ-IM, wherein IM is an immolator moiety, and L₃ₐ is any remainder of the L₃ linker. In embodiments, L₃ is an immolator moiety, IM.

In some embodiments, cD comprises a cereblon-binding, E3 ubiquitin ligase ligand, E3UL. In some embodiments, cD is E3UL-cDₐ, wherein E3UL is the cereblon-binding, E3 ubiquitin ligase ligand of the cereblon degrader moiety, cD, and cDₐ is any remainder of the cD.

In some embodiments, cDₐ is TPL-L₂-, wherein TPL is a target protein ligand and L₂ is a degrader linker. In some embodiments, TPL comprises a ligand that binds BRD4.

Exemplary embodiments of cDLI have a structure of Formula II′:

X-L₃ₐ-IM-E3UL-cDₐ II′

In some embodiments, the cereblon degrader moiety (cD) of the cDLI is linked through an aminal group to the antibody linker (L₃).

In some embodiments, L₃ comprises an immolator moiety, IM, selected from:

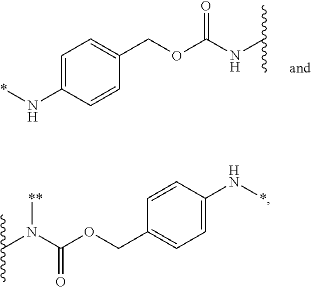

wherein
* indicates the point of attachment to any remainder of the L₃ linker, L₃ₐ,
** indicates the point of attachment to cDₐ, and
the wavy line indicates the point of attachment to E3UL. Exemplary embodiments of cDLI have a structure of Formula II-A

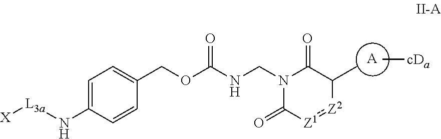

wherein
X is a thiol-reactive group;
L₃a is a linker;
ring A is selected from C₆-C₂₀ aryl, C₃-C₂₀ carbocyclyl, C₃-C₂₀ heterocyclyl, and C₃-C₂₀ heteroaryl;
the dashed line [symbol] indicates an optional double bond;
Z¹ is selected from C(R¹)₂, CR, N, and NR^{1a}, and
Z² is selected from C(R²)₂, CR², N, and NR^{2a}, wherein
R¹ and R² are each independently selected from the group consisting of H, F, Cl, Br, I, —CN, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}, —NR^{a}R^{b}, —C(═O)NH(C₁-C₆ alkyldiyl)-NR^{a}R^{b}, —C(═O)OR^{a}, —NR^{a}R^{b}, —NO₂, ═O, —OR^{a}, —OC(═O)R^{a}, —SR^{a}, —S(O)R^{a}, —S(O)₂R^{a}, —S(O)₂NR^{a}, and —S(O)₃H;
R^{1a} and R^{2a} are each independently selected from the group consisting of H, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}, —NR^{a}R^{b}, —C(═O)NH(C₁-C₆ alkyldiyl)-NR^{a}R^{b}, —C(═O)OR^{a}, —NR^{a}R^{b}, —OR^{a}, —S(O)R^{a}, —S(O)₂R^{a}, —S(O)₂NR^{a}, and —S(O)₃H; or
(i) two geminal R¹ or two geminal R² form a 3-6 membered carbocyclyl or heterocyclyl spiro group, or
(ii) R¹ and R², R^{1a} and R², R¹ and R^{2a}, or R^{1a} and R^{2a} form a fused 5- or 6-membered aryl, carbocyclyl, heterocyclyl, or heteroaryl group, wherein
R^{a} and R^{b} are each independently selected from H, OH, C₁-C₆ alkyl, phenyl, and benzyl, wherein phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
wherein each alkyl, alkyldiyl, alkenyl, alkynyl, aryl, carbocyclyl, heterocyclyl, and heteroaryl is independently and optionally substituted with one or more groups selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H;
cDₐ is the remainder of a cereblon degrader moiety; and
p is an integer from 1 to 14.

In some embodiments, Z¹ is CR¹, and Z² is CR², and R¹ and R² are each H.

In some embodiments, ring A is C₃-C₂₀ heteroaryl.

In some embodiments, ring A is isoindoline substituted with ═O.

In some embodiments, cDₐ is TPL-L₂-, wherein TPL is a target protein ligand and L₂ is a degrader linker. In some embodiments, TPL comprises a ligand that binds BRD4.

In some embodiments, the cDLI of Formula II has the structure of Formula II-A′

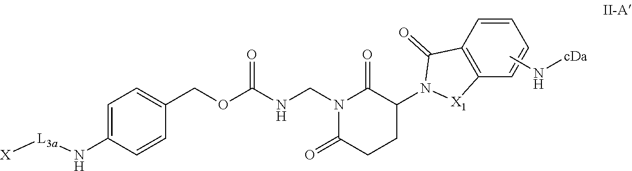

wherein
X is a thiol-reactive group;
L₃ₐ is a linker; and
X¹ is selected from CH₂ and C(═O).

In some embodiments, L₃ₐ is a protease-cleavable, non-peptide linker.

In some embodiments, X-L₃ₐ has the structure of Formula L₃-A

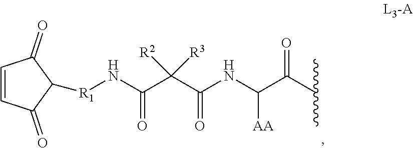

wherein
R¹ is selected from the group consisting of C₁-C₁₂ alkylene, C₁-C₁₂ alkylene-C(═O), C₁-C₁₂ alkylene-NH, (CH₂CH₂O)ᵣ, C₁-C₁₂ alkylene-NH, (CH₂CH₂O)ᵣ—C(═O), (CH₂CH₂O)ᵣ—C(═O), (CH₂CH₂O)ᵣ—CH₂, and C₁-C₁₂ alkylene-NHC(═O)CH₂CH(thiophen-3-yl), wherein
r is an integer ranging from 1 to 10, and
C₁-C₁₂ alkylene is optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H;
R² and R³ together form a C₃-C₇ cycloalkyl ring optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H; and
AA is a side chain of an amino acid selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, and citrulline.

In some embodiments, AA is selected from H, —CH₃, —CH₂(C₆H₅), —CH₂CH₂CH₂CH₂NH₂, —CH₂CH₂CH₂NHC(NH)NH₂, —CH₂CH(CH₃)₂, and —CH₂CH₂CH₂NHC(O)NH₂.

In some embodiments, R¹ is C₅ alkylene.

In some embodiments, R² and R³ together form a C₄ cycloalkyl ring.

In some embodiments, AA is —CH₃ or —CH₂CH₂CH₂NHC(O)NH₂.

In some embodiments,
R¹ is C₅ alkylene;
R² and R³ together form a C₄ cycloalkyl ring; and
AA is —CH₃ or —CH₂CH₂CH₂NHC(O)NH₂.

In some embodiments, cDa comprises (i) a target protein ligand covalently attached to a degrader linker, or (ii) a molecular glue moiety. In some embodiments, cDₐ is TPL-L₂-, wherein TPL is a target protein ligand and L₂ is a degrader linker. In some embodiments, TPL comprises a ligand that binds BRD4.

Exemplary embodiments of cDLI have a structure of Formula II-B

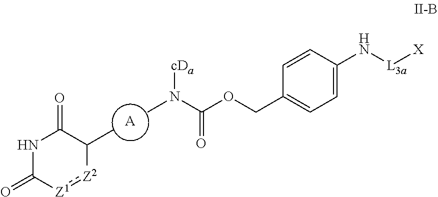

wherein
X is a thiol-reactive group;
L₃ₐ is a linker;
ring A is selected from C₆-C₂₀ aryl, C₃-C₂₀ carbocyclyl, C₂-C₂₀ heterocyclyl, and C₁-C₂₀ heteroaryl;
the dashed line [symbol] indicates an optional double bond;
Z¹ is selected from C(R¹)₂, CR¹, N, and NR^{1a}, and
Z² is selected from C(R²)₂, CR², N, and NR^{2a}, wherein
R¹ and R² are each independently selected from the group consisting of H, F, Cl, Br, I, —CN, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —NO₂, ═O, —OR^{a}, —OC(═O)R^{a}, —SR^{a}, —S(O)R^{a}, —S(O)₂R^{a}, —S(O)₂NR^{a}, and —S(O)₃H;
R^{1a} and R^{2a} are independently selected from the group consisting of H, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —ORₐ, —S(O)R^{a}, —S(O)₂R^{a}, —S(O)₂NR^{a}, and —S(O)₃H; or
(i) two geminal R¹ or two geminal R² form a 3-6 membered carbocyclyl or heterocyclyl spiro group, or
(ii) R¹ and R², R^{1a} and R², R¹ and R^{2a}, or R^{1a} and R^{2a} form a fused 5- or 6-membered aryl, carbocyclyl, heterocyclyl, or heteroaryl group, wherein
R^{a} and R^{b} are independently selected from H, OH, C₁-C₆ alkyl, phenyl, and benzyl, where phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
wherein each alkyl, alkyldiyl, alkenyl, alkynyl, aryl, carbocyclyl, heterocyclyl, and heteroaryl is independently and optionally substituted with one or more groups selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H; and
cDₐ is the remainder of a cereblon degrader moiety.

In some embodiments, Z¹ is CR¹, and Z² is CR², and R¹ and R² are each H.

In some embodiments, ring A is C₃-C₂₀ heteroaryl.

In some embodiments, ring A is isoindoline substituted with ═O.

In some embodiments, cDₐ is TPL-L₂-, wherein TPL is a target protein ligand and L₂ is a degrader linker. In some embodiments, TPL comprises a ligand that binds BRD4.

In some embodiments, the cDLI of Formula II-B includes the structure of Formula II-B′

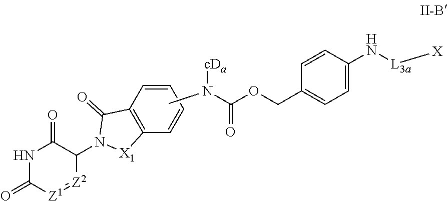

X is a thiol-reactive group;
L₃ₐ is a linker; and
X¹ is selected from CH₂ and C(═O).

In some embodiments, L₃ₐ is a protease-cleavable, non-peptide linker.

In some embodiments, X-L₃ₐ has the structure of Formula L₃-A

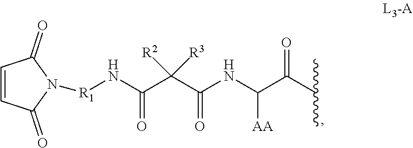

wherein
R¹ is selected from the group consisting of C₁-C₁₂ alkylene, C₁-C₁₂ alkylene-C(═O), C₁-C₁₂ alkylene-NH, (CH₂CH₂O)ᵣ, C₁-C₁₂ alkylene-NH, (CH₂CH₂O)ᵣ—C(═O), (CH₂CH₂O)ᵣ—C(═O), (CH₂CH₂O)ᵣ—CH₂, and C₁-C₁₂ alkylene-NHC(═O)CH₂CH(thiophen-3-yl), wherein
r is an integer ranging from 1 to 10, and
C₁-C₁₂ alkylene is optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H;
R² and R³ together form a C₃-C₇ cycloalkyl ring optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H; and
AA is a side chain of an amino acid selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, and citrulline.

In some embodiments, AA is selected from H, —CH₃, —CH₂(C₆H₅), —CH₂CH₂CH₂CH₂NH₂, —CH₂CH₂CH₂NHC(NH)NH₂, —CH₂CH(CH₃)₂, and —CH₂CH₂CH₂NHC(O)NH₂.

In some embodiments, R¹ is C₅ alkylene.

In some embodiments, R² and R³ together form a C₄ cycloalkyl ring.

In some embodiments, AA is —CH₃ or —CH₂CH₂CH₂NHC(O)NH₂.

In some embodiments:
R¹ is C₅ alkylene;
R² and R³ together form a C₄ cycloalkyl ring; and
AA is —CH₃ or —CH₂CH₂CH₂NHC(O)NH₂.

In some embodiments, cDa comprises (i) a target protein ligand covalently attached to a degrader linker, or (ii) a molecular glue moiety. In some embodiments, cDₐ is TPL-L₂-, wherein TPL is a target protein ligand and L₂ is a degrader linker. In some embodiments, TPL comprises a ligand that binds BRD4.

Exemplary embodiments of cDLI have a structure of Formula II″:

X-IM-E3UL-cDₐ II″

In embodiments, X-IM comprises

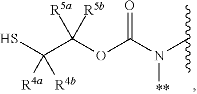

wherein
** indicates the point of attachment to cDₐ, and the wavy line indicates the point of attachment to E3UL.

In such embodiments, R^{4a}, R^{4b}, R^{5a}, and R^{5a} are independently selected from H and C₁-C₆ alkyl, or R^{4a} and R^{4b} together with the carbon atom to which they are bound form a three-, four-, or five-membered cycloalkyl or heterocyclyl, optionally substituted with F, Cl, and C₁-C₆ alkyl, wherein C₁-C₆ alkyl is independently and optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H.

Exemplary embodiments of cDLI have a structure of Formula II-C

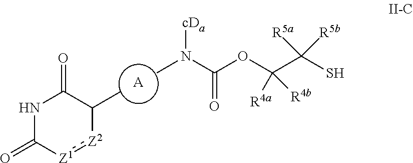

wherein
R^{4a}, R^{4b}, R^{5a}, and R^{5a} are each independently selected from H and C₁-C₆ alkyl, or R^{4a} and R^{4b} together with the carbon atom to which they are bound form a three-, four-, or five-membered cycloalkyl or heterocyclyl, optionally substituted with F, Cl, and C₁-C₆ alkyl, where C₁-C₆ alkyl is independently and optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H;
ring A is selected from C₆-C₂₀ aryl, C₃-C₂₀ carbocyclyl, C₂-C₂₀ heterocyclyl, and C₁-C₂₀ heteroaryl;
the dashed line [symbol] indicates an optional double bond;
Z¹ is selected from C(R¹)₂, CR¹, N, and NR^{1a}, and
Z² is selected from C(R²)₂, CR², N, and NR^{2a}, wherein
R¹ and R² are each independently selected from the group consisting of H, F, Cl, Br, I, —CN, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —NO₂, ═O, —OR^{a}, —OC(═O)R^{a}, —SR^{a}, —S(O)R^{a}, —S(O)₂R^{a}, —S(O)₂NR^{a}, and —S(O)₃H;
R^{1a} and R^{2a} are independently selected from the group consisting of H, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —ORₐ, —S(O)R^{a}, —S(O)₂R^{a}, —S(O)₂NR^{a}, and —S(O)₃H; or
(i) two geminal R¹ or two geminal R² form a 3-6 membered carbocyclyl or heterocyclyl spiro group, or
(ii) R¹ and R², R^{1a} and R², R¹ and R^{2a}, or R^{1a} and R^{2a} form a fused 5- or 6-membered aryl, carbocyclyl, heterocyclyl, or heteroaryl group, wherein
R^{a} and R^{b} are independently selected from H, OH, C₁-C₆ alkyl, phenyl, and benzyl, where phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
wherein each alkyl, alkyldiyl, alkenyl, alkynyl, aryl, carbocyclyl, heterocyclyl, and heteroaryl is independently and optionally substituted with one or more groups selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H; and
cDₐ is the remainder of a cereblon degrader moiety.

In some embodiments, Z¹ is CR¹, and Z² is CR², and R¹ and R² are each H.

In some embodiments, ring A is C₃-C₂₀ heteroaryl.

In some embodiments, ring A is isoindoline substituted with ═O or oxo.

In some embodiments, cDₐ is TPL-L₂-, wherein TPL is a target protein ligand and L₂ is a degrader linker. In some embodiments, TPL comprises a ligand that binds BRD4.

In some embodiments, the cDLI of Formula II-C includes the structure of Formula II-C′

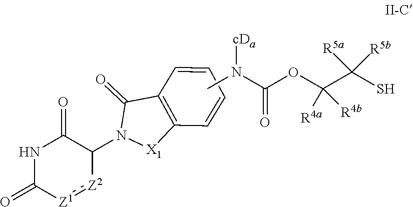

wherein
R^{4a}, R^{4b}, R^{5a}, and R^{5a} are each independently selected from H and C₁-C₆ alkyl, or R^{4a} and R^{4b} together with the carbon atom to which they are bound form a three-, four-, or five-membered cycloalkyl or heterocyclyl, optionally substituted with F, Cl, and C₁-C₆ alkyl, where C₁-C₆ alkyl is independently and optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H;
ring A is selected from C₆-C₂₀ aryl, C₃-C₂₀ carbocyclyl, C₂-C₂₀ heterocyclyl, and C₁-C₂₀ heteroaryl;
the dashed line [symbol] indicates an optional double bond;
Z¹ is selected from C(R¹)₂, CR¹, N, and NR^{1a}, and
Z² is selected from C(R²)₂, CR², N, and NR^{2a}, wherein
R¹ and R² are each independently selected from the group consisting of H, F, Cl, Br, I, —CN, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —NO₂, ═O, —OR^{a}, —OC(═O)R^{a}, —SR^{a}, —S(O)R^{a}, —S(O)₂R^{a}, —S(O)₂NR^{a}, and —S(O)₃H;
R^{1a} and R^{2a} are independently selected from the group consisting of H, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —ORₐ, —S(O)R^{a}, —S(O)₂R^{a}, —S(O)₂NR^{a}, and —S(O)₃H; or
(i) two geminal R¹ or two geminal R² form a 3-6 membered carbocyclyl or heterocyclyl spiro group, or
(ii) R¹ and R², R^{1a} and R², R¹ and R^{2a}, or R^{1a} and R^{2a} form a fused 5- or 6-membered aryl, carbocyclyl, heterocyclyl, or heteroaryl group, wherein
R^{a} and R^{b} are independently selected from H, OH, C₁-C₆ alkyl, phenyl, and benzyl, where phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
wherein each alkyl, alkyldiyl, alkenyl, alkynyl, aryl, carbocyclyl, heterocyclyl, and heteroaryl is independently and optionally substituted with one or more groups selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H; and
X¹ is selected from CH₂ and C(═O); and
cDₐ is the remainder of a cereblon degrader moiety.

In some embodiments, cDa comprises (i) a target protein ligand covalently attached to a degrader linker, or (ii) a molecular glue moiety. In some embodiments, cDₐ is TPL-L₂-, wherein TPL is a target protein ligand and L₂ is a degrader linker. In some embodiments, TPL comprises a ligand that binds BRD4.

### Cereblon-Binding, E3 Ubiquitin Ligase Ligands

The cereblon-binding, E3 ubiquitin ligase ligand (E3UL) is a moiety that binds to cereblon in the E3 ubiquitin ligase complex.

In some embodiments, the E3UL comprises a glutarimide group.

In some embodiments, the cereblon degrader moiety (cD) of an antibody conjugate (cDAC) has a structure selected from the formulae:

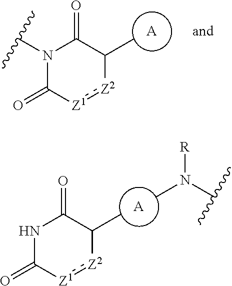

wherein the wavy line indicates the point of attachment to the antibody linker L₁ of Formula I or the linker L₃ of Formula II, and the dashed line indicates an optional double bond;
Z¹ is selected from C(R¹)₂, CR¹, N, and NR^{1a};
Z² is selected from C(R²)₂, CR², N, and NR^{2a};
R is selected from H and C₁-C₆ alkyl;
R¹ and R² are independently selected from the group consisting of H, F, Cl, Br, I, —CN, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —NO₂, ═O, —OR^{a}, —OC(═O)R^{a}, —SR^{a}, —S(O)R^{a}, —S(O)₂R^{a}, —S(O)₂NR^{a}, and —S(O)₃H;
R^{1a} and R^{2a} are independently selected from the group consisting of H, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —ORₐ, —S(O)R^{a}, —S(O)₂R^{a}, —S(O)₂NR^{a}, and —S(O)₃H;
or (i) two geminal R¹ or two geminal R² form a 3-6 membered carbocyclyl or heterocyclyl spiro group, or (ii) R¹ and R², R^{1a} and R², R¹ and R^{2a}, or R^{1a} and R^{2a} form a fused 5- or 6-membered aryl, carbocyclyl, heterocyclyl, or heteroaryl group;
R^{a} and R^{b} are independently selected from H, OH, C₁-C₆ alkyl, phenyl, and benzyl, where phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl, and
A is selected from C₆-C₂₀ aryl, C₃-C₂₀ carbocyclyl, C₂-C₂₀ heterocyclyl, and C₁-C₂₀ heteroaryl;
wherein any alkyl, alkyldiyl, alkenyl, alkynyl, aryl, carbocyclyl, heterocyclyl, and heteroaryl, are independently and optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H.

In some embodiments, Z¹ and Z² are each CR¹, and R¹ and R² are each H.

In some embodiments, R is H.

In some embodiments, A is C₆-C₂₀ aryl.

In some embodiments,
Z¹ and Z² are each CR¹, wherein R¹ and R² are each H;
R is H; and
A is C₆-C₂₀ aryl.

In some embodiments, the E3UL of the cereblon degrader moiety (cD) of an antibody conjugate (cDAC) has a structure selected from the formulae:

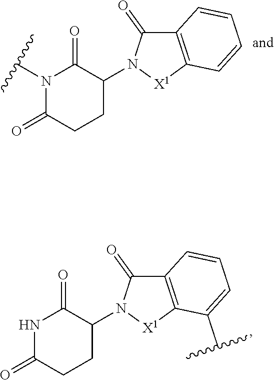

wherein X¹ is selected from CH₂ and C(═O); and the wavy line indicates the point of attachment of the antibody linker L₁ of Formula I, the linker L₃ of Formula II and/or the degrader linker L₂ of the cereblon degrader moiety cD.

In some embodiments, X¹ is C(═O).

### Antibody Linkers

The antibody linker (L₁) is a bifunctional linker that covalently attaches the antibody (Ab) to the cereblon degrader moiety (cD). The disclosed antibody linkers provide stability of the cDAC in the bloodstream and while allowing efficient cleavage upon internalization into targeted cells. The specific design of the antibody linker influence aspects of cDAC pharmacology including drug stability into circulation, tumor cell permeability, drug-to-antibody ratio (DAR) i.e. the number of payload molecules carried by each antibody), and extent of the bystander effect.

The disclosed antibody linkers may comprise a cleavable non-peptidic peptidomimetic unit (PM). The PM may be a substrate for lysosomal proteases although not containing a peptide (WO 2015/095227; WO 2015/095124; WO 2015/095223). For example, the cyclobutane-1,1-dicarboxamide-containing peptidomimetic linker is hydrolyzed predominantly by cathepsin B while the valine-citrulline dipeptide linker is not. Antibody-drug conjugates bearing the PM linker may be as efficacious and stable in vivo as those with a dipeptide linker (Wei et al, (2018) J. Med. Chem. 61:989-1000).

In some embodiments, L₁ is a protease-cleavable, non-peptide linker having the formula:

-Str-PM-IM-
wherein Str is a stretcher unit covalently attached to the antibody; PM is a peptidomimetic unit, and IM is an immolator unit covalently attached to the cereblon degrader moiety.

In some embodiments, Str has the formula:

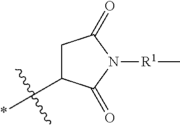

wherein
* indicates the point of attachment on the succinimidyl ring to a cysteine thiol of the antibody, and
R¹ is selected from the group consisting of C₁-C₁₂ alkylene, C₁-C₁₂ alkylene-C(═O), C₁-C₁₂ alkylene-NH, (CH₂CH₂O)ᵣ, C₁-C₁₂ alkylene-NH, (CH₂CH₂O)ᵣ—C(═O), (CH₂CH₂O)ᵣ—C(═O), (CH₂CH₂O)ᵣ—CH₂, and C₁-C₁₂ alkylene-NHC(═O)CH₂CH(thiophen-3-yl), where r is an integer ranging from 1 to 10 and C₁-C₁₂ alkylene is optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H.

In some embodiments, Str is selected from the following:

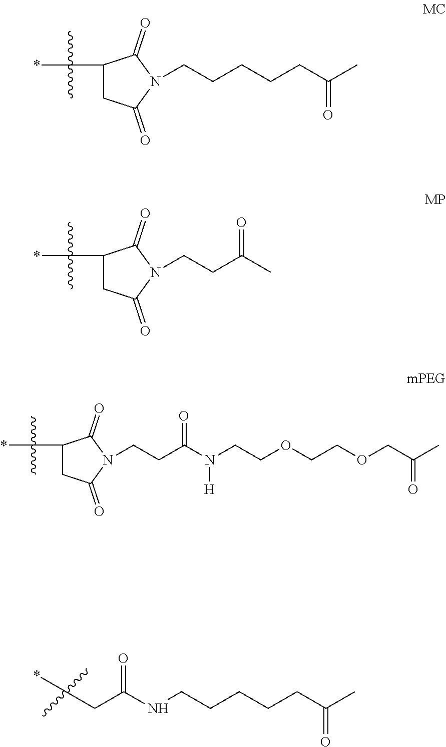

wherein * indicates the point of attachment to a cysteine thiol of the antibody.

In some embodiments of R¹, the C₁-C₁₂ alkylene is C₁-C₅ alkylene.

In some embodiments, R¹ is (CH₂)₅ or C₅ alkylene.

In some embodiments, PM has the formula:

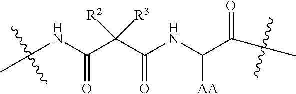

where R² and R³ together form a C₃-C₇ cycloalkyl ring optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H, and
AA is a side chain of an amino acid selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, and citrulline.

In some embodiments, AA is selected from H, —CH₃, —CH₂(C₆H₅), —CH₂CH₂CH₂CH₂NH₂, —CH₂CH₂CH₂NHC(NH)NH₂, —CH₂CH(CH₃)₂, and —CH₂CH₂CH₂NHC(O)NH₂.

In some embodiments, R² and R³ together form a C₄ cycloalkyl ring; and AA is —CH₃.

In some embodiments, R² and R³ together form a C₄ cycloalkyl ring; and AA is —CH₂CH₂CH₂NHC(O)NH₂.

In some embodiments, Formula I comprises an immolator moiety selected from:

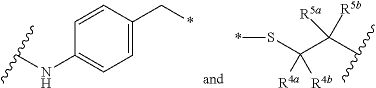

wherein * indicates the point of attachment to the remainder of L₁, and the wavy line indicates point of attachment to cD;
R^{4a}, R^{4b}, R^{5a}, and R^{5a} are independently selected from H and C₁-C₆ alkyl, or R^{4a} and R^{4b} together with the carbon atom to which they are bound form a three-, four-, or five-membered cycloalkyl or heterocyclyl, optionally substituted with F, Cl, and C₁-C₆ alkyl; and
C₁-C₆ alkyl is independently and optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H.

In some embodiments, the cereblon degrader moiety (cD) of the cDAC is linked through an aminal group of the nitrogen atom of the glutarimide group of the cD to the antibody linker (L₁).

In some embodiments, the IM-cD of an antibody conjugate (cDAC) comprises a structure selected from:

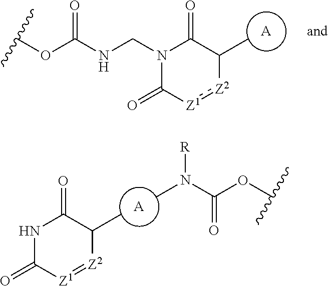

wherein the wavy line indicates the point of attachment to the remainder of the linker, L₁, of a heterobifunctional cD of Formula I or the remainder of the linker, L₃, of a molecular glue cD of Formula II.

In some embodiments, Z¹ and Z² are each CR¹, and R¹ and R² are each H.

In some embodiments, R is H.

In some embodiments, A is C₆-C₂₀ aryl.

In some embodiments,
Z¹ and Z² are each CR¹, wherein R¹ and R² are each H;
R is H; and
A is C₆-C₂₀ aryl.

In some embodiments, IM comprises a group selected from 4-aminobenzyl, 4-aminobenzyloxycarbonyl, and (4-aminobenzyl)methylcarbamate.

In some embodiments, L₁ forms a disulfide linkage with a cysteine thiol of the antibody.

In some embodiments, Formula I is selected from the formulae:

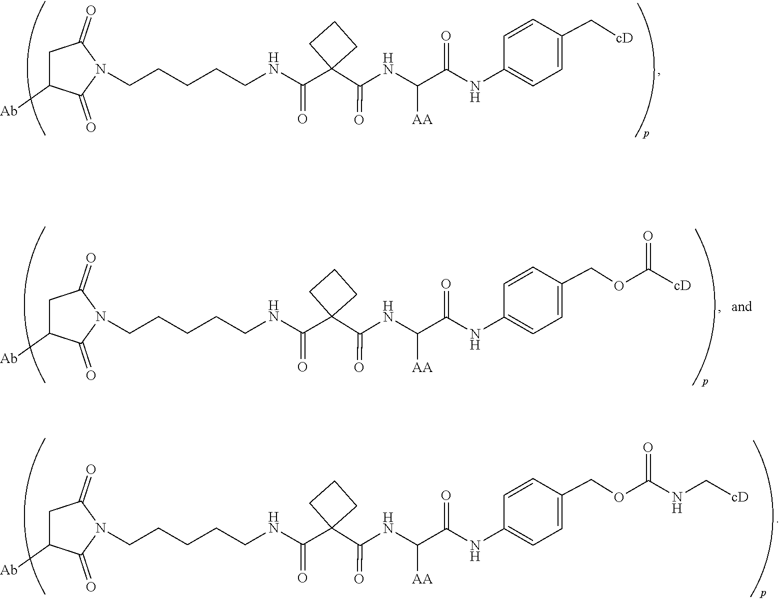

In some embodiments, L₁ is a oinker having the formula:

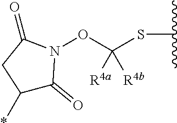

wherein
R^{4a} and R^{4b} are each independently selected from H and C₁-C₆ alkyl, or R^{4a} and R^{4b}, together with the carbon atom to which they are bound, form a three-, four-, or five-membered cycloalkyl or heterocyclyl, optionally substituted with F, Cl, and C₁-C₆ alkyl, where C₁-C₆ alkyl is independently and optionally substituted with one or more groups selected from F, Cl, —CN, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H; and
* indicates the point of attachment to a cysteine thiol of the antibody, and the wavy line indicates the attachment to the cereblon degrader moiety.
In some embodiments, L₁ is selected from the formulae:

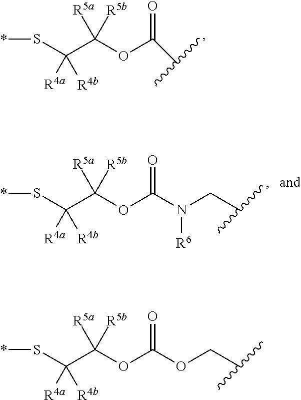

wherein
R^{4a}, R^{4b}, R^{5a}, and R^{5a} are each independently selected from H and C₁-C₆ alkyl, or R^{4a} and R^{4b}, together with the carbon atom to which they are bound, form a three-, four-, or five-membered cycloalkyl or heterocyclyl, optionally substituted with F, Cl, and C₁-C₆ alkyl, where C₁-C₆ alkyl is independently and optionally substituted with one or more groups selected from F, Cl, —CN, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H;
R⁶ is selected from H and C₁-C₆ alkyl; and
* indicates the sulfur is conjugated to a cysteine thiol of the antibody to form a disulfide linkage, and the wavy line indicates the attachment to the cereblon degrader moiety.

In some embodiments, R^{4a} and R^{4b} are each —CH₃.

In some embodiments, R^{5a} and R^{5b} are each H.

In some embodiments, R⁶ is H.

In some embodiments, R^{4a} and R^{4b} are each —CH₃, R^{5a} and R^{5b} are each H, and R⁶ is H.

### Cereblon Degrader Moieties

In embodiments, the cD is a bivalent heterobifunctional cD or a molecular glue cD. In some embodiments, the cereblon degrader moiety (cD) has the formula:

E3UL-cDₐ

wherein
E3UL is the cereblon-binding, E3 ubiquitin ligase ligand;
cDₐ is a molecular glue moiety; or
cDₐ is TPL-L₂-, wherein
   TPL is the target protein ligand, and
   L₂ is the degrader linker.

### Bivalent Cereblon Degrader Moieties

In embodiments, cD comprises a cereblon-binding, E3 ubiquitin ligase ligand (E3UL) covalently attached to a target protein ligand (TPL) by a degrader linker (L₂) to form a bivalent heterobifunctional cD. In embodiments, the cereblon degrader moiety (cD) has the formula:

TPL-L₂-E3UL
wherein:
TPL is a target protein ligand;
E3UL is the cereblon-binding, E3 ubiquitin ligase ligand;
L₂ is a degrader linker; and
one of TPL, E3UL and L₂ is attached to L₁.

### Target Protein Ligands

A target-protein ligand (TPL) is a moiety that binds to a protein of interest to be tagged and degraded by the E3 ubiquitin ligase/proteasome system. The TPL is covalently attached to the cereblon-binding, E3 ubiquitin ligase ligand by the degrader linker.

An exemplary target protein of the cereblon degrader antibody conjugate (cDAC) is BRD4. BRD4 is a member of the bromodomain and extra terminal domain (BET) family and is an attractive target in a variety of pathological situations, particularly cancer including solid tumors and hematological malignancies. Prostate and AML (acute myeloid leukemia) cell lines show sensitivity to inhibition of BRD4 (S. E. Lochrin, et al (2014) Canc. Biol. Ther. 15 (12):1583-1585).

Additional exemplary target proteins of the cereblon degrader antibody conjugate (cDAC) include but are not limited to GSPT1, BET, BRM (SMARCA2), KRAS, and SHP2 (Wang, C. et al (2021) Eur J Med Chem. 225:113749).

In some embodiments, a TPL has the structure of the formula:

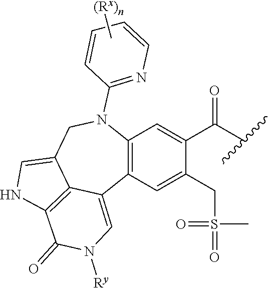

wherein
R^{x} is selected from F, Cl, and Br; and n is 0, 1, 2 or 3;
R^{y} is selected from H and C₁-C₆ alkyl; and
the wavy line indicates the point of attachment to L₂.

In some embodiments, a TPL has the structure:

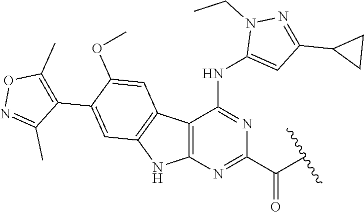 [SMILES: *C(=O)c1nc(Nc2cc(C3CC3)nn2CC)c2c(n1)[nH]c1cc(-c3c(C)noc3C)c(OC)cc12]

where the wavy line indicates the point of attachment to L₂.

In some embodiments, a TPL has the structure:

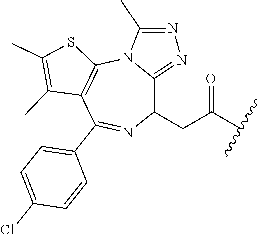 [SMILES: *C(=O)CC1N=C(c2ccc(Cl)cc2)c2c(sc(C)c2C)-n2c(C)nnc21]

where the wavy line indicates the point of attachment to L₂.

An exemplary target protein of the cereblon degrader antibody conjugate (cDAC) is GSPT1 (G1 To S phase transition protein 1 homologue), a translation termination factor (Huber, A. et al (2022) ACS Med. Chem. Lett., 13:1311-1320; Powell, C. E. et al (2020) ACS Chem. Biol. 15:2722-2730; Matyskiela, M. E. (2016) Nature 535(7611):252-257). GSPT1 is upregulated in many cancers, particularly hematopoietic malignancies, and acute leukemia cells have been shown to be highly sensitive to GSPT1 degradation. GSPT1 is therefore a potential drug target for future chemotherapies (Matyskiela, M. E. et al, (2016) Nature 535 (7611), 252-7; Surka, C.; et al, (2021) Blood 137 (5) 661-677; Takwale, A. D. et al (2022) Bioorganic Chemistry 127:105923; Hansen J D, et al (2021) J Med Chem. 64(4):1835-1843).

### Degrader Linkers

The degrader linker (L₂) is any suitable bifunctional or trifunctional linker unit that covalently attaches to the target protein ligand (TPL) and the cereblon-binding, E3 ubiquitin ligase ligand (E3UL). The degrader linker may be covalently attached to the antibody linker L₁ to form the cereblon degrader antibody conjugate (cDAC).

In some embodiments, L₂ is selected from:
—N(R′)—(C₁-C₁₂ alkyldiyl)-N(R′)—,
—N(R′)—(C₂-C₁₂ alkenyldiyl)-N(R′)—,
—N(R′)—(C₂-C₁₂ alkynyldiyl)-N(R′)—,
—N(R′)—(C₁-C₁₂ alkyldiyl)-C(═O)—(N(R′)—,
—N(R′)—(C₁-C₁₂ alkyldiyl)-(N(R′)—C(═O)CH₂O—,
—N(R′)—(C₁-C₁₂ alkyldiyl)-(N(R′)—C(═O)CH₂N(R′)—,
—N(R′)—(C₁-C₁₂ alkyldiyl)-C(═O)—(N(R′)—(C₁-C₁₂ alkyldiyl)-N(R′)—,
—N(R′)—(C₁-C₆ alkyldiyl)-O—(C₁-C₆ alkyldiyl)-N(R′)—,
—N(R′)—(CH₂CH₂O)ₙ—N(R′)—(CH₂CH₂O)ₙ—, where n is an integer from 1 to 4,
C₁-C₁₂ alkyldiyl,
C₂-C₁₂ alkenyldiyl, and
C₂-C₁₂ alkynyldiyl,
where R′ is selected from H, C₁-C₆ alkyldiyl, and a point of attachment to L₁; and where
alkyldiyl, alkenyldiyl, and alkynyldiyl are optionally substituted with one or more substituents selected from F, Cl, —CN, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H.

### Bivalent Cereblon Degrader Compounds

Exemplary heterobifunctional cereblon degrader compounds were prepared and characterized, and are shown in Table 1.

TABLE 1
Bivalent Cereblon Degrader Compounds (cD)

| cD | | |
|---|---|---|
| No. | Structure | MW |
| 1 | 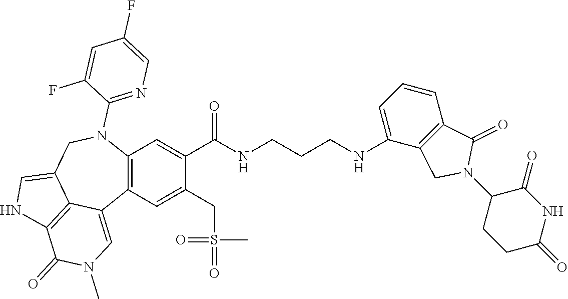 [SMILES: Cn1cc2c3c(c[nH]c3c1=O)CN(c1ncc(F)cc1F)c1cc(C(=O)NCCCNc3cccc4c3CN(C3CCC(=O)NC3=O)C4=O)c(CS(C)(=O)=O)cc1-2] | 798.2 |
| 2 | 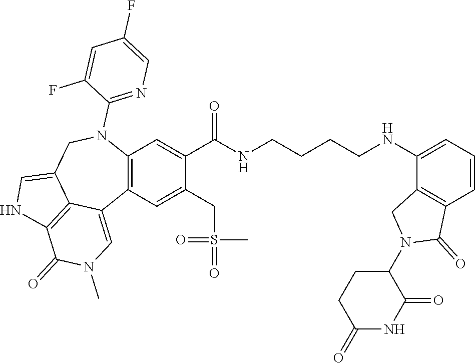 [SMILES: Cn1cc2c3c(c[nH]c3c1=O)CN(c1ncc(F)cc1F)c1cc(C(=O)NCCCCNc3cccc4c3CN(C3CCC(=O)NC3=O)C4=O)c(CS(C)(=O)=O)cc1-2] | 812.3 |
| 3 | 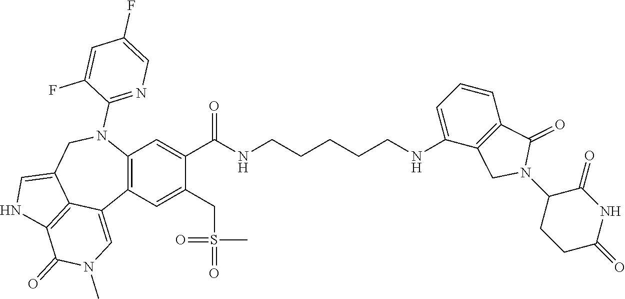 [SMILES: Cn1cc2c3c(c[nH]c3c1=O)CN(c1ncc(F)cc1F)c1cc(C(=O)NCCCCCNc3cccc4c3CN(C3CCC(=O)NC3=O)C4=O)c(CS(C)(=O)=O)cc1-2] | 826.9 |
| 4 | 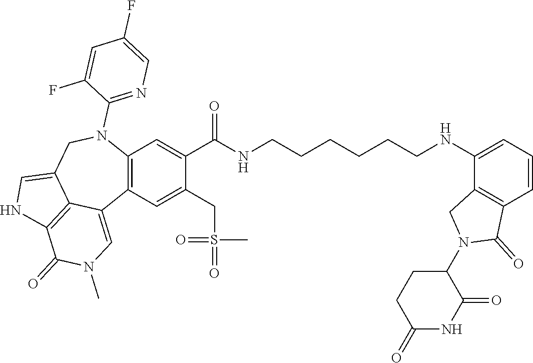 [SMILES: Cn1cc2c3c(c[nH]c3c1=O)CN(c1ncc(F)cc1F)c1cc(C(=O)NCCCCCCNc3cccc4c3CN(C3CCC(=O)NC3=O)C4=O)c(CS(C)(=O)=O)cc1-2] | 840.9 |
| 5 | 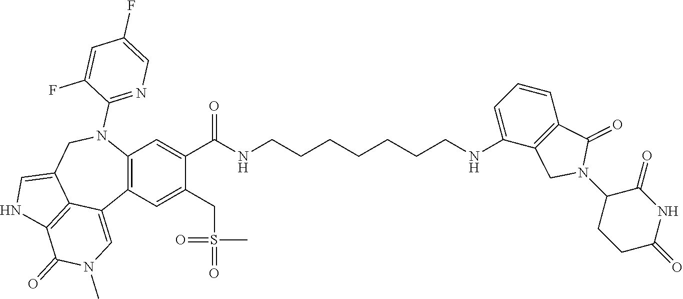 [SMILES: Cn1cc2c3c(c[nH]c3c1=O)CN(c1ncc(F)cc1F)c1cc(C(=O)NCCCCCCCNc3cccc4c3CN(C3CCC(=O)NC3=O)C4=O)c(CS(C)(=O)=O)cc1-2] | 854.9 |
| 6 | 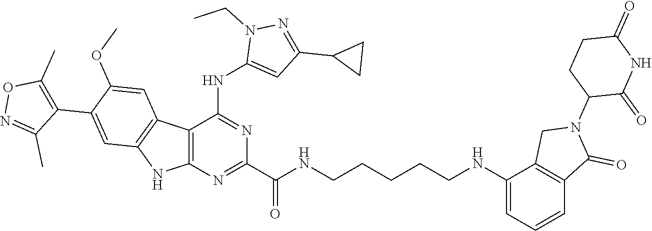 [SMILES: CCn1nc(C2CC2)cc1Nc1nc(C(=O)NCCCCCNc2cccc3c2CN(C2CCC(=O)NC2=O)C3=O)nc2[nH]c3cc(-c4c(C)noc4C)c(OC)cc3c12] | 813.9 |
| 7 | 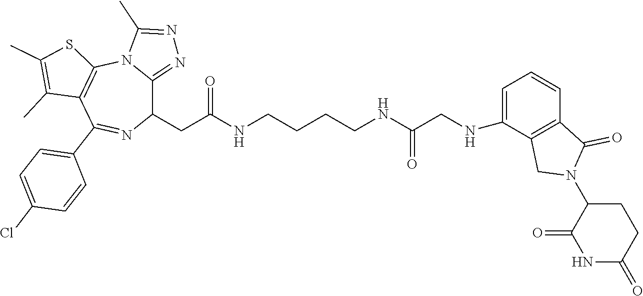 [SMILES: Cc1sc2c(c1C)C(c1ccc(Cl)cc1)=NC(CC(=O)NCCCCNC(=O)CNc1cccc3c1CN(C1CCC(=O)NC1=O)C3=O)c1nnc(C)n1-2] | 770.3 |
| 8 | 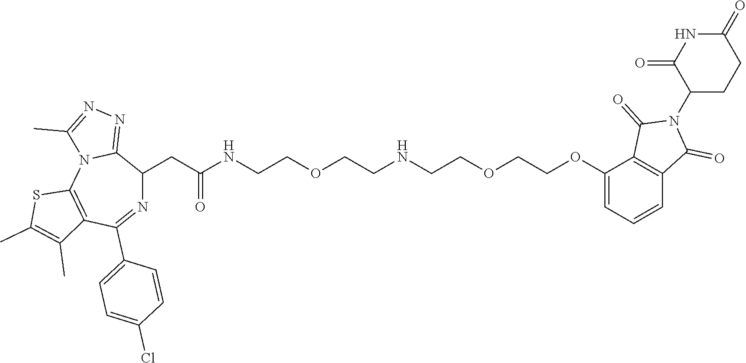 [SMILES: Cc1sc2c(c1C)C(c1ccc(Cl)cc1)=NC(CC(=O)NCCOCCNCCOCCOc1cccc3c1C(=O)N(C1CCC(=O)NC1=O)C3=O)c1nnc(C)n1-2] | 831.3 |
| 9 | 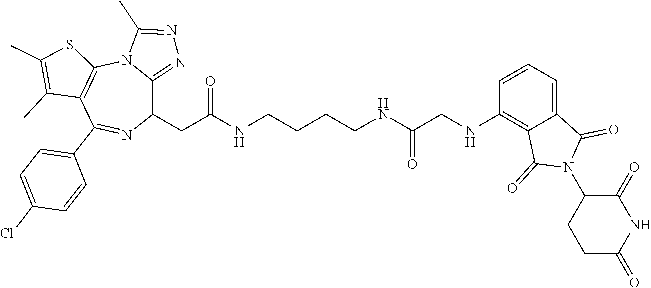 [SMILES: Cc1sc2c(c1C)C(c1ccc(Cl)cc1)=NC(CC(=O)NCCCCNC(=O)CNc1cccc3c1C(=O)N(C1CCC(=O)NC1=O)C3=O)c1nnc(C)n1-2] | 784.3 |
| 10 | 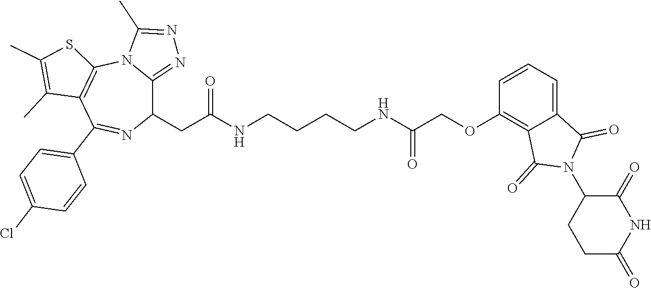 [SMILES: Cc1sc2c(c1C)C(c1ccc(Cl)cc1)=NC(CC(=O)NCCCCNC(=O)COc1cccc3c1C(=O)N(C1CCC(=O)NC1=O)C3=O)c1nnc(C)n1-2] | 785.3 |

### Molecular Glue Cereblon Degrader Moieties

In embodiments, cereblon degrader moiety (cD) of an antibody conjugate (cDAC) is a molecular glue cereblon degrader moiety. In such embodiments, the molecular glue cereblon degrader moiety (cD) comprises an E3UL covalently attached to a molecular glue moiety (cDₐ) to form a molecular glue cD.

In some embodiments, the molecular glue cD comprises a structure selected from the formulae:

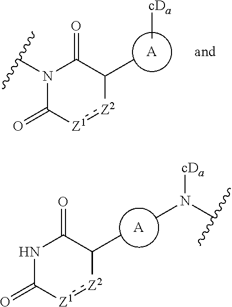

wherein the wavy line indicates the point of attachment to the antibody linker L₁ of Formula I or the linker L₃ of Formula II;
cDₐ is a molecular glue moiety;
the dashed line indicates an optional double bond;
Z¹ is selected from C(R¹)₂, CR¹, N, and NR^{1a};
Z² is selected from C(R²)₂, CR², N, and NR^{2a};
R¹ and R² are independently selected from the group consisting of H, F, Cl, Br, I, —CN, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —NO₂, ═O, —OR^{a}, —OC(═O)R^{a}, —SR^{a}, —S(O)R^{a}, —S(O)₂R^{a}, —S(O)₂NR^{a}, and —S(O)₃H;
R^{1a} and R^{2a} are independently selected from the group consisting of H, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —ORₐ, —S(O)R^{a}, —S(O)₂R^{a}, —S(O)₂NR^{a}, and —S(O)₃H;
or (i) two geminal R¹ or two geminal R² form a 3-6 membered carbocyclyl or heterocyclyl spiro group, or (ii) R¹ and R², R^{1a} and R², R¹ and R^{2a}, or R^{1a} and R^{2a} form a fused 5- or 6-membered aryl, carbocyclyl, heterocyclyl, or heteroaryl group;
R^{a} and R^{b} are independently selected from H, OH, C₁-C₆ alkyl, phenyl, and benzyl, where phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl, and
A is selected from C₆-C₂₀ aryl, C₃-C₂₀ carbocyclyl, C₂-C₂₀ heterocyclyl, and C₁-C₂₀ heteroaryl;
alkyl, alkyldiyl, alkenyl, alkynyl, aryl, carbocyclyl, heterocyclyl, and heteroaryl, are independently and optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H.

In some embodiments, Z¹ and Z² are each CR¹, and R¹ and R² are each H.

In some embodiments, R is H.

In some embodiments, A is C₆-C₂₀ aryl.

In some embodiments,
Z¹ and Z² are each CR¹, wherein R¹ and R² are each H;
R is H; and
A is C₆-C₂₀ aryl.

In some embodiments, the molecular glue cD comprises a structure selected from the formulae:

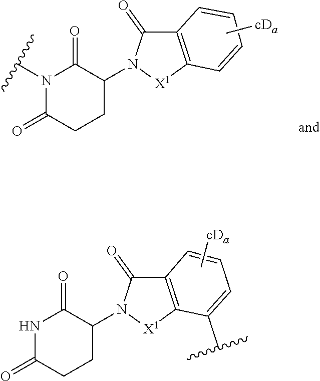

wherein
the wavy line indicates the point of attachment of the antibody linker L₁ of Formula I or the linker L₃ of Formula II;
cDₐ is a molecular glue moiety; and
X¹ is selected from CH₂ and C(═O).

In some embodiments, X¹ is C(═O).

### Cereblon Degrader-Linker Intermediates

A cereblon degrader-linker intermediate (cDLI) is a reagent for the process of making a cereblon degrader antibody conjugate (cDAC) by conjugation with a thiol-containing antibody. The cereblon degrader-linker intermediate has a thiol-reactive functional group (X). The thiol-reactive functional group (X) is covalently attached to the cereblon degrader moiety (cD) by a linker (L₃).

In some embodiments, a cereblon degrader-linker intermediate has the structure of Formula II:

X-L₃-cD II
wherein:
X is a thiol-reactive group;
L₃ is a linker selected from:
(i) a protease-cleavable, non-peptide linker having the formula:

-Str-PM-Y—
wherein Str is a stretcher unit covalently attached to X,
PM is a peptidomimetic unit, and
Y is a spacer unit covalently attached to cD;
(ii) a disulfide linker selected from the formulae:

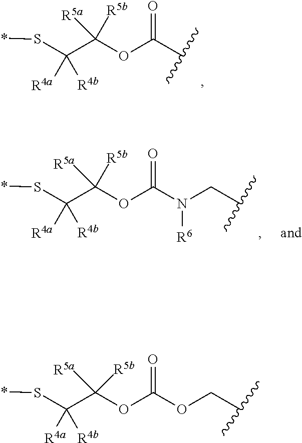

and
(iii) a linker having the formula:

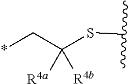

wherein * indicates the point of attachment to X,
R^{4a}, R^{4b}, R^{5a}, and R^{5a} are independently selected from H and C₁-C₆ alkyl, or R^{4a} and R^{4b} together with the carbon atom to which they are bound form a three-, four-, or five-membered cycloalkyl or heterocyclyl, optionally substituted with F, Cl, and C₁-C₆ alkyl;
R⁶ is selected from H and C₁-C₆ alkyl,
the wavy line indicates the attachment to cD,
C₁-C₆ alkyl is independently and optionally substituted with one or more groups selected from F, Cl, —CN, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H; and
cD is a cereblon degrader moiety having the formula:

TPL-L₂-E3UL
wherein:
TPL is a target protein ligand;
E3UL is a cereblon-binding, E3 ubiquitin ligase ligand;
L₂ is a degrader linker; and
one of TPL, E3UL and L₂ is attached to L₁; or
cD is a molecular glue.

In some embodiments, the attachment of L₃ to cD comprises a carbamate (—OC(O)NH—) or methylcarbamate (—OC(O)NHCH₂—) group.

In some embodiments, X is selected from a group consisting of maleimide, bromoacetamide, toluenesulfonyl sulfide, and 2-pyridyldisulfide where the pyridyl is optionally substituted with one or two nitro groups.

In some embodiments, the cereblon degrader-linker intermediate has the formula:

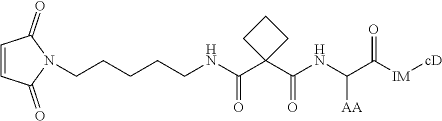

wherein IM comprises a group selected from 4-aminobenzyl, 4-aminobenzyloxycarbonyl, and (4-aminobenzyl)methylcarbamate, and
AA is a side chain of an amino acid selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, and citrulline.

In some embodiments, AA is selected from H, —CH₃, —CH₂(C₆H₅), —CH₂CH₂CH₂CH₂NH₂, —CH₂CH₂CH₂NHC(NH)NH₂, —CH₂CH(CH₃)₂, and —CH₂CH₂CH₂NHC(O)NH₂.

In some embodiments, AA is —CH₃ or —CH₂CH₂CH₂NHC(O)NH₂.

In some embodiments, the cereblon degrader-linker intermediate is selected from the formulae:

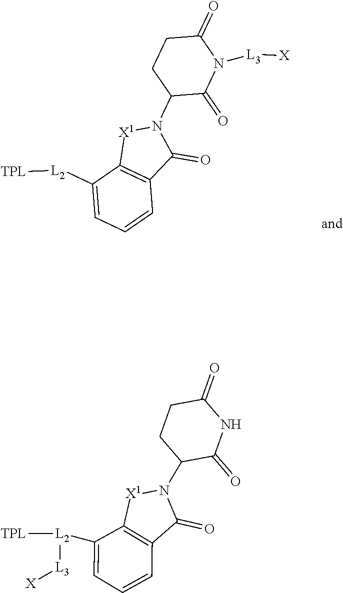

wherein X¹ is selected from CH₂ and C(═O).

In some embodiments, the cereblon degrader-linker intermediate includes the formula:

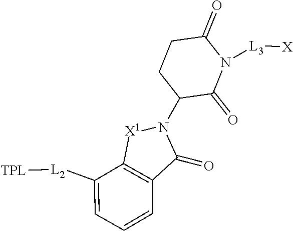

wherein L₃ is a protease-cleavable, non-peptide linker having the formula:

-Str-PM-IM-
wherein Str is a stretcher unit covalently attached to X,
PM is a peptidomimetic unit, and
IM is an immolator unit covalently attached to cD and has the formula:

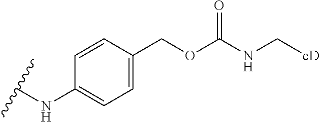

where the wavy line is the attachment to PM.

In some embodiments, the cereblon degrader-linker intermediate includes the formula:

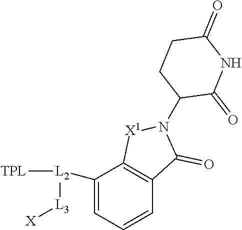

wherein L₃ is a protease-cleavable, non-peptide linker having the formula:

-Str-PM-IM-
wherein Str is a stretcher unit covalently attached to X,
PM is a peptidomimetic unit, and
IM is an immolator unit covalently attached to L₂ of cD and has the formula:

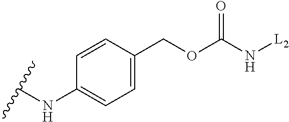

where the wavy line is the attachment to PM.

Certain cDLI in Table 2 were prepared which did not possess the necessary properties of stability, cleavage efficiency, and conjugation efficiency with antibodies.

TABLE 2
Cereblon degrader-linker intermediates (cDLI) with poor stability and/or
conjugation inefficiency

| cDLI | | |
|---|---|---|
| No. | Structure | Name |
| 1 | 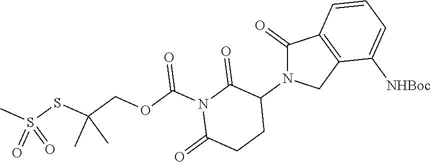 [SMILES: CC(C)(C)OC(=O)Nc1cccc2c1CN(C1CCC(=O)N(C(=O)OCC(C)(C)SS(C)(=O)=O)C1=O)C2=O] | 2-methyl-2- ((methyl- sulfonyl) thio)propyl 3-(4-((tert- butoxy- carbonyl) amino)-1- oxoisoindolin- 2-yl)-2,6- |
| | | dioxo- |
| | | piperidine- |
| | | 1-carboxylate |
| 2 | 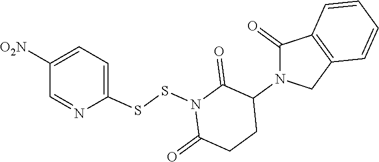 [SMILES: O=C1CCC(N2Cc3ccccc3C2=O)C(=O)N1SSc1ccc([N+](=O)[O-])cn1] | 1-((5- nitropyridin- 2-yl) disulfaneyl)- 3-(1- oxoisoindolin- 2-yl) piperidine- 2,6-dione |
| 3 | 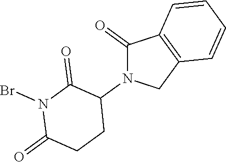 [SMILES: O=C1CCC(N2Cc3ccccc3C2=O)C(=O)N1Br] | 1-bromo-3-(1- oxoisoindolin- 2- yl)piperidine- 2,6-dione |
| 4 | 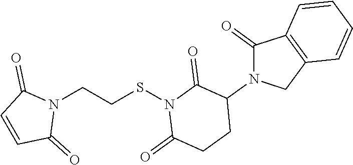 [SMILES: O=C1C=CC(=O)N1CCSN1C(=O)CCC(N2Cc3ccccc3C2=O)C1=O] | 1-((2-(2,5- dioxo- 2,5-dihydro- 1H-pyrrol-1- yl)ethyl) thio)-3-(1- oxoisoindolin- 2- yl)piperidine- 2,6-dione |
| 5 | 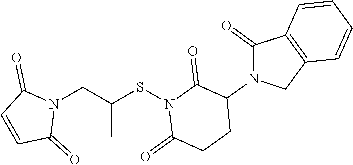 [SMILES: CC(CN1C(=O)C=CC1=O)SN1C(=O)CCC(N2Cc3ccccc3C2=O)C1=O] | 1-((1-(2,5- dioxo- 2,5-dihydro- 1H-pyrrol-1- yl)propan- 2-yl)thio)- 3-(1- oxoisoindolin- 2- yl)piperidine- 2,6-dione |
| 6 | 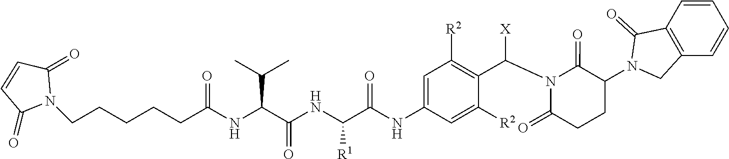 | 6-(2,5- dioxo-2,5- dihydro- 1H-pyrrol- 1-yl)-N-((2S)- 1-(((2S)-1- ((4-((2,6- dioxo-3-(1- oxoisoindolin- 2-yl) |
| | | piperidin-1- |
| | R¹ = CH₃, citrulline; R² = H, OCH₃, X = H, CH₃ | yl)methyl) |
| | | phenyl) |
| | | amino)-1- |
| | | oxopropan- |
| | | 2-yl) |
| | | amino)-3- |
| | | methyl-1- |
| | | oxobutan-2-yl) |
| | | hexanamide |
| | | (R¹ = CH₃, |
| | | R² = H, X = H) |
| 7 | 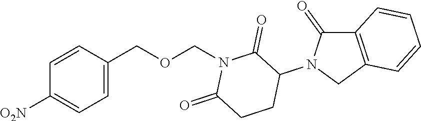 [SMILES: O=C1CCC(N2Cc3ccccc3C2=O)C(=O)N1COCc1ccc([N+](=O)[O-])cc1] | 1-(((4- nitrobenzyl) oxy)methyl)- 3-(1- oxoisoindolin- 2- yl)piperidine- 2,6-dione |
| 8 | 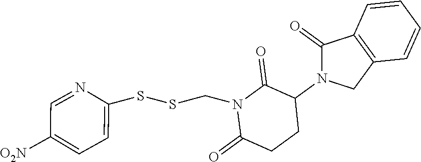 [SMILES: O=C1CCC(N2Cc3ccccc3C2=O)C(=O)N1CSSc1ccc([N+](=O)[O-])cn1] | 1-(((5- nitropyridin- 2- yl)disulfaneyl) methyl)-3-(1- oxoiso- indolin-2- yl)piperidine- 2,6-dione |
| 9 | 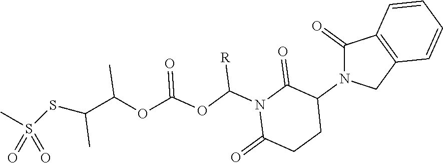 | S-(3-((((2,6- dioxo-3-(1- oxoisoindolin- 2- yl)piperidin-1- yl)methoxy) carbonyl) oxy)butan-2- yl)methane- sulfonothioate |
| | R = H, CH₃ | |
| 10 | 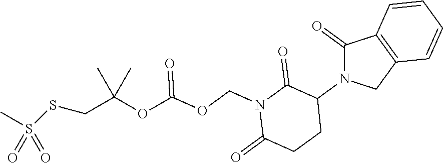 [SMILES: CC(C)(CSS(C)(=O)=O)OC(=O)OCN1C(=O)CCC(N2Cc3ccccc3C2=O)C1=O] | S-(2-((((2,6- dioxo-3-(1- oxoisoindolin- 2-yl) piperidin-1- yl)methoxy) carbonyl) oxy)-2- methylpropyl) methane- sulfonothioate |

The sulfonyl-thio cDLI-1 compound failed to react with an antibody under conditions described in Example 102, including pH 8.5 with 3 and 10 equivalents for 3 hours and overnight. Analysis by mass spectrometry (LC/MS) showed none of the expected conjugate product cDAC, opening of the glutarimide ring, addition of water (+18 mass units), and addition of Tris buffer to the glutarimide ring. The carbamate functional group formed by the glutarimide nitrogen in cDLI-1 is too unstable under these conditions for conjugation.

Sulfenamide cDLI-2 and bromo-lenalidomide cDLI-3 compounds did not conjugate to antibodies. Maleimide-sulfenamide compounds cDLI-4 and cDLI-5 failed to conjugate to cysteine-mutant antibodies and were not stable in whole blood.

Various substituted peptide linker cDLI-6 conjugated to antibodies but failed to cleave in the presence of proteases. The (S,S) valine-alanine and (S,S) valine-citrulline versions of cDLI-6 were tested, along with phenyl and dimethoxyphenyl versions, and with and without a methyl group adjacent to the glutarimide nitrogen, all gave cDAC that did not cleave to release a cereblon-degrader moiety or its metabolite.

The nitro group of para-nitrobenzyloxymethyl lenalidomide cDLI-7 was reduced to amine in a model study. No cleavage of the para-aminobenzyloxy group was observed by the appearance of lenalidomide was detected. The disulfide group of para-nitropyridyl disulfidemethyl lenalidomide cDLI-8 was reduced in a model study. No cleavage of the disulfide group was observed by the appearance of lenalidomide was detected.

Sulfonyl-thio cDLI-9, with and without methyl adjacent to the glutarimide nitrogen, cleaved to release detectable lenalidomide but was not stable, generating hydrolysis products.

Both sulfonyl-thio cDLI-9 and cDLI-10 failed to conjugate with antibodies under different conditions, pH 8.5, 3 and 10 equivalents to antibody, and from 3 hours to overnight reaction times. Hydrolysis of the carbonate bond was observed by LC/MS.

In some embodiments, the cereblon degrader-linker intermediate includes TPL selected from the formulae:

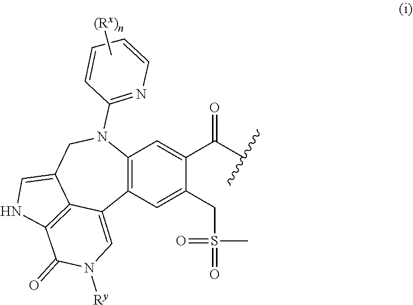

wherein R^{x} is selected from F, Cl, and Br, and n is 0, 1, 2 or 3; R^{y} is selected from H and C₁-C₆ alkyl;

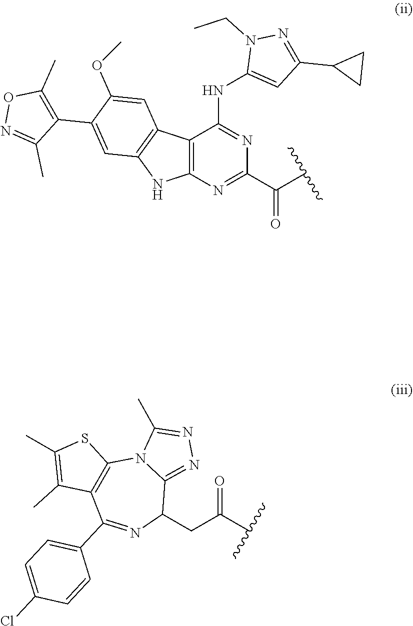 [SMILES: *C(=O)c1nc(Nc2cc(C3CC3)nn2CC)c2c(n1)[nH]c1cc(-c3c(C)noc3C)c(OC)cc12 ; *C(=O)CC1N=C(c2ccc(Cl)cc2)c2c(sc(C)c2C)-n2c(C)nnc21]

where the wavy lines indicate the point of attachment of L₂.

Exemplary cereblon degrader-linker intermediates (cDLI) are selected from:

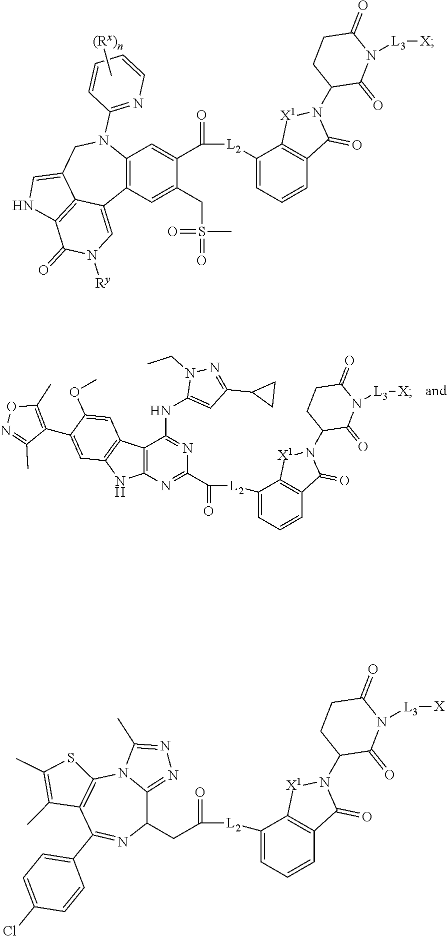

wherein R^{x} is selected from F, Cl, and Br, and n is 0, 1, 2 or 3; R^{y} is selected from H and C₁-C₆ alkyl, and X¹ is selected from CH₂ and C(═O).

In some embodiments, the cereblon degrader-linker intermediate includes wherein L₂ is selected from:
—N(R)—(C₁-C₁₂ alkyldiyl)-N(R)—,
—N(R)—(C₂-C₁₂ alkenyldiyl)-N(R)—,
—N(R)—(C₂-C₁₂ alkynyldiyl)-N(R)—,
—N(R)—(C₁-C₁₂ alkyldiyl)-C(═O)—(N(R)—,
—N(R)—(C₁-C₁₂ alkyldiyl)-C(═O)—(N(R)—(C₁-C₁₂ alkyldiyl)-N(R)—,
(C₁-C₆ alkyldiyl)-O—(C₁-C₆ alkyldiyl)-,
C₁-C₁₂ alkyldiyl,
C₂-C₁₂ alkenyldiyl, and
C₂-C₁₂ alkynyldiyl,
where alkyldiyl, alkenyldiyl, and alkynyldiyl are optionally substituted with one or more groups selected from F, Cl, —OH, —OCH₃, —OCH₂CH₃, —OCH₂CH₂OCH₃, —OCH₂CH₂OH, —OCH₂CH₂N(CH₃)₂, and R is selected from H, C₁-C₆ alkyldiyl, and a point of attachment to L₃.

The cereblon degrader-linker intermediates (cDLI) in Table 3 were prepared which possessed the necessary properties of stability, cleavage efficiency, and conjugation efficiency with antibodies. Each cDLI in Table 3 was characterized by NMR and shown to have sufficient purity and the correct mass by LC/MS.

TABLE 3
Examples of cereblon degrader-linker intermediates (cDLI)

| cDLI | | |
|---|---|---|
| No. | Structure | MW |
| 1 | 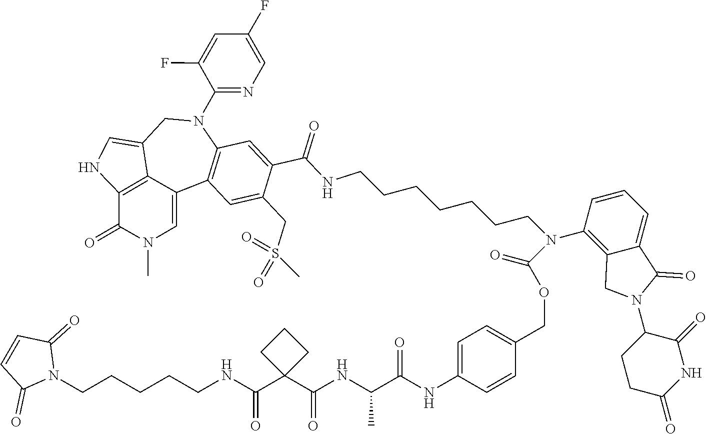 [SMILES: C[C@H](NC(=O)C1(C(=O)NCCCCCN2C(=O)C=CC2=O)CCC1)C(=O)Nc1ccc(COC(=O)N(CCCCCCCNC(=O)c2cc3c(cc2CS(C)(=O)=O)-c2cn(C)c(=O)c4[nH]cc(c24)CN3c2ncc(F)cc2F)c2cccc3c2CN(C2CCC(=O)NC2=O)C3=O)cc1] | 1365.5 |
| 2 | 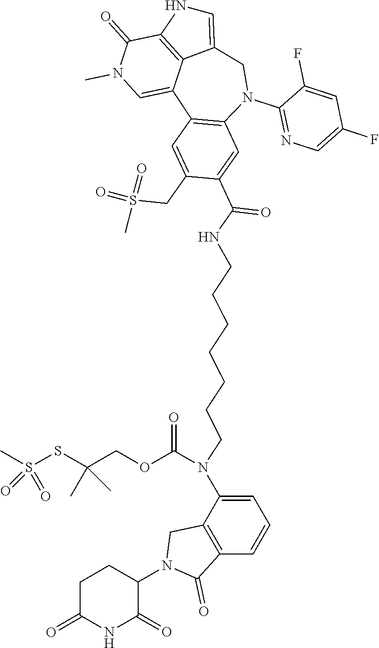 [SMILES: Cn1cc2c3c(c[nH]c3c1=O)CN(c1ncc(F)cc1F)c1cc(C(=O)NCCCCCCCN(C(=O)OCC(C)(C)SS(C)(=O)=O)c3cccc4c3CN(C3CCC(=O)NC3=O)C4=O)c(CS(C)(=O)=O)cc1-2] | 1065.2 |
| 3 | 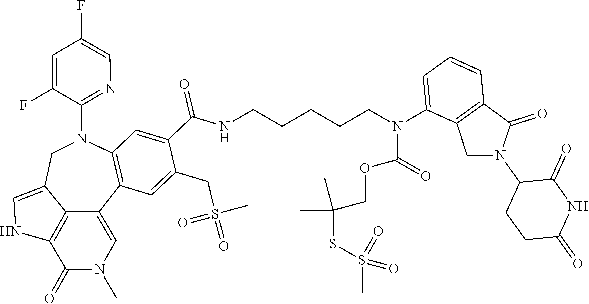 [SMILES: Cn1cc2c3c(c[nH]c3c1=O)CN(c1ncc(F)cc1F)c1cc(C(=O)NCCCCCN(C(=O)OCC(C)(C)SS(C)(=O)=O)c3cccc4c3CN(C3CCC(=O)NC3=O)C4=O)c(CS(C)(=O)=O)cc1-2] | 1037.1 |
| 4 | 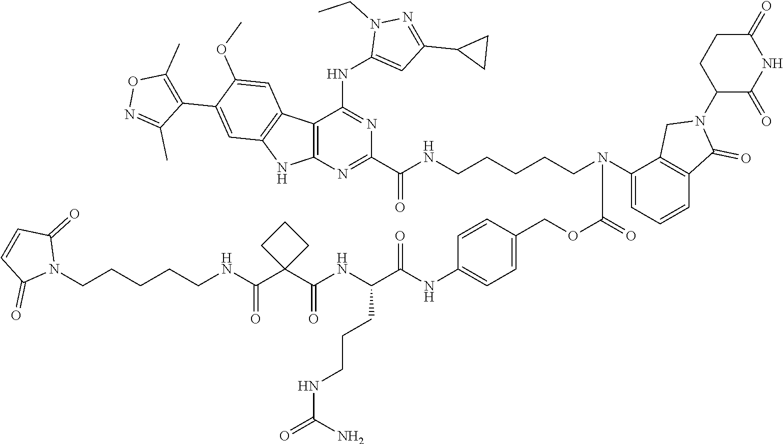 [SMILES: CCn1nc(C2CC2)cc1Nc1nc(C(=O)NCCCCCN(C(=O)OCc2ccc(NC(=O)[C@H](CCCNC(N)=O)NC(=O)C3(C(=O)NCCCCCN4C(=O)C=CC4=O)CCC3)cc2)c2cccc3c2CN(C2CCC(=O)NC2=O)C3=O)nc2[nH]c3cc(-c4c(C)noc4C)c(OC)cc3c12] | 1410.6 |
| 5 | 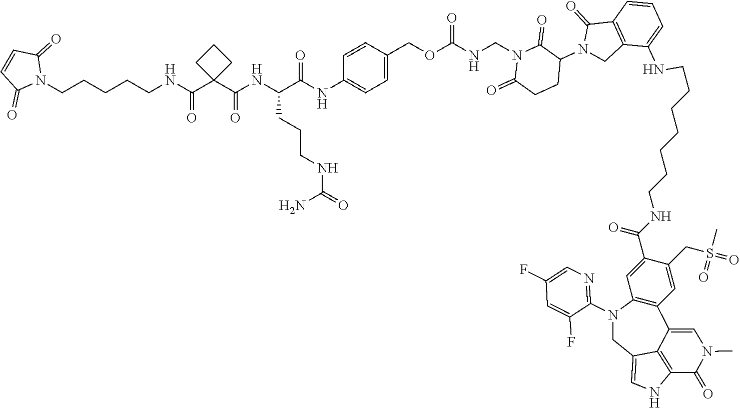 [SMILES: Cn1cc2c3c(c[nH]c3c1=O)CN(c1ncc(F)cc1F)c1cc(C(=O)NCCCCCCCNc3cccc4c3CN(C3CCC(=O)N(CNC(=O)OCc5ccc(NC(=O)[C@H](CCCNC(N)=O)NC(=O)C6(C(=O)NCCCCCN7C(=O)C=CC7=O)CCC6)cc5)C3=O)C4=O)c(CS(C)(=O)=O)cc1-2] | 1480.6 |
| 6 | 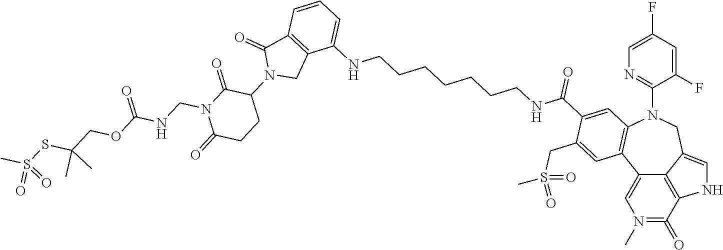 | 1094.2 |
| 7 | 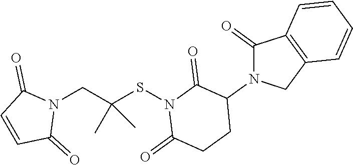 [SMILES: CC(C)(CN1C(=O)C=CC1=O)SN1C(=O)CCC(N2Cc3ccccc3C2=O)C1=O] | 427.5 |

### Antibodies

The cereblon degrader antibody conjugate (cDAC) provided herein comprises an antibody. Included in the scope of the embodiments of the antibody are functional variants of antibody constructs and antigen binding domains described herein.

The antibody portion of a cDAC can target a cell that expresses an antigen whereby the antigen specific cDAC is delivered intracellularly to the target cell, typically through endocytosis. While a cDAC that comprise an antibody directed to an antigen that is not found on the cell surface may result in less specific intracellular delivery of the cereblon-degrader moiety into the cell, the cDAC may still undergo pinocytosis. cDACs and methods of their use described herein advantageously utilize antibody recognition at the cellular surface and/or endocytosis of the cDAC to deliver the cereblon degrader moiety portion inside cells.

### Trastuzumab, Anti-HER2 Antibody

In certain embodiments, immunoconjugates (e.g., cDACs) described herein comprise anti-HER2 antibodies. In some embodiments, an anti-HER2 antibody of the cDAC comprises a humanized anti-HER2 antibody, e.g., huMAb4D5-1, huMAb4D5-2, huMAb4D5-3, huMAb4D5-4, huMAb4D5-5, huMAb4D5-6, huMAb4D5-7 and huMAb4D5-8, as described in Table 3 of U.S. Pat. No. 5,821,337, which is specifically incorporated by reference herein. Those antibodies contain human framework regions with the complementarity-determining regions of a murine antibody (4D5) that binds to HER2. The humanized antibody huMAb4D5-8 is also referred to as trastuzumab, commercially available under the tradename HERCEPTIN™ (Genentech, Inc.).

Trastuzumab (CAS 180288-69-1, HERCEPTIN®, huMAb4D5-8, rhuMAb HER2, Genentech) is a recombinant DNA-derived, IgG1 kappa, monoclonal antibody that is a humanized version of a murine anti-HER2 antibody (4D5) that selectively binds with high affinity in a cell-based assay (Kd=5 nM) to the extracellular domain of HER2 (U.S. Pat. Nos. 5,677,171; 5,821,337; 6,054,297; 6,165,464; 6,339,142; 6,407,213; 6,639,055; 6,719,971; 6,800,738; 7,074,404; Coussens et al (1985) Science 230:1132-9; Slamon et al (1989) Science 244:707-12; Slamon et al (2001) New Engl. J. Med. 344:783-792).

In some embodiments, the antibody construct or antigen binding domain comprises the CDR regions of trastuzumab. In some embodiments, the anti-HER2 antibody further comprises the framework regions of the trastuzumab. In some embodiments, the anti-HER2 antibody further comprises one or both variable regions of trastuzumab.

### 7C2, Anti-HER2 Antibody

Anti-HER2 murine antibody 7C2 binds to an epitope in domain I of HER2. See, e.g., PCT Publication No. WO 98/17797. This epitope is distinct from the epitope bound by trastuzumab, which binds to domain IV of HER2, and the epitope bound by pertuzumab, which binds to domain II of HER2. By binding domain IV, trastuzumab disrupts ligand-independent HER2-HER3 complexes, thereby inhibiting downstream signaling (e.g. PI3K/AKT). In contrast, pertuzumab binding to domain II prevents ligand-driven HER2 interaction with other HER family members (e.g. HER3, HER1 or HER4), thus also preventing downstream signal transduction. Binding of MAb 7C2 to domain I does not result in interference of trastuzumab or pertuzumab binding to domains IV and II, respectively, thereby offering the potential of combining a MAb 7C2 ADC (antibody drug conjugate) with trastuzumab, trastuzumab emtansine (T-DM1), and/or pertuzumab. Murine antibody 7C2, 7C2.B9, is described in WO 1998/017797. An anti-HER2 7C2 humanized antibody is disclosed in WO 2016/040723.

In some embodiments, an anti-HER2 antibody of the cDAC described herein comprises a humanized 7C2 anti-HER2 antibody. A humanized 7C2 antibody is an anti-HER2 antibody.

In some embodiments, the cDAC described herein comprises an anti-HER2 antibody comprising at least one, two, three, four, five, or six HVRs selected from (a) HVR—H1 comprising the amino acid sequence of SEQ ID NO: 6; (b) HVR—H2 comprising the amino acid sequence of SEQ ID NO: 7, 11, or 12; (c) HVR—H3 comprising the amino acid sequence of SEQ ID NO: 8 or 13; (d) HVR-L1 comprising the amino acid sequence of SEQ ID NO: 3; (e) HVR-L2 comprising the amino acid sequence of SEQ ID NO: 4; and (f) HVR-L3 comprising the amino acid sequence of SEQ ID NO: 5. In some embodiments the cDAC described herein comprises an anti-HER2 antibody comprising at least one, two, three, four, five, or six HVRs selected from (a) HVR—H1 comprising the amino acid sequence of SEQ ID NO: 6; (b) HVR—H2 comprising the amino acid sequence of SEQ ID NO: 7; (c) HVR—H3 comprising the amino acid sequence of SEQ ID NO: 8; (d) HVR-L1 comprising the amino acid sequence of SEQ ID NO: 3; (e) HVR-L2 comprising the amino acid sequence of SEQ ID NO: 4; and (f) HVR-L3 comprising the amino acid sequence of SEQ ID NO: 5.

In one aspect the cDAC described herein comprises an antibody that comprises at least one, at least two, or all three VH HVR sequences selected from (a) HVR—H1 comprising the amino acid sequence of SEQ ID NO: 6; (b) HVR—H2 comprising the amino acid sequence of SEQ ID NO: 7, 11, or 12; and (c) HVR—H3 comprising the amino acid sequence of SEQ ID NO: 8 or 13. In one aspect, the cDAC described herein comprises an antibody that comprises at least one, at least two, or all three VH HVR sequences selected from (a) HVR—H1 comprising the amino acid sequence of SEQ ID NO: 6; (b) HVR—H2 comprising the amino acid sequence of SEQ ID NO: 7; and (c) HVR—H3 comprising the amino acid sequence of SEQ ID NO: 8. In a further embodiment, the antibody comprises (a) HVR—H1 comprising the amino acid sequence of SEQ ID NO: 6; (b) HVR—H2 comprising the amino acid sequence of SEQ ID NO: 7, 11, or 12; and (c) HVR—H3 comprising the amino acid sequence of SEQ ID NO: 8 or 13. In a further embodiment, the antibody comprises (a) HVR—H1 comprising the amino acid sequence of SEQ ID NO: 6; (b) HVR—H2 comprising the amino acid sequence of SEQ ID NO: 7; and (c) HVR—H3 comprising the amino acid sequence of SEQ ID NO: 8.

In another aspect, the cDAC described herein comprises an antibody that comprises at least one, at least two, or all three VL HVR sequences selected from (a) HVR-L1 comprising the amino acid sequence of SEQ ID NO: 3; (b) HVR-L2 comprising the amino acid sequence of SEQ ID NO: 4; and (c) HVR-L3 comprising the amino acid sequence of SEQ ID NO: 5. In one embodiment, the antibody comprises (a) HVR-L1 comprising the amino acid sequence of SEQ ID NO: 3; (b) HVR-L2 comprising the amino acid sequence of SEQ ID NO: 4; and (c) HVR-L3 comprising the amino acid sequence of SEQ ID NO: 5.

In another aspect, the cDAC described herein comprises an antibody comprising (a) a VH domain comprising at least one, at least two, or all three VH HVR sequences selected from (i) HVR—H1 comprising the amino acid sequence of SEQ ID NO: 6, (ii) HVR—H2 comprising the amino acid sequence of SEQ ID NO: 7, 11, or 12, and (iii) HVR—H3 comprising an amino acid sequence selected from SEQ ID NO: 8 or 13; and (b) a VL domain comprising at least one, at least two, or all three VL HVR sequences selected from (i) HVR-L1 comprising the amino acid sequence of SEQ ID NO: 3, (ii) HVR-L2 comprising the amino acid sequence of SEQ ID NO: 4, and (c) HVR-L3 comprising the amino acid sequence of SEQ ID NO: 5. In another aspect, the cDAC described herein comprises: (a) a VH domain comprising at least one, at least two, or all three VH HVR sequences selected from (i) HVR—H1 comprising the amino acid sequence of SEQ ID NO: 6, (ii) HVR—H2 comprising the amino acid sequence of SEQ ID NO: 7, and (iii) HVR—H3 comprising an amino acid sequence selected from SEQ ID NO: 8; and (b) a VL domain comprising at least one, at least two, or all three VL HVR sequences selected from (i) HVR-L1 comprising the amino acid sequence of SEQ ID NO: 3, (ii) HVR-L2 comprising the amino acid sequence of SEQ ID NO: 4, and (c) HVR-L3 comprising the amino acid sequence of SEQ ID NO: 5.

In another aspect, the cDAC described herein comprises an antibody that comprises (a) HVR—H1 comprising the amino acid sequence of SEQ ID NO: 6; (b) HVR—H2 comprising the amino acid sequence of SEQ ID NO: 7, 11, or 12; (c) HVR—H3 comprising the amino acid sequence of SEQ ID NO: 8 or 13; (d) HVR-L1 comprising the amino acid sequence of SEQ ID NO: 3; (e) HVR-L2 comprising the amino acid sequence of SEQ ID NO: 4; and (f) HVR-L3 comprising the amino acid sequence of SEQ ID NO: 5. In another aspect, the cDAC described herein comprises an antibody that comprises (a) HVR—H1 comprising the amino acid sequence of SEQ ID NO: 6; (b) HVR—H2 comprising the amino acid sequence of SEQ ID NO: 7; (c) HVR—H3 comprising the amino acid sequence of SEQ ID NO: 8; (d) HVR-L1 comprising the amino acid sequence of SEQ ID NO: 3; (e) HVR-L2 comprising the amino acid sequence of SEQ ID NO: 4; and (f) HVR-L3 comprising the amino acid sequence of SEQ ID NO: 5.

In any of the above embodiments, an anti-HER2 antibody of an antibody-drug conjugate is humanized. In one embodiment, an anti-HER2 antibody of an antibody-drug conjugate comprises HVRs as in any of the above embodiments, and further comprises a human acceptor framework, e.g. a human immunoglobulin framework or a human consensus framework.

In another aspect, an anti-HER2 antibody of an antibody-drug conjugate comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 18. In certain embodiments, a VH sequence having at least 90%, 91%, 92%, 93%, 94%, 95%, 96%, 97%, 98%, or 99% identity to the amino acid sequence of SEQ ID NO: 2 contains substitutions (e.g., conservative substitutions), insertions, or deletions relative to the reference sequence, but an anti-HER2 antibody comprising that sequence retains the ability to bind to HER2. In certain embodiments, a total of 1 to 10 amino acids have been substituted, inserted and/or deleted in SEQ ID NO: 2. In certain embodiments, a total of 1 to 5 amino acids have been substituted, inserted and/or deleted in SEQ ID NO: 2. In certain embodiments, substitutions, insertions, or deletions occur in regions outside the HVRs (i.e., in the FRs). Optionally, the anti-HER2 antibody comprises the VH sequence of SEQ ID NO: 2, including post-translational modifications of that sequence. In a particular embodiment, the VH comprises one, two or three HVRs selected from: (a) HVR—H1 comprising the amino acid sequence of SEQ ID NO: 6, (b) HVR—H2 comprising the amino acid sequence of SEQ ID NO: 7, and (c) HVR—H3 comprising the amino acid sequence of SEQ ID NO: 8.

In another aspect, an anti-HER2 antibody of an antibody-drug conjugate is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 1. In certain embodiments, a VL sequence having at least 90%, 91%, 92%, 93%, 94%, 95%, 96%, 97%, 98%, or 99% identity to the amino acid sequence of SEQ ID NO: 1 contains substitutions (e.g., conservative substitutions), insertions, or deletions relative to the reference sequence, but an anti-HER2 antibody comprising that sequence retains the ability to bind to HER2. In certain embodiments, a total of 1 to 10 amino acids have been substituted, inserted and/or deleted in SEQ ID NO: 1. In certain embodiments, a total of 1 to 5 amino acids have been substituted, inserted and/or deleted in SEQ ID NO: 1. In certain embodiments, the substitutions, insertions, or deletions occur in regions outside the HVRs (i.e., in the FRs). Optionally, the anti-HER2 antibody comprises the VL sequence of SEQ ID NO: 1, including post-translational modifications of that sequence. In a particular embodiment, the VL comprises one, two or three HVRs selected from (a) HVR-L1 comprising the amino acid sequence of SEQ ID NO: 3; (b) HVR-L2 comprising the amino acid sequence of SEQ ID NO: 4; and (c) HVR-L3 comprising the amino acid sequence of SEQ ID NO: 5.

In another aspect, an antibody-drug conjugate comprising an anti-HER2 antibody is provided, wherein the antibody comprises a VH as in any of the embodiments provided above, and a VL as in any of the embodiments provided above.

In one embodiment, an antibody-drug conjugate comprising an antibody is provided, wherein the antibody comprises the VH and VL sequences in SEQ ID NO: 2 and SEQ ID NO: 1, respectively, including post-translational modifications of those sequences.

In one embodiment, an antibody-drug conjugate comprising an antibody is provided, wherein the antibody comprises the humanized 7C2.v2.2.LA (hu7C2) K149C kappa light chain sequence of SEQ ID NO: 14

In one embodiment, an antibody-drug conjugate comprising an antibody is provided, wherein the antibody comprises the Hu7C2 A118C IgG1 heavy chain sequence of SEQ ID NO: 15.

In a further aspect, provided herein are antibody-drug conjugates comprising antibodies that bind to the same epitope as an anti-HER2 antibody provided herein. For example, in certain embodiments, an immunoconjugate is provided, comprising an antibody that binds to the same epitope as an anti-HER2 antibody comprising a VH sequence of SEQ ID NO: 2 and a VL sequence of SEQ ID NO: 1, respectively.

In some embodiments, the anti-HER2 antibody of the cDACs according to any of the above embodiments is a monoclonal antibody, including a human antibody. In one embodiment, an anti-HER2 antibody of an immunoconjugate is an antibody fragment, e.g., a Fv, Fab, Fab′, scFv, diabody, or F(ab′)₂ fragment. In another embodiment, an immunoconjugate comprises an antibody that is a substantially full length antibody, e.g., an IgG1 antibody, IgG2a antibody or other antibody class or isotype as defined herein. In some embodiments, the anti-HER2 antibody is a full length antibody.

TABLE of
humanized 7C2 anti-HER2 antibody sequences

| SEQ. | | |
|---|---|---|
| ID NO. | Description | Sequence |
| 1 | Humanized | DIVMTQSPDS LAVSLGERAT INCRASQSVS GSRFTYMHWY |
| | 7C2.v2.2.LA | QQKPGQPPKL LIKYASILES GVPDRESGSG SGTDETLTIS |
| | (“hu7C2”) | SLQAEDVAVY YCQHSWEIPP WTFGQGTKVE IK |
| | light chain | |
| | variable | |
| | region | |
| 2 | Humanized | EVQLVQSGAE VKKPGASVKV SCKASGYSFT GYWMNWVRQA |
| | 7C2.v2.2.LA | PGQGLEWIGM IHPLDAEIRA NQKFRDRVTI TVDTSTSTAY |
| | (“hu7C2”) | LELSSLRSED TAVYYCARGT YDGGFEYWGQ GTLVTVSS |
| | heavy chain | |
| | variable | |
| | region | |
| 3 | hu7C2 HVR- | RASQSVSGSRFTYMH |
| | L1 | |
| 4 | hu7C2 HVR- | YASILES |
| | L2 | |
| 5 | hu7C2 HVR- | QHSWEIPPWT |
| | L3 | |
| 6 | hu7C2 HVR- | GYWMN |
| | H1 | |
| 7 | hu7C2 HVR- | MIHPLDAEIRANQKERD |
| | H2 | |
| 8 | hu7C2 HVR- | GTYDGGFEY |
| | H3 | |
| 9 | Humanized | DIVMTQSPDS LAVSLGERAT INCRASQSVS GSRFTYMHWY |
| | 7C2.v2.2.LA | QQKPGQPPKL LIKYASILES GVPDRESGSG SGTDETLTIS |
| | (hu7C2) | SLQAEDVAVY YCQHSWEIPP WTFGQGTKVE IKRTVAAPSV |
| | kappa light | FIFPPSDEQL KSGTASVVCL LNNFYPREAK VQWKVDNALQ |
| | chain | SGNSQESVTE QDSKDSTYSL SSTLTLSKAD YEKHKVYACE |
| | | VTHQGLSSPV TKSENRGEC |
| 10 | Humanized | EVQLVQSGAE VKKPGASVKV SCKASGYSFT GYWMNWVRQA |
| | 7C2.v2.2.LA | PGQGLEWIGM IHPLDAEIRA NQKERDRVTI TVDTSTSTAY |
| | (hu7C2) | LELSSLRSED TAVYYCARGT YDGGFEYWGQ GTLVTVSSAS |
| | IgG1 heavy | TKGPSVFPLA PSSKSTSGGT AALGCLVKDY FPEPVTVSWN |
| | chain | SGALTSGVHT FPAVLQSSGL YSLSSVVTVP SSSLGTQTYI |
| | | CNVNHKPSNT KVDKKVEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSREEMT |
| | | KNQVSLTCLV KGFYPSDIAV EWESNGQPEN NYKTTPPVLD |
| | | SDGSFFLYSK STVDKSRWQQ GNVFSCSVMH EALHNHYTQK |
| | | SLSLSPGK |
| 11 | Hu7C2. | MIHPMDSEIRANQKERD |
| | v2.1.S53M | |
| | HVR-H2 | |
| 12 | Hu7C2. | MIHPLDSEIRANQKERD |
| | v2.1.S53L | |
| | HVR-H2 | |
| 13 | Hu7C2. | GTYDGGFKY |
| | v2.1.E101K | |
| | HVR-H3 | |
| 14 | Humanized | DIVMTQSPDS LAVSLGERAT INCRASQSVS GSRFTYMHWY |
| | 7C2.v2.2.LA | QQKPGQPPKL SIKYASILES GVPDRESGSG SGTDETLTIS |
| | (hu7C2) | SLQAEDVAVY YCQHSWEIPP WTFGQGTKVE IKRTVAAPSV |
| | K149C kappa | FIFPPSDEQL KSGTASVVCL LNNFYPREAK VQWCVDNALQ |
| | light chain | SGNSQESVTE QDSKDSTYSL SSTLTLSKAD YEKHKVYACE |
| | | VTHQGLSSPV TKSFNRGEC |
| 15 | Humanized | EVQLVQSGAE VKKPGASVKV SCKASGYSFT GYWMNWVRQA |
| | 7C2.v2.2.LA | PGQGLEWIGM IHPLDAEIRA NQKFRDRVTI TVDTSTSTAY |
| | (hu7C2) | LELSSLRSED TAVYYCARGT YDGGFEYWGQ GTLVTVSSCS |
| | A118C IgG1 | TKGPSVFPLA PSSKSTSGGT AALGCLVKDY FPEPVTVSWN |
| | heavy chain | SGALTSGVHT FPAVLQSSGL YSLSSVVTVP SSSLGTQTYI |
| | | CNVNHKPSNT KVDKKVEPKS CDKTHTCPPC PAPELLGGPS |
| | | VFLFPPKPKD TLMISRTPEV TCVVVDVSHE DPEVKENWYV |
| | | DGVEVHNAKT KPREEQYNST YRVVSVLTVL HQDWLNGKEY |
| | | KCKVSNKALP APIEKTISKA KGQPREPQVY TLPPSREEMT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |

### Anti-CD33 Antibodies

The anti-CD33 antibody 15G15.33 of cDAC in Table 4 and Table 5 comprises three light chain hypervariable regions (HVR-L1, HVR-L2 and HVR-L3) and three heavy chain hypervariable regions (HVR—H1, HVR—H2 and HVR—H3), SEQ TD NO:16-21.

HVR-L1
(SEQ ID NO: 16)
RSSQSLLHSNGYNYLD
HVR-L2
(SEQ ID NO: 17)
LGVNSVS
HVR-L3
(SEQ ID NO: 18)
MQALQTPWT
HVR-H1
(SEQ ID NO: 19)
NHAIS
HVR-H2
(SEQ ID NO: 20)
GIIPIFGTANYAQKFQG
HVR-H3
(SEQ ID NO: 21)
EWADVED

The anti-CD33 antibody 15G15.33 of cDAC in Table 4 and Table 5 comprises the light chain variable region of SEQ ID NO:22 and/or the heavy chain variable region of SEQ ID NO:23.

(SEQ ID NO: 23)
EIVLTQSPLSLPVTPGEPASISCRSSQSLLHSNGYNYLDWY
LQKPGQSPQLLIYLGVNSVSGVPDRFSGSGSGTDFTLKISR
VEAEDVGVYYCMQALQTPWTFGQGTKVEIK
(SEQ ID NO: 22)
QVQLVQSGAEVKKPGSSVKVSCKASGGIFSNHAISWVRQAP
GQGLEWMGGIIPIFGTANYAQKFQGRVTITADESTSTAFME
LSSLRSEDTAVYYCAREWADVEDIWGQGTMVTVSS

The anti-CD33 antibody 9C3 comprises three light chain hypervariable regions (HVR-L1, HVR-L2 and HVR-L3) and three heavy chain hypervariable regions (HVR—H1, HVR—H2 and HVR—H3), SEQ ID NO:24-29, and following VL and VH sequences SEQ ID NO:30-37.

9C3-HVR L1
(SEQ ID NO: 24)
RASQGIRNDLG
9C3-HVR L2
(SEQ ID NO: 25)
AASSLQS
9C3-HVR L3
(SEQ ID NO: 26)
LQHNSYPWT
9C3-HVR H1
(SEQ ID NO: 27)
GNYMS
9C3-HVR H2
(SEQ ID NO: 28)
LIYSGDSTYYADSVKG
9C3-HVR H3
(SEQ ID NO: 29)
DGYYVSDMVV
9C3 V_{L}
(SEQ ID NO: 30)
DIQMTQSPSSLSASVGDRVTITCRASQGIRNDLGWYQQKPGKAPK
RLIYAASSLQSGVPSRESGSGSGTEFTLTISSLQPEDFATYYCLQ
HNSYPWTFGQGTKLEIK
9C3 V_{H}
(SEQ ID NO: 31)
EVQLVESGGALIQPGGSLRLSCVASGFTISGNYMSWVRQAPGKGL
EWVSLIYSGDSTYYADSVKGRENISRDISKNTVYLQMNSLRVEDT
AVYYCVRDGYYVSDMVVWGKGTTVTVSS
9C3.2 V_{L}
(SEQ ID NO: 32)
DIQMTQSPSSLSASVGDRVTITCRASQGIRNDLGWYQQKPGKAPK
RLIYAASSLQSGVPSRESGSGSGTEFTLTISSLQPEDFATYYCLQ
HNSYPWTFGQGTKLEIK
9C3.2 VH
(SEQ ID NO: 33)
EVQLVESGGALIQPGGSLRLSCVASGFTISGNYMSWVRQAPGKGL
EWVSLIYSGDSTYYADSVKGRFTISRDISKNTVYLQMNSLRVEDT
AVYYCVRDGYYVSDMVVWGKGTTVTVSS
9C3.3 V_{L}
(SEQ ID NO: 34)
DIQMTQSPSSLSASVGDRVTITCRASQGIRNDLGWYQQKPGKAPK
RLIYAASSLQSGVPSRFSGSGSGTEFTLTISSLQPEDFATYYCLQ
HNSYPWTFGQGTKLEIK
9C3.3 V_{H}
(SEQ ID NO: 35)
EVQLVESGGALIQPGGSLRLSCVASGFTISGNYMSWVRQAPGKGL
EWVSLIYSGDSTYYADSVKGRESISRDISKNTVYLQMNSLRVEDT
AVYYCVRDGYYVSDMVVWGKGTTVTVSS
9C3.4 V_{L}
(SEQ ID NO: 36)
DIQMTQSPSSLSASVGDRVTITCRASQGIRNDLGWYQQKPGKAPK
RLIYAASSLQSGVPSRFSGSGSGTEFTLTISSLQPEDFATYYCLQ
HNSYPWTFGQGTKLEIK
9C3.4 V_{H}
(SEQ ID NO: 37)
EVQLVESGGALIQPGGSLRLSCVASGFTISGNYMSWVRQAPGKGL
EWVSLIYSGDSTYYADSVKGRFAISRDISKNTVYLQMNSLRVEDT
AVYYCVRDGYYVSDMVVWGKGTTVTVSS

In some embodiments, the cDAC described herein comprises an anti-CD33 antibody comprising at least one, two, three, four, five, or six HVRs selected from (a) HVR—H1 comprising the amino acid sequence of SEQ ID NO:27; (b) HVR—H2 comprising the amino acid sequence of SEQ ID NO:28; (c) HVR—H3 comprising the amino acid sequence of SEQ ID NO:29; (d) HVR-L1 comprising the amino acid sequence of SEQ ID NO:24; (e) HVR-L2 comprising the amino acid sequence of SEQ ID NO:25; and (f) HVR-L3 comprising the amino acid sequence of SEQ ID NO:26.

In one aspect the cDAC described herein comprises an antibody comprising at least one, at least two, or all three VH HVR sequences selected from (a) HVR—H1 comprising the amino acid sequence of SEQ ID NO:27; (b) HVR—H2 comprising the amino acid sequence of SEQ ID NO:28; and (c) HVR—H3 comprising the amino acid sequence of SEQ ID NO:29. In one embodiment, the antibody comprises HVR—H3 comprising the amino acid sequence of SEQ ID NO:29. In another embodiment, the antibody comprises HVR—H3 comprising the amino acid sequence of SEQ ID NO:29 and HVR-L3 comprising the amino acid sequence of SEQ ID NO:26. In a further embodiment, the antibody comprises HVR—H3 comprising the amino acid sequence of SEQ ID NO:29, HVR-L3 comprising the amino acid sequence of SEQ ID NO:26, and HVR—H2 comprising the amino acid sequence of SEQ ID NO:28. In a further embodiment, the antibody comprises (a) HVR—H1 comprising the amino acid sequence of SEQ ID NO:27; (b) HVR—H2 comprising the amino acid sequence of SEQ ID NO:28; and (c) HVR—H3 comprising the amino acid sequence of SEQ ID NO:29.

In another aspect, the cDAC described herein comprises at least one, at least two, or all three VL HVR sequences selected from (a) HVR-L1 comprising the amino acid sequence of SEQ ID NO:24; (b) HVR-L2 comprising the amino acid sequence of SEQ ID NO:25; and (c) HVR-L3 comprising the amino acid sequence of SEQ ID NO:26. In one embodiment, the antibody comprises (a) HVR-L1 comprising the amino acid sequence of SEQ ID NO:24; (b) HVR-L2 comprising the amino acid sequence of SEQ ID NO:25; and (c) HVR-L3 comprising the amino acid sequence of SEQ ID NO:26.

In another aspect, the anti-CD33 antibody comprises (a) a VH domain comprising at least one, at least two, or all three VH HVR sequences selected from (i) HVR—H1 comprising the amino acid sequence of SEQ ID NO:27, (ii) HVR—H2 comprising the amino acid sequence of SEQ ID NO:28, and (iii) HVR—H3 comprising an amino acid sequence selected from SEQ ID NO:29; and (b) a VL domain comprising at least one, at least two, or all three VL HVR sequences selected from (i) HVR-L1 comprising the amino acid sequence of SEQ ID NO:24, (ii) HVR-L2 comprising the amino acid sequence of SEQ ID NO:25, and (c) HVR-L3 comprising the amino acid sequence of SEQ ID NO:26.

In another aspect, the cDAC described herein comprises: (a) HVR—H1 comprising the amino acid sequence of SEQ ID NO:27; (b) HVR—H2 comprising the amino acid sequence of SEQ ID NO:28; (c) HVR—H3 comprising the amino acid sequence of SEQ ID NO:29; (d) HVR-L1 comprising the amino acid sequence of SEQ ID NO:24; (e) HVR-L2 comprising the amino acid sequence of SEQ ID NO:25; and (f) HVR-L3 comprising the amino acid sequence of SEQ ID NO:26.

In any of the above embodiments, an anti-CD33 antibody is humanized. In one embodiment, an anti-CD33 antibody comprises HVRs as in any of the above embodiments, and further comprises a human acceptor framework, e.g. a human immunoglobulin framework or a human consensus framework. In certain embodiments, the human acceptor framework is the human VL kappa I consensus (VL_{KI}) framework and/or the VH framework VH₁. In certain embodiments, the human acceptor framework is the human VL kappa I consensus (VL_{KI}) framework and/or the VH framework VH₁ comprising any one of the following mutations.

In another aspect, an anti-CD33 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:31, SEQ ID NO:33, SEQ ID NO:35, and/or SEQ ID NO:37. In certain embodiments, a VH sequence having at least 90%, 91%, 92%, 93%, 94%, 95%, 96%, 97%, 98%, or 99% identity to the amino acid sequence of SEQ ID NO:31, SEQ ID NO:33, SEQ ID NO:35, and/or SEQ ID NO:37 contains substitutions (e.g., conservative substitutions), insertions, or deletions relative to the reference sequence, but an anti-CD33 antibody comprising that sequence retains the ability to bind to CD33. In certain embodiments, a total of 1 to 10 amino acids have been substituted, inserted and/or deleted in SEQ ID NO:31, SEQ ID NO:33, SEQ ID NO:35, and/or SEQ ID NO:37. In certain embodiments, a total of 1 to 5 amino acids have been substituted, inserted and/or deleted in SEQ ID NO:35, SEQ ID NO:33, SEQ ID NO:35, and/or SEQ ID NO:37. In certain embodiments, substitutions, insertions, or deletions occur in regions outside the HVRs (i.e., in the FRs). Optionally, the anti-CD33 antibody comprises the VH sequence of SEQ ID NO:31, SEQ ID NO:33, SEQ ID NO:35, and/or SEQ ID NO:37, including post-translational modifications of that sequence. In a particular embodiment, the VH comprises one, two or three HVRs selected from: (a) HVR—H1 comprising the amino acid sequence of SEQ ID NO:27, (b) HVR—H2 comprising the amino acid sequence of SEQ ID NO:28, and (c) HVR—H3 comprising the amino acid sequence of SEQ ID NO:29.

In another aspect, an anti-CD33 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:30, SEQ ID NO:32, SEQ ID NO:34, and/or SEQ ID NO:36. In certain embodiments, a VL sequence having at least 90%, 91%, 92%, 93%, 94%, 95%, 96%, 97%, 98%, or 99% identity to the amino acid sequence of SEQ ID NO:30, SEQ ID NO:32, SEQ ID NO:34, and/or SEQ ID NO:36 contains substitutions (e.g., conservative substitutions), insertions, or deletions relative to the reference sequence, but an anti-CD33 antibody comprising that sequence retains the ability to bind to CD33. In certain embodiments, a total of 1 to 10 amino acids have been substituted, inserted and/or deleted in SEQ ID NO:30, SEQ ID NO:32, SEQ ID NO:34, and/or SEQ ID NO:36. In certain embodiments, a total of 1 to 5 amino acids have been substituted, inserted and/or deleted in SEQ ID NO:30, SEQ ID NO:32, SEQ ID NO:34, and/or SEQ ID NO:36. In certain embodiments, the substitutions, insertions, or deletions occur in regions outside the HVRs (i.e., in the FRs). Optionally, the anti-CD33 antibody comprises the VL sequence of SEQ ID NO:30, SEQ ID NO:32, SEQ ID NO:34, and/or SEQ ID NO:36, including post-translational modifications of that sequence. In a particular embodiment, the VL comprises one, two or three HVRs selected from (a) HVR-L1 comprising the amino acid sequence of SEQ ID NO:24; (b) HVR-L2 comprising the amino acid sequence of SEQ ID NO:25; and (c) HVR-L3 comprising the amino acid sequence of SEQ ID NO:26.

In another aspect, an anti-CD33 antibody is provided, wherein the antibody comprises a VH as in any of the embodiments provided above, and a VL as in any of the embodiments provided above.

In one embodiment, the antibody comprises the VH and VL sequences in SEQ ID NO:31 and SEQ ID NO:30, respectively, including post-translational modifications of those sequences. In one embodiment, the antibody comprises the VH and VL sequences in SEQ ID NO:33 and SEQ ID NO:32, respectively, including post-translational modifications of those sequences. In one embodiment, the antibody comprises the VH and VL sequences in SEQ ID NO:35 and SEQ ID NO:34, respectively, including post-translational modifications of those sequences. In one embodiment, the antibody comprises the VH and VL sequences in SEQ ID NO:37 and SEQ ID NO:36, respectively, including post-translational modifications of those sequences.

In a further aspect, provided herein are antibodies that bind to the same epitope as an anti-CD33 antibody provided herein. For example, in certain embodiments, an antibody is provided that binds to the same epitope as an anti-CD33 antibody comprising a VH sequence of SEQ ID NO:31, SEQ ID NO:33, SEQ ID NO:35, and/or SEQ ID NO:37 and a VL sequence of SEQ ID NO:30, SEQ ID NO:32, SEQ ID NO:34, and/or SEQ ID NO:36, respectively.

In some embodiments, the anti-CD33 antibody is a monoclonal antibody, including a human antibody. In one embodiment, an anti-CD33 antibody is an antibody fragment, e.g., a Fv, Fab, Fab′, scFv, diabody, or F(ab′)₂ fragment. In another embodiment, the antibody is a substantially full length antibody, e.g., an IgG1 antibody, IgG2a antibody or other antibody class or isotype as defined herein. In some embodiments, the anti-CD33 antibody is a full length antibody.

In a further aspect, an anti-CD33 antibody according to any of the above embodiments may incorporate any of the features, singly or in combination, as described below.

### Cysteine Engineered Antibody Variants

In certain embodiments, it may be desirable to create cysteine engineered antibodies, e.g., a “THIOMAB™” or TDC, in which one or more residues of an antibody are substituted with cysteine residues. In particular embodiments, the substituted residues occur at sites of the antibody that are available for conjugation. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: K149 (Kabat numbering) of the light chain; V205 (Kabat numbering) of the light chain; A118 (EU numbering) of the heavy chain; A140 (EU numbering) of the heavy chain; L174 (EU numbering) of the heavy chain; Y373 (EU numbering) of the heavy chain; and S400 (EU numbering) of the heavy chain Fc region. In specific embodiments, the antibodies described herein comprise the HC-A140C (EU numbering) cysteine substitution. In specific embodiments, the antibodies described herein comprise the LC-K149C (Kabat numbering) cysteine substitution. In specific embodiments, the antibodies described herein comprise the HC-A118C (EU numbering) cysteine substitution. Cysteine engineered antibodies may be generated as described, e.g., in U.S. Pat. No. 7,521,541.

In certain embodiments, the antibody comprises one of the following heavy chain cysteine substitutions:

| | | EU | Kabat |
|---|---|---|---|
| Chain | | Mutation | Mutation |
| (HC/LC) | Residue | Site # | Site # |
| HC | T | 114 | 110 |
| HC | A | 140 | 136 |
| HC | L | 174 | 170 |
| HC | L | 179 | 175 |
| HC | T | 187 | 183 |
| HC | T | 209 | 205 |
| HC | V | 262 | 258 |
| HC | G | 371 | 367 |
| HC | Y | 373 | 369 |
| HC | E | 382 | 378 |
| HC | S | 424 | 420 |
| HC | N | 434 | 430 |
| HC | Q | 438 | 434 |

In certain embodiments, the antibody comprises one of the following light chain cysteine substitutions:

| | | EU | Kabat |
|---|---|---|---|
| Chain | | Mutation | Mutation |
| (HC/LC) | Residue | Site # | Site # |
| LC | I | 106 | 106 |
| LC | R | 108 | 108 |
| LC | R | 142 | 142 |
| LC | K | 149 | 149 |
| LC | V | 205 | 205 |

A nonlimiting exemplary hu7C2.v2.2.LA light chain (LC) K149C THIOMAB™ has the heavy chain and light chain amino acid sequences of SEQ ID NOs: 10 and 14, respectively. A nonlimiting exemplary hu7C2.v2.2.LA heavy chain (HC) A118C THIOMAB™ has the heavy chain and light chain amino acid sequences of SEQ ID NOs: 15 and 9, respectively.

### Antibody Targets

In some embodiments, the antibody of a cereblon degrader antibody conjugate (cDAC) is capable of binding to one or more tumor-associated antigens (TAA), cell-surface receptors, and immune-specific antigens to confer specificity to the targeting of the cereblon degrader antibody conjugate and enable safe and systemic delivery of an active drug moiety.

Certain tumor-associated antigens are known in the art, and can be prepared for use in generating antibodies using methods and information which are well known in the art. In attempts to discover effective cellular targets for cancer diagnosis and therapy, researchers have sought to identify transmembrane or otherwise tumor-associated polypeptides that are specifically expressed on the surface of one or more particular type(s) of cancer cell as compared to on one or more normal non-cancerous cell(s). Often, such tumor-associated polypeptides are more abundantly expressed on the surface of the cancer cells as compared to on the surface of the non-cancerous cells. The identification of such tumor-associated cell surface antigen polypeptides allows more specificity in targeting cancer cells for destruction via antibody-based therapies.

Examples of TAAs include, but are not limited to, those listed below including (1)-(55). For convenience, information relating to these antigens, all of which are known in the art, is listed below and includes names, alternative names, Genbank accession numbers and primary reference(s), following nucleic acid and protein sequence identification conventions of the National Center for Biotechnology Information (NCBI). Nucleic acid and protein sequences corresponding to TAAs listed below including (1)-(55) are available in public databases such as GenBank. TAAs targeted by antibodies include all amino acid sequence variants and isoforms possessing at least about 70%, 80%, 85%, 90%, or 95% sequence identity relative to the sequences identified in the cited references, and/or which exhibit substantially the same biological properties or characteristics as a TAA having a sequence found in the cited references. For example, a TAA having a variant sequence generally is able to bind specifically to an antibody that binds specifically to the TAA with the corresponding sequence listed. The sequences and disclosure in the reference specifically recited herein are expressly incorporated by reference.

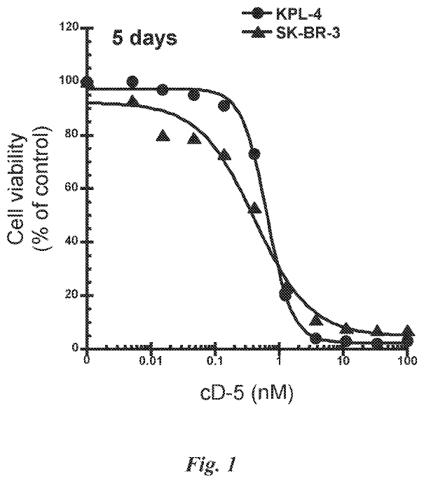

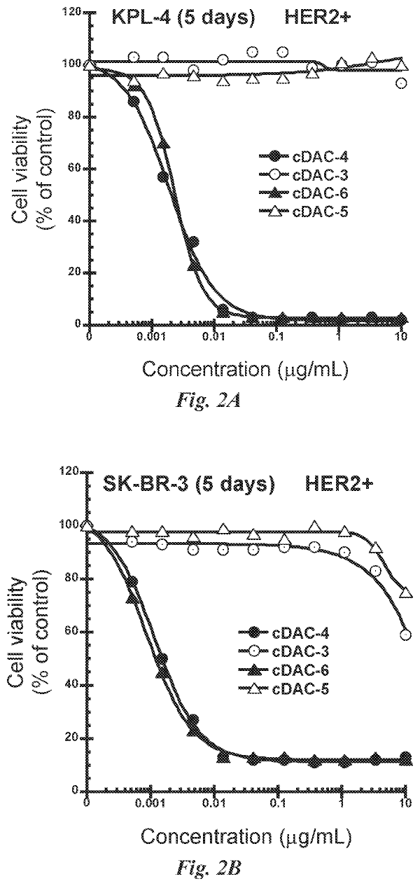

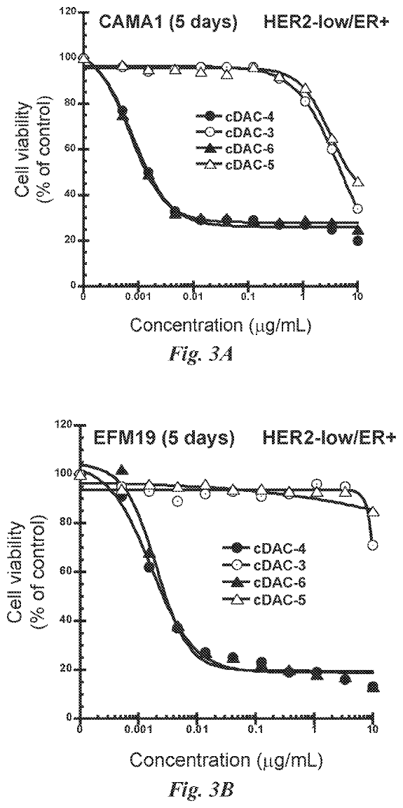

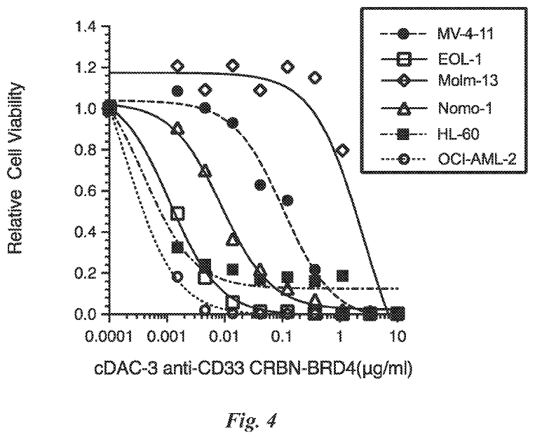

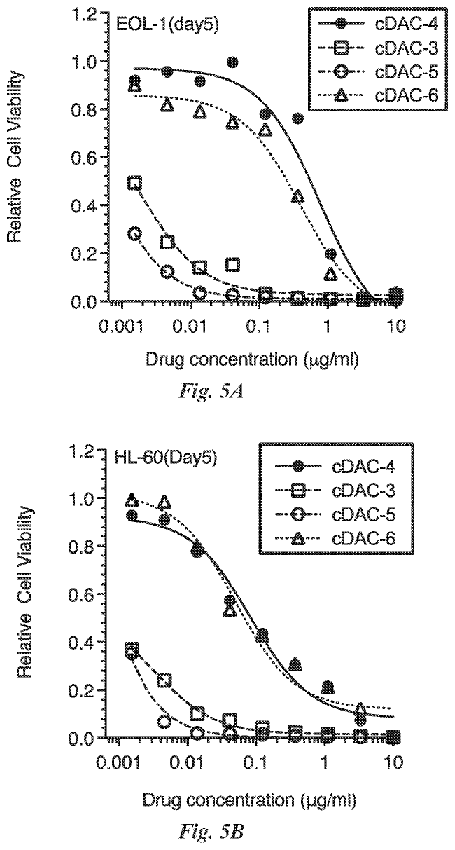

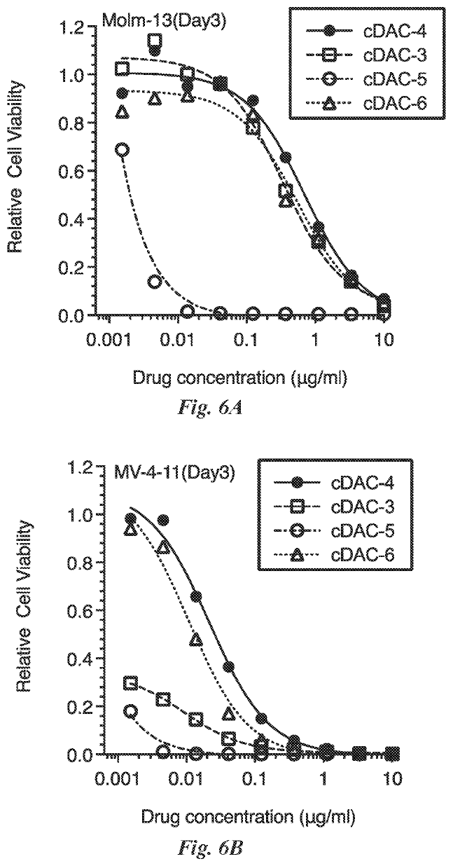

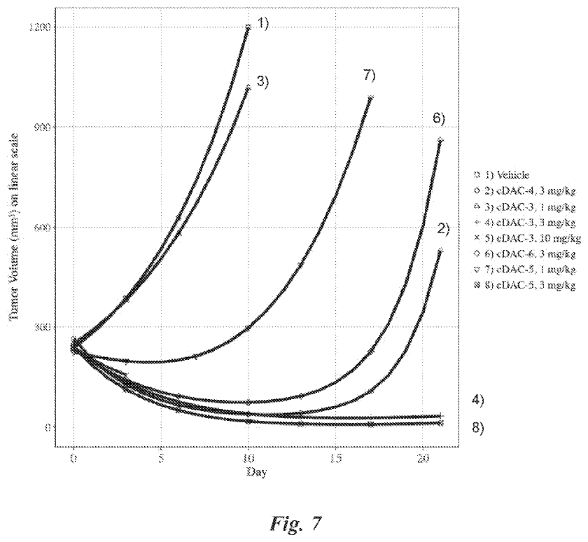

The sequences and disclosure in the references specifically recited herein are expressly incorporated by reference.
(1) BMPR1B (bone morphogenetic protein receptor-type IB, Genbank accession no. NM_001203) ten Dijke, P., et al. Science 264 (5155):101-104 (1994), Oncogene 14 (11):1377-1382 (1997)); WO2004063362 (Claim 2); WO2003042661 (Claim 12); US2003134790-A1 (Page 38-39); WO2002102235 (Claim 13; Page 296); WO2003055443 (Page 91-92); WO200299122 (Example 2; Page 528-530); WO2003029421 (Claim 6); WO2003024392 (Claim 2; FIG. 112); WO200298358 (Claim 1; Page 183); WO200254940 (Page 100-101); WO200259377 (Page 349-350); WO200230268 (Claim 27; Page 376); WO200148204 (Example; FIG. 4) NP_001194 bone morphogenetic protein receptor, type IB/pid=NP_001194.1—Cross-references: MIM:603248; NP_001194.1; AY065994.
(2) E16 (LAT1, SLC7A5, Genbank accession no. NM_003486) Biochem. Biophys. Res. Commun. 255 (2), 283-288 (1999), Nature 395 (6699):288-291 (1998), Gaugitsch, H. W., et al. (1992) J. Biol. Chem. 267 (16):11267-11273); WO2004048938 (Example 2); WO2004032842 (Example IV); WO2003042661 (Claim 12); WO2003016475 (Claim 1); WO200278524 (Example 2); WO200299074 (Claim 19; Page 127-129); WO200286443 (Claim 27; Pages 222, 393); WO2003003906 (Claim 10; Page 293); WO200264798 (Claim 33; Page 93-95); WO200014228 (Claim 5; Page 133-136); US2003224454 (FIG. 3); WO2003025138 (Claim 12; Page 150); NP_003477 solute carrier family 7 (cationic amino acid transporter, y+ system), member 5/pid=NP_003477.3—Homo sapiens Cross-references: MIM:600182; NP_003477.3; NM_015923; NM_003486_1.
(3) STEAP1 (six transmembrane epithelial antigen of prostate, Genbank accession no. NM_012449) Cancer Res. 61 (15), 5857-5860 (2001), Hubert, R. S., et al. (1999) Proc. Natl. Acad. Sci. U.S.A. 96 (25):14523-14528); WO2004065577 (Claim 6); WO2004027049 (FIG. 1L); EP1394274 (Example 11); WO2004016225 (Claim 2); WO2003042661 (Claim 12); US2003157089 (Example 5); US2003185830 (Example 5); US2003064397 (FIG. 2); WO200289747 (Example 5; Page 618-619); WO2003022995 (Example 9; FIG. 13A, Example 53; Page 173, Example 2; FIG. 2A); NP_036581 six transmembrane epithelial antigen of the prostate Cross-references: MIM:604415; NP_036581.1; NM_012449_1.
(4) 0772P (CA125, MUC16, Genbank accession no. AF361486) J. Biol. Chem. 276 (29):27371-27375 (2001)); WO2004045553 (Claim 14); WO200292836 (Claim 6; FIG. 12); WO200283866 (Claim 15; Page 116-121); US2003124140 (Example 16); U.S. Pat. No. 798959. Cross-references: GI:34501467; AAK74120.3; AF361486_1.
(5) MPF (MPF, MSLN, SMR, megakaryocyte potentiating factor, mesothelin, Genbank accession no. NM_005823) Yamaguchi, N., et al. Biol. Chem. 269 (2), 805-808 (1994), Proc. Natl. Acad. Sci. U.S.A. 96 (20):11531-11536 (1999), Proc. Natl. Acad. Sci. U.S.A. 93 (1):136-140 (1996), J. Biol. Chem. 270 (37):21984-21990 (1995)); WO2003101283 (Claim 14); (WO2002102235 (Claim 13; Page 287-288); WO2002101075 (Claim 4; Page 308-309); WO200271928 (Page 320-321); WO9410312 (Page 52-57); Cross-references: MIM:601051; NP_005814.2; NM_005823_1.
(6) Napi3b (NAPI-3B, NPTIIb, SLC34A2, solute carrier family 34 (sodium phosphate), member 2, type II sodium-dependent phosphate transporter 3b, Genbank accession no. NM_006424) J. Biol. Chem. 277 (22):19665-19672 (2002), Genomics 62 (2):281-284 (1999), Field, J. A., et al. (1999) Biochem. Biophys. Res. Commun. 258 (3):578-582); WO2004022778 (Claim 2); EP1394274 (Example 11); WO2002102235 (Claim 13; Page 326); EP875569 (Claim 1; Page 17-19); WO200157188 (Claim 20; Page 329); WO2004032842 (Example IV); WO200175177 (Claim 24; Page 139-140); Cross-references: MIM:604217; NP_006415.1; NM_006424_1.
(7) Sema 5b (FLJ10372, KIAA1445, Mm.42015, SEMA5B, SEMAG, Semaphorin 5b H log, sema domain, seven thrombospondin repeats (type 1 and type 1-like), transmembrane domain (TM) and short cytoplasmic domain, (semaphorin) 5B, Genbank accession no. AB040878) Nagase T., et al. (2000) DNA Res. 7 (2):143-150); WO2004000997 (Claim 1); WO2003003984 (Claim 1); WO200206339 (Claim 1; Page 50); WO200188133 (Claim 1; Page 41-43, 48-58); WO2003054152 (Claim 20); WO2003101400 (Claim 11); Accession: Q9P283; EMBL; AB040878; BAA95969.1. Genew; HGNC:10737.
(8) PSCA hlg (2700050C12Rik, C530008O16Rik, RIKEN cDNA 2700050C12, RIKEN cDNA 2700050C12 gene, Genbank accession no. AY358628); Ross et al. (2002) Cancer Res. 62:2546-2553; US2003129192 (Claim 2); US2004044180 (Claim 12); US2004044179 (Claim 11); US2003096961 (Claim 11); US2003232056 (Example 5); WO2003105758 (Claim 12); US2003206918 (Example 5); EP1347046 (Claim 1); WO2003025148 (Claim 20); Cross-references: GI:37182378; AAQ88991.1; AY358628_1.
(9) ETBR (Endothelin type B receptor, Genbank accession no. AY275463); Nakamuta M., et al. Biochem. Biophys. Res. Commun. 177, 34-39, 1991; Ogawa Y., et al. Biochem. Biophys. Res. Commun. 178, 248-255, 1991; Arai H., et al. Jpn. Circ. J. 56, 1303-1307, 1992; Arai H., et al. J. Biol. Chem. 268, 3463-3470, 1993; Sakamoto A., Yanagisawa M., et al. Biochem. Biophys. Res. Commun. 178, 656-663, 1991; Elshourbagy N. A., et al. J. Biol. Chem. 268, 3873-3879, 1993; Haendler B., et al. J. Cardiovasc. Pharmacol. 20, sl-S4, 1992; Tsutsumi M., et al. Gene 228, 43-49, 1999; Strausberg R. L., et al. Proc. Natl. Acad. Sci. U.S.A. 99, 16899-16903, 2002; Bourgeois C., et al. J. Clin. Endocrinol. Metab. 82, 3116-3123, 1997; Okamoto Y., et al. Biol. Chem. 272, 21589-21596, 1997; Verheij J. B., et al. Am. J. Med. Genet. 108, 223-225, 2002; Hofstra R. M. W., et al. Eur. J. Hum. Genet. 5, 180-185, 1997; Puffenberger E. G., et al. Cell 79, 1257-1266, 1994; Attie T., et al., Hum. Mol. Genet. 4, 2407-2409, 1995; Auricchio A., et al. Hum. Mol. Genet. 5:351-354, 1996; Amiel J., et al. Hum. Mol. Genet. 5, 355-357, 1996; Hofstra R. M. W., et al. Nat. Genet. 12, 445-447, 1996; Svensson P. J., et al. Hum. Genet. 103, 145-148, 1998; Fuchs S., et al. Mol. Med. 7, 115-124, 2001; Pingault V., et al. (2002) Hum. Genet. 111, 198-206; WO2004045516 (Claim 1); WO2004048938 (Example 2); WO2004040000 (Claim 151); WO2003087768 (Claim 1); WO2003016475 (Claim 1); WO2003016475 (Claim 1); WO200261087 (FIG. 1); WO2003016494 (FIG. 6); WO2003025138 (Claim 12; Page 144); WO200198351 (Claim 1; Page 124-125); EP522868 (Claim 8; FIG. 2); WO200177172 (Claim 1; Page 297-299); US2003109676; U.S. Pat. No. 6,518,404 (FIG. 3); U.S. Pat. No. 5,773,223 (Claim 1a; Col 31-34); WO2004001004.
(10) MSG783 (RNF124, hypothetical protein FLJ20315, Genbank accession no. NM_017763); WO2003104275 (Claim 1); WO2004046342 (Example 2); WO2003042661 (Claim 12); WO2003083074 (Claim 14; Page 61); WO2003018621 (Claim 1); WO2003024392 (Claim 2; FIG. 93); WO200166689 (Example 6); Cross-references: LocusID:54894; NP_060233.2; NM_017763_1.
(11) STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein, Genbank accession no. AF455138) Lab. Invest. 82 (11):1573-1582 (2002)); WO2003087306; US2003064397 (Claim 1; FIG. 1); WO200272596 (Claim 13; Page 54-55); WO200172962 (Claim 1; FIG. 4B); WO2003104270 (Claim 11); WO2003104270 (Claim 16); US2004005598 (Claim 22); WO2003042661 (Claim 12); US2003060612 (Claim 12; FIG. 10); WO200226822 (Claim 23; FIG. 2); WO200216429 (Claim 12; FIG. 10); Cross-references: GI:22655488; AAN04080.1; AF455138_1.
(12) TrpM4 (BR22450, FLJ20041, TRPM4, TRPM4B, transient receptor potential cation channel, subfamily M, member 4, Genbank accession no. NM_017636) Xu, X. Z., et al. Proc. Natl. Acad. Sci. U.S.A. 98 (19):10692-10697 (2001), Cell 109 (3):397-407 (2002), J. Biol. Chem. 278 (33):30813-30820 (2003)); US2003143557 (Claim 4); WO200040614 (Claim 14; Page 100-103); WO200210382 (Claim 1; FIG. 9A); WO2003042661 (Claim 12); WO200230268 (Claim 27; Page 391); US2003219806 (Claim 4); WO200162794 (Claim 14; FIG. 1A-D); Cross-references: MIM:606936; NP_060106.2; NM_017636_1.
(13) CRIPTO (CR, CR1, CRGF, CRIPTO, TDGF1, teratocarcinoma-derived growth factor, Genbank accession no. NP_003203 or NM_003212) Ciccodicola, A., et al. EMBO J. 8 (7):1987-1991 (1989), Am. J. Hum. Genet. 49 (3):555-565 (1991)); US2003224411 (Claim 1); WO2003083041 (Example 1); WO2003034984 (Claim 12); WO200288170 (Claim 2; Page 52-53); WO2003024392 (Claim 2; FIG. 58); WO200216413 (Claim 1; Page 94-95, 105); WO200222808 (Claim 2; FIG. 1); U.S. Pat. No. 5,854,399 (Example 2; Col 17-18); U.S. Pat. No. 5,792,616 (FIG. 2); Cross-references: MIM:187395; NP_003203.1; NM_003212_1.
(14) CD21 (CR2 (Complement receptor 2) or C3DR (C3d/Epstein Barr virus receptor) or Hs.73792 Genbank accession no. M26004) Fujisaku et al. (1989) J. Biol. Chem. 264 (4):2118-2125); Weis J. J., et al. J. Exp. Med. 167, 1047-1066, 1988; Moore M., et al. Proc. Natl. Acad. Sci. U.S.A. 84, 9194-9198, 1987; Barel M., et al. Mol. Immunol. 35, 1025-1031, 1998; Weis J. J., et al. Proc. Natl. Acad. Sci. U.S.A. 83, 5639-5643, 1986; Sinha S. K., et al. (1993) J. Immunol. 150, 5311-5320; WO2004045520 (Example 4); US2004005538 (Example 1); WO2003062401 (Claim 9); WO2004045520 (Example 4); WO9102536 (FIGS. 9.1-9.9); WO2004020595 (Claim 1); Accession: P20023; Q13866; Q14212; EMBL; M26004; AAA35786.1.
(15) CD79b (CD79B, CD79β, IGb (immunoglobulin-associated beta), B29, Genbank accession no. NM_000626 or 11038674) Proc. Natl. Acad. Sci. U.S.A. (2003) 100 (7):4126-4131, Blood (2002) 100 (9):3068-3076, Muller et al. (1992) Eur. J. Immunol. 22 (6):1621-1625); WO2004016225 (claim 2, FIG. 140); WO2003087768, US2004101874 (claim 1, page 102); WO2003062401 (claim 9); WO200278524 (Example 2); US2002150573 (claim 5, page 15); U.S. Pat. No. 5,644,033; WO2003048202 (claim 1, pages 306 and 309); WO 99/558658, U.S. Pat. No. 6,534,482 (claim 13, FIG. 17A/B); WO200055351 (claim 11, pages 1145-1146); Cross-references: MIM:147245; NP 000617.1; NM_000626_1.
(16) FcRH2 (IFGP4, IRTA4, SPAP1A (SH2 domain containing phosphatase anchor protein 1a), SPAP1B, SPAP1C, Genbank accession no. NM_030764, AY358130) Genome Res. 13 (10):2265-2270 (2003), Immunogenetics 54 (2):87-95 (2002), Blood 99 (8):2662-2669 (2002), Proc. Natl. Acad. Sci. U.S.A. 98 (17):9772-9777 (2001), Xu, M. J., et al. (2001) Biochem. Biophys. Res. Commun. 280 (3):768-775; WO2004016225 (Claim 2); WO2003077836; WO200138490 (Claim 5; FIG. 18D-1-18D-2); WO2003097803 (Claim 12); WO2003089624 (Claim 25); Cross-references: MIM:606509; NP_110391.2; NM_030764_1.
(17) HER2 (ErbB2, Genbank accession no. M11730) Coussens L., et al. Science (1985) 230(4730):1132-1139); Yamamoto T., et al. Nature 319, 230-234, 1986; Semba K., et al. Proc. Natl. Acad. Sci. U.S.A. 82, 6497-6501, 1985; Swiercz J. M., et al. J. Cell Biol. 165, 869-880, 2004; Kuhns J. J., et al. J. Biol. Chem. 274, 36422-36427, 1999; Cho H.-S., et al. Nature 421, 756-760, 2003; Ehsani A., et al. (1993) Genomics 15, 426-429; WO2004048938 (Example 2); WO2004027049 (FIG. 11); WO2004009622; WO2003081210; WO2003089904 (Claim 9); WO2003016475 (Claim 1); US2003118592; WO2003008537 (Claim 1); WO2003055439 (Claim 29; FIG. 1A-B); WO2003025228 (Claim 37; FIG. 5C); WO200222636 (Example 13; Page 95-107); WO200212341 (Claim 68; FIG. 7); WO200213847 (Page 71-74); WO200214503 (Page 114-117); WO200153463 (Claim 2; Page 41-46); WO200141787 (Page 15); WO200044899 (Claim 52; FIG. 7); WO200020579 (Claim 3; FIG. 2); U.S. Pat. No. 5,869,445 (Claim 3; Col 31-38); WO9630514 (Claim 2; Page 56-61); EP1439393 (Claim 7); WO2004043361 (Claim 7); WO2004022709; WO200100244 (Example 3; FIG. 4); Accession: P04626; EMBL; M11767; AAA35808.1. EMBL; M11761; AAA35808.1.
(18) NCA (CEACAM6, Genbank accession no. M18728); Barnett T., et al. Genomics 3, 59-66, 1988; Tawaragi Y., et al. Biochem. Biophys. Res. Commun. 150, 89-96, 1988; Strausberg R. L., et al. Proc. Natl. Acad. Sci. U.S.A. 99:16899-16903, 2002; WO2004063709; EP1439393 (Claim 7); WO2004044178 (Example 4); WO2004031238; WO2003042661 (Claim 12); WO200278524 (Example 2); WO200286443 (Claim 27; Page 427); WO200260317 (Claim 2); Accession: P40199; Q14920; EMBL; M29541; AAA59915.1. EMBL; M18728.
(19) MDP (DPEP1, Genbank accession no. BC017023) Proc. Natl. Acad. Sci. U.S.A. 99 (26):16899-16903 (2002)); WO2003016475 (Claim 1); WO200264798 (Claim 33; Page 85-87); JP05003790 (FIG. 6-8); WO9946284 (FIG. 9); Cross-references: MIM:179780; AAH17023.1; BC017023_1.
(20) IL20Rα (IL20Ra, ZCYTOR7, Genbank accession no. AF184971); Clark H. F., et al. Genome Res. 13, 2265-2270, 2003; Mungall A. J., et al. Nature 425, 805-811, 2003; Blumberg H., et al. Cell 104, 9-19, 2001; Dumoutier L., et al. J. Immunol. 167, 3545-3549, 2001; Parrish-Novak J., et al. J. Biol. Chem. 277, 47517-47523, 2002; Pletnev S., et al. (2003) Biochemistry 42:12617-12624; Sheikh F., et al. (2004) J. Immunol. 172, 2006-2010; EP1394274 (Example 11); US2004005320 (Example 5); WO2003029262 (Page 74-75); WO2003002717 (Claim 2; Page 63); WO200222153 (Page 45-47); US2002042366 (Page 20-21); WO200146261 (Page 57-59); WO200146232 (Page 63-65); WO9837193 (Claim 1; Page 55-59); Accession: Q9UHF4; Q6UWA9; Q96SH8; EMBL; AF184971; AAF01320.1.
(21) Brevican (BCAN, BEHAB, Genbank accession no. AF229053) Gary S. C., et al. Gene 256, 139-147, 2000; Clark H. F., et al. Genome Res. 13, 2265-2270, 2003; Strausberg R. L., et al. Proc. Natl. Acad. Sci. U.S.A. 99, 16899-16903, 2002; US2003186372 (Claim 11); US2003186373 (Claim 11); US2003119131 (Claim 1; FIG. 52); US2003119122 (Claim 1; FIG. 52); US2003119126 (Claim 1); US2003119121 (Claim 1; FIG. 52); US2003119129 (Claim 1); US2003119130 (Claim 1); US2003119128 (Claim 1; FIG. 52); US2003119125 (Claim 1); WO2003016475 (Claim 1); WO200202634 (Claim 1).
(22) EphB2R (DRT, ERK, Hek5, EPHT3, Tyro5, Genbank accession no. NM_004442) Chan, J. and Watt, V. M., Oncogene 6 (6), 1057-1061 (1991) Oncogene 10 (5):897-905 (1995), Annu. Rev. Neurosci. 21:309-345 (1998), Int. Rev. Cytol. 196:177-244 (2000)); WO2003042661 (Claim 12); WO200053216 (Claim 1; Page 41); WO2004065576 (Claim 1); WO2004020583 (Claim 9); WO2003004529 (Page 128-132); WO200053216 (Claim 1; Page 42); Cross-references: MIM:600997; NP_004433.2; NM_004442_1.
(23) ASLG659 (B7 h, Genbank accession no. AX092328) US20040101899 (Claim 2); WO2003104399 (Claim 11); WO2004000221 (FIG. 3); US2003165504 (Claim 1); US2003124140 (Example 2); US2003065143 (FIG. 60); WO2002102235 (Claim 13; Page 299); US2003091580 (Example 2); WO200210187 (Claim 6; FIG. 10); WO200194641 (Claim 12; FIG. 7b); WO200202624 (Claim 13; FIG. 1A-1B); US2002034749 (Claim 54; Page 45-46); WO200206317 (Example 2; Page 320-321, Claim 34; Page 321-322); WO200271928 (Page 468-469); WO200202587 (Example 1; FIG. 1); WO200140269 (Example 3; Pages 190-192); WO200036107 (Example 2; Page 205-207); WO2004053079 (Claim 12); WO2003004989 (Claim 1); WO200271928 (Page 233-234, 452-453); WO 0116318.
(24) PSCA (Prostate stem cell antigen precursor, Genbank accession no. AJ297436) Reiter R. E., et al. Proc. Natl. Acad. Sci. U.S.A. 95, 1735-1740, 1998; Gu Z., et al. Oncogene 19, 1288-1296, 2000; Biochem. Biophys. Res. Commun. (2000) 275(3):783-788; WO2004022709; EP1394274 (Example 11); US2004018553 (Claim 17); WO2003008537 (Claim 1); WO200281646 (Claim 1; Page 164); WO 2003003906 (Claim 10; Page 288); WO 200140309 (Example 1; FIG. 17); US 2001055751 (Example 1; FIG. 1b); WO 200032752 (Claim 18; FIG. 1); WO 1998/51805 (Claim 17; Page 97); WO 1998/51824 (Claim 10; Page 94); WO 1998/40403 (Claim 2; FIG. 1B); Accession: 043653; EMBL; AF043498; AAC39607.1.
(25) GEDA (Genbank accession No. AY260763); AAP14954 lipoma HMGIC fusion-partner-like protein/pid=AAP14954.1—Homo sapiens Species: Homo sapiens (human) WO2003054152 (Claim 20); WO2003000842 (Claim 1); WO2003023013 (Example 3, Claim 20); US2003194704 (Claim 45); Cross-references: GI:30102449; AAP14954.1; AY260763_1.
(26) BAFF-R (B cell-activating factor receptor, BLyS receptor 3, BR3, Genbank accession No. AF 116456); BAFF receptor/pid=NP_443177.1—Homo sapiens Thompson, J. S., et al. Science 293 (5537), 2108-2111 (2001); WO2004058309; WO2004011611; WO2003045422 (Example; Page 32-33); WO2003014294 (Claim 35; FIG. 6B); WO2003035846 (Claim 70; Page 615-616); WO200294852 (Col 136-137); WO200238766 (Claim 3; Page 133); WO200224909 (Example 3; FIG. 3); Cross-references: MIM:606269; NP_443177.1; NM_052945_1; AF132600.
(27) CD22 (B-cell receptor CD22-B isoform, BL-CAM, Lyb-8, Lyb8, SIGLEC-2, FLJ22814, Genbank accession No. AK026467); Wilson et al. (1991) J. Exp. Med. 173:137-146; WO2003072036 (Claim 1; FIG. 1); Cross-references: MIM:107266; NP_001762.1; NM_001771_1.
(28) CD79a (CD79A, CD79α, immunoglobulin-associated alpha, a B cell-specific protein that covalently interacts with Ig beta (CD79B) and forms a complex on the surface with Ig M molecules, transduces a signal involved in B-cell differentiation), pI: 4.84, MW: 25028 TM: 2 [P] Gene Chromosome: 19q13.2, Genbank accession No. NP_001774.10) WO2003088808, US20030228319; WO2003062401 (claim 9); US2002150573 (claim 4, pages 13-14); WO9958658 (claim 13, FIG. 16); WO9207574 (FIG. 1); U.S. Pat. No. 5,644,033; Ha et al. (1992) J. Immunol. 148(5):1526-1531; Mueller et al. (1992) Eur. J. Biochem. 22:1621-1625; Hashimoto et al. (1994) Immunogenetics 40(4):287-295; Preud'homme et al. (1992) Clin. Exp. Immunol. 90(1):141-146; Yu et al. (1992) J. Immunol. 148 (2) 633-637; Sakaguchi et al. (1988) EMBO J. 7(11):3457-3464.
(29) CXCR5 (Burkitt's lymphoma receptor 1, a G protein-coupled receptor that is activated by the CXCL13 chemokine, functions in lymphocyte migration and humoral defense, plays a role in HIV-2 infection and perhaps development of AIDS, lymphoma, myeloma, and leukemia); 372 aa, pI: 8.54 MW: 41959 TM: 7 [P] Gene Chromosome: 11q23.3, Genbank accession No. NP_001707.1) WO 2004040000; WO2004/015426; US2003105292 (Example 2); U.S. Pat. No. 6,555,339 (Example 2); WO 2002/61087 (FIG. 1); WO200157188 (Claim 20, page 269); WO200172830 (pages 12-13); WO 2000/22129 (Example 1, pages 152-153, Example 2, pages 254-256); WO 199928468 (claim 1, page 38); U.S. Pat. No. 5,440,021 (Example 2, col 49-52); WO9428931 (pages 56-58); WO 1992/17497 (claim 7, FIG. 5); Dobner et al. (1992) Eur. J. Immunol. 22:2795-2799; Barella et al. (1995) Biochem. J. 309:773-779.
(30) HLA-DOB (Beta subunit of MHC class II molecule (Ia antigen) that binds peptides and presents them to CD4+T lymphocytes); 273 aa, pI: 6.56 MW: 30820 TM: 1 [P] Gene Chromosome: 6p21.3, Genbank accession No. NP_002111.1) Tonnelle et al. (1985) EMBO J. 4(11):2839-2847; Jonsson et al. (1989) Immunogenetics 29(6):411-413; Beck et al. (1992) J. Mol. Biol. 228:433-441; Strausberg et al. (2002) Proc. Natl. Acad. Sci USA 99:16899-16903; Servenius et al. (1987) J. Biol. Chem. 262:8759-8766; Beck et al. (1996) J. Mol. Biol. 255:1-13; Naruse et al. (2002) Tissue Antigens 59:512-519; WO9958658 (claim 13, FIG. 15); U.S. Pat. No. 6,153,408 (Col 35-38); U.S. Pat. No. 5,976,551 (col 168-170); U.S. Pat. No. 6,011,146 (col 145-146); Kasahara et al. (1989) Immunogenetics 30(1):66-68; Larhammar et al. (1985) J. Biol. Chem. 260(26):14111-14119.
(31) P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability); 422 aa), pI: 7.63, MW: 47206 TM: 1 [P] Gene Chromosome: 17p13.3, Genbank accession No. NP_002552.2) Le et al. (1997) FEBS Lett. 418 (1-2):195-199; WO2004047749; WO2003072035 (claim 10); Touchman et al. (2000) Genome Res. 10:165-173; WO200222660 (claim 20); WO2003093444 (claim 1); WO2003087768 (claim 1); WO2003029277 (page 82).
(32) CD72 (B-cell differentiation antigen CD72, Lyb-2), pI: 8.66, MW: 40225 TM: 1 [P] Gene Chromosome: 9p13.3, Genbank accession No. NP_001773.1) WO2004042346 (claim 65); WO 2003/026493 (pages 51-52, 57-58); WO 2000/75655 (pages 105-106); Von Hoegen et al. (1990) J. Immunol. 144(12):4870-4877; Strausberg et al. (2002) Proc. Natl. Acad. Sci USA 99:16899-16903.
(33) LY64 (Lymphocyte antigen 64 (RP105), type I membrane protein of the leucine rich repeat (LRR) family, regulates B-cell activation and apoptosis, loss of function is associated with increased disease activity in patients with systemic lupus erythematosus); 661 aa, pI: 6.20, MW: 74147 TM: 1 [P] Gene Chromosome: 5q12, Genbank accession No. NP_005573.1) US2002193567; WO9707198 (claim 11, pages 39-42); Miura et al. (1996) Genomics 38(3):299-304; Miura et al. (1998) Blood 92:2815-2822; WO2003083047; WO9744452 (claim 8, pages 57-61); WO200012130 (pages 24-26).
(34) FcRH1 (Fc receptor-like protein 1, a putative receptor for the immunoglobulin Fc domain that contains C2 type Ig-like and ITAM domains, may have a role in B-lymphocyte differentiation); 429 aa, pI: 5.28, MW: 46925 TM: 1 [P] Gene Chromosome: 1921-1922, Genbank accession No. NP_443170.1) WO2003077836; WO200138490 (claim 6, FIG. 18E-1-18-E-2); Davis et al. (2001) Proc. Natl. Acad. Sci USA 98(17):9772-9777; WO2003089624 (claim 8); EP1347046 (claim 1); WO2003089624 (claim 7).
(35) IRTA2 (Immunoglobulin superfamily receptor translocation associated 2, a putative immunoreceptor with possible roles in B cell development and lymphomagenesis; deregulation of the gene by translocation occurs in some B cell malignancies); 977 aa, pI: 6.88 MW: 106468 TM: 1 [P] Gene Chromosome: 1q21, Genbank accession No. Human:AF343662, AF343663, AF343664, AF343665, AF369794, AF397453, AK090423, AK090475, AL834187, AY358085; Mouse:AK089756, AY158090, AY506558; NP_112571.1. WO2003024392 (claim 2, FIG. 97); Nakayama et al. (2000) Biochem. Biophys. Res. Commun. 277(1):124-127; WO2003077836; WO200138490 (claim 3, FIG. 18B-1-18B-2).
(36) TENB2 (TMEFF2, tomoregulin, TPEF, HPP1, TR, putative transmembrane proteoglycan, related to the EGF/heregulin family of growth factors and follistatin); 374 aa, NCBI Accession: AAD55776, AAF91397, AAG49451, NCBI RefSeq: NP_057276; NCBI Gene: 23671; OMIM: 605734; SwissProt Q9UIK5; Genbank accession No. AF179274; AY358907, CAF85723, CQ782436 WO 2004074320; JP 2004113151; WO 2003042661; WO2003009814; EP1295944 (pages 69-70); WO 200230268 (page 329); WO 200190304; US2004249130; US 2004022727; WO 2004063355; US 2004197325; US2003232350; US2004005563; US 2003124579; Horie et al. (2000) Genomics 67:146-152; Uchida et al. (1999) Biochem. Biophys. Res. Commun. 266:593-602; Liang et al. (2000) Cancer Res. 60:4907-12; Glynne-Jones et al. (2001) Int J Cancer. October 15; 94(2):178-84.
(37) PMEL17 (silver hornolog; SILV; D12S53E; PMEL17; SI; SIL); ME20; gp100) BC001414; BT007202; M32295; M77348; NM_006928; McGlinchey, R. P. et al. (2009) Proc. Natl. Acad. Sci. U.S.A. 106 (33), 13731-13736; Kummer, M. P. et al. (2009) J. Biol. Chem. 284 (4), 2296-2306.
(38) TMEFF1 (transmembrane protein with EGF-like and two follistatin-like domains 1; Tomoregulin-1); H7365; C9orf2; C90RF2; U19878; X83961; NM_080655; NM_003692; Harms, P. W. (2003) Genes Dev. 17 (21), 2624-2629; Gery, S. et al. (2003) Oncogene 22 (18):2723-2727.
(39) GDNF-Ral (GDNF family receptor alpha 1; GFRA1; GDNFR; GDNFRA; RETL1; TRNR1; RET1L; GDNFR-alpha1; GFR-ALPHA-1); U95847; BC014962; NM_145793 NM_005264; Kim, M. H. et al. (2009) Mol. Cell. Biol. 29 (8), 2264-2277; Treanor, J. J. et al. (1996) Nature 382 (6586):80-83.
(40) Ly6E (lymphocyte antigen 6 complex, locus E; Ly67, RIG-E, SCA-2, TSA-1); NP_002337.1; NM_002346.2; de Nooij-van Dalen, A. G. et al. (2003) Int. J. Cancer 103 (6), 768-774; Zammit, D. J. et al. (2002) Mol. Cell. Biol. 22 (3):946-952.
(41) TMEM46 (shisa homolog 2 (Xenopus laevis); SHISA2); NP_001007539.1; NM_001007538.1; Furushima, K. et al. (2007) Dev. Biol. 306 (2), 480-492; Clark, H. F. et al. (2003) Genome Res. 13 (10):2265-2270.
(42) Ly6G6D (lymphocyte antigen 6 cormplex, locus G6D; Ly6-D, MEGT1); NP_067079.2; NM_021246.2; Mallya, M. et al. (2002) Genomics 80 (1):113-123; Ribas, G. et al. (1999) J. Immunol. 163 (1):278-287.
(43) LGR5 (leucine-rich repeat-containing G protein-coupled receptor 5; GPR49, GPR67); NP_003658.1; NM_003667.2; Salanti, G. et al. (2009) Am. J. Epidemiol. 170 (5):537-545; Yamamoto, Y. et al. (2003) Hepatology 37 (3):528-533.
(44) RET (ret proto-oncogene; MEN2A; HSCR1; MEN2B; MTC1; PTC; CDHF12; Hs.168114; RET51; RET-ELE1); NP_066124.1; NM_020975.4; Tsukamoto, H. et al. (2009) Cancer Sci. 100 (10):1895-1901; Narita, N. et al. (2009) Oncogene 28 (34):3058-3068.
(45) LY6K (lymphocyte antigen 6 complex, locus K; LY6K; HSJ001348; FLJ35226); NP_059997.3; NM_017527.3; Ishikawa, N. et al. (2007) Cancer Res. 67 (24):11601-11611; de Nooij-van Dalen, A. G. et al. (2003) Int. J. Cancer 103 (6):768-774.
(46) GPR19 (G protein-coupled receptor 19; Mm.4787); NP_006134.1; NM_006143.2; Montpetit, A. and Sinnett, D. (1999) Hum. Genet. 105 (1-2):162-164; O'Dowd, B. F. et al. (1996) FEBS Lett. 394 (3):325-329.
(47) GPR54 (KISS1 receptor; KISSIR; GPR54; HOT7T175; AXOR12); NP_115940.2; NM_032551.4; Navenot, J. M. et al. (2009) Mol. Pharmacol. 75 (6):1300-1306; Hata, K. et al. (2009) Anticancer Res. 29 (2):617-623.
(48) ASPHD1 (aspartate beta-hydroxylase domain containing 1; LOC253982); NP_859069.2; NM_181718.3; Gerhard, D. S. et al. (2004) Genome Res. 14 (10B):2121-2127.
(49) Tyrosinase (TYR; OCAIA; OCA1A; tyrosinase; SHEP3); NP_000363.1; NM_000372.4; Bishop, D. T. et al. (2009) Nat. Genet. 41 (8):920-925; Nan, H. et al. (2009) Int. J. Cancer 125 (4):909-917.
(50) TMEM118 (ring finger protein transmembrane 2; RNFT2; FLJ14627); NP_001103373.1; NM_001109903.1; Clark, H. F. et al. (2003) Genome Res. 13 (10):2265-2270; Scherer, S. E. et al. (2006) Nature 440 (7082):346-351.
(51) GPR172A (G protein-coupled receptor 172A; GPCR41; FLJ11856; D15Ertd747e); NP_078807.1; NM_024531.3; Ericsson, T. A. et al. (2003) Proc. Natl. Acad. Sci. U.S.A. 100 (11):6759-6764; Takeda, S. et al. (2002) FEBS Lett. 520 (1-3):97-101.
(52) CD33, a member of the sialic acid binding, immunoglobulin-like lectin family, is a 67-kDa glycosylated transmembrane protein. CD33 is expressed on most myeloid and monocytic leukemia cells in addition to committed myelomonocytic and erythroid progenitor cells. It is not seen on the earliest pluripotent stem cells, mature granulocytes, lymphoid cells, or nonhematopoietic cells (Sabbath et al., (1985) J. Clin. Invest. 75:756-56; Andrews et al., (1986) Blood 68:1030-5). CD33 contains two tyrosine residues on its cytoplasmic tail, each of which is followed by hydrophobic residues similar to the immunoreceptor tyrosine-based inhibitory motif (ITIM) seen in many inhibitory receptors.
(53) CLL-1 (CLEC12A, MICL, and DCAL2), encodes a member of the C-type lectin/C-type lectin-like domain (CTL/CTLD) superfamily. Members of this family share a common protein fold and have diverse functions, such as cell adhesion, cell-cell signaling, glycoprotein turnover, and roles in inflammation and immune response. The protein encoded by this gene is a negative regulator of granulocyte and monocyte function. Several alternatively spliced transcript variants of this gene have been described, but the full-length nature of some of these variants has not been determined. This gene is closely linked to other CTL/CTLD superfamily members in the natural killer gene complex region on chromosome 12p13 (Drickamer K (1999) Curr. Opin. Struct. Biol. 9 (5):585-90; van Rhenen A, et al., (2007) Blood 110 (7):2659-66; Chen C H, et al. (2006) Blood 107 (4):1459-67; Marshall A S, et al. (2006) Eur. J. Immunol. 36 (8):2159-69; Bakker A B, et al. (2005) Cancer Res. 64 (22):8443-50; Marshall A S, et al. (2004) J. Biol. Chem. 279 (15):14792-802). CLL-1 has been shown to be a type II transmembrane receptor comprising a single C-type lectin-like domain (which is not predicted to bind either calcium or sugar), a stalk region, a transmembrane domain and a short cytoplasmic tail containing an ITIM motif.
(54) TROP2 (tumor-associated calcium signal transducer 2) is a transmembrane glycoprotein encoded by the TACSTD2 gene (Linnenbach A J, et al (1993) Mol Cell Biol. 13(3): 1507-15; Calabrese G, et al (2001) Cylogenet Cell Genet. 92 (1-2): 164-5). TROP2 is an intracellular calcium signal transducer that is differentially expressed in many cancers. It signals cells for self-renewal, proliferation, invasion, and survival. It has stem cell-like qualities. TROP2 is expressed in many normal tissues, though in contrast, it is overexpressed in many cancers (Ohmachi T, et al., (2006) Clin. Cancer Res., 12(10), 3057-3063; Muhlmann G, et al., (2009) J. Clin. Pathol., 62(2), 152-158; Fong D, et al., (2008) Br. J. Cancer, 99(8), 1290-1295; Fong D, et al., (2008) Mod. Pathol., 21(2), 186-191; Ning S, et al., (2013) Neurol. Sci., 34(10), 1745-1750). Overexpression of TROP2 is of prognostic significance. Several ligands have been proposed that interact with TROP2. TROP2 signals the cells via different pathways and it is transcriptionally regulated by a complex network of several transcription factors.

Human TROP2 (TACSTD2: tumor-associated calcium signal transducer 2, GA733-1, EGP-1, M1S1; hereinafter, referred to as hTROP2) is a single-pass transmembrane type 1 cell membrane protein consisting of 323 amino acid residues. While the presence of a cell membrane protein involved in immune resistance, which is common to human trophoblasts and cancer cells (Faulk W P, et al. (1978), Proc. Natl. Acad. Sci. 75(4):1947-1951), has previously been suggested, an antigen molecule recognized by a monoclonal antibody against a cell membrane protein in a human choriocarcinoma cell line was identified and designated as TROP2 as one of the molecules expressed in human trophoblasts (Lipinski M, et al. (1981), Proc. Natl. Acad. Sci. 78(8), 5147-5150). This molecule was also designated as tumor antigen GA733-1 recognized by a mouse monoclonal antibody GA733 (Linnenbach A J, et al., (1989) Proc. Natl. Acad. Sci. 86(1), 27-31) obtained by immunization with a gastric cancer cell line or an epithelial glycoprotein (EGP-1; Basu A, et al., Int. J. Cancer, 62 (4), 472-479 (1995)) recognized by a mouse monoclonal antibody RS7-3G11 obtained by immunization with non-small cell lung cancer cells. In 1995, however, the TROP2 gene was cloned, and all of these molecules were confirmed to be identical molecules (Fornaro M, et al., (1995) Int. J. Cancer, 62(5), 610-618). The DNA sequence and amino acid sequence of hTROP2 are available on a public database and can be referred to, for example, under Accession Nos. NM_002353 and NP_002344 (NCBI).

In response to such information suggesting the association with cancer, a plurality of anti-hTROP2 antibodies have been established so far and studied for their antitumor effects. Among these antibodies, there is disclosed, for example, an unconjugated antibody that exhibits in itself antitumor activity in nude mouse xenograft models (WO 2008/144891; WO 2011/145744; WO 2011/155579; WO 2013/077458) as well as an antibody that exhibits antitumor activity as ADC with a cytotoxic drug (WO 2003/074566; WO 2011/068845; WO 2013/068946; U.S. Pat. No. 7,999,083). However, the strength or coverage of their activity is still insufficient, and there are unsatisfied medical needs for hTROP2 as a therapeutic target.

TROP2 expression in cancer cells has been correlated with drug resistance. Several strategies target TROP2 on cancer cells that include antibodies, antibody fusion proteins, chemical inhibitors, nanoparticles, etc. The in vitro studies and pre-clinical studies, using these various therapeutic treatments, have resulted in significant inhibition of tumor cell growth both in vitro and in vivo in mice. Clinical studies have explored the potential application of Trop2 as both a prognostic biomarker and as a therapeutic target to reverse resistance.
(55) CD123 (IL-4, IL3RA, IL3ry, IL3RAY, interleukin-3 receptor) is a protein found on cells which helps transmit the signal of interleukin-3, a soluble cytokine important in the immune system. The gene coding for the receptor is located in the pseudoautosomal region of the X and Y chromosomes. The receptor belongs to the type I cytokine receptor family and is a heterodimer with a unique alpha chain paired with the common beta (beta c or CD131) subunit. The gene for the alpha subunit is 40 kilobases long and has 12 exons. CD123 is the 70 kD transmembrane a chain of the IL-3 receptor. Alone, CD123 binds IL-3 with low affinity; when CD123 associates with CDw131 (common β chain), it binds IL-3 with high affinity. CD123 does not transduce intracellular signals upon binding IL-3 and requires the R chain for this function. CD123 serves as a diagnostic, prognostic and therapeutic marker in some hematologic malignancies, especially acute leukemia CD123 and TCF4 coexpression by immunohistochemistry is highly specific and sensitive for blastic plasmacytoid dendritic cell neoplasm (BPDCN) (Sun Q, et al. (1996) Blood 87:83; Herling M, et al. (2003) Blood 101:5007; Charles N, et al. (2010) Nat. Med. 16:701; Martin-Gayo E, et al. (2010) Blood 115:5366; Testa U, et al. (2019) Cancers 11(9):1358-1388; Shi M, et al. (2019) Cardiovasc Hematol Disord Drug Targets 19(3):195-204).

Exemplary embodiments of the antibody target are HER2 and CD33.

In embodiments, the antibody has a free cysteine thiol group available for conjugation with an electrophilic group of a cereblon degrader-linker intermediate (cDLI).

The thiol-containing antibody may be a native cysteine thiol or a reduced intrachain or interchain disulfide amino acid residue.

The thiol-containing antibody may be a cysteine-engineered antibody where one or more cysteine residues have been introduced by mutagenesis based on known techniques and provide for site-specific conjugation of the cereblon degrader-linker intermediate through cysteine substitutions at sites where the engineered cysteines are available for conjugation while not perturbing immunoglobulin folding and assembly or altering antigen binding and effector functions (Junutula, et al., (2008) Nature Biotech., 26(8):925-932; Doman et al. (2009) Blood 114(13):2721-2729; Shen, B. et al (2012) Nat. Biotechnol. 30(2):184-189; Sukumaran et al (2015) Pharm Res 32:1884-1893 U.S. Pat. Nos. 7,521,541; 7,723,485; US 2012/0121615; WO 2009/052249). One, two, three or more cysteine amino acids may be introduced into the cysteine-engineered antibody.

A cDAC may be formed by conjugating one or more antibody cysteine thiol groups to a molar excess of a cereblon degrader-linker intermediate (cDLI) of Formula II. Due to their symmetrical structure, a cysteine-engineered IgG antibody may allow conjugation of up to two cDLI with each mutant cysteine site. For example, a cysteine-engineered antibody with one mutant cysteine site allows conjugation of up to two cDLI to give a theoretical maximum DAR of 2. A cysteine-engineered antibody with two mutant cysteine sites allows conjugation of up to four cDLI to give a theoretical maximum DAR of 4. A cysteine-engineered antibody with three mutant cysteine sites allows conjugation of up to six cDLI to give a theoretical maximum DAR of 6.

Cysteine thiols are reactive nucleophiles at neutral pH, unlike most amines which are protonated and less nucleophilic near pH 7. Since free thiol (RSH, sulfhydryl) groups are relatively reactive, proteins with cysteine residues often exist in their oxidized form as disulfide-linked oligomers or have internally bridged disulfide groups. Antibody cysteine thiol groups are generally more reactive, i.e. more nucleophilic, towards electrophilic conjugation reagents than antibody amine or hydroxyl groups. Engineering in cysteine thiol groups by the mutation of various amino acid residues of a protein to cysteine amino acids is potentially problematic, particularly in the case of unpaired (free Cys) residues or those which are relatively accessible for reaction or susceptible to oxidation. In concentrated solutions of the protein, whether in the periplasm of E. coli, culture supernatants, or partially or completely purified protein, unpaired Cys residues on the surface of the protein can pair and oxidize to form intermolecular disulfides, and hence protein dimers or multimers. Disulfide dimer formation renders the new Cys unreactive for conjugation to a drug, ligand, or other label. Furthermore, if the protein oxidatively forms an intramolecular disulfide bond between the newly engineered Cys and an existing Cys residue, both Cys groups are unavailable for active site participation and interactions. Furthermore, the protein may be rendered inactive or non-specific, by mis-folding or loss of tertiary structure (Zhang et al. (2002) Anal. Biochem. 311:1-9).

In some embodiments, cysteine-engineered antibodies may have a reactive cysteine thiol residue introduced at a site on the light chain, such as the 149-lysine site (LC K149C), or on the heavy chain such as the 122-serine site (HC S122C), as numbered by Kabat numbering. In other embodiments, the cysteine-engineered antibodies have a cysteine residue introduced at the 118-alanine site (EU numbering) of the heavy chain (HC A118C). This mutation site is alternatively numbered 121 by Sequential numbering or 114 by Kabat numbering. In other embodiments, the cysteine-engineered antibodies have a mutant cysteine residue introduced in: (i) the light chain at G64C, R142C, K188C, L201C, T129C, S114C, V205C, or E105C according to Kabat numbering; (ii) the heavy chain at D101C, A140C, L177C, V184C, T205C, or S122C according to Kabat numbering; or (iii) other cysteine-mutant antibodies, as described in Bhakta, S. et al, (2013) “Engineering THIOMABs for Site-Specific Conjugation of Thiol-Reactive Linkers”, Laurent Ducry (ed.), Antibody-Drug Conjugates, Methods in Molecular Biology, vol. 1045, pages 189-203; WO 2011/156328; U.S. Pat. No. 9,000,130. In other embodiments, the cysteine-engineered antibody comprises one or more cysteine mutations selected from HC A118C, LC K149C, HC A140C, LC V205C, LC S121C, HC L174C, HC L177C, HC Y373C.

In certain embodiments, an antibody provided herein may be further modified to contain additional nonproteinaceous moieties that are known in the art and readily available. The moieties suitable for derivatization of the antibody include, but are not limited to, water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, polypropylene oxide/ethylene oxide co-polymers, polyoxyethylated polyols (e.g., glycerol), polyvinyl alcohol, and mixtures thereof. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In some embodiments, cDAC is a mixture of the cereblon degrader antibody conjugate compounds, wherein the average drug loading per antibody in the mixture of cereblon degrader antibody conjugate compounds is about 2 to about 6.

The present disclosure includes all reasonable combinations, and permutations of the features, of the Formulae I-II embodiments.

Drug loading is represented by p, the number of cereblon degrader moieties (CD) per antibody (Ab) in a cereblon degrader antibody conjugate (cDAC) of Formula I. Loading (p) may range from 1 to about 8 CD moieties per antibody. cDAC of Formula I include mixtures or collections of antibodies conjugated with a range of cD moieties, from 1 to about 8. In some embodiments, the number of cD moieties that can be conjugated to an antibody is limited by the number of reactive or available amino acid side chain residues such as lysine and cysteine. In some embodiments, free cysteine residues are introduced into the antibody amino acid sequence by the methods described herein. In such aspects, p may be 1, 2, 3, 4, 5, 6, 7, or 8, and ranges thereof, such as from 1 to 8 or from 2 to 6. Exemplary cDACs of Formula I include, but are not limited to, antibodies that have 1, 2, 3, or 4 engineered cysteine amino acids (Lyon, R. et al. (2012) Methods in Enzym. 502:123-138). In some embodiments, one or more free cysteine residues are already present in an antibody forming intra-chain and inter-chain disulfide bonds (native disulfide groups), without the use of engineering, in which case the existing free, reduced cysteine residues may be used to conjugate the antibody to a drug. In some embodiments, an antibody is exposed to reducing conditions prior to conjugation of the antibody in order to generate one or more free native cysteine residues.

For some cDACs, p may be limited by the number of attachment sites on the antibody. For example, where the attachment is a cysteine thiol, as in certain exemplary embodiments described herein, an antibody may have only one or a limited number of cysteine thiol groups, or may have only one or a limited number of sufficiently reactive thiol groups, to which the drug may be attached. In other embodiments, one or more lysine amino groups in the antibody may be available and reactive for conjugation with a cereblon degrader-linker intermediate of Formula II. In certain embodiments, higher drug loading, e.g. p>5, may cause aggregation, insolubility, toxicity, or loss of cellular permeability of certain antibody-drug conjugates. In certain embodiments, the average cD loading for an cDAC ranges from 1 to about 8; from about 2 to about 6; or from about 3 to about 5. In certain embodiments, an antibody is subjected to denaturing conditions to reveal reactive nucleophilic groups such as lysine or cysteine.

The loading (drug/antibody ratio) of an cDAC may be controlled in different ways, and for example, by: (i) limiting the molar excess of the cereblon degrader-linker intermediate compound relative to antibody, (ii) limiting the conjugation reaction time or temperature, and (iii) partial or limiting reductive denaturing conditions for optimized antibody reactivity.

It is to be understood that where more than one nucleophilic group of the antibody reacts with a drug, then the resulting product is a mixture of cDAC compounds with a distribution of one or more drug moieties attached to an antibody. The average number of drugs per antibody (DAR) may be calculated from the mixture by a dual ELISA antibody assay, which is specific for antibody and specific for the drug. Individual cDAC molecules may be identified in the mixture by mass spectroscopy and separated by HPLC, e.g. hydrophobic interaction chromatography (see, e.g., McDonagh et al. (2006) Prot. Engr. Design & Selection 19(7):299-307; Hamblett et al, (2004) Clin. Cancer Res. 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous cDAC with a single loading value may be isolated from the conjugation mixture by electrophoresis or chromatography.

In some embodiments, the cereblon degrader moiety of a cDAC has the formula:

TPL-L₂-E3UL
wherein:
TPL is a target protein ligand;
E3UL is the cereblon-binding, E3 ubiquitin ligase ligand;
L₂ is a degrader linker; and
one of TPL, E3UL and L₂ is attached to L₁; or
the cereblon degrader moiety is a molecular glue.

In some embodiments, TPL has the formula:

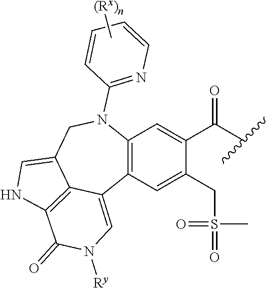

wherein
R^{x} is selected from F, Cl, and Br, and n is 0, 1, 2 or 3;
R^{y} is selected from H and C₁-C₆ alkyl; and
the wavy line indicates the point of attachment of L₂.

In some embodiments, TPL has the formula:

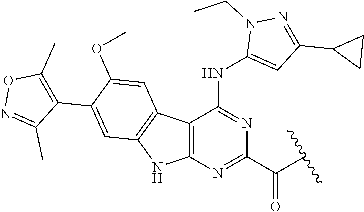 [SMILES: *C(=O)c1nc(Nc2cc(C3CC3)nn2CC)c2c(n1)[nH]c1cc(-c3c(C)noc3C)c(OC)cc12]

where the wavy line indicates the point of attachment of L₂.

In some embodiments, TPL has the formula:

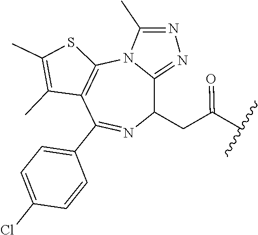 [SMILES: *C(=O)CC1N=C(c2ccc(Cl)cc2)c2c(sc(C)c2C)-n2c(C)nnc21]

where the wavy line indicates the point of attachment of L₂.

In some embodiments, TPL targets BRD4, GSPT1, BET, BRM (SMARCA2), KRAS, and SHP2.

In some embodiments, E3UL comprises a glutarimide group.

In some embodiments, E3UL is selected from:

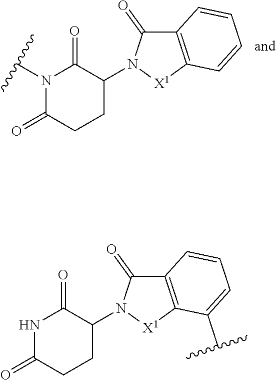

wherein X¹ is selected from CH₂ and C(═O); and the wavy line indicates the point of attachment of L₁ or L₂.

In some embodiments, the cereblon degrader antibody conjugate has the formula:

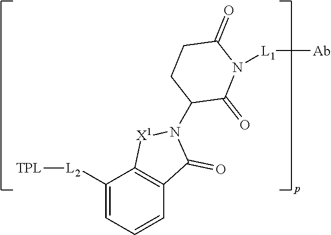

wherein L₁ is a protease-cleavable, non-peptide linker having the formula:

-Str-PM-IM-
wherein Str is a stretcher unit covalently attached to X,
PM is a peptidomimetic unit, and
IM is an immolator unit covalently attached to the glutarimide group of E3UL and has the formula:

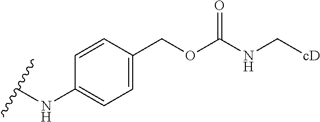

where the wavy line is the attachment to PM.

In some embodiments, the cereblon degrader antibody conjugate has the formula:

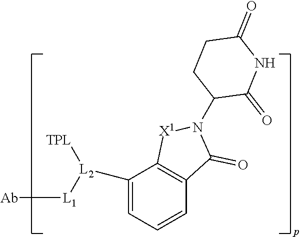

wherein L₁ is a protease-cleavable, non-peptide linker having the formula:

-Str-PM-IM-
wherein Str is a stretcher unit covalently attached to X,
PM is a peptidomimetic unit, and
IM is an immolator unit covalently attached to L₂ of cD and has the formula:

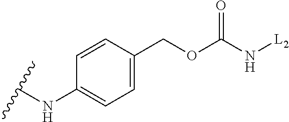

where the wavy line is the attachment to PM.

In some embodiments, L₂ is selected from:
—N(R)—(C₁-C₁₂ alkyldiyl)-N(R)—,
—N(R)—(C₂-C₁₂ alkenyldiyl)-N(R)—,
—N(R)—(C₂-C₁₂ alkynyldiyl)-N(R)—,
—N(R)—(C₁-C₁₂ alkyldiyl)-C(═O)—(N(R)—,
—N(R)—(C₁-C₁₂ alkyldiyl)-(N(R)—C(═O)CH₂O—,
—N(R)—(C₁-C₁₂ alkyldiyl)-(N(R)—C(═O)CH₂N(R)—,
—N(R)—(C₁-C₁₂ alkyldiyl)-C(═O)—(N(R)—(C₁-C₁₂ alkyldiyl)-N(R)—,
—N(R)—(C₁-C₆ alkyldiyl)-O—(C₁-C₆ alkyldiyl)-N(R)—,
—N(R)—(CH₂CH₂O)ₙ—N(R)—(CH₂CH₂O)ₙ—, where n is an integer from 1 to 4,
C₁-C₁₂ alkyldiyl,
C₂-C₁₂ alkenyldiyl, and
C₂-C₁₂ alkynyldiyl,
where R is selected from H, C₁-C₆ alkyldiyl, and a point of attachment to L₁; and
alkyldiyl, alkenyldiyl, and alkynyldiyl are optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H.

In some embodiments, a cDAC of Formula I is selected from:

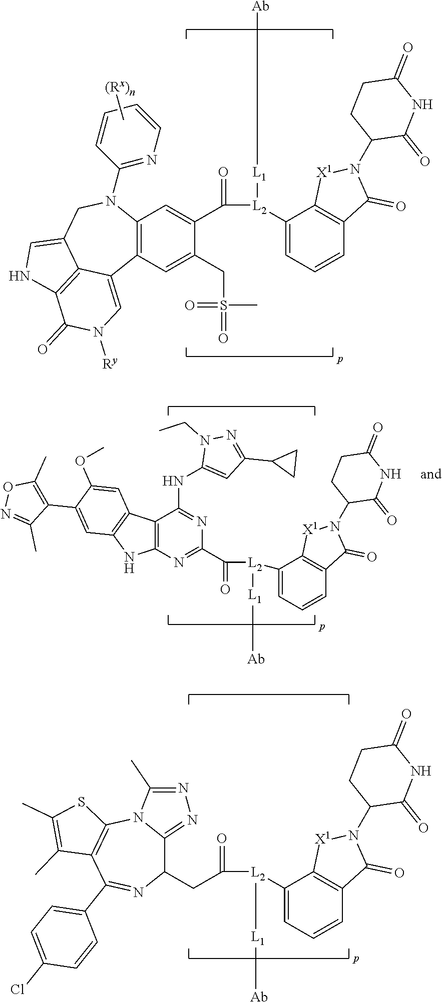

wherein R^{x} is selected from F, Cl, Br, n is 0, 1, 2 or 3; R^{y} is selected from H and C₁-C₆ alkyl, and X¹ is selected from CH₂ and C(═O).

In some embodiments, Formula I is selected from the formulae:

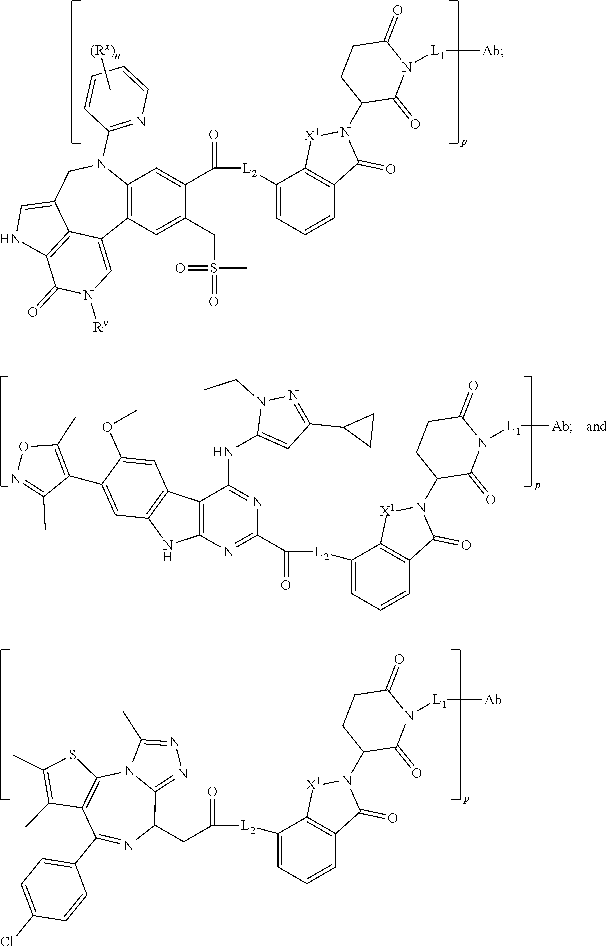

wherein R^{x} is selected from F, Cl, and Br, and n is 0, 1, 2 or 3; R^{y} is selected from H and C₁-C₆ alkyl, and X¹ is selected from CH₂ and C(═O).

In some embodiments, a cDAC of Formula I is selected from:

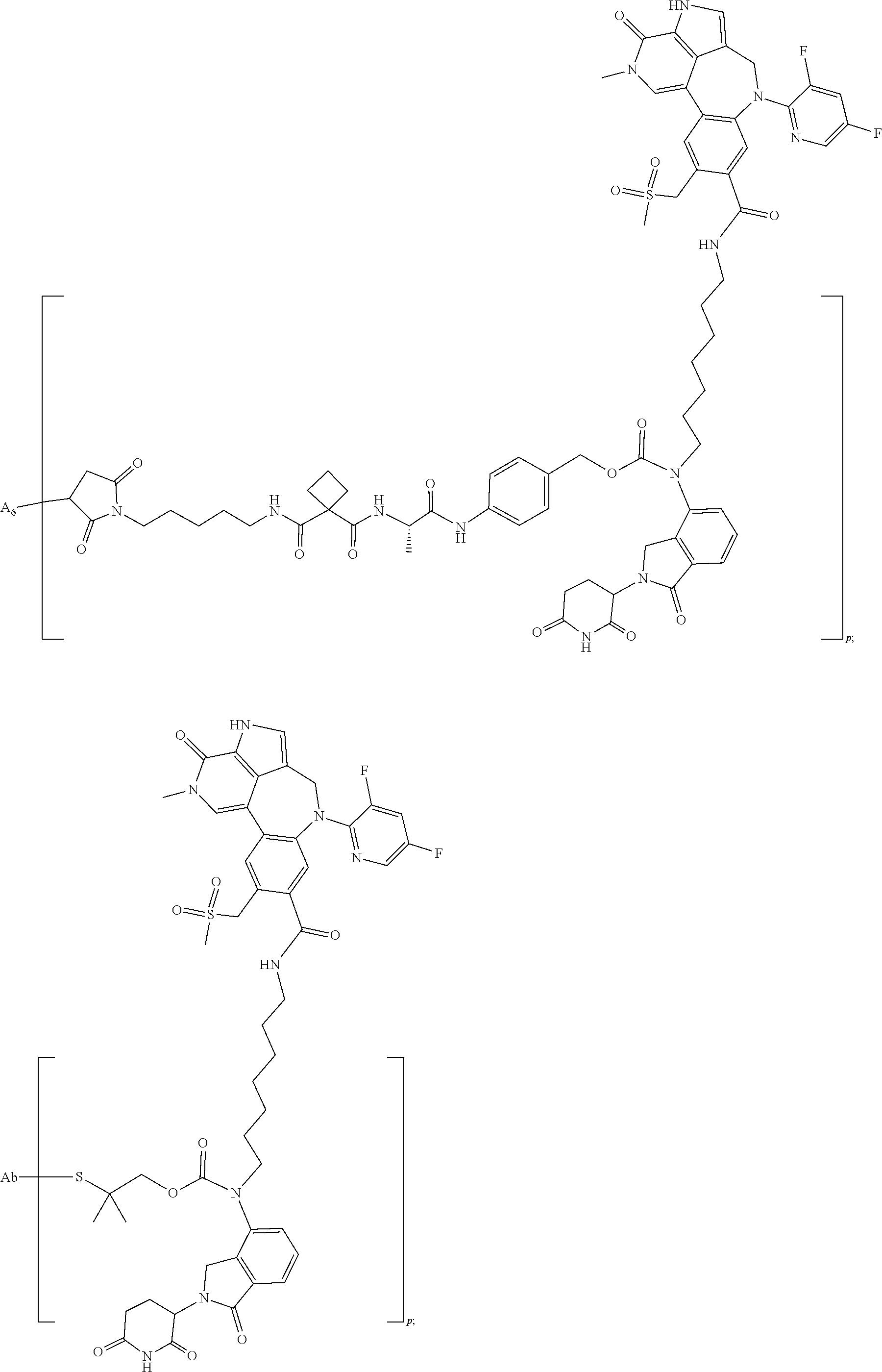

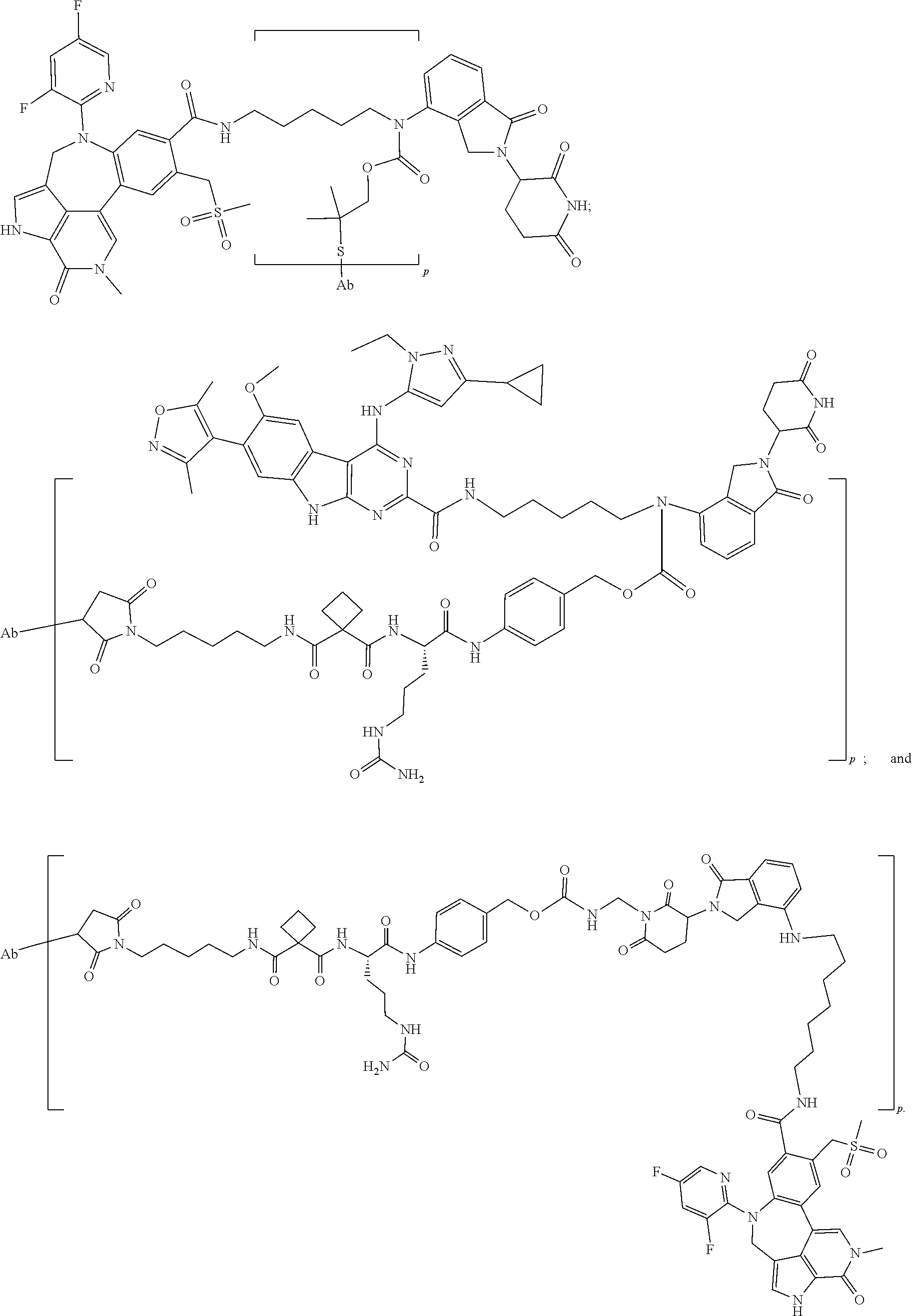

In some embodiments, p of cDAC is 1, 2, 3, 4, 5, or 6.

Table 4 shows exemplary cDACs prepared with cereblon degrader-linker intermediates from Table 2 and their assay data.

TABLE 4
Examples of cereblon degrader antibody conjugates (cDAC)

| | | Table 3 cereblon | |
|---|---|---|---|
| | | degrader-linker | |
| cDAC | Antibody/Target | intermediate | |
| No. | Cys mutant | (CDLI) | DAR |
| 1 | trastuzumab/HER2 | 7 | 2 |
| | LC:K149C | | |
| | HER2 | | |
| 2 | Trastuzumab/HER2 | 7 | 2 |
| | HC A140C | | |
| | HER2 | | |
| 3 | CD33 | 1 | 6 |
| | HC:L174C | | |
| | HC:Y373C | | |
| | LC:K149C | | |
| 4 | 7C2/HER2 | 1 | 6 |
| | HC:L174C | | |
| | HC:Y373C | | |
| | LC:K149C | | |
| 5 | CD33 | 5 | 6 |
| | HC:L174C | | |
| | HC:Y373C | | |
| | LC:K149C | | |
| 6 | 7C2/HER2 | 5 | 6 |
| | HC:L174C | | |
| | HC:Y373C | | |
| | LC:K149C | | |

Biological Activity of cDAC

Generally, the cytotoxic or cytostatic activity of a cereblon degrader antibody conjugate (cDAC) is measured by exposing mammalian cells having receptor proteins, e.g. HER2, to the antibody of the cDAC in a cell culture medium; culturing the cells for a period from about 6 hours to about 5 days and measuring cell viability. Cell-based in vitro assays were used to measure viability (proliferation), cytotoxicity, and induction of apoptosis (caspase activation) of the cDAC described herein.

The in vitro potency of cDACs described herein was measured by a cell proliferation assay such as described in Example 103. The cDACs described herein showed surprising and unexpected potency in inhibition of tumor cell proliferation. Potency of the cDACs was correlated with target antigen expression of the cells. The tested conjugates are capable of binding to the specific antigen expressed on the surface of cells and causing the death of those cells in vitro.

The CellTiter-Glo® Luminescent Cell Viability Assay is a commercially available (Promega Corp., Madison, WI), homogeneous assay method based on the recombinant expression of Coleoptera luciferase (U.S. Pat. Nos. 5,583,024; 5,674,713; 5,700,670). This cell proliferation assay determines the number of viable cells in culture based on quantitation of the ATP present, an indicator of metabolically active cells (Crouch et al (1993) J. Immunol. Meth. 160:81-88; U.S. Pat. No. 6,602,677). The CellTiter-Glo® Assay was conducted in 96 well format, making it amenable to automated high-throughput screening (HTS) (Cree et al (1995) AntiCancer Drugs 6:398-404). The homogeneous assay procedure involves adding the single reagent (CellTiter-Glo® Reagent) directly to cells cultured in serum-supplemented medium. Cell washing, removal of medium and multiple pipetting steps are not required. The system detects as few as 15 cells/well in a 384-well format in 10 minutes after adding reagent and mixing. The cells may be treated continuously with the cDAC, or they may be treated and separated from the cDAC. Generally, cells treated briefly, i.e. 3 hours, showed the same potency effects as continuously treated cells. The assay may be conducted in 96- or 384-well format, making it amenable to automated high-throughput screening (HTS). See Cree et al. (1995) AntiCancer Drugs 6:398-404. The assay procedure involves adding a single reagent (CellTiter-Glo® Reagent) directly to cultured cells. This results in cell lysis and generation of a luminescent signal produced by a luciferase reaction. The luminescent signal is proportional to the amount of ATP present, which is directly proportional to the number of viable cells present in culture. Data can be recorded by luminometer or CCD camera imaging device. The luminescence output is expressed as relative light units (RLU).

The homogeneous “add-mix-measure” format results in cell lysis and generation of a luminescent signal proportional to the amount of ATP present. The amount of ATP is directly proportional to the number of cells present in culture. The CellTiter-Glo® Assay generates a “glow-type” luminescent signal, produced by the luciferase reaction, which has a half-life generally greater than five hours, depending on cell type and medium used. Viable cells are reflected in relative luminescence units (RLU). The substrate, Beetle Luciferin, is oxidatively decarboxylated by recombinant firefly luciferase with concomitant conversion of ATP to AMP and generation of photons.

Cell-based in vitro assays are used to measure viability (proliferation), cytotoxicity, and induction of apoptosis (caspase activation) of the cDAC described herein. Generally, the cytotoxic or cytostatic activity of a cDAC is measured by: exposing mammalian cells expressing antigen such as HER2, ER (estrogen receptor) or CD33 polypeptide to cDAC in a cell culture medium; culturing the cells for a period from about 6 hours to about 5 days; and measuring cell viability.

FIG. 1 shows an anti-proliferative effect of in vitro potency by a BRD4-cereblon degrader against KPL-4 and SK-BR-3 cells at 5 days. Cell viability as percent of control is plotted in a graph versus the concentration of cereblon degrader compound cD-5 from Table 1 (nM). The IC₅₀against KPL-4 was 0.65 nM. The IC₅₀against SK—BR03 was 0.41 nM. These results demonstrate significant potency of the cereblon degrader compound.

FIG. 2A shows anti-proliferative effects of in vitro potency by treatment after 5 days of HER2+ KPL-4 cells with anti-HER2 7C2 and anti-CD33 BRD4-cereblon degrader antibody conjugates cDAC-3, cDAC-4, cDAC-5, and cDAC-6 from Table 4. Cell viability as percent of control is plotted in a graph versus the concentration of cDAC (μg/mL). FIG. 2B shows anti-proliferative effects of in vitro potency by treatment after 5 days of HER2+SK-BR-3 cells with anti-HER2 7C2 and anti-CD33 BRD4-cereblon degrader antibody conjugates cDAC-3, cDAC-4, cDAC-5, and cDAC-6 from Table 3. Cell viability as percent of control is plotted in a graph versus the concentration of cDAC (μg/mL).

FIG. 3A shows anti-proliferative effects of in vitro potency by treatment after 5 days of HER2−low/ER+ CAMA1 cells with anti-HER2 7C2 and anti-CD33 BRD4-cereblon degrader antibody conjugates cDAC-3, cDAC-4, cDAC-5, and cDAC-6 from Table 4. Cell viability as percent of control is plotted in a graph versus the concentration of cDAC (μg/mL). FIG. 3B shows anti-proliferative effects of in vitro potency by treatment after 5 days of HER2−low/ER+ EFM19 cells with anti-HER2 7C2 and anti-CD33 BRD4-cereblon degrader antibody conjugates cDAC-3, cDAC-4, cDAC-5, and cDAC-6 from Table 4. Cell viability as percent of control is plotted in a graph versus the concentration of cDAC (μg/mL). Table 5 shows that the anti-HER2 cDACs are active in both HER2+ and HER2−low breast cancer cell lines whereas the off-target anti-CD33 cDACs are not active.

TABLE 5
In vitro potency of BRD4-cereblon
degrader antibody conjugates (cDAC)

| | | | | | CAMA1 | EFM19 |
|---|---|---|---|---|---|---|
| | | | SKBR3 | KPL-4 | (HER2− | (HER2− |
| | | | (HER2+) | (HER2+) | low) | low) |
| cDAC | | | IC50 | IC50 | IC50 | IC50 |
| No. | Target | DAR | (ng/mL) | (ng/mL) | (ng/mL) | (ng/mL) |
| 3 | CD33 | 6 | >10,000 | >10,000 | >10,000 | >10,000 |
| 4 | HER2 | 6 | 1.2 | 2.0 | 0.7 | 1.7 |
| | (7C2) | | | | | |
| 5 | CD33 | 6 | >10,000 | >10,000 | >10,000 | >10,000 |
| 6 | HER2 | 6 | 0.9 | 2.3 | 0.7 | 2.0 |
| | (7C2) | | | | | |

FIG. 4 shows anti-proliferative effects of in vitro potency by treatment after 7 days of AML cell lines with anti-CD33 BRD4-cereblon degrader antibody conjugate cDAC-3. AL cell lines were MV-4-11, EOL-1, Molm-13, Nomo-1, HL-60, and OC-AML-2. Cell viability as percent of control is plotted in a graph versus the concentration of cDAC (μg/mL). Table 6 shows that cDAC-3 is active in inhibiting various AML cell lines

TABLE 6
In vitro potency of anti-CD33 BRD4-cereblon degrader
antibody conjugate cDAC-3 in various AML cell lines

| AML cell line | EC50 |
|---|---|
| MV-4-11 | 0.1058 |
| EOL-1 | 0.001122 |
| Molm-13 | 2.590 |
| Nomo-1 | 0.008795 |
| HL-60 | 0.0003994 |
| OCI-AML-2 | 0.0001918 |

FIG. 5A shows anti-proliferative effects of in vitro potency by treatment after 5 days of EOL-1 AML cells with anti-HER2 7C2 and anti-CD33 BRD4-cereblon degrader antibody conjugates cDAC-3, cDAC-4, cDAC-5, and cDAC-6 from Table 4. Cell viability as percent of control is plotted in a graph versus the concentration of cDAC (μg/mL). FIG. 5B shows anti-proliferative effects of in vitro potency by treatment after 5 days of HL-60 AML cells with anti-HER2 7C2 and anti-CD33 BRD4-cereblon degrader antibody conjugates cDAC-3, cDAC-4, cDAC-5, and cDAC-6 from Table 4. Cell viability as percent of control is plotted in a graph versus the concentration of cDAC (μg/mL). Table 6 shows EC50 values for the cDAC of FIGS. 5A and 5B.

FIG. 6A shows anti-proliferative effects of in vitro potency by treatment after 3 days of Molm-13 AML cells with anti-HER2 7C2 and anti-CD33 BRD4-cereblon degrader antibody conjugates cDAC-3, cDAC-4, cDAC-5, and cDAC-6 from Table 4. Cell viability as percent of control is plotted in a graph versus the concentration of cDAC (μg/mL). FIG. 6B shows anti-proliferative effects of in vitro potency by treatment after 3 days of MV-4-11 AML cells with anti-HER2 7C2 and anti-CD33 BRD4-cereblon degrader antibody conjugates cDAC-3, cDAC-4, cDAC-5, and cDAC-6 from Table 4. Cell viability as percent of control is plotted in a graph versus the concentration of cDAC (μg/mL).

Table 7 shows EC50 values for the potency of in vitro potency of BRD4-cereblon degrader antibody conjugates cDAC-3, cDAC-5, and cDAC-6 and in AML cell lines from FIGS. 5A, 5B, 6A, and 6B. cDAC-3 and cDAC-5 comprise thio human anti-CD33 antibodies, whereas cDAC-4 and cDAC-6 comprise thio human anti-7C2 (HER2) antibodies. It can be seen that targeted cDAC3 and cDAC-5 have a concentration-dependent effects on inhibiting AML cells with CD33 receptors whereas untargeted cDAC-4 and cDAC-6 showed the expected lower potencies.

TABLE 7
In vitro potency (EC₅₀ nM) of BRD4-cereblon degrader antibody
conjugates cDAC3-6 in AML cell lines from FIGS. 5A, 5B, 6A, 6B

| | cDAC-3 | cDAC-4 | cDAC-5 | cDAC-6 |
|---|---|---|---|---|
| | CD33 | HER2 (7C2) | CD33 | HER2 (7C2) |
| Cell line | EC50 nM | EC50 nM | EC50 nM | EC50 nM |
| EOL-1 | 0.002 | 0.8 | 0.0005 | 0.4 |
| HL-60 | 0.003 | 0.08 | 7.5e−008 | 0.04 |
| Molm-13 | 0.3 | 0.7 | 7.6e−008 | 0.6 |
| MV-4-11 | <0.001 | 0.02 | <0.001 | 0.01 |

A cell killing assay on AML cells treated by CD33 BRD4-cereblon degrader antibody conjugates cDAC3 and cDAC-5, and 7C2 BRD4-cereblon degrader antibody conjugates cDAC4 and cDAC-6 gave the IC50 values (ng/ml) in Table 8. It can be seen that targeted cDAC3 and cDAC-5 have a concentration-dependent effect on AML cells with CD33 receptors whereas untargeted cDAC-4 and cDAC-6 have the expected lower potency.

TABLE 8
Cell killing assay of BRD4-cereblon degrader antibody conjugates
cDAC3-6 in AML cell lines measured in IC50 (ng/ml)

| | cDAC-3 | cDAC-4 | cDAC-5 | cDAC-6 |
|---|---|---|---|---|
| | CD33 | HER2 (7C2) | CD33 | HER2 (7C2) |
| Cell | IC50 | IC50 | IC50 | IC50 |
| line | (ng/ml) | (ng/ml) | (ng/ml) | (ng/ml) |
| MV-4-11 | 0.39 | 14.45 | 1.7 | 6.4 |
| EOL-1 | 2.5 | >100 | 2 | >100 |
| HL-60 | 0.58 | >100 | 2.1 | >100 |

The in vitro anti-proliferative effects of the exemplary cDACs indicate that cDAC described herein are biologically active comprised of a broad variety of antibodies, including those binding to the tumor-associated antigens and cell surface receptor proteins described herein. The exemplary cDACs of Table 3 comprise antibodies binding to tumor-associated antigens HER2 and CD33. HER2 is highly expressed at a level of several million copy numbers per cell in certain solid tumors such as breast cancer and gastric cancer. CD33 is expressed in a far lower copy number of about 10,00 per cell in hematological malignancies such as leukemia and lymphoma. The mechanisms of recycling and internalization differ between the HER2 and CD33 cell surface proteins. Thus, the demonstration of significant in vitro potency of the exemplary cDACs comprising HER2 and CD33 reasonably suggests that the cDACs described herein comprising other antibodies besides anti-HER2 and anti-CD33 will be similarly biologically active.

The in vivo efficacy of cDAC were measured in tumor xenograft studies in mice (Examples 104-105). The cDAC described herein showed surprising and unexpected, target-dependent and dose-dependent potency in inhibition of tumor growth. Efficacy of the cDACs may be correlated with target antigen expression of the tumor cells.

The efficacy of the cDACs provided herein is measured in vivo by implanting allografts or xenografts of cancer cells in rodents and treating the tumors with cDAC. Variable results are to be expected depending on the cell line, the specificity of antibody binding of the cDAC to receptors present on the cancer cells, dosing regimen, and other factors. The in vivo efficacy of the cDAC can be measured using a transgenic explant mouse model expressing moderate to high levels of a tumor-associated antigen, including HER2-expressing KPL4, and CD22-expressing BJAB. Subjects may be treated once with cDAC and monitored over 3-6 weeks to measure the time to tumor doubling, log cell kill, and tumor shrinkage. Follow up dose-response and multi-dose experiments may be conducted.

For example, the in vivo efficacy of an anti-HER2 cDAC described herein can be measured by a high expressing HER2 transgenic explant mouse model (Phillips et al (2008) Cancer Res. 68:9280-90). An allograft is propagated from the Fo5 mmtv transgenic mouse which does not respond to, or responds poorly to, HERCEPTIN® (Genentech, Inc.) therapy. Subjects are treated once or more with cDAC at certain dose levels (mg/kg) and placebo buffer control (Vehicle) and monitored over two weeks or more to measure the time to tumor doubling, log cell kill, and tumor shrinkage, conducted according to Examples 104-105.

FIG. 7 shows the in vivo efficacies of anti-CD33 BRD4-cereblon degrader antibody conjugates cDAC-3, cDAC-4, cDAC-5, and cDAC-6 at the following doses in reducing tumor volume over time (21 days) in a HL-60 xenograft mice model.
1) Vehicle (Histidine Buffer #8), 100 μL, IV once
2) cDAC-4, 3 mg/kg IV once
3) cDAC-3, 1 mg/kg IV once
4) cDAC-3, 3 mg/kg IV once
5) cDAC-3, 10 mg/kg IV once
6) cDAC-6, 3 mg/kg IV once
7) cDAC-5, 1 mg/kg IV once
8) cDAC-5, 3 mg/kg IV once

As shown in FIG. 7, the anti-CD33 BRD4-cereblon degrader antibody conjugates cDAC-3 and cDAC-5 from Table 4 showed clear dose-dependent activity of inhibiting tumor growth in the HL-60 human leukemia cell line in mice, conducted according to Example 105. At 1 mg/kg dose, cDAC-5 showed higher efficacy than cDAC-3 (line 7 vs. line 3 in Figure). The amine of the glutarimide group of the cereblon degrader moiety of cDAC-5 is linked to the antibody linker through an aminal structure (Table 3, cDLI-5), whereas the indolinone group of the cereblon degrader moiety of cDAC-3 is linked to the antibody linker through a carbamate group (Table 3, cDLI-1). At 3 mg/kg dose, both cDAC-3 and cDAC-5 (line 4 and line 8 in FIG. 7, respectively) resulted in tumor volume below the limit of quantification. In contrast, FIG. 7 also illustrates that tumors from the non-target HER2-controls cDAC-4 and cDAC-6 at the matched 3 mg/kg groups (line 2 and line 6, respectively) had an initial response, but eventually grew out by the end of study (>21 days). This contrast demonstrated the target-specific efficacy of the BRD4-cereblon degrader antibody conjugates provided herein (e.g., cDAC-3 and cDAC-5). The LALA-PG mutation within the Fc domain of the anti-CD33 antibody ablates Fc-FcR mediated effector functions without affecting desired affinity (Schlothauer, T. et al (2016) Protein Engineering, Design & Selection, 29(10):457-466). In addition, it was shown that doses of 3 mg/kg or lower of the cereblon degrader antibody conjugates provided herein (e.g., cDAC-3 and cDAC-5) were tolerated in mice.

In vivo and whole blood stability of cDAC can be measured and assessed according to standard assays, including Example 104.

The stability of cDAC-4 and cDAC-6 were measured in buffer, Cynomolgus monkey whole blood, Human whole blood, Mouse whole blood, and Rat whole blood according to the whole blood assay of Example 104. At certain time points, samples were subject to capture by the biotinylated extra-cellular domain (ECD) of HER2 antigen immobilized on streptavidin magnetic beads. After washing the beads, the samples were eluted and analyzed by LC/MS (liquid chromatography/mass spectrometry). Identification and characterization by mass and LC elution profile allowed for determination of the average DAR (drug to antibody ratio). Table 9 shows that both cDAC-4 and cDAC-6 were stable over 24 hours at room temperature in all media, cDAC-4 slightly more stable than cDAC-6.

TABLE 9
Buffer and whole blood (WB) stability in various species of
BRD4-cereblon degrader antibody conjugates cDAC-4 and cDAC-6

| | cDAC-4 | cDAC-4 | cDAC-6 | cDAC-6 |
|---|---|---|---|---|
| | avg DAR | avg DAR | avg DAR | avg DAR |
| media | at 0 hr | at 24 hr | at 0 hr | at 24 hr |
| Buffer | 5.82 | 5.88 | 5.88 | 5.80 |
| Cyno WB | 5.93 | 5.87 | 5.91 | 5.56 |
| Human WB | 5.94 | 5.91 | 5.93 | 5.88 |
| Mouse WB | 5.84 | 5.77 | 5.89 | 5.87 |
| Rat WB | 5.95 | 5.87 | 5.92 | 5.89 |

### Pharmaceutical Compositions

In another aspect, provided herein are compositions, e.g., a pharmaceutically or pharmacologically acceptable composition or formulation, comprising an cereblon degrader antibody conjugate (cDAC) or a plurality of cDACs as described herein and a pharmaceutically or pharmacologically acceptable carrier.

A cDAC can be formulated for parenteral administration, such as intradermal, subcutaneous (subcut), intramuscular (IM), or intravenous (IV) injections, infusion, or administration into a body cavity or lumen of an organ. Alternatively, the cDACs described herein can be injected into otherwise placed into a specific site of the body, such as a tumor. Compositions for injection will commonly comprise a solution of the cDAC dissolved in a pharmaceutically acceptable carrier. Among the acceptable vehicles and solvents that can be employed are water and an isotonic solution of one or more salts such as sodium chloride, e.g., Ringer's solution. In addition, sterile fixed oils can conventionally be employed as a solvent or suspending medium. For this purpose, any bland fixed oil can be employed, including synthetic monoglycerides or diglycerides. In addition, fatty acids such as oleic acid can likewise be used in the preparation of injectables. These compositions desirably are sterile and generally free of undesirable matter. These compositions can be sterilized by conventional, well known sterilization techniques. The compositions can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

The composition may contain any suitable concentration of the cDACs. The concentration of the cDAC in the composition can vary widely, and will be selected primarily based on fluid volumes, viscosities, body weight, and the like, in accordance with the particular mode of administration selected and the patient's needs. In certain embodiments, the concentration of an cDAC in a solution formulation for injection will range from about 0.1% (w/w) to about 10% (w/w).

Methods of Treating Cancer with Cereblon Degrader Antibody Conjugates

By inducing target-specific degradation of tumor-associated proteins and conferring specificity to minimize off-target toxicity effects, the cereblon degrader antibody conjugate (cDAC) provided herein may be useful in the treatment of diseases and disorders such as cancer. The cDAC direct a tumor-associated antigen-binding antibody to a cell that expresses the antigen and deliver a cereblon-degrading (cD) moiety to the target cell. A target protein is ubiquitinated and subsequently degraded.

Provided herein are methods for treating cancer with a pharmaceutical composition of the cereblon degrader antibody conjugates (cDAC) provided herein. The method includes administering a therapeutically effective amount of ancDAC as described herein to a subject in need thereof, such as a patient that has cancer and is in need of treatment for the cancer. The method includes administering a therapeutically effective amount of a cDAC selected from Table 3.

In certain embodiments, the disclosed cDACs include those with anticancer activity. The cDAC selectively delivers an effective dose of an active form of the cereblon degrader moiety to tumor tissue, whereby greater selectivity (i.e., a lower efficacious dose) may be achieved while increasing the therapeutic index (“therapeutic window”) relative to an unconjugated cereblon degrader compound.

It is contemplated that the disclosed cDACs may be used to treat various hyperproliferative diseases or disorders, e.g. characterized by the overexpression of a tumor antigen. Exemplary hyperproliferative disorders include benign or malignant solid tumors and hematological disorders such as leukemia and lymphoid malignancies.

Examples of cancer to be treated herein include, but are not limited to, carcinoma, lymphoma, blastoma, sarcoma, leukemia or lymphoid malignancies including acute myeloid leukemia, squamous cell cancer, epithelial squamous cell cancer, lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, and head and neck cancer.

In another aspect, a cDAC for use as a medicament is provided. In certain embodiments, also provided herein are cDACs described herein for use in a method of treating an individual comprising administering to the individual an effective amount of the cDAC. In one such embodiment, the method further comprises administering to the individual an effective amount of at least one additional therapeutic agent, e.g., as described herein.

In a further aspect, also provided herein are uses of a cDAC described herein in the manufacture or preparation of a medicament. In one embodiment, the medicament is for treatment of cancer, the method comprising administering to an individual having cancer an effective amount of the medicament. In one such embodiment, the method further comprises administering to the individual an effective amount of at least one additional therapeutic agent, e.g., as described herein.

Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein. The cDAC dose can range from about 5 mg/kg (body weight) to about 50 mg/kg, from about 10 μg/kg to about 5 mg/kg, or from about 100 μg/kg to about 1 mg/kg. The cDAC dose can be about 100, 200, 300, 400, or 500 μg/kg. The cDAC dose can be about 1, 2, 3, 4, 5, 6, 7, 8, 9, or 10 mg/kg. The cDAC dose can also be outside of these ranges, depending on the particular conjugate as well as the type and severity of the cancer or disorder being treated. Frequency of administration can range from a single dose to multiple doses per week, or more frequently. In some embodiments, the cDAC is administered from about once per month to about five times per week. In some embodiments, the cDAC is administered once per week.

The disclosed cDACs can be used either alone or in combination with other therapeutic agents in a therapy regimen. cDAC may be administered concurrently in a regimen with one or more other drugs during the same treatment cycle, on the same day of treatment as the one or more other drugs, and, optionally, at the same time as the one or more other drugs. For instance, for cancer therapies given every 3 weeks, the concurrently administered drugs are each administered on day-1 of a 3-week cycle. For instance, a cDAC may be co-administered with at least one additional therapeutic agent, such as a chemotherapeutic agent. Such combination therapies encompass combined administration (where two or more therapeutic agents are included in the same or separate formulations), and separate administration, in which case, administration of the cDAC can occur prior to, simultaneously, and/or following, administration of the additional therapeutic agent. cDACs can also be used in combination with radiation therapy.

The disclosed cDACs may be useful in treating a HER2-positive (HER2+) cancer comprising cancer cells which have higher than normal levels of HER2. Examples of HER2-positive cancer include HER2-positive breast cancer and HER2-positive gastric cancer. Optionally, HER2-positive cancer has an immunohistochemistry (IHC) score of 2+ or 3+ by in situ hybridization (ISH) amplification ratio. The term “HER2-positive cell” refers to a cell that expresses HER2 on its surface. cDAC may also be useful in treating HER2−low tumor types.

### EXAMPLES

### Example 1 Synthesis of 1-(5-aminopentyl)-1H-pyrrole-2,5-dione hydrochloride, 1

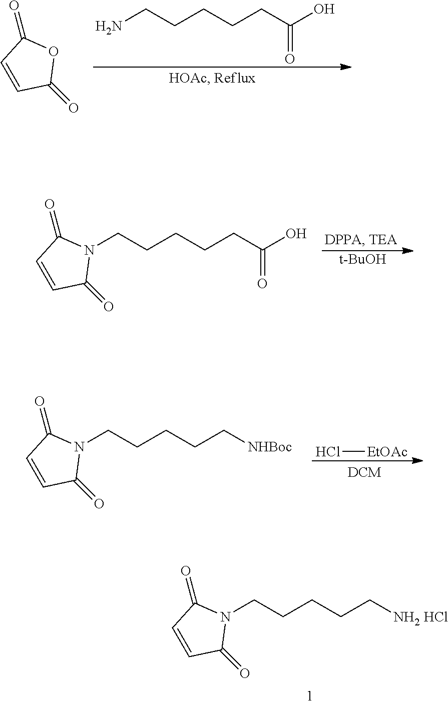

Following the procedures of WO 2017/214024, incorporated by reference herein, maleic anhydride, furan-2,5-dione (150 g, 1.53 mol) was added to a stirred solution of 6-aminohexanoic acid (201 g, 1.53 mol) in HOAc (1000 mL). After the mixture was stirred at r.t. for 2 h, it was heated at reflux for 8 h. The organic solvents were removed under reduced pressure and the residue was extracted with EtOAc (500 mL×3), washed with H₂O. The combined organic layers was dried over Na₂SO₄ and concentrated to give the crude product. It was washed with petroleum ether to give 6-(2,5-dioxo-2,5-dihydro-1H-pyrrol-1-yl)hexanoic acid as white solid (250 g, 77.4%). DPPA (130 g, 473 mmol) and TEA (47.9 g, 473 mmol) was added to a solution of 6-(2,5-dioxo-2,5-dihydro-1H-pyrrol-1-yl)hexanoic acid (100 g, 473 mmol) in t-BuOH (200 mL). The mixture was heated at reflux for 8 h under N₂. The mixture was concentrated, and the residue was purified by column chromatography on silica gel (PE:EtOAc=3:1) to give tert-butyl 5-(2,5-dioxo-2,5-dihydro-1H-pyrrol-1-yl)pentylcarbamate (13 g, 10%). To a solution of tert-butyl 5-(2,5-dioxo-2,5-dihydro-1H-pyrrol-1-yl)pentylcarbamate (28 g, 992 mmol) in anhydrous EtOAc (30 mL) was added HCl/EtOAc (50 mL) dropwise. After the mixture was stirred at r.t. for 5 h, it was filtered and the solid was dried to give 1-(5-aminopentyl)-1H-pyrrole-2,5-dione hydrochloride, 1 (16 g, 73.7%). ¹H NMR (400 MHz, DMSO-d₆): δ 8.02 (s, 2H), 6.99 (s, 2H), 3.37-3.34 (m, 2H), 2.71-2.64 (m, 2H), 1.56-1.43 (m, 4H), 1.23-1.20 (m, 2H).

### Example 2 Synthesis of (S)-1-(1-(4-(hydroxymethyl)phenylamino)-1-oxo-5-ureidopentan-2-ylcarbamoyl)cyclobutanecarboxylic acid, 2

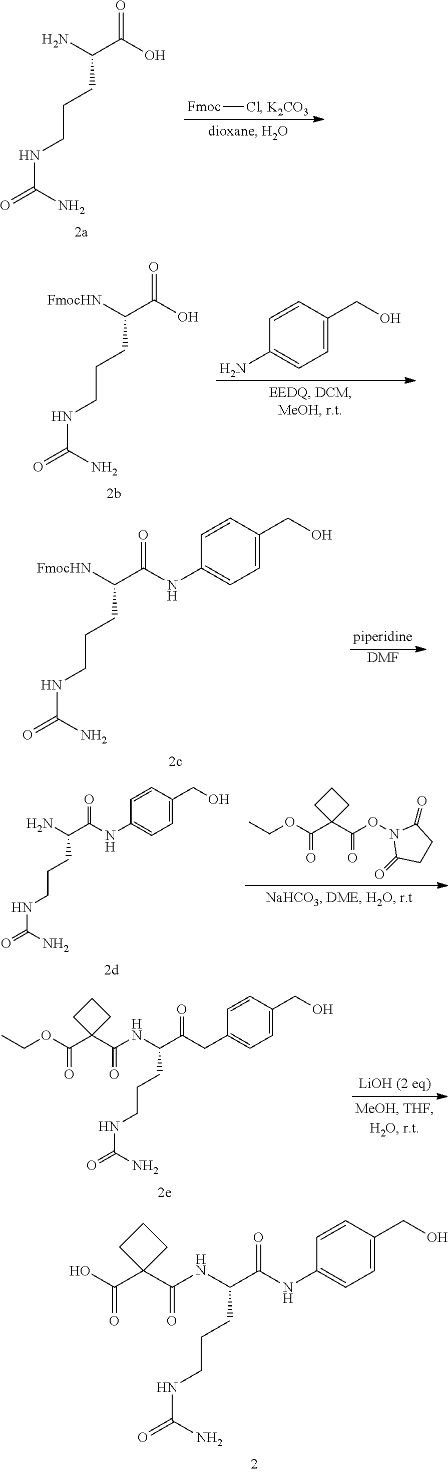

To a mixture of (S)-2-amino-5-ureidopentanoic acid 2a (17.50 g, 0.10 mol) in a mixture of dioxane and H₂O (50 mL/75 mL) was added K₂CO₃ (34.55 g, 0.25 mol). Fmoc-Cl (30.96 g, 0.12 mol) was added slowly at 0 °C. The reaction mixture was warmed to r.t. over 2 h. Organic solvent was removed under reduced pressure, and the water slurry was adjusted to pH=3 with 6 M HCl solution, and extracted with EtOAc (100 mL×3). The organic layer was dried over Na₂SO₄, filtered, and concentrated under reduced pressure to give (S)-2-((((9H-fluoren-9-yl)methoxy)carbonyl)amino)-5-ureidopentanoic acid 2b (38.0 g, 95.6%). 2b is commercially available.

To a solution of 2b (4 g, 10 mmol) in a mixture of DCM and MeOH (100 mL/50 mL) were added (4-aminophenyl)methanol (1.6 g, 13 mmol, 1.3 eq) and 2-Ethoxy-1-ethoxycarbonyl-1,2-dihydroquinoline, EEDQ, Sigma-Aldrich CAS Reg. No. 16357-59-8 (3.2 g, 13 mmol, 1.3 eq). After the mixture was stirred at r.t. for 16 h under N₂, it was concentrated to give a brown solid. MTBE (200 mL) was added and it was stirred at 15 °C for 2 h. The solid was collected by filtration, washed with MTBE (50 mL×2) to give (S)-(9H-fluoren-9-yl)methyl (1-((4-(hydroxymethyl)phenyl)amino)-1-oxo-5-ureidopentan-2-yl)carbamate 2c as an orange solid (4.2 g, 84%). LCMS (ESI): m/z 503.0 [M+1].

To a stirred solution of 2c (4.2 g, 8.3 mmol) in dry DMF (20 ml) was added piperidine (1.65 mL, 17 mmol, 2 eq) dropwise at r.t. The mixture was stirred at r.t. for 30 min, and solid precipitate formed. Dry DCM (50 mL) was added, and the mixture became transparent immediately. The mixture was stirred at r.t. for another 30 min, and LCMS showed 10e was consumed. It was concentrated to dryness under reduced pressure (make sure no piperidine remained), and the residue was partitioned between EtOAc and H₂O (50 mL/20 mL). Aqueous phase was washed with EtOAc (50 mL×2) and concentrated to give (S)-2-amino-N-(4-(hydroxymethyl)phenyl)-5-ureidopentanamide 2d as an oily residual (2.2 g, 94%) (contained small amount of DMF).

Commercially available 1,1-cyclobutanedicarboxylic acid, 1,1-diethyl ester (CAS Reg. No. 3779-29-1) was converted by limited saponification with aqueous base to the half acid/ester 1,1-cyclobutanedicarboxylic acid, 1-ethyl ester (CAS Reg No. 54450-84-9) and activation with a coupling reagent such as TBTU (0-(Benzotriazol-1-yl)-N,N,N′,N′-tetramethyluronium tetrafluoroborate, also called: N,N,N′,N′-Tetramethyl-O-(benzotriazol-1-yl)uronium tetrafluoroborate, CAS No. 125700-67-6, Sigma-Aldrich B-2903), and N-hydroxysuccinimide to the NHS ester, 1-(2,5-dioxopyrrolidin-1-yl) 1-ethyl cyclobutane-1,1-dicarboxylate.

To a solution of 1-(2,5-dioxopyrrolidin-1-yl) 1-ethyl cyclobutane-1,1-dicarboxylate (8 g, 29.7 mmol) in DME (50 mL) was added a solution of 2d (6.0 g, 21.4 mmol) and NaHCO₃ (7.48 g, 89.0 mmol) in water (30 mL). After the mixture was stirred at r.t. for 16 h, it was concentrated to dryness under reduced pressure and the residue was purified by column chromatography (DCM:MeOH=10:1) to give (S)-ethyl 1-((1-(4-(hydroxymethyl)phenyl)-2-oxo-6-ureidohexan-3-yl)carbamoyl)cyclobutanecarboxylate 2e as white solid (6.4 g, 68.7%). LCMS (ESI): m/z 435.0 [M+1]

To a stirred solution of 2e (6.4 g, 14.7 mmol) in a mixture of THF and MeOH (20 mL/10 mL) was added a solution of LiOH H₂O (1.2 g, 28.6 mmol) in H₂O (20 mL) at r.t. After the reaction mixture was stirred at r.t. for 16 h, solvent was removed under reduced pressure, the residue obtained was purified by prep-HPLC to give (S)-1-(1-(4-(hydroxymethyl)phenylamino)-1-oxo-5-ureidopentan-2-ylcarbamoyl)cyclobutanecarboxylic acid 2 (3.5 g, yield: 58.5%). LCMS (ESI): m/z 406.9 [M+1]. ¹H NMR (400 MHz, Methanol-d₄) δ 8.86 (d, J=8.4 Hz, 2H), 8.51 (d, J=8.4 Hz, 2H), 5.88-5.85 (m, 1H), 5.78 (s, 2H), 4.54-4.49 (m, 3H), 4.38-4.32 (m, 1H), 3.86-3.75 (m, 1H), 3.84-3.80 (m, 2H), 3.28-3.21 (m, 1H), 3.30-3.24 (m, 1H), 3.00-2.80 (m, 1H), 2.37-2.28 (m, 2H).

### Example 3 Synthesis of S)—N-(5-(2,5-dioxo-2,5-dihydro-1H-pyrrol-1-yl)pentyl)-N-(1-(4-(hydroxymethyl)phenylamino)-1-oxo-5-ureidopentan-2-yl)cyclobutane-1,1-dicarboxamide, 3

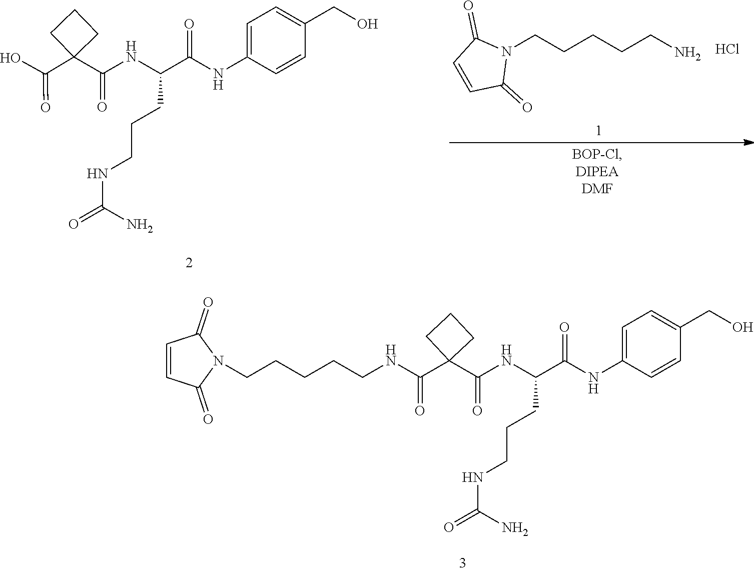 [SMILES: NC(=O)NCCC[C@H](NC(=O)C1(C(=O)O)CCC1)C(=O)Nc1ccc(CO)cc1 ; NCCCCCN1C(=O)C=CC1=O ; Cl ; NC(=O)NCCC[C@H](NC(=O)C1(C(=O)NCCCCCN2C(=O)C=CC2=O)CCC1)C(=O)Nc1ccc(CO)cc1]

Diisopropylethylamine, DIPEA (1.59 g, 12.3 mmol) and bis(2-oxo-3-oxazolidinyl)phosphinic chloride, BOP—Cl (CAS Reg. No. 68641-49-6, Sigma-Aldrich, 692 mg, 2.71 mmol) was added to a solution of (S)-1-(1-(4-(hydroxymethyl)phenylamino)-1-oxo-5-ureidopentan-2-ylcarbamoyl)cyclobutanecarboxylic acid 2 (1 g, 2.46 mmol) in DMF (10 mL) at 0 °C, followed by 1-(5-aminopentyl)-1H-pyrrole-2,5-dione hydrochloride 1 (592 mg, 2.71 mmol). The mixture was stirred at 0 °C for 0.5 h. The reaction mixture was quenched with citric acid solution (10 mL), extracted with DCM/MeOH (10:1). The organic layer was dried and concentrated, and the residue was purified by column chromatography on silica gel (DCM:MeOH=10:1) to give to give 3 (1.0 g, 71%), also referred to as MC-CBDK-cit-PAB-OH. LCMS (ESI): M+H⁺=571.28. ¹H NMR (400 MHz, DMSO-d₆): δ 10.00 (s, 1H), 7.82-7.77 (m, 2H), 7.53 (d, J=8.4 Hz, 2H), 7.19 (d, J=8.4 Hz, 2H), 6.96 (s, 2H), 5.95 (t, J=6.4 Hz, 1H), 5.39 (s, 2H), 5.08 (t, J=5.6 Hz, 1H), 4.40-4.35 (m, 3H), 4.09 (d, J=4.8 Hz, 1H), 3.01 (d, J=3.2 Hz, 2H), 3.05-2.72 (m, 4H), 2.68-2.58 (m, 3H), 2.40-2.36 (m, 4H), 1.72-1.70 (m, 3H), 1.44-1.42 (m, 1H), 1.40-1.23 (m, 6H), 1.21-1.16 (m, 4H).

### Example 4 Synthesis of (S)—N-(1-(4-(chloromethyl)phenylamino)-1-oxo-5-ureidopentan-2-yl)-N-(5-(2,5-dioxo-2,5-dihydro-1H-pyrrol-1-yl)pentyl)cyclobutane-1,1-dicarboxamide, 4

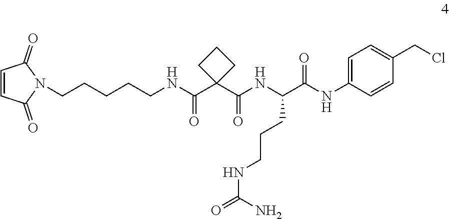 [SMILES: NC(=O)NCCC[C@H](NC(=O)C1(C(=O)NCCCCCN2C(=O)C=CC2=O)CCC1)C(=O)Nc1ccc(CCl)cc1]

A solution of 3 (2.0 g, 3.5 mmol) in N,N-dimethylformamide, DMF or N-methylpyrrolidone, NMP (50 mL) was treated with thionyl chloride, SOCl₂ (1.25 g, 10.5 mmol) in portions dropwise at 0 °C. The reaction remained yellow. The reaction was monitored by LC/MS indicating >90% conversion. After the reaction mixture was stirred at 20 °C for 30 min or several hours, it was diluted with water (50 mL) and extracted with EtOAc (50 mL×3). The organic layer was dried, concentrated and purified by flash column (DCM:MeOH=20:1) to form 4, also referred to as MC-CBDK-cit-PAB-Cl as a gray solid. LCMS: (5-95, AB, 1.5 min), 0.696 min, m z=589.0 [M+1]⁺.

### Example 5 Synthesis of (S)-4-(2-(1-(5-(2,5-dioxo-2,5-dihydro-1H-pyrrol-1-yl)pentylcarbamoyl)cyclobutanecarboxamido)-5-ureidopentanamido)benzyl 4-nitrophenyl carbonate, 5

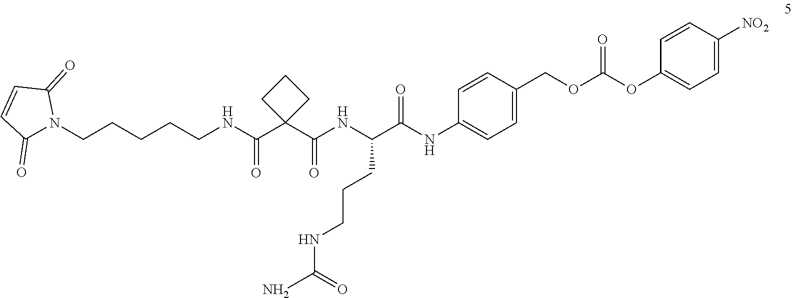 [SMILES: NC(=O)NCCC[C@H](NC(=O)C1(C(=O)NCCCCCN2C(=O)C=CC2=O)CCC1)C(=O)Nc1ccc(COC(=O)Oc2ccc([N+](=O)[O-])cc2)cc1]

To a solution of 3 in anhydrous DMF was added diisopropylethylamine (DIEA), followed by PNP carbonate (bis(4-nitrophenyl) carbonate). The reaction solution was stirred at room temperature (r.t.) for 4 hours and the mixture was purified by prep-HPLC to afford 5. LCMS (ESI): M+H⁺=736.29.

### Example 6 Synthesis of 4-nitrophenyl 2-(pyridin-2-yldisulfanyl)ethyl carbonate, 6

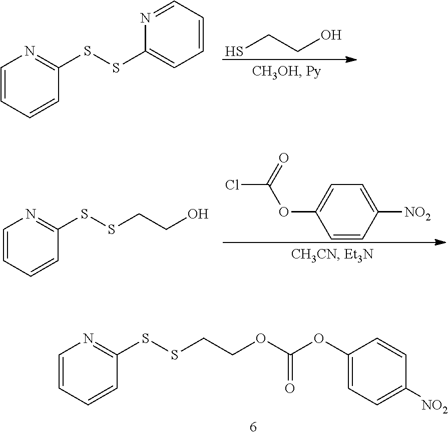

Following the procedures of WO 2016/040825, incorporated herein, 1,2-di(pyridin-2-yl)disulfane and 2-mercaptoethanol were reacted in pyridine and methanol at room temperature to give 2-(pyridin-2-yldisulfanyl)ethanol. Acylation with 4-nitrophenyl carbonochloridate in triethylamine and acetonitrile gave 4-nitrophenyl 2-(pyridin-2-yldisulfanyl)ethyl carbonate 6.

### Example 7 Synthesis of 2-((5-nitropyridin-2-yl)disulfanyl)ethanamine hydrochloride

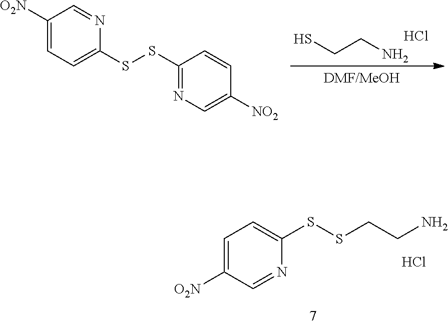

To a mixture of 1,2-bis(5-nitropyridin-2-yl)disulfane (1.0 g, 3.22 mmol) in anhydrous DMF/MeOH (25 mL/25 mL) was added HOAc (0.1 mL), followed by 2-aminoethanethiol hydrochloride (183 mg, 1.61 mmol). After the reaction mixture was stirred at r.t. overnight, it was concentrated under vacuum to remove the solvent, and the residue was washed with DCM (30 mL×4) to afford 7 as pale yellow solid (300 mg, 69.6%). ¹H NMR (400 MHz, DMSO-d₆) δ 9.28 (d, J=2.4 Hz, 1H), 8.56 (dd, J=8.8, 2.4 Hz, 1H), 8.24 (s, 4H), 8.03 (d, J=8.8 Hz, 1H), 3.15-3.13 (m, 2H), 3.08-3.06 (m, 2H)

### Example 8 Synthesis of 4-nitrophenyl 2-((5-nitropyridin-2-yl)disulfanyl)ethyl carbonate, 8

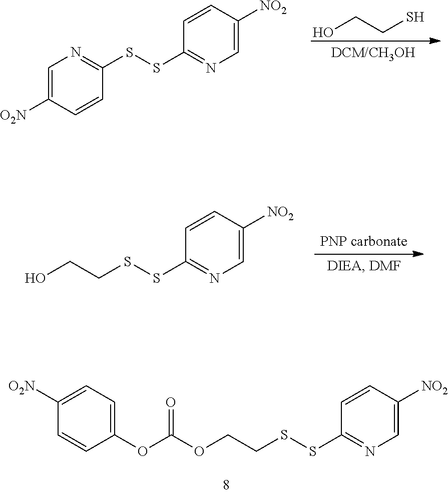

A solution of 1,2-bis(5-nitropyridin-2-yl)disulfane (9.6 g, 30.97 mmol) and 2-mercaptoethanol (1.21 g, 15.49 mmol) in anhydrous DCM/CH₃OH (250 mL/250 mL) was stirred at r.t. under N₂ for 24 h. After the mixture was concentrated under vacuum, and the residue was diluted with DCM (300 mL). Manganese oxide, MnO₂ (10 g) was added and the mixture was stirred at r.t. for another 0.5 h. The mixture was purified by column chromatography on silica gel (DCM/MeOH=100/1 to 100/1) to afford 2-((5-nitropyridin-2-yl)disulfanyl)ethanol (2.2 g, 61.1%) as brown oil. ¹H NMR (400 MHz, CDCl₃) δ 9.33 (d, J=2.8 Hz, 1H), 8.38-8.35 (dd, J=9.2, 2.8 Hz, 1H), 7.67 (d, J=9.2 Hz, 1H), 4.10 (t, J=7.2 Hz, 1H), 3.81-3.76 (q, 2H), 3.01 (t, J=5.2 Hz, 2H).

To a solution of 2-((5-nitropyridin-2-yl)disulfanyl)ethanol (500 mg, 2.15 mmol) in anhydrous DMF (10 mL) was added DIEA (834 mg, 6.45 mmol), followed by PNP carbonate (bis(4-nitrophenyl) carbonate, 1.31 g, 4.31 mmol). The reaction solution was stirred at r.t for 4 h and the mixture was purified by prep-HPLC (FA) to afford 8 (270 mg, 33.1%) as light brown oil. ¹H NMR (400 MHz, CDCl₃) δ 9.30 (d, J=2.4 Hz, 1H), 8.43-8.40 (dd, J=8.8, 2.4 Hz, 1H), 8.30-8.28 (m, 2H), 7.87 (d, J=8.8 Hz, 1H), 7.39-7.37 (m, 2H), 4.56 (t, J=6.4 Hz, 2H), 3.21 (t, J=6.4 Hz, 2H).

### Example 9 Synthesis of 2-((5-nitropyridin-2-yl)disulfanyl)propan-1-amine, 9

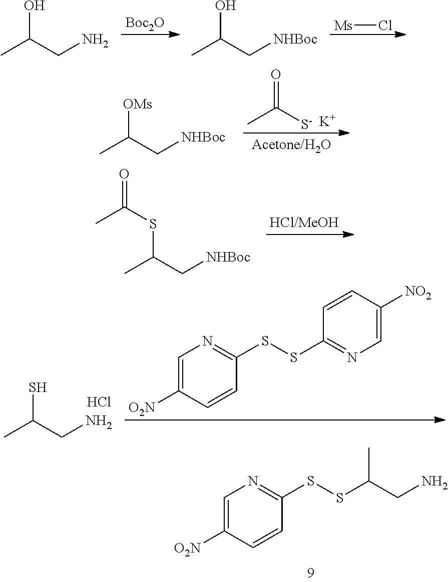

To a stirred solution of 1-aminopropan-2-ol (10 g, 133 mmol) in MeOH (360 mL) and H₂O (40 mL) was added Boc₂O (37 g, 169 mmol). After the reaction mixture was stirred at r.t. for 5 h, it was concentrated and purified by chromatography (EtOAc/PE=10%-50%) to give tert-butyl 2-hydroxypropylcarbamate as a colorless oil (19.8 g, yield: 85%).

To a stirred solution of tert-butyl 2-hydroxypropylcarbamate (10 g, 57 mmol) and Et₃N (17 g, 171 mmol) in DCM (130 mL) was added a solution of MsCl (methanesulfonyl chloride, 13 g, 114 mmol). After the reaction mixture was stirred at r.t. for 4 h, it was washed with ice water (200 mL×3) and brine (200 mL). The organic layer was concentrated to give 1-(tert-butoxycarbonylamino)propan-2-yl methanesulfonate as a red oil (12 g, yield: 83%).

To a stirred solution of 1-(tert-butoxycarbonylamino)propan-2-yl methanesulfonate (6 g, 23.7 mmol) in acetone (70 mL) was added a solution of potassium thioacetate (potassium ethanethioate, 5.4 g, 47.3 mmol) in H₂O (100 mL). The reaction mixture was stirred at 60 °C for 12 h. The mixture was concentrated and extracted with DCM (200 ml×2). The combined organic layers were concentrated and purified by chromatography to give S-1-(tert-butoxycarbonylamino)propan-2-yl ethanethioate as a red solid (1.1 g, yield: 20%). ¹H NMR (400 MHz, CDCl3-d) 1.30 (d, J=7.09 Hz, 3H) 1.44 (s, 9H) 2.33 (s, 3H) 3.16-3.42 (m, 2H) 3.58-3.71 (m, 1H)

To a stirred solution of S-1-(tert-butoxycarbonylamino)propan-2-yl ethanethioate (500 mg, 2.15 mmol) in MeOH (5 mL) was added HCl/MeOH (10 mL) dropwise. After the reaction mixture was stirred at r.t. for 3 h, it was concentrated to give 1-aminopropane-2-thiol hydrochloride, used directly in the next step.

To a solution of 1,2-bis(5-nitropyridin-2-yl)disulfane (1.33 g, 4.3 mmol) in DCM (35 mL) was added a solution of 1-aminopropane-2-thiol hydrochloride (273 mg, 2.15 mmol). The mixture was stirred at 15 °C for 12 h. MnO₂ (374.1 mg, 4.3 mmol) was added to the mixture and stirred at 15 °C for 10 min. The solid was washed with DCM (100 mL) and MeOH (30 mL×3). The solution was concentrated to give 9 as a yellow solid (300 mg, 57%). LCMS (ESI): RT=0.546 min, M+H⁺=245.7.

### Example 10 Synthesis of 4-nitrobenzyl ((2,6-dioxo-3-(1-oxoisoindolin-2-yl)piperidin-1-yl)methyl)carbamate, 10

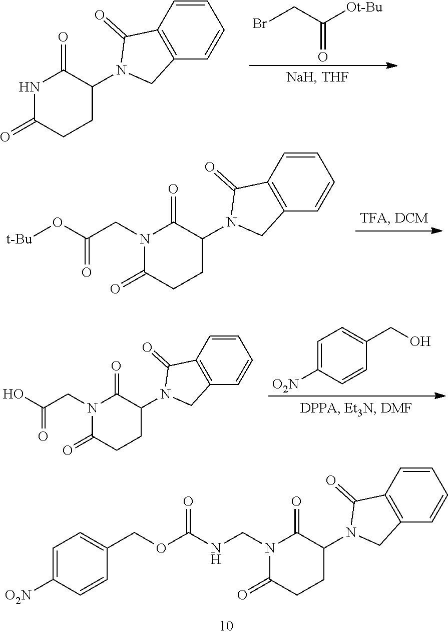

### Example 11 Synthesis of 2-((5-nitropyridin-2-yl)disulfaneyl)ethyl ((2,6-dioxo-3-(1-oxoisoindolin-2-yl)piperidin-1-yl)methyl)carbamate, 11

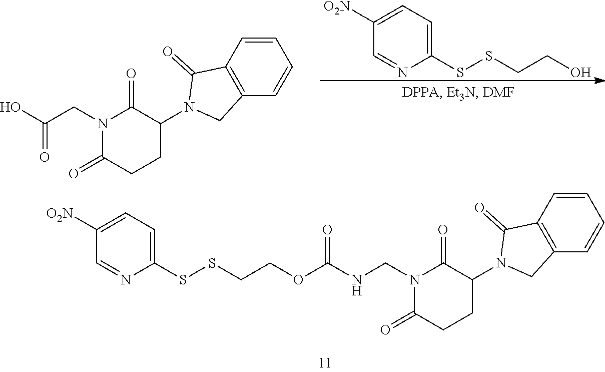

### Example cD-1 Synthesis of 4-(3,5-difluoropyridin-2-yl)-N-(3-((2-(2,6-dioxopiperidin-3-yl)-1-oxoisoindolin-4-yl)amino)propyl)-10-methyl-7-((methylsulfonyl)methyl)-11-oxo-3,4,10,11-tetrahydro-1H-1,4,10-triazadibenzo[cd,f]azulene-6-carboxamide, cD-1

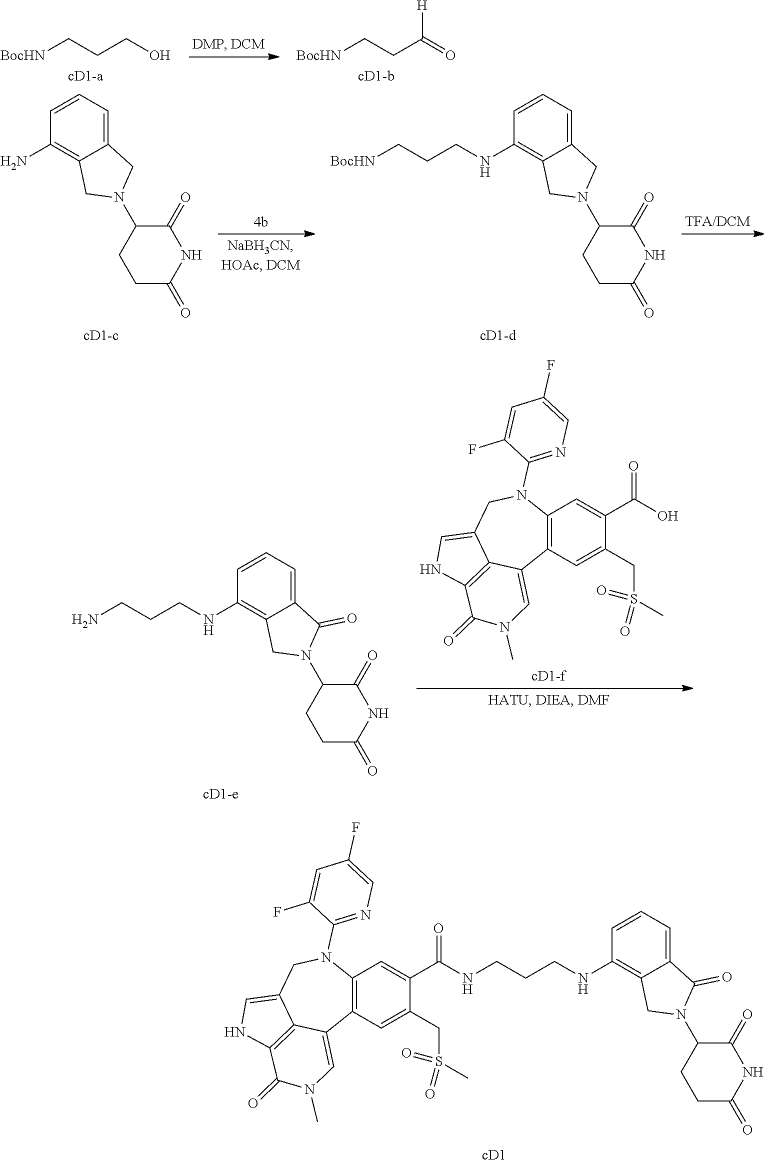

### Example cD-2 Synthesis of 4-(3,5-difluoropyridin-2-yl)-N-(4-((2-(2,6-dioxopiperidin-3-yl)-1-oxoisoindolin-4-yl)amino)butyl)-10-methyl-7-((methylsulfonyl)methyl)-11-oxo-3,4,10,11-tetrahydro-1H-1,4,10-triazadibenzo[cd,f]azulene-6-carboxamide, cD-2

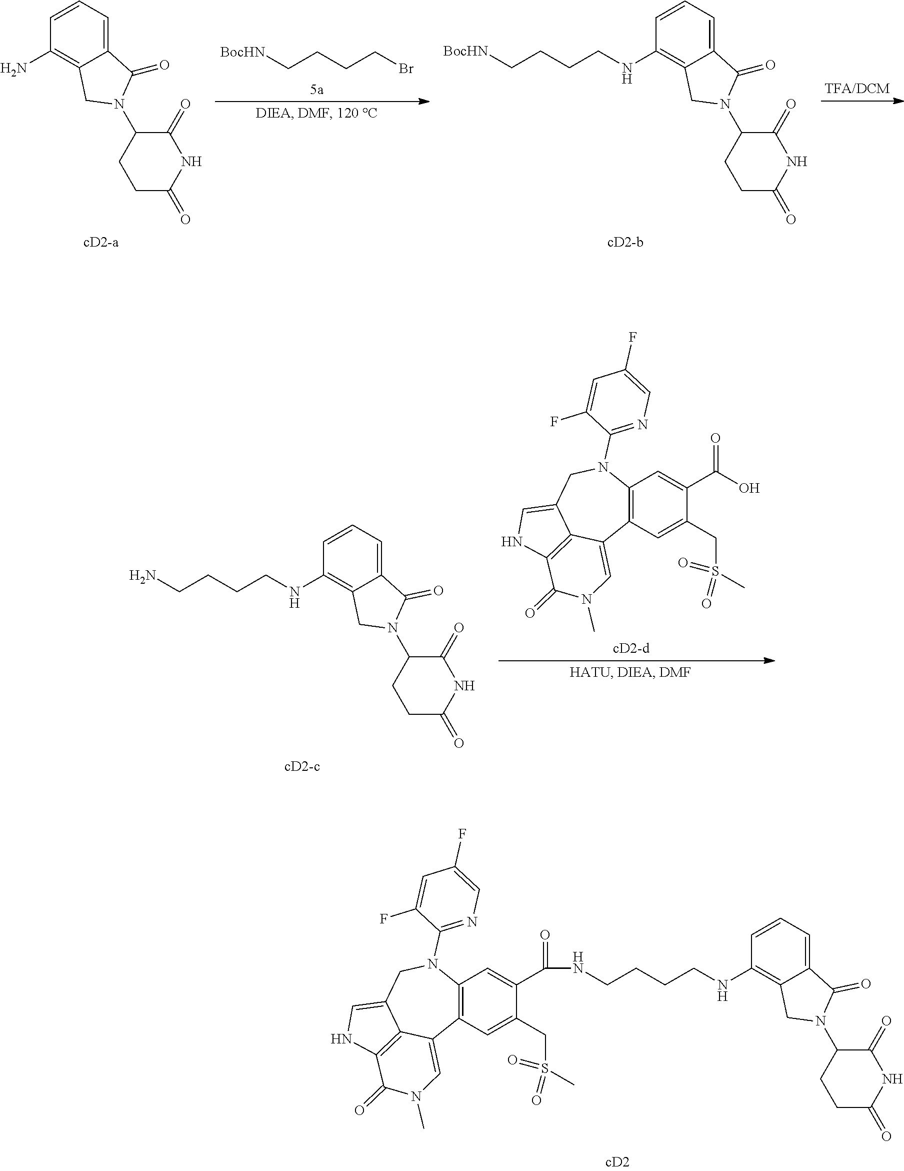 [SMILES: Nc1cccc2c1CN(C1CCC(=O)NC1=O)C2=O ; CC(C)(C)OC(=O)NCCCCBr ; CC(C)(C)OC(=O)NCCCCNc1cccc2c1CN(C1CCC(=O)NC1=O)C2=O ; NCCCCNc1cccc2c1CN(C1CCC(=O)NC1=O)C2=O ; Cn1cc2c3c(c[nH]c3c1=O)CN(c1ncc(F)cc1F)c1cc(C(=O)O)c(CS(C)(=O)=O)cc1-2 ; Cn1cc2c3c(c[nH]c3c1=O)CN(c1ncc(F)cc1F)c1cc(C(=O)NCCCCNc3cccc4c3CN(C3CCC(=O)NC3=O)C4=O)c(CS(C)(=O)=O)cc1-2]

### Example cD-3 Synthesis of 4-(3,5-difluoropyridin-2-yl)-N-(5-((2-(2,6-dioxopiperidin-3-yl)-1-oxoisoindolin-4-yl)amino)pentyl)-10-methyl-7-((methylsulfonyl)methyl)-11-oxo-3,4,10,11-tetrahydro-1H-1,4,10-triazadibenzo[cd,f]azulene-6-carboxamide, cD-3

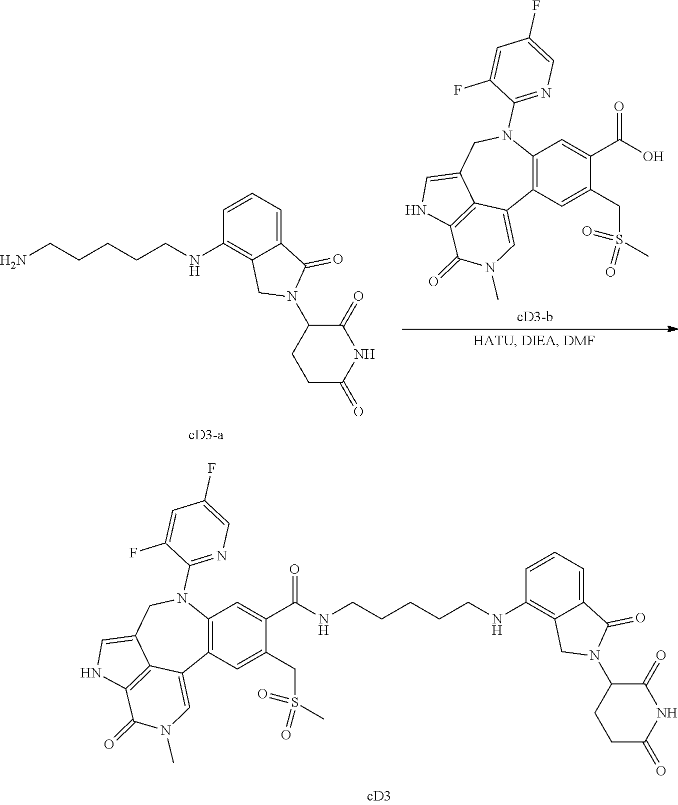 [SMILES: NCCCCCNc1cccc2c1CN(C1CCC(=O)NC1=O)C2=O ; Cn1cc2c3c(c[nH]c3c1=O)CN(c1ncc(F)cc1F)c1cc(C(=O)O)c(CS(C)(=O)=O)cc1-2 ; Cn1cc2c3c(c[nH]c3c1=O)CN(c1ncc(F)cc1F)c1cc(C(=O)NCCCCCNc3cccc4c3CN(C3CCC(=O)NC3=O)C4=O)c(CS(C)(=O)=O)cc1-2]

### Example cD-4 Synthesis of 4-(3,5-difluoropyridin-2-yl)-N-(6-((2-(2,6-dioxopiperidin-3-yl)-1-oxoisoindolin-4-yl)amino)hexyl)-10-methyl-7-((methylsulfonyl)methyl)-11-oxo-3,4,10,11-tetrahydro-1H-1,4,10-triazadibenzo[cd,f]azulene-6-carboxamide, cD-4

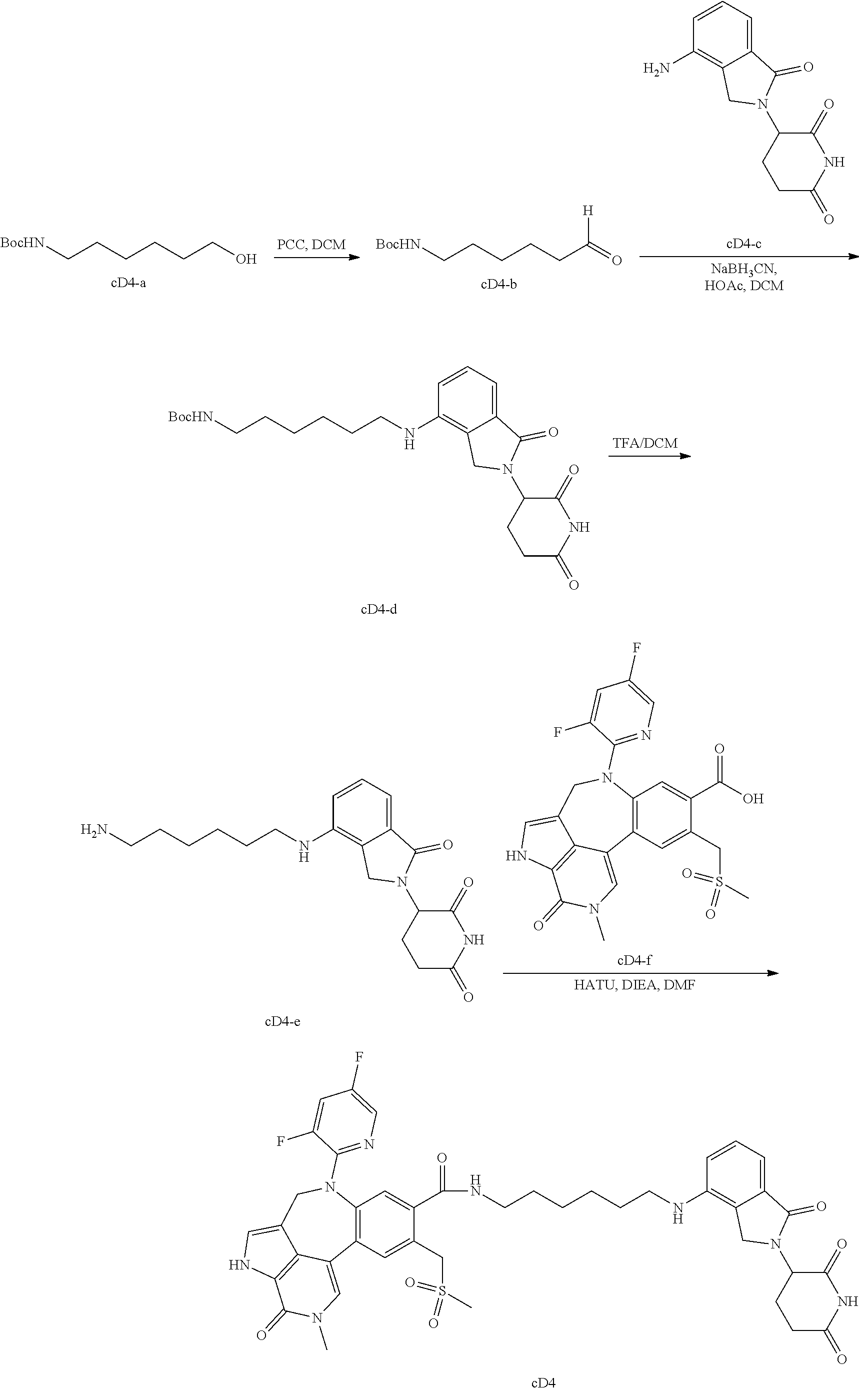

### Example cD-5 Synthesis of 4-(3,5-difluoropyridin-2-yl)-N-(7-((2-(2,6-dioxopiperidin-3-yl)-1-oxoisoindolin-4-yl)amino)heptyl)-10-methyl-7-((methylsulfonyl)methyl)-11-oxo-3,4,10,11-tetrahydro-1H-1,4,10-triazadibenzo[cd,f]azulene-6-carboxamide, cD-5

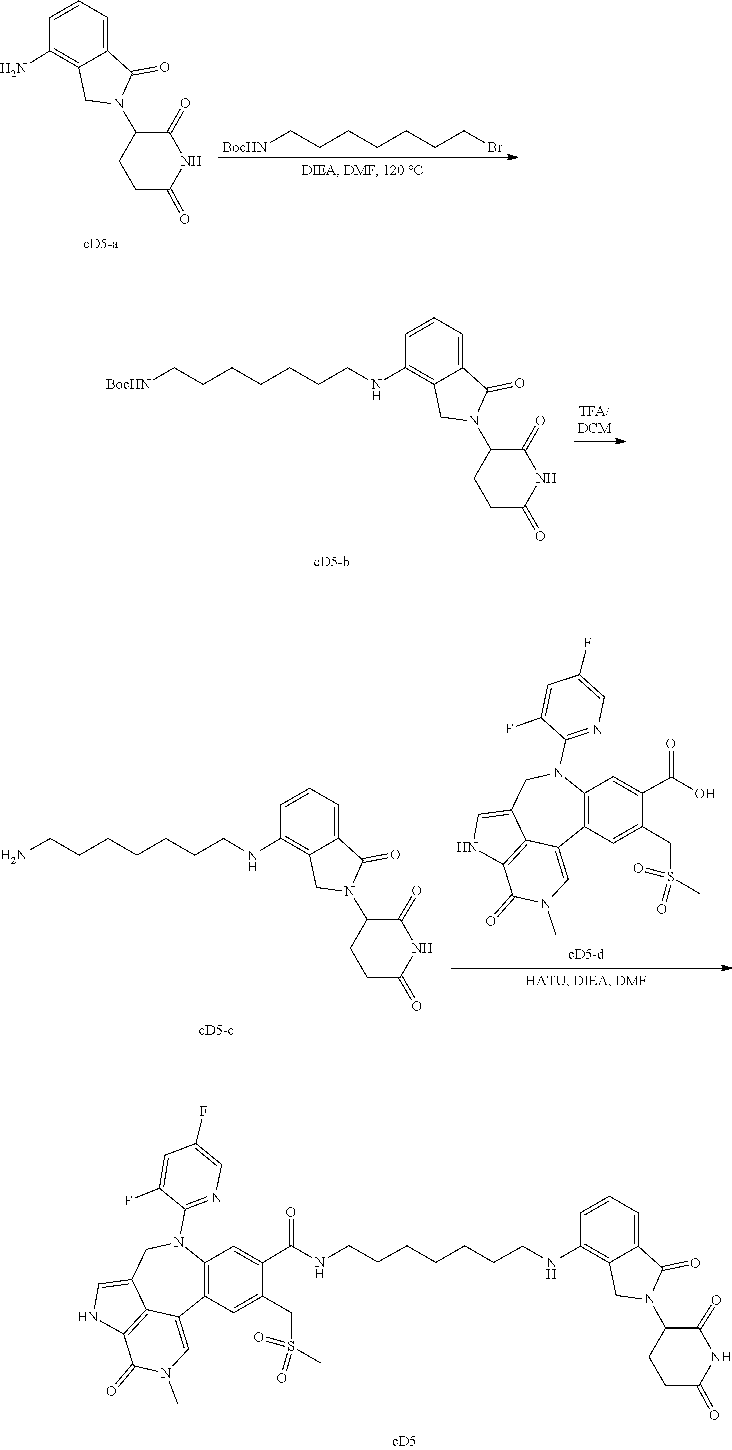 [SMILES: Nc1cccc2c1CN(C1CCC(=O)NC1=O)C2=O ; CC(C)(C)OC(=O)NCCCCCCCBr ; CC(C)(C)OC(=O)NCCCCCCCNc1cccc2c1CN(C1CCC(=O)NC1=O)C2=O ; NCCCCCCCNc1cccc2c1CN(C1CCC(=O)NC1=O)C2=O ; Cn1cc2c3c(c[nH]c3c1=O)CN(c1ncc(F)cc1F)c1cc(C(=O)O)c(CS(C)(=O)=O)cc1-2 ; Cn1cc2c3c(c[nH]c3c1=O)CN(c1ncc(F)cc1F)c1cc(C(=O)NCCCCCCCNc3cccc4c3CN(C3CCC(=O)NC3=O)C4=O)c(CS(C)(=O)=O)cc1-2]

### Example cD-6 Synthesis of 4-((3-cyclopropyl-1-ethyl-1H-pyrazol-5-yl)amino)-7-(3,5-dimethylisoxazol-4-yl)-N-(5-((2-(2,6-dioxopiperidin-3-yl)-1-oxoisoindolin-4-yl)amino)pentyl)-6-methoxy-9H-pyrimido[4,5-b]indole-2-carboxamide, cD-6

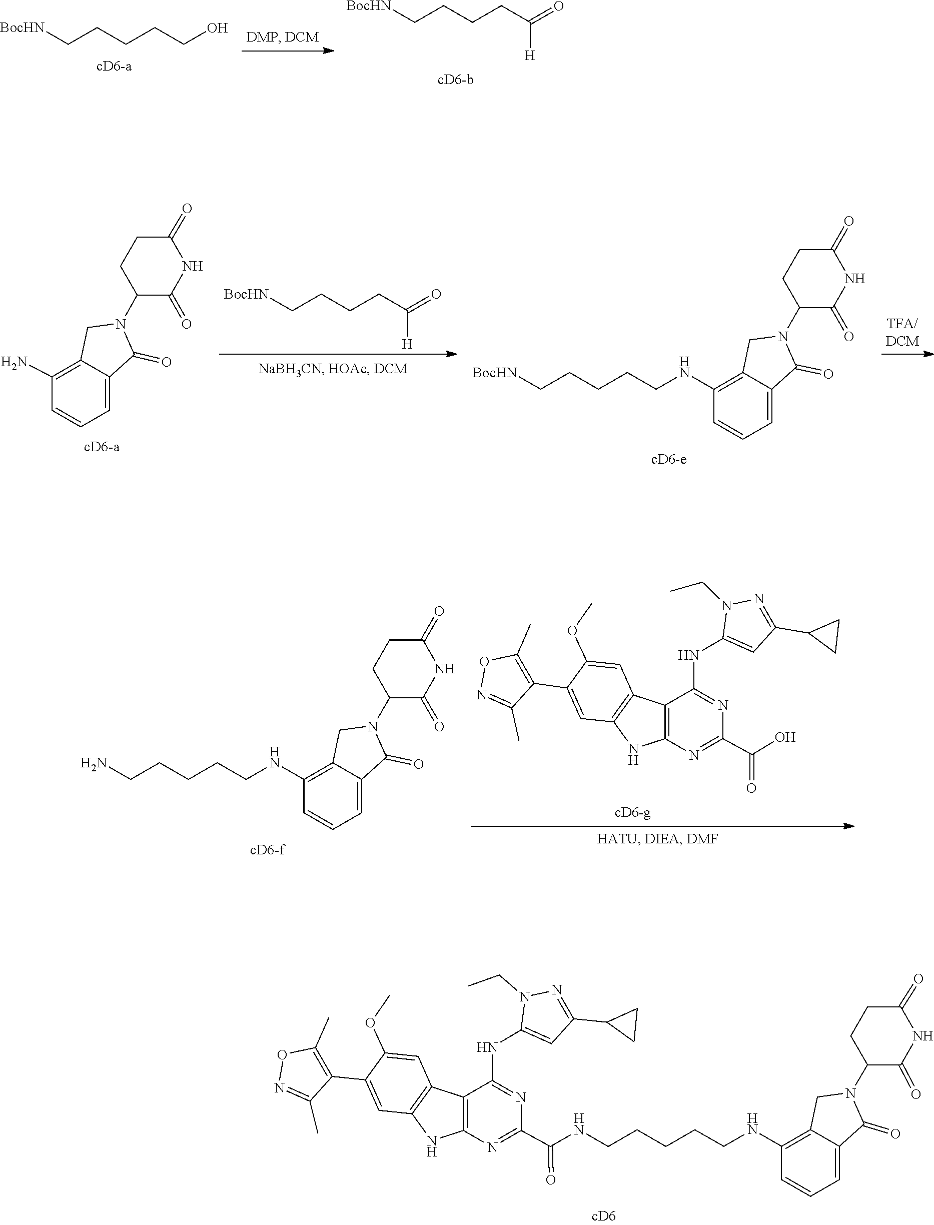

### Example cDLI-1 Synthesis of 4-((S)-2-(1-((5-(2,5-dioxo-2,5-dihydro-1H-pyrrol-1-yl)pentyl)carbamoyl)cyclobutane-1-carboxamido)propanamido)benzyl (7-(4-(3,5-difluoropyridin-2-yl)-10-methyl-7-((methylsulfonyl)methy)-11-oxo-3,4,10,11-tetrahydro-1H-1,4,10-triazadibenzo[cd,f]azulene-6-carboxamido)heptyl) (2-(2,6-dioxopiperidin-3-yl)-1-oxoisoindolin-4-yl)carbamate, cDLI-1

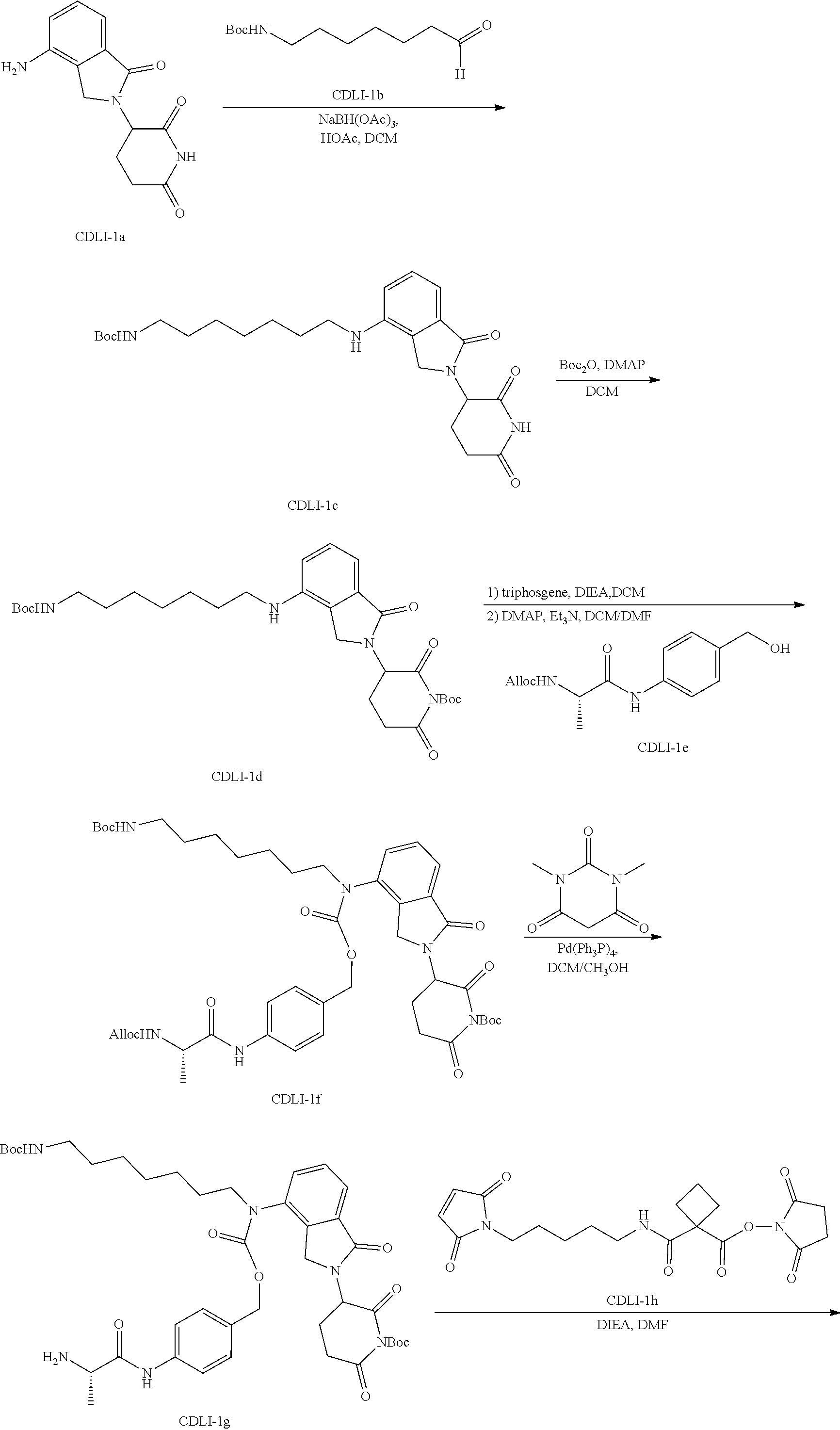

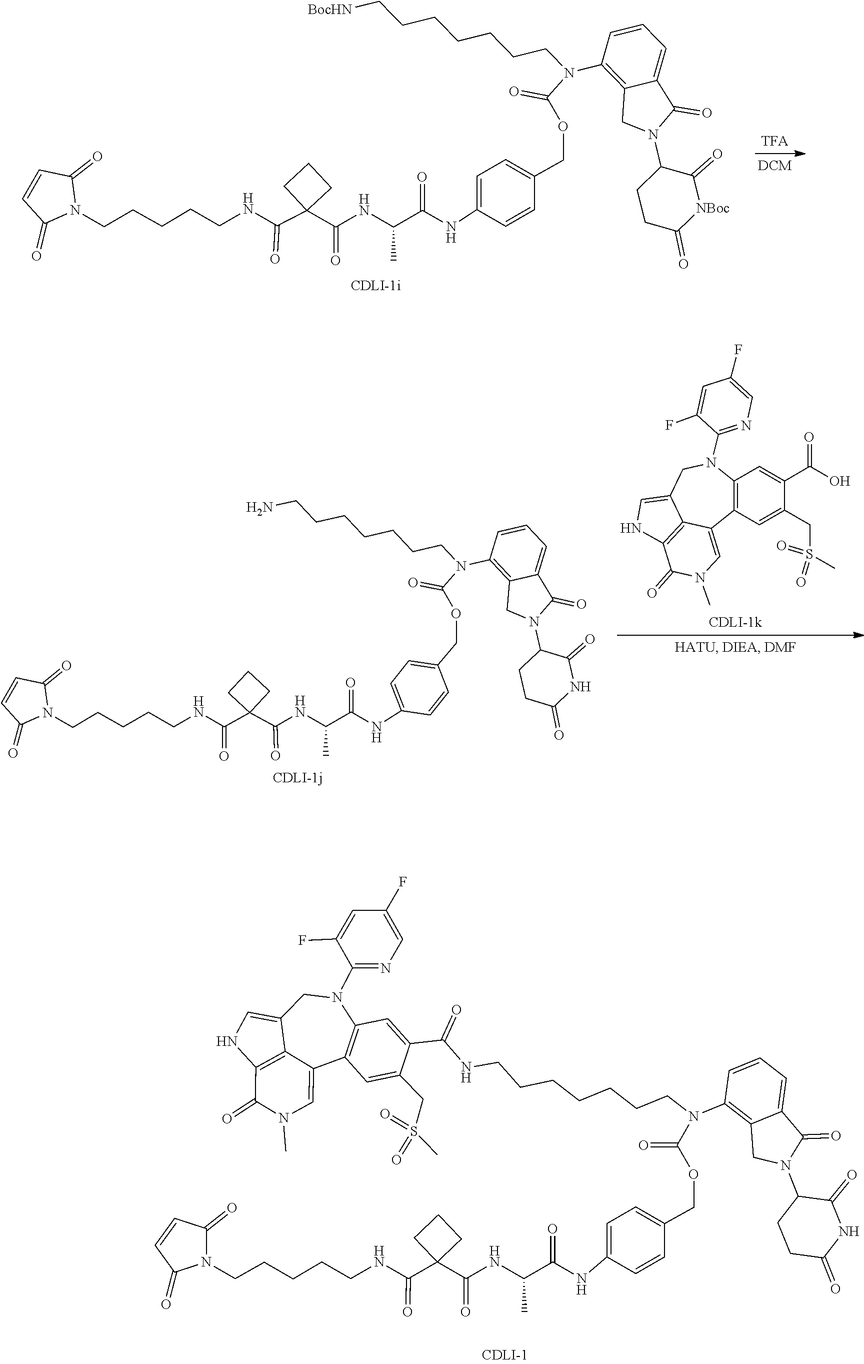

### Preparation of tert-butyl (7-((2-(2,6-dioxopiperidin-3-yl)-1-oxoisoindolin-4-yl)amino)heptyl)carbamate, cDLI-1c

To a solution of tert-butyl (7-oxoheptyl)carbamate, cDLI-1b (636.9 mg, 2.78 mmol) and 3-(4-amino-1-oxoisoindolin-2-yl)piperidine-2,6-dione, cDLI-1a (600.00 mg, 2.31 mmol) in anhydrous dichloromethane (50 mL) was added acetic acid (0.02 mL, 0.26 mmol). The mixture was stirred at 25 °C for 2 hrs. Then to the mixture was added NaBH(OAc)₃ (1226.2 mg, 5.79 mmol) and the mixture was stirred at 25 °C for 12 hrs. TLC (60% EtOAc in petroleum ether, R_{f}=0.5) indicated the reaction was completed. The reaction mixture was washed with water (30 mL×2), the organic layer was dried over anhydrous sodium sulfate, filtered, and concentrated under reduced pressure to give the crude, which was purified by column chromatography on silica gel (solvent gradient: 0-6% methanol in dichloromethane) to afford cDLI-1c (0.70 g, 64%) as pale yellow oil. LCMS (5-95, AB, 1.5 min): RT=0.892 min, m/z=495.2 [M+Na]⁺.

### Preparation of tert-butyl 3-(4-((7-((tert-butoxycarbonyl)amino)heptyl)amino)-1-oxoisoindolin-2-yl)-2,6-dioxopiperidine-1-carboxylate, cDLI-1d

To a solution of cDLI-1c (300.00 mg, 0.63 mmol) and di-tert-butyl decarbonate, Boc₂₀ (207.8 mg, 0.95 mmol) in Dichloromethane (20 mL) was added 4-dimethylaminopyridine (116.3 mg, 0.95 mmol) and triethylamine (0.13 mL, 0.95 mmol). The mixture was stirred at 25 °C for 2 hrs. TLC (60% EtOAc in petroleum ether, R_{f}=0.6) indicated the reaction was completed. The mixture was diluted with Dichloromethane (45 mL), and washed with aqueous citric acid (15 mL), water (15 mL), sat brine (15 mL). The organic layer was concentrated and purified by flash column (eluting with 0-60% EtOAc in petroleum ether) to afford cDLI-1d (300 mg, 83%) as pale yellow solid.

### Preparation of tert-butyl 3-(4-((((4-((S)-2-(((allyloxy)carbonyl)amino)propanamido) benzyl)oxy)carbonyl)(7-((tert-butoxycarbonyl)amino)heptyl)amino)-1-oxoisoindolin-2-yl)-2,6-dioxopiperidine-1-carboxylate, cDLI-1f

To a mixture of triphosgene (150.00 mg, 0.51 mmol) and 4 A molecular sieves in dichloromethane (10 mL) was added a solution of N,N-diisopropylethylamine (228.10 uL, 1.3 mmol) and cDLI-1d (250.00 mg, 0.44 mmol) in dichloromethane (10 mL). The mixture was stirred at 25 °C for 1 h. TLC (5% MeOH in DCM, R_{f}=0.6) indicated the reaction was completed. The mixture was concentrated and used for next step directly. To the crude product (277.27 mg, 0.44 mmol, theoretical), was added allyl (S)-(1-((4-(hydroxymethyl)phenyl)amino)-1-oxopropan-2-yl)carbamate, cDLI-1e (243 mg, 0.87 mmol) and 4 A molecular sieve in dichloromethane (5 mL) and N,N-dimethylformamide, DMF (1 mL) was added along with triethylamine (0.18 mL, 1.31 mmol) and 4-dimethylaminopyridine, DMAP (160.00 mg, 1.31 mmol). The mixture was stirred at 35 °C for 12 hrs. TLC (10% MeOH in DCM, R_{f}=0.5) indicated the reaction was completed. The mixture was filtrated and diluted with DCM (50 mL), washed with sat. citric acid (10 mL), sat. Brine (10 mL). The organic layer was concentrated in vacuum and purified by flash column (eluting 0-10% MeOH in DCM) to afford cDLI-1f (75 mg, 19.6%) as a pale yellow solid. LCMS (10-80, AB, 7.0 min): RT=4.491 min, m/z=877.5 [M+H]⁺.

### Preparation of tert-butyl 3-(4-((((4-((S)-2-aminopropanamido)benzyl)oxy)carbonyl)(7-((tert-butoxycarbonyl)amino)heptyl)amino)-1-oxoisoindolin-2-yl)-2,6-dioxopiperidine-1-carboxylate, cDLI-1g

To a solution of cDLI-1f (75.00 mg, 0.09 mmol) and 1,3-dimethylpyrimidine-2,4,6 (1H,3H,5H)-trione, also known as 1,3-dimethylbarbituric acid (66.8 mg, 0.43 mmol) in dichloromethane (3 mL) and methanol (3 mL) was added Pd(PPh₃)₄ (19.8 mg, 0.02 mmol) at 25 °C. The reaction mixture was stirred under nitrogen atmosphere at 25 °C for 3 hrs. TLC (10% MeOH in DCM, R_{f}=0.3) indicated the reaction was completed. The mixture was filtered and the filtrate was concentrated to give the crude product, which was purified by Pre-TLC (10% MeOH in DCM) to afford cDLI-1g (30 mg, 44.2%) as a white solid. LCMS (5-95, AB, 1.5 min): RT=0.883 min, m/z=793.4 [M+H]⁺.

### Preparation of tert-butyl 3-(4-((7-((tert-butoxycarbonyl)amino)heptyl)(((4-((S)-2-(1-((5-(2,5-dioxo-2,5-dihydro-1H-pyrrol-1-yl)pentyl)carbamoyl)cyclobutane-1-carboxamido)propanamido)benzyl)oxy)carbonyl)amino)-1-oxoisoindolin-2-yl)-2,6-dioxopiperidine-1-carboxylate, cDLI-1i

To a solution of cDLI-1g (30.00 mg, 0.04 mmol) and 2,5-dioxopyrrolidin-1-yl 1-((5-(2,5-dioxo-2,5-dihydro-1H-pyrrol-1-yl)pentyl)carbamoyl)cyclobutane-1-carboxylate, cDLI-1 h (46.0 mg, 0.11 mmol) in N,N-dimethylformamide (1 mL) was added N,N-diisopropylethylamine (14.7 mg, 0.11 mmol). The reaction mixture was stirred at 20 °C for 2 hrs. TLC (10% MeOH in DCM, R_{f}=0.5) indicated the reaction was completed. The mixture was concentrated and purified by pre-TLC (10% MeOH in DCM) to give cDLI-1i (10 mg, 24.4%) as a white solid. LCMS (10-80, AB, 7.0 min): RT=4.728 min, m/z=1083.7 [M+H]⁺.

### Preparation of 4-((S)-2-(1-((5-(2,5-dioxo-2,5-dihydro-1H-pyrrol-1-yl)pentyl)carbamoyl)cyclobutane-1-carboxamido)propanamido)benzyl (7-aminoheptyl)(2-(2,6-dioxopiperidin-3-yl)-1-oxoisoindolin-4-yl)carbamate, cDLI-1j

To a solution of cDLI-1i (10.00 mg, 0.01 mmol) in dichloromethane, DCM (1 mL) was added trifluoroacetic acid, TFA (0.2 mL, 0.13 mmol). The mixture was stirred at 25 °C for 1 h. TLC indicated the reaction was completed. The mixture was concentrated to afford cDLI-1j (9.20 mg, 100%) as a pale yellow solid.

### Preparation of cDLI-1

To a solution of 4-(3,5-difluoropyridin-2-yl)-10-methyl-7-((methylsulfonyl)methyl)-11-oxo-3,4,10,11-tetrahydro-1H-1,4,10-triazadibenzo[cd,f]azulene-6-carboxylic acid, cDLI-1k (10.0 mg, 0.02 mmol) and 1-[bis(dimethylamino)methylene]-1H-1,2,3-triazolo[4,5-b]pyridinium 3-oxide hexafluorophosphate; hexafluorophosphate azabenzotriazole tetramethyl uronium, HATU, CAS Reg. No. 148893-10-1 (8.00 mg, 0.02 mmol) in N,N-dimethylformamide (1 mL) was added N,N-diisopropylethylamine (0.01 mL, 0.05 mmol). The mixture was stirred at 25 °C for 5 min. Then cDLI-1j (9.20 mg, 0.01 mmol) was added. Then the mixture was stirred at 25 °C for 1 hour. LCMS (10-80AB/7.0 min): RT=3.840 min, [M+H]+ 1365.3 showed 16% of desired product. Then the mixture was filtered and the filtrate was sent to Pre-HPLC (acetonitrile 30-60/0.225% FA in water) to afford cDLI-1 (1.90 mg, 13.1%) as a pale yellow solid. LCMS (5-95, AB, 1.5 min): RT (220/254 nm)=0.88 min, m/z=1365.4 [M+H]⁺.

### Example cDLI-5 Synthesis of 4-((S)-2-(1-((5-(2,5-dioxo-2,5-dihydro-1H-pyrrol-1-yl)pentyl)carbamoyl)cyclobutane-1-carboxamido)-5-ureidopentanamido)benzyl ((3-(4-((7-(7-(3,5-difluoropyridin-2-yl)-2-methyl-O-((methylsulfonyl)methyl)-3-oxo-3,4,6,7-tetrahydro-2H-2,4,7-triazadibenzo[cd,f]azulene-9-carboxamido)heptyl)amino)-1-oxoisoindolin-2-yl)-2,6-dioxopiperidin-1-yl)methyl)carbamate, cDLI-5

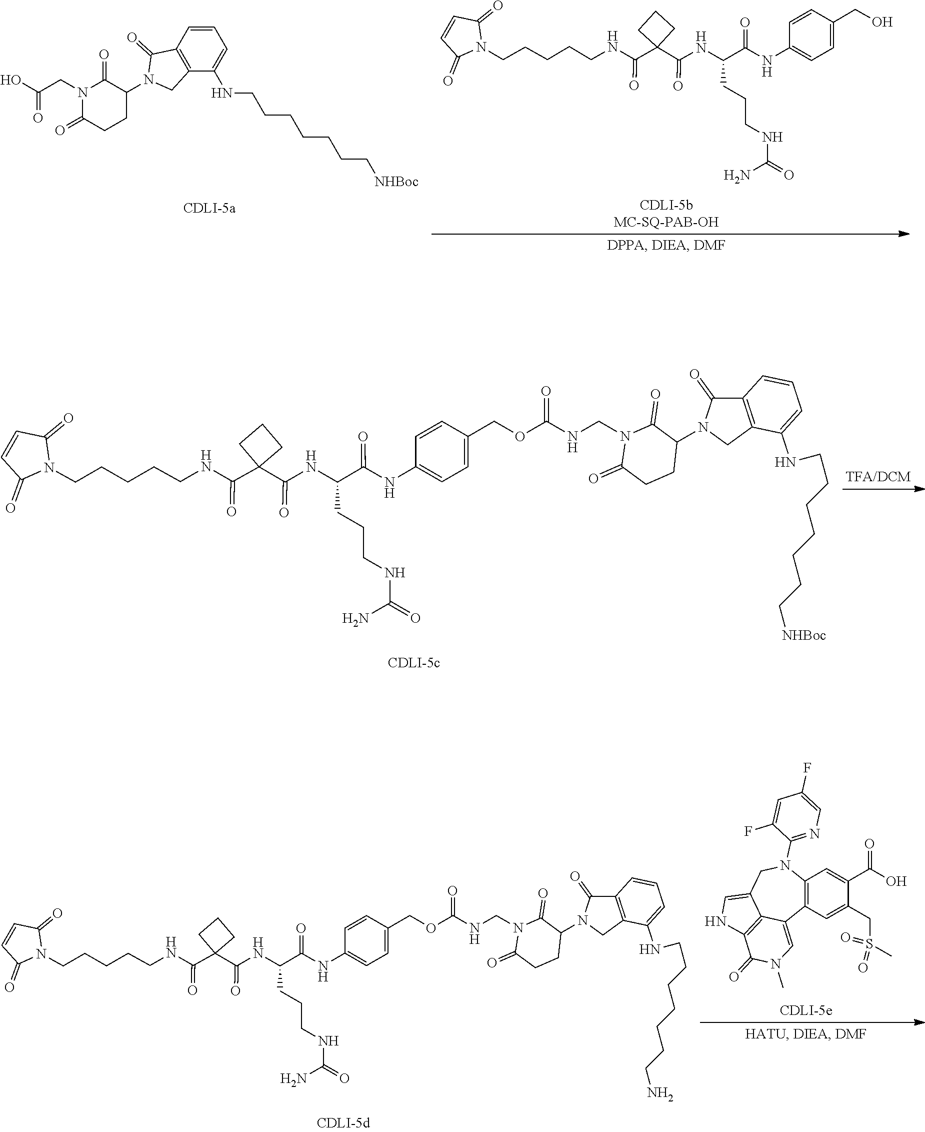 [SMILES: CC(C)(C)OC(=O)NCCCCCCCNc1cccc2c1CN(C1CCC(=O)N(CC(=O)O)C1=O)C2=O ; NC(=O)NCCC[C@H](NC(=O)C1(C(=O)NCCCCCN2C(=O)C=CC2=O)CCC1)C(=O)Nc1ccc(CO)cc1 ; CC(C)(C)OC(=O)NCCCCCCCNc1cccc2c1CN(C1CCC(=O)N(CNC(=O)OCc3ccc(NC(=O)[C@H](CCCNC(N)=O)NC(=O)C4(C(=O)NCCCCCN5C(=O)C=CC5=O)CCC4)cc3)C1=O)C2=O ; NCCCCCCCNc1cccc2c1CN(C1CCC(=O)N(CNC(=O)OCc3ccc(NC(=O)[C@H](CCCNC(N)=O)NC(=O)C4(C(=O)NCCCCCN5C(=O)C=CC5=O)CCC4)cc3)C1=O)C2=O ; Cn1cc2c3c(c[nH]c3c1=O)CN(c1ncc(F)cc1F)c1cc(C(=O)O)c(CS(C)(=O)=O)cc1-2]

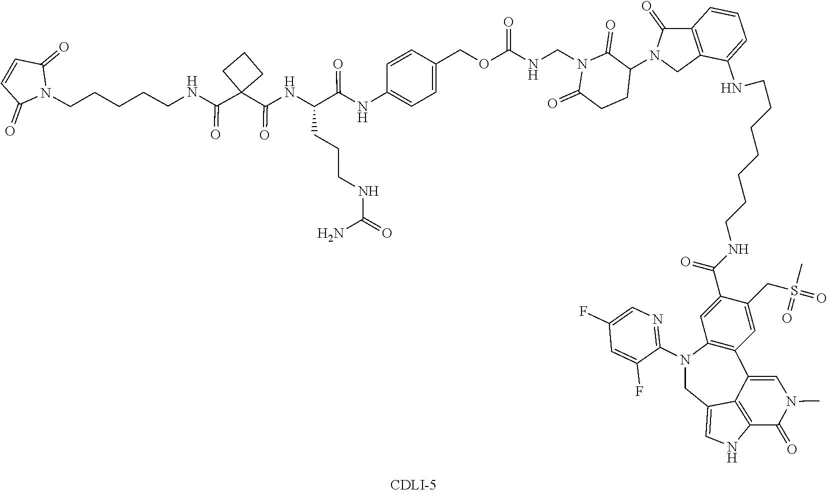 [SMILES: Cn1cc2c3c(c[nH]c3c1=O)CN(c1ncc(F)cc1F)c1cc(C(=O)NCCCCCCCNc3cccc4c3CN(C3CCC(=O)N(CNC(=O)OCc5ccc(NC(=O)[C@H](CCCNC(N)=O)NC(=O)C6(C(=O)NCCCCCN7C(=O)C=CC7=O)CCC6)cc5)C3=O)C4=O)c(CS(C)(=O)=O)cc1-2]

### Preparation of 4-((S)-2-(1-((5-(2,5-dioxo-2,5-dihydro-1H-pyrrol-1-yl)pentyl)carbamoyl)cyclobutane-1-carboxamido)-5-ureidopentanamido)benzyl ((3-(4-((7-((tert-butoxycarbonyl)amino)heptyl)amino)-1-oxoisoindolin-2-yl)-2,6-dioxopiperidin-1-yl)methyl)carbamate, cDLI-5c

To a solution of diphenylphosphoryl azide, DPPA (0.05 mL, 0.22 mmol), 2-(3-(4-((7-((tert-butoxycarbonyl)amino)heptyl)amino)-1-oxoisoindolin-2-yl)-2,6-dioxopiperidin-1-yl)acetic acid, cDLI-5a (50.0 mg, 0.09 mmol) in N,N-dimethylformamide, DMF (2 mL) was added N,N-diisopropylethylamine, DIEA (0.08 mL, 0.47 mmol). The mixture was stirred at 25 °C for 5 min., then (S)—N-(5-(2,5-dioxo-2,5-dihydro-1H-pyrrol-1-yl)pentyl)-N-(1-((4-(hydroxymethyl)phenyl)amino)-1-oxo-5-ureidopentan-2-yl)cyclobutane-1,1-dicarboxamide, cDLI5b (107.6 mg, 0.19 mmol) was added. The mixture was stirred at 90 °C for 1 hour. LCMS (10-80, AB/7.0 min): R_{T}=3.735 min, m/z=1098.3[M+H]⁺ showed 18% of desired product. Then the mixture was filtered and the filtrate was sent to pre-HPLC (acetonitrile 30-60/0.225% FA in water) to afford cDLI-5c (20 mg, 19.3%) as a pale yellow solid. LCMS (5-95, AB, 1.5 min): R_{T}=0.909 min, m/z=1098.7 [M+H]⁺.

### Preparation of 4-((S)-2-(1-((5-(2,5-dioxo-2,5-dihydro-1H-pyrrol-1-yl)pentyl)carbamoyl)cyclobutane-1-carboxamido)-5-ureidopentanamido)benzyl ((3-(4-((7-aminoheptyl)amino)-1-oxoisoindolin-2-yl)-2,6-dioxopiperidin-1-yl)methyl)carbamate, cDLI-5d

A mixture of cDLI-5c (15.0 mg, 0.01 mmol) in 5% trifluoroacetic acid in hexafluoroisopropanol, HFIP (1 mL) was stirred at 25 °C for 1 h. The mixture was concentrated to afford cDLI-5d as TFA salt (15 mg, 99%) as a white solid. LCMS (5-95, AB, 1.5 min): R_{T}=0.756 min, m/z=998.7[M+H]⁺.

### Preparation of cDLI-5

To a solution of cDLI-5d (13.5 mg, 0.03 mmol) in N,N-dimethylformamide (1 mL) was added HATU (11.8 mg, 0.03 mmol) and N,N-diisopropylethylamine (0.01 mL, 0.07 mmol). The mixture was stirred at 25 °C for 5 min. then 4-(3,5-difluoropyridin-2-yl)-10-methyl-7-((methylsulfonyl)methyl)-11-oxo-3,4,10,11-tetrahydro-1H-1,4,10-triazadibenzo[cd,f]azulene-6-carboxylic acid, cDLI-5e (15.0 mg, 0.01 mmol) was added. The mixture was stirred at 25 °C for 1 hour. LCMS (5-95AB/1.5 min): RT=0.870 min, m/z=741.3[M/2+H]⁺ showed 18% of desired product. Then the mixture was filtered and the filtrate was sent to pre-HPLC (acetonitrile 30-60/0.225% FA in water) to afford cDLI-5 (6.4 mg, 31.4%) as a white solid. LCMS (5-95, AB, 1.5 min): RT (220/254 nm)=0.863 min, m/z=1480.9[M+H]⁺.

### Example cDLI-6 Synthesis of S-(1-((((3-(4-((7-(7-(3,5-difluoropyridin-2-yl)-2-methyl-10-((methylsulfonyl)methyl)-3-oxo-3,4,6,7-tetrahydro-2H-2,4,7-triazadibenzo[cd,f]azulene-9-carboxamido)heptyl)amino)-1-oxoisoindolin-2-yl)-2,6-dioxopiperidin-1-yl)methyl)carbamoyl)oxy)-2-methylpropan-2-yl) methanesulfonothioate, cDLI-6

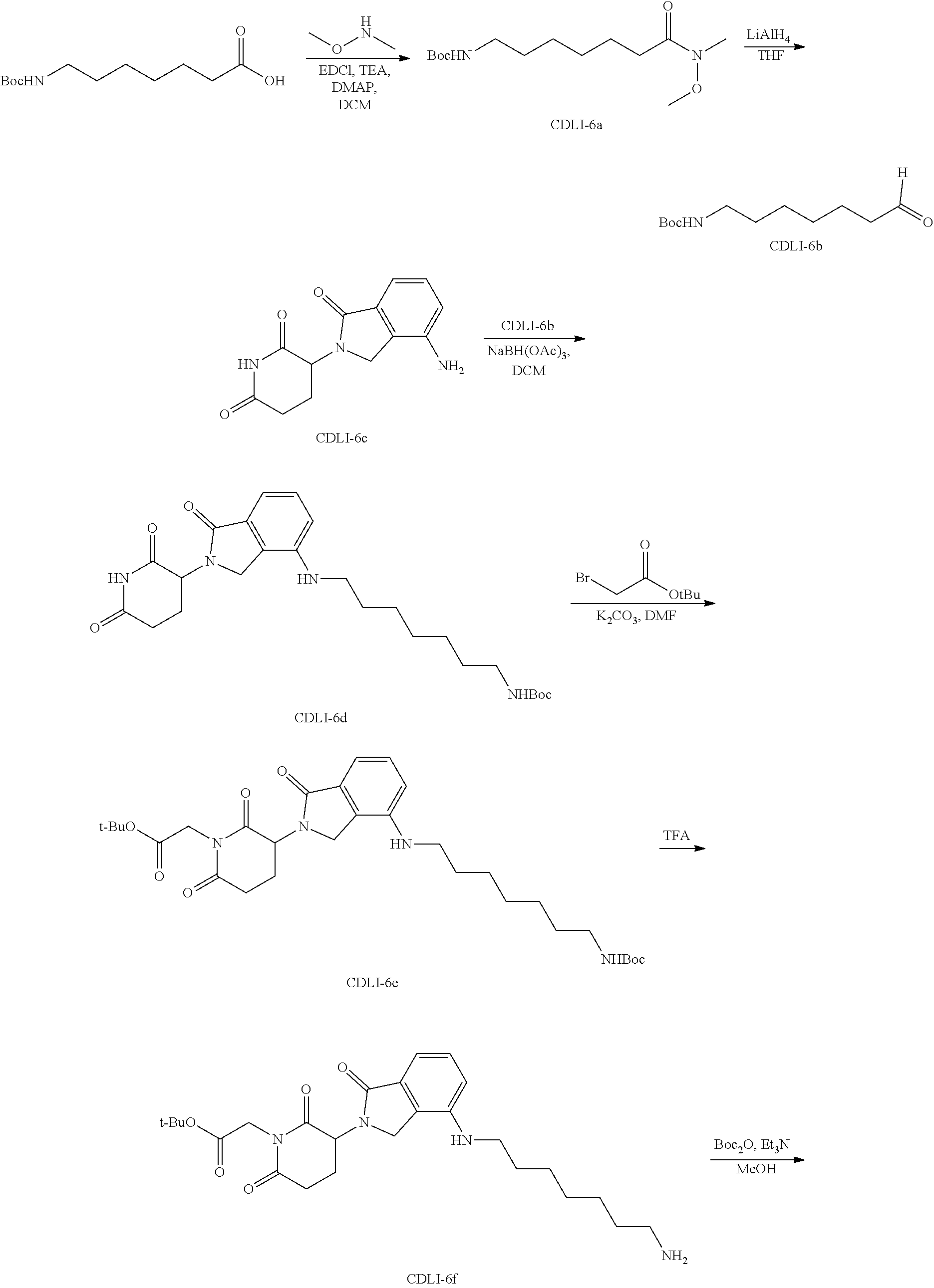

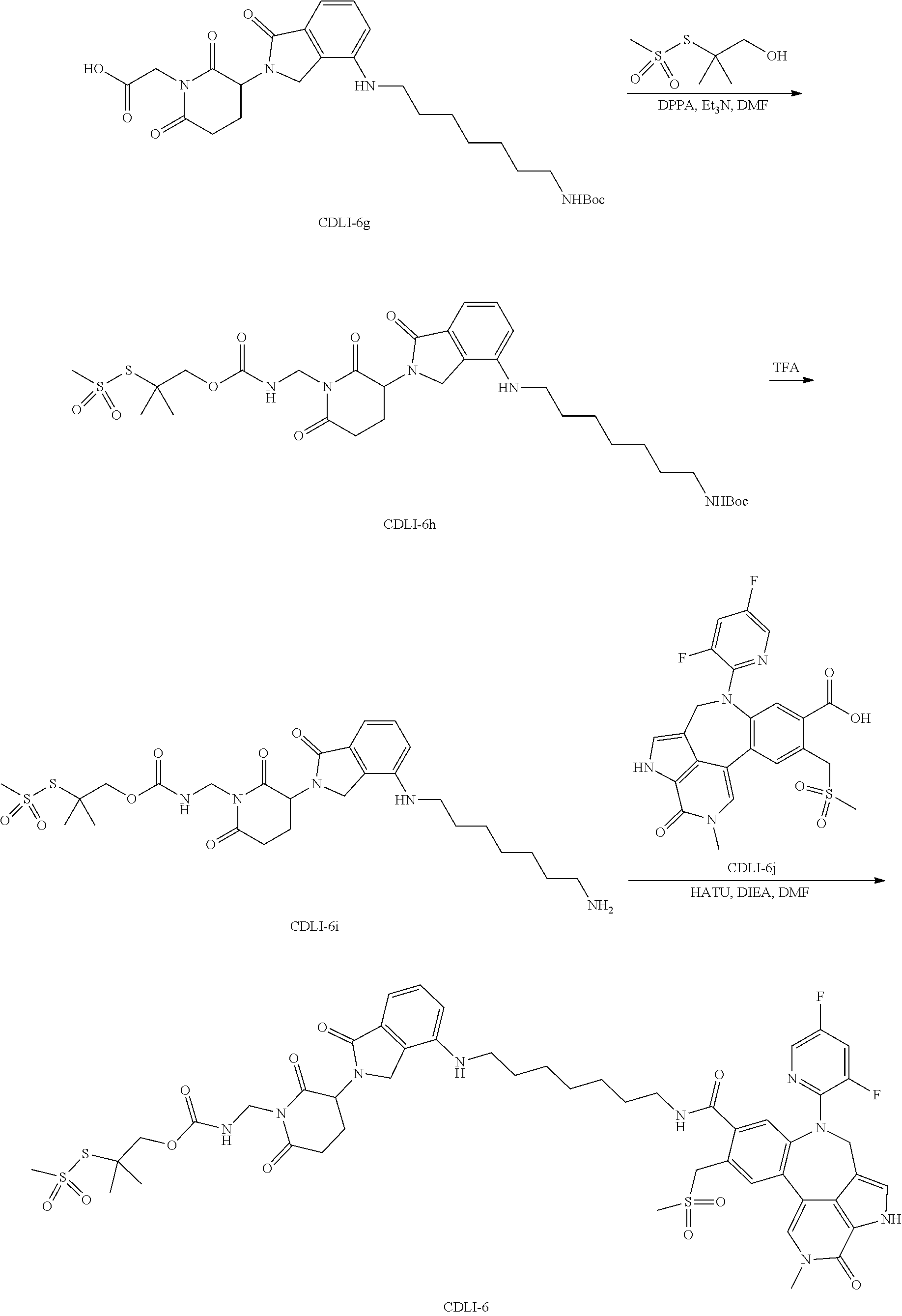

### Preparation of tert-butyl (7-(methoxy(methyl)amino)-7-oxoheptyl)carbamate, cDLI-6a

To a solution of 7-((tert-butoxycarbonyl)amino)heptanoic acid (5.0 g, 20.38 mmol) in dichloromethane (20 mL) were added EDCI (5.86 g, 30.57 mmol) and triethylamine (7.91 mL, 61.14 mmol), followed by N,O-dimethylhydroxylamine hydrochloride (0.87 g, 8.97 mmol), 4-dimethylaminopyridine (0.10 g, 0.82 mmol). The mixture was stirred at 25 °C for 16 hrs. The TLC (50% ethyl acetate in petroleum ether, R_{f}=0.8) indicated the reaction was completed. The reaction mixture was poured into water (50 mL), extracted with DCM (50 mL×3). The combined organic layers were washed with saturated ammonium chloride (100 mL), dried over Na₂SO₄, filtered and concentrated to give the crude product, which was purified by column chromatography (0-30% ethyl acetate in petroleum ether, R_{f}=0.8) to give cDLI-6a (2 g, 85%) as colorless oil. LCMS (5-95, AB, 1.5 min): R_{T}=0.772 min, m/z=189.1 [M−100+H]⁺. ¹H NMR (400 MHz, chloroform-d): 6=4.52 (br s, 1H), 3.68 (s, 3H), 3.18 (s, 3H), 3.11 (d, J=6.0 Hz, 2H), 2.41 (t, J=7.2 Hz, 2H), 1.70-1.59 (m, 2H), 1.52-1.42 (m, 11H), 1.39-1.32 (m, 4H)

### Preparation of tert-butyl (7-oxoheptyl)carbamate, cDLI-6b

To a solution of cDLI-6a (500.00 mg, 1.73 mmol) in tetrahydrofuran (12 mL) was added lithium aluminum hydride, LiAlH₄ (98.70 mg, 2.60 mmol) at −78 °C. The mixture was allowed to warm to 0 °C and stirred at this temperature for 30 mins. TLC (30% EtOAc in petroleum ether, R_{f}=0.5) indicated the reaction was completed. Saturated aqueous NH₄Cl was slowly added, under stirring at 0 °C, and the mixture was filtered and extracted with ethyl acetate (30 mL*3). The organic layer was washed with H₂O (20 mL) and brine, dried over Na₂SO₄, filtered and concentrated under reduced pressure to afford cDLI-6b (390.00 mg, 98%) as colorless oil, which was used for the next directly.

### Preparation of tert-butyl (7-((2-(2,6-dioxopiperidin-3-yl)-1-oxoisoindolin-4-yl)amino)heptyl)carbamate, cDLI-6d

To a solution of 3-(4-amino-1-oxoisoindolin-2-yl)piperidine-2,6-dione, lenalidomide (CAS Reg. No. 191732-72-6) cDLI-6c (360.00 mg, 1.39 mmol) and cDLI-6b (382.11 mg, 1.67 mmol) in N,N-dimethylformamide (3 mL) was added acetic acid (0.01 mL, 0.16 mmol). The mixture was stirred at 25 °C for 4 h. Then to the mixture was added Sodium triacetoxyborohydride, STAB, NaBH(OAc)₃ (735.73 mg, 3.47 mmol) and stirred at 25 °C for 12 h. LCMS (5-95AB/1.5 min): R_{T}=0.889 min, m/z=373.1[M−100+H]⁺ showed 28% of desired product. The reaction mixture was washed with water (30 mL×2), the organic layer was dried over anhydrous sodium sulfate, filtered, and concentrated under reduced pressure, the resulted residue was purified by column chromatography on silica gel (solvent gradient: 0-6% methanol in dichloromethane) to afford cDLI-6d (360 mg, 55%) as a pale yellow solid. LCMS (5-95, AB, 1.5 min): RT=0.887 min, m/z=495.3 [M+Na]⁺.

### Preparation of tert-butyl 2-(3-(4-((7-((tert-butoxycarbonyl)amino)heptyl)amino)-1-oxoisoindolin-2-yl)-2,6-dioxopiperidin-1-yl)acetate, cDLI-6e

To a mixture of cDLI-6d (370.00 mg, 0.78 mmol) in N,N-dimethylformamide (20 mL) was added K₂CO₃ (173.2 mg, 1.25 mmol) and tert-butyl-bromoacetate (0.15 mL, 1.02 mmol), the mixture was stirred at 25 °C for 2 hrs. TLC (10% MeOH in DCM, R_{f}=0.8) indicated the reaction was completed. The mixture was filtrated, added EtOAc (60 mL) and washed with water (20 mL). The organic layer was dried with Na₂SO₄, filtered and concentrated to give the crude product, which was purified by chromatography on silica eluting with 0-1.5% MeOH in DCM to afford cDLI-6e (320.00 mg, 70%) as a white solid. LCMS (5-95, AB, 1.5 min): RT=0.990 min, m/z=587.4 [M+H]⁺.

### Preparation of tert-butyl 2-(3-(4-((7-aminoheptyl)amino)-1-oxoisoindolin-2-yl)-2,6-dioxopiperidin-1-yl)acetate, cDLI-6f

To a mixture of cDLI-6e (320 mg, 0.55 mmol) in dichloromethane (3 mL) was added trifluoroacetic acid (3.0 mL, 38.94 mmol), the mixture was stirred at 25 °C for 2 h. LCMS (5-95AB/1.5 min): RT=0.705 min, m/z=431.2 [M+H]⁺ showed 70% of desired product. The mixture was concentrated to afford cDLI-6f (296.00 mg, 99.7%) as yellow oil.

### Preparation of 2-(3-(4-((7-((tert-butoxycarbonyl)amino)heptyl)amino)-1-oxoisoindolin-2-yl)-2,6-dioxopiperidin-1-yl)acetic acid, cDLI-6g

To a solution of cDLI-6f (296.00 mg, 0.54 mmol) in methanol (5 mL) was added Boc₂₀ (0.19 mL, 0.82 mmol) and triethylamine, Et₃N (0.23 mL, 1.63 mmol) at 25 °C, the mixture was stirred at 25 °C for 12 hrs. LCMS (5-95AB/1.5 min): RT=0.903 min, m/z=531.3[M+H]+ showed 70% of the desired product. The mixture was concentrated and purified by reverse phase chromatography (Xtimate C18® (Welch Materials, Inc.) 150*25 mm*5 um, acetonitrile 60-80.6/0.225% FA in water) to afford cDLI-6g (110 mg, 38%) as a white solid. LCMS (5-95, AB, 1.5 min): RT=0.895 min, m/z=553.3[M+Na]⁺.

### Preparation of S-(1-((((3-(4-((7-((tert-butoxycarbonyl)amino)heptyl)amino)-1-oxoisoindolin-2-yl)-2,6-dioxopiperidin-1-yl)methyl)carbamoyl)oxy)-2-methylpropan-2-yl) methanesulfonothioate, cDLI-6 h

To a solution of cDLI-6 h (70.00 mg, 0.13 mmol) and diphenylphosphoryl azide, DPPA (0.03 mL, 0.13 mmol) in toluene (5 mL) was added S-(1-hydroxy-2-methylpropan-2-yl) methanesulfonothioate (48.6 mg, 0.26 mmol), followed by triethylamine (0.03 mL, 0.20 mmol). The mixture was stirred at 25 °C for 10 minutes and heated to 90 °C for 3 hours under N₂ atmosphere. TLC (10% MeOH in DCM, R_{f}=0.6) indicated the reaction was completed. The mixture was filtered and the organic layer was concentrated in vacuum. The resulting mixture was diluted with EtOAc (40 mL) and washed with water (20 mL), the organic layer was dried with Na₂SO₄, concentrated and purified by pre-TLC (10% MeOH in DCM, R_{f}=0.6) to afford cDLI-6 h (40 mg, 43%) as white solid. LCMS (5-95, AB, 1.5 min): RT=0.958 min, m/z=734.4[M+H]⁺.

### Preparation of S-(1-((((3-(4-((7-aminoheptyl)amino)-1-oxoisoindolin-2-yl)-2,6-dioxopiperidin-1-yl)methyl)carbamoyl)oxy)-2-methylpropan-2-yl) methanesulfonothioate, cDLI-6i

To a mixture of cDLI-6 h (32.00 mg, 0.04 mmol) in dichloromethane (0.50 mL) was added trifluoroacetic acid (0.5 mL, 6.49 mmol), the mixture was stirred at 25 °C for 1 h. The mixture was concentrated to afford TFA salt of cDLI-6i (32 mg, 98.1%) as a yellow solid.

LCMS (5-95, AB, 1.5 min): RT=0.758 min, m/z=612.3 [M+H]⁺.

### Preparation of 4-(3,5-difluoropyridin-2-yl)-10-methyl-7-((methylsulfonyl)methyl)-11-oxo-3,4,10,11-tetrahydro-1H-1,4,10-triazadibenzo[cd,f]azulene-6-carboxylic acid, cDLI-6j

To a solution of methyl 4-(3,5-difluoropyridin-2-yl)-10-methyl-7-((methylsulfonyl)methyl)-11-oxo-3,4,10,11-tetrahydro-1H-1,4,10-triazadibenzo[cd,f]azulene-6-carboxylate (100.0 mg, 0.19 mmol) in tetrahydrofuran (10 mL), methanol (10 mL) and water (2.5 mL) was added lithium hydroxide monohydrate (118.2 mg, 1.94 mmol). The mixture was stirred at 40 °C for 16 hrs. TLC (10% methanol in dichloromethane, R_{f}=0.3) indicated the reaction was completed. To the mixture was added water (20 mL), the aqueous layer was acidified with 2M HCl to pH=3, then extracted with EtOAc (30 mL*4), the organic layer was dried over Na₂SO₄, filtered and concentrated to afford cDLI-6j (95 mg, 98%) as a yellow solid. LCMS (5-95, AB, 1.5 min): RT=0.770 min, m/z=501.2[M+H]⁺.

### Preparation of cDLI-6

To a solution of cDLI-6j (44.1 mg, 0.09 mmol) in N,N-dimethylformamide (1 mL) was added HATU (38.6 mg, 0.10 mmol) and N,N-Diisopropylethylamine (0.04 mL, 0.22 mmol). The mixture was stirred at 25 °C for 5 min. Then cDLI-6i 3 (32.0 mg, 0.04 mmol) was added. The mixture was stirred at 25 °C for 1 hour. Then the mixture was filtered and the filtrate was sent for Pre-HPLC (acetonitrile 30-60/0.225% FA in water) to afford cDLI-6 (6.1 mg, 12%) as a pale yellow solid. LCMS (5-95, AB, 1.5 min): RT (220/254 nm)=0.893 min, m/z=1094.5 [M+H]⁺.

### Example 101 Preparation of Cysteine Engineered Antibodies

For large scale antibody production, antibodies were produced in CHO cells. Vectors coding for VL and VH are transfected into CHO cells and IgG purified from cell culture media by protein affinity chromatography.

As initially isolated, the engineered cysteine residues in antibodies exist as mixed disulfides with cellular thiols (e.g., glutathione) and are thus unavailable for conjugation. Partial reduction of these antibodies (e.g., with DTT), purification, and reoxidation with dehydroascorbic acid (DHAA) gives antibodies with free cysteine sulfhydryl groups available for conjugation, as previously described (Junutula et al. (2008) Nat. Biotechnol. 26:925-932; US 2011/0301334). Briefly, the antibodies were combined with a cereblon degrader-linker intermediate to allow conjugation to the free cysteine residues of the antibody. After several hours, the cereblon degrader antibody conjugate is purified.

Under certain conditions, the cysteine engineered antibodies were made reactive for conjugation with the cereblon degrader-linker intermediate by treatment with a reducing agent such as DTT (Cleland's reagent, dithiothreitol) or TCEP (tris(2-carboxyethyl)phosphine hydrochloride (Getz et al, (1999) Anal. Biochem. 273:73-80; Soltec Ventures, Beverly, MA) in 50 mM Tris pH 7.5 with 2 mM EDTA for 3 hrs at 37 °C or overnight at room temperature. Full length, cysteine engineered monoclonal antibodies (THIOMAB™) expressed in CHO cells (Gomez et al, (2010) Biotechnology and Bioeng. 105(4):748-760; Gomez et al, (2010) Biotechnol. Prog. 26:1438-1445) were reduced, for example with about a 50 fold excess of DTT overnight at room temperature to reduce disulfide bonds which may form between the newly introduced cysteine residues and the cysteine present in the culture media. The reduced THIOMAB™ was diluted and loaded onto a HiTrap S column in 10 mM sodium acetate, pH 5, and eluted with PBS containing 0.3M sodium chloride. Alternatively, the antibody was acidified by addition of 1/20^{th} volume of 10% acetic acid, diluted with 10 mM succinate pH 5, loaded onto the column and then washed with 10 column volumes of succinate buffer. The column was eluted with 50 mM Tris pH7.5, 2 mM EDTA.

Light chain amino acids are numbered according to Kabat (Kabat et al., Sequences of proteins of immunological interest, (1991) 5th Ed., US Dept of Health and Human Service, National Institutes of Health, Bethesda, MD). Heavy chain amino acids are numbered according to the EU numbering system (Edelman et al, (1969) Proc. Natl. Acad. of Sci. 63(1):78-85), except where noted as the Kabat system. Single letter amino acid abbreviations are used.

Full length, cysteine engineered monoclonal antibodies (THIOMAB™) expressed in CHO cells bear cysteine adducts (cystines) or glutathionylated on the engineered cysteines due to cell culture conditions. To liberate the reactive thiol groups of the engineered cysteines, the THIOMAB™ was dissolved in 500 mM sodium borate and 500 mM sodium chloride at about pH 8.0 and reduced with about a 50-100 fold excess of 1 mM TCEP for about 1-2 hrs at 37 °C Alternatively, DTT was used as reducing agent. The formation of inter-chain disulfide bonds was monitored either by non-reducing SDS-PAGE or by denaturing reverse phase HPLC PLRP column chromatography. The reduced THIOMAB™ was diluted and loaded onto a HiTrap SP FF column in 10 mM sodium acetate, pH 5, and eluted with PBS containing 0.3M sodium chloride, or 50 mM Tris-Cl, pH 7.5 containing 150 mM sodium chloride.

Disulfide bonds were reestablished between cysteine residues present in the parent Mab by carrying out reoxidation. The eluted reduced THIOMAB™ was treated with 15× or 2 mM dehydroascorbic acid (dhAA) at pH 7 for about 3 hours or for about 3 hrs in 50 mM Tris-Cl, pH 7.5, or with 200 nM to 2 mM aqueous copper sulfate (CuSO₄) at room temperature overnight. Other oxidants, i.e. oxidizing agents, and oxidizing conditions, which are known in the art may be used. Ambient air oxidation may also be effective. This mild, partial reoxidation step forms intrachain disulfides efficiently with high fidelity. The buffer was exchanged by elution over Sephadex G25 resin and eluted with PBS with 1 mM DTPA. The thiol/antibody value was checked by determining the reduced antibody concentration from the absorbance at 280 nm of the solution and the thiol concentration by reaction with DTNB (Aldrich, Milwaukee, WI) and determination of the absorbance at 412 nm.

Liquid chromatography/Mass Spectrometric Analysis was performed on a TSQ Quantum Triple Quadrupole™ mass spectrometer with extended mass range (Thermo Electron, San Jose California). Samples were chromatographed on a PRLP-S®, 1000 A, microbore column (50 mm×2.1 mm, Polymer Laboratories, Shropshire, UK) heated to 75 °C A linear gradient from 30-40% B (solvent A: 0.05% TFA in water, solvent B: 0.04% TFA in acetonitrile) was used and the eluent was directly ionized using the electrospray source. Data was collected by the Xcalibur® data system and deconvolution was performed using ProMass® (Novatia, LLC, New Jersey). Prior to LC/MS analysis, antibodies or conjugates (50 micrograms) were treated with PNGase F (2 units/ml; PROzyme, San Leandro, CA) for 2 hours at 37 °C to remove N-linked carbohydrates.

Hydrophobic Interaction Chromatography (HIC) samples were injected onto a Butyl HIC NPR column (2.5 micron particle size, 4.6 mm×3.5 cm) (Tosoh Bioscience) and eluted with a linear gradient from 0 to 70% B at 0.8 ml/min (A: 1.5 M ammonium sulfate in 50 mM potassium phosphate, pH 7, B: 50 mM potassium phosphate pH 7, 20% isopropanol). An Agilent 1100 series HPLC system equipped with a multi wavelength detector and Chemstation software was used to resolve and quantitate antibody species with different ratios of drugs per antibody.

### Example 102 Conjugation of Cereblon Degrader-Linker Intermediates (cDLI) and Antibodies

After the reduction and reoxidation procedures of Example 101, the cysteine-engineered antibody (THIOMAB™) was dissolved in PBS (phosphate buffered saline) buffer and chilled on ice. An excess, from about 1.5 molar to 20 equivalents, of a cereblon degrader-linker intermediate, activated with a thiol-reactive group such as pyridyl disulfide, maleimide, or bromoacetamide, was dissolved in DMSO, diluted in acetonitrile and water, and added to the chilled, reduced, and reoxidized antibody in PBS. Typically the cereblon degrader-linker intermediate was added from a DMSO stock at a concentration of about 20 mM in 50 mM Tris, pH 8, to the antibody and monitored until the reaction is complete from about 1 to about 24 hours as determined by LC-MS analysis of the reaction mixture. When the reaction was complete, an excess of a capping reagent such as ethyl maleimide was added to quench the reaction and cap any unreacted antibody thiol groups. The conjugation mixture may be loaded and eluted through a HiTrap SP FF column to remove excess drug and other impurities. The reaction mixture was concentrated by centrifugal ultrafiltration and the resulting cysteine engineered cereblon degrader antibody conjugate (cDAC) was purified and desalted by elution through G25 resin in PBS, filtered through 0.2 μm filters under sterile conditions, and frozen for storage.

For example, the crude cDAC was applied to a cation exchange column after dilution with 20 mM sodium succinate, pH 5. The column was washed with at least 10 column volumes of 20 mM sodium succinate, pH 5, and the antibody was eluted with PBS. The cDAC were formulated into 20 mM His/acetate, pH 5, with 240 mM sucrose using gel filtration columns. The cDAC was characterized by UV spectroscopy to determine protein concentration, analytical SEC (size-exclusion chromatography) for aggregation analysis and LC-MS before and after treatment with Lysine C endopeptidase.

Size exclusion chromatography is performed using a Shodex KW802.5 column in 0.2M potassium phosphate pH 6.2 with 0.25 mM potassium chloride and 15% IPA at a flow rate of 0.75 ml/min. Aggregation state of the cDAC was determined by integration of eluted peak area absorbance at 280 nm.

LC-MS analysis may be performed using an Agilent QTOF 6520 ESI instrument. As an example, the cDAC is treated with 1:500 w/w Endoproteinase Lys C (Promega) in Tris, pH 7.5, for 30 min at 37 °C. The resulting cleavage fragments are loaded onto a 1000 Å (Angstrom), 8 m (micron) PLRP-S (highly cross-linked polystyrene) column heated to 80 °C and eluted with a gradient of 30% B to 40% B in 5 minutes. Mobile phase A was H₂O with 0.05% TFA and mobile phase B was acetonitrile with 0.04% TFA. The flow rate was 0.5 ml/min. Protein elution was monitored by UV absorbance detection at 280 nm prior to electrospray ionization and MS analysis. Chromatographic resolution of the unconjugated Fc fragment, residual unconjugated Fab and drugged Fab was usually achieved. The obtained m/z spectra were deconvoluted using Mass Hunter™ software (Agilent Technologies) to calculate the mass of the antibody fragments.

### Example 103 In Vitro Cell Proliferation Assay

Efficacy of the cDAC was measured by a cell proliferation assay employing the following protocol (CELLTITER GLO™ Luminescent Cell Viability Assay, Promega Corp. Technical Bulletin TB288; Mendoza et al. (2002) Cancer Res. 62:5485-5488):
1. An aliquot of 40 μl of cell culture containing about 4000 cells (HER-expressing SK-BR-3, KPL-4, CAMA1, EFM19, MV-4-11, EOL-1, Molm-13, Nomo-1, HL-60, and OCI-AML-2) in medium was deposited in each well of a 384-well, opaque-walled plate.
2. Control wells were prepared containing medium and without cells.
3. cDAC (n=3) was added to the experimental wells and incubated for 3 to 5 days.
4. The plates were equilibrated to room temperature for approximately 30 minutes.
5. A volume of CELLTITER GLO™ Reagent equal to the volume of cell culture medium present in each well was added.
6. The contents were mixed for 15 minutes on an orbital shaker to induce cell lysis.
7. The plate was incubated at room temperature for 5 minutes to stabilize the luminescence signal.
8. Luminescence was recorded and reported in graphs as % activity where RLU (relative luminescence units) was normalized to controls (no antibody control minus no cell control).

Data was plotted and illustrated in FIGS. 1, 2A-2B, 3A-3B, 4, 5A-5B, and 6A-6B as individual points for each replicate (n=3) for each antibody. The protocol is a modification of the CELLTITER GLO™ Luminescent Cell. Cell lines may be grown in media including RPMI-1640, 20% HI—FBS, 2 mM L-Glutamine.

### Example 104 Whole Blood Stability Assay

Whole blood incubation: Matrix collected in lithium heparin-containing tubes (was shipped by the vendor (BioIVT, Westbury NY). Unfrozen plasma and whole blood were collected in the afternoon and shipped cold (2-8 °C) overnight so as to arrive within 18 h of collection, while frozen plasma was collected and shipped frozen with normal delivery conditions. cDAC source material was formulated to 1 mg/mL in Buffer (1×PBS [pH 7.4], 0.5% bovine serum albumin, 15 parts per million Proclin™) and then further diluted to a final concentration of 100 μg/mL. Once mixed, 150 μL of the whole blood/Buffer stability samples was aliquoted into two separate sets of tubes for the two different time-points. The 0 h time-points were then placed in a −80 °C Whole blood samples were generated, with two aliquots of 150 μL for 0 and 24 h time-points for whole blood. The 0 h samples were immediately placed in a −80 °C freezer and while 24 h were shaken (about 700 rpm) in 37 °C incubator. Aliquots were collected at 24 h stored in −80 °C freezer until affinity capture LC-MS was performed. The matrices used to generate the samples were mouse (CB17 SCID), rat (Sprague-Dawley), monkey (cynomolgus) and human.

In vitro stability sample analysis: Streptavdin (SA)-coated magnetic beads (Thermo Fisher Scientific, catalog #60210) were washed 2× with HBS-EP buffer (GE Healthcare Life Sciences, catalog #BR-1001-88), then mixed with either biotinylated extracellular domain of target (e.g., human HER2) or anti-idiotypic antibody for specific capture or biotinylated human IgG for generic capture using a KingFisher Flex (Thermo Fisher Scientific) and incubated for 2 h at room temperature with gentle agitation. The SA-bead/Biotin-capture probe complex was then washed 2× with HBS-EP buffer, mixed with cDAC or precursor stability samples pre-diluted 1:16 with HBS-EP buffer and incubated for 2 h at room temperature with gentle agitation. After the 2 h, the SA-bead/Biotin-capture probe/sample complex was washed 2× with HBS-EP buffer, and then deglycosylated via overnight incubation with PNGase F (New England Biolabs, catalog #P0704B). The SA-bead/Biotin-capture probe/sample complex was then washed 2× with HBS-EP buffer, followed by 2× washes of water (Optima™ LC/MS Grade, Fisher Chemical, catalog #W6-1) and finally 1× wash with 10% acetonitrile. The beads were placed in 30% acetonitrile/0.1% formic acid for elution for 30 min at room temperature with gentle agitation before the beads were collected. The eluted samples were then loaded on to the LC-MS (Thermo Scientific Q-Exactive Plus) for analysis. 10 μL of cDAC samples was injected and loaded onto a Waters C4 column (1000 μm×10 cm) maintained at 65 °C. The cDAC was separated on the column using a Waters Acquity UPLC system at a flow rate of 20 μL/min with the following gradient: 20% B (100% acetonitrile+0.1% formic acid) at 0-2 min; 35% B at 2.5 min; 65% B at 5 min; 95% B at 5.5 min; 5% B at 6 min. The column was directly coupled for online detection with a Thermo Scientific Q-Exactive Plus mass spectrometer operated in positive electrospray ionization mode with an acquisition mass range from m/z 500 to 4000 Da.

### Example 105 Tumor Growth Inhibition, In Vivo Efficacy in CD33 Expressing HL-60 Mice

Tumors were established and allowed to grow in CD33 expressing, HL-60 mice to 150-200 mm³ in volume (as measured using calipers) before a single treatment on day 0. Tumor volume was measured using calipers according to the formula: V (mm³)=0.5A×B², where A and B are the long and short diameters, respectively. Mice were euthanized before tumor volume reached 3000 mm³ or when tumors showed signs of impending ulceration. Data collected from each experimental group (10 mice per group) is expressed as mean±SE.

Alternatively, the Fo5 mouse mammary tumor model was employed to evaluate the in vivo efficacy of the anti-HER2 cereblon degrader antibody conjugates (cDAC) after single dose intravenous injections, and as described previously (Phillips G D L, Li G M, Dugger D L, et al. Targeting HER2-Positive Breast Cancer with Trastuzumab-DM1, an Antibody-Cytotoxic Drug Conjugate. (2008) Cancer Res. 68:9280-90), incorporated by reference herein. Anti-HER2 cDACs were tested with the Fo5 model, a transgenic mouse model in which the human HER2 gene is over-expressed in mammary epithelium under transcriptional regulation of the murine mammary tumor virus promoter (MMTV-HER2). The HER2 over-expression causes spontaneous development of a mammary tumor. The mammary tumor of one of these founder animals (founder #5 [Fo5]) is propagated in subsequent generations of FVB mice by serial transplantation of tumor fragments (~2×2 mm in size). All studies are conducted in accordance with the Guide for the Care and Use of Laboratory Animals. Each cDAC (single dose) is dosed in nine animals intravenously at the start of the study, and 14 days post-transplant. Initial tumor size is about 200 mm³ volume.

Other mammary fat pad transplant efficacy models may be employed as described (Chen et al. (2007) Cancer Res. 67:4924-4932), evaluating tumor volume after a single intravenous dose and using tumors excised from a mouse bearing an intraperitoneal tumor, then serially passaged into the mammary fat pads of recipient mice.

All references, including publications, patent applications, and patents, cited herein are hereby incorporated by reference to the same extent as if each reference were individually and specifically indicated to be incorporated by reference and were set forth in its entirety herein.

1. The cereblon degrader antibody conjugate of claim 56 having the structure of Formula I-A 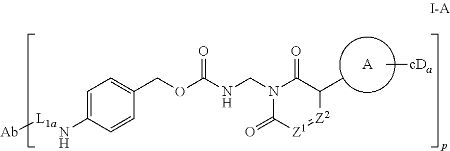
wherein
Ab is an antibody;
L₁ₐ is an antibody linker;
ring A is selected from C₆-C₂₀ aryl, C₃-C₂₀ carbocyclyl, C₃-C₂₀ heterocyclyl, and C₃-C₂₀ heteroaryl;
the dashed line [symbol] indicates an optional double bond;
Z¹ is selected from C(R¹)₂, CR¹, N, and NR^{1a}, and
Z² is selected from C(R²)₂, CR², N, and NR^{2a}, wherein
R¹ and R² are each independently selected from the group consisting of H, F, Cl, Br, I, —CN, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}, —NR^{a}R^{b}, —C(═O)NH(C₁-C₆ alkyldiyl)-NR^{a}R^{b}, —C(═O)OR^{a}, —NR^{a}R^{b}, —NO₂, ═O, —OR^{a}, —OC(═O)R^{a}, —SR^{a}, —S(O)R^{a}, —S(O)₂R^{a}, —S(O)₂NR^{a}, and —S(O)₃H;
R^{1a} and R^{2a} are each independently selected from the group consisting of H, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —OR^{a}, —S(O)R^{a}, —S(O)₂R^{a}, —S(O)₂NR^{a}, and —S(O)₃H; or
(i) two geminal R¹ or two geminal R² form a 3-6 membered carbocyclyl or heterocyclyl spiro group, or
(ii) R¹ and R², R^{1a} and R², R¹ and R^{2a}, or R^{1a} and R^{2a} form a fused 5- or 6-membered aryl, carbocyclyl, heterocyclyl, or heteroaryl group, wherein
R^{a} and R^{b} are each independently selected from H, OH, C₁-C₆ alkyl, phenyl, and benzyl, wherein phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
wherein each alkyl, alkyldiyl, alkenyl, alkynyl, aryl, carbocyclyl, heterocyclyl, and heteroaryl is independently and optionally substituted with one or more groups selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H;
cDa is the remainder of a cereblon degrader moiety; and
p is an integer from 1 to 14.

2-4. (canceled)

5. The cereblon degrader antibody conjugate of claim 1, having the structure of Formula I-A′ 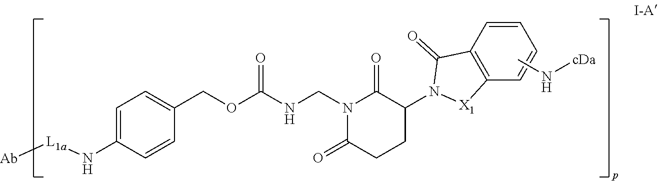
wherein X¹ is selected from CH₂ and C(═O).

6-10. (canceled)

11. The cereblon degrader antibody conjugate of claim 1, wherein L₁ₐ has the structure of Formula L₁-A 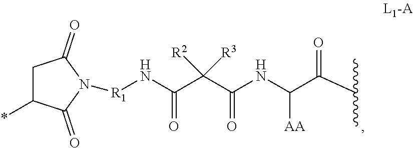
wherein
* indicates the point of attachment to a cysteine thiol of the Ab;
R¹ is selected from the group consisting of C₁-C₁₂ alkylene, C₁-C₁₂ alkylene-C(═O), C₁-C₁₂ alkylene-NH, (CH₂CH₂O)ᵣ, C₁-C₁₂ alkylene-NH, (CH₂CH₂O)ᵣ—C(═O), (CH₂CH₂O)ᵣ—C(═O), (CH₂CH₂O)ᵣ—CH₂, and C₁-C₁₂ alkylene-NHC(═O)CH2CH(thiophen-3-yl), wherein
r is an integer ranging from 1 to 10, and
C₁-C₁₂ alkylene is optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H;
R² and R³ together form a C₃-C₇ cycloalkyl ring optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H; and
AA is a side chain of an amino acid selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, and citrulline.

12. The cereblon degrader antibody conjugate of claim 11, wherein AA is selected from H, —CH₃, —CH₂(C₆H₅), —CH₂CH₂CH₂CH₂NH₂, —CH₂CH₂CH₂NHC(NH)NH₂, —CH₂CH(CH₃)₂, and —CH₂CH₂CH₂NHC(O)NH₂.

13. The cereblon degrader antibody conjugate of claim 11, wherein R¹ is C₅ alkylene.

14. The cereblon degrader antibody conjugate of claim 11, wherein R² and R³ together form a C₄ cycloalkyl ring.

15. (canceled)

16. The cereblon degrader antibody conjugate of claim 11, wherein
R¹ is C₅ alkylene;
R² and R³ together form a C₄ cycloalkyl ring; and
AA is —CH₃ or —CH₂CH₂CH₂NHC(O)NH₂.

17-18. (canceled)

19. The cereblon degrader antibody conjugate of claim 56 having the structure of Formula I-B 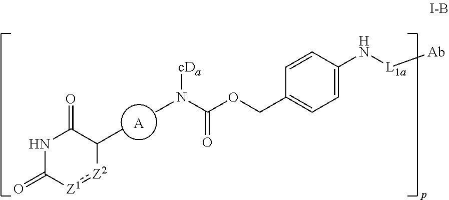
wherein
Ab is an antibody;
L₁ₐ is an antibody linker;
ring A is selected from C₆-C₂₀ aryl, C₃-C₂₀ carbocyclyl, C₂-C₂₀ heterocyclyl, and C₁-C₂₀ heteroaryl;
the dashed line [symbol] indicates an optional double bond;
Z¹ is selected from C(R¹)₂, CR¹, N, and NR^{1a}, and
Z² is selected from C(R²)₂, CR², N, and NR^{2a}, wherein
R¹ and R² are each independently selected from the group consisting of H, F, Cl, Br, I, —CN, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}, —NR^{a}R^{b}, —C(═O)NH(C₁-C₆ alkyldiyl)-NR^{a}R^{b}, —C(═O)OR^{a}, —NR^{a}R^{b}, —NO₂, ═O, —OR^{a}, —OC(═O)R^{a}, —SR^{a}, —S(O)R^{a}, —S(O)₂R^{a}, —S(O)₂NR^{a}, and —S(O)₃H;
R^{1a} and R^{2a} are independently selected from the group consisting of H, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —OR^{a}, —S(O)R^{a}, —S(O)₂R^{a}, —S(O)₂NR^{a}, and —S(O)₃H; or
(i) two geminal R¹ or two geminal R² form a 3-6 membered carbocyclyl or heterocyclyl spiro group, or
(ii) R¹ and R², R^{1a} and R², R¹ and R^{2a}, or R^{1a} and R^{2a} form a fused 5- or 6-membered aryl, carbocyclyl, heterocyclyl, or heteroaryl group, wherein
R^{a} and R^{b} are independently selected from H, OH, C₁-C₆ alkyl, phenyl, and benzyl, where phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
wherein each alkyl, alkyldiyl, alkenyl, alkynyl, aryl, carbocyclyl, heterocyclyl, and heteroaryl is independently and optionally substituted with one or more groups selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H;
cDa is the remainder of a cereblon degrader moiety; and
p is an integer from 1 to 14.

20-22. (canceled)

23. The cereblon degrader antibody conjugate of claim 19, having the structure of Formula I-B′ 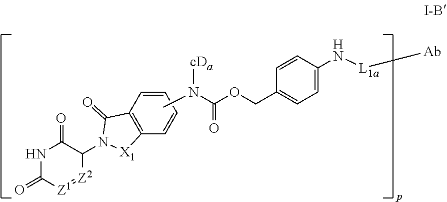
wherein X¹ is selected from CH₂ and C(═O).

24-36. (canceled)

37. The cereblon degrader antibody conjugate of claim 56 having the structure of Formula I-C 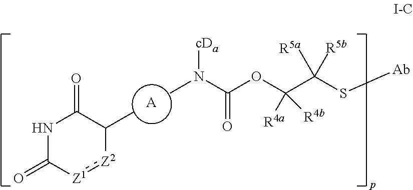
wherein
Ab is an antibody;
R^{4a}, R^{4b}, R^{5a}, and R^{5a} are each independently selected from H and C₁-C₆ alkyl, or R^{4a} and R^{4b} together with the carbon atom to which they are bound form a three-, four-, or five-membered cycloalkyl or heterocyclyl, optionally substituted with F, Cl, and C₁-C₆ alkyl, where C₁-C₆ alkyl is independently and optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H;
ring A is selected from C₆-C₂₀ aryl, C₃-C₂₀ carbocyclyl, C₂-C₂₀ heterocyclyl, and C₁-C₂₀ heteroaryl;
the dashed line [symbol] indicates an optional double bond;
Z¹ is selected from C(R¹)₂, CR¹, N, and NR^{1a}, and
Z² is selected from C(R²)₂, CR², N, and NR^{2a}, wherein
R¹ and R² are each independently selected from the group consisting of H, F, Cl, Br, I, —CN, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}, —NR^{a}R^{b}, —C(═O)NH(C₁-C₆ alkyldiyl)-NR^{a}R^{b}, —C(═O)OR^{a}, —NR^{a}R^{b}, —NO₂, ═O, —OR^{a}, —OC(═O)R^{a}, —SR^{a}, —S(O)R^{a}, —S(O)₂R^{a}, —S(O)₂NR^{a}, and —S(O)₃H;
R^{1a} and R^{2a} are independently selected from the group consisting of H, C₁-C₁₂ alkyl, C₂-C₁₂ alkenyl, C₂-C₁₂ alkynyl, (C₁-C₆ alkyldiyl)-(C₆-C₂₀ aryl), —(C₁-C₆ alkyldiyl)-NR^{a}R^{b}, —(C₁-C₆ alkyldiyl)-OR^{a}, (C₁-C₆ alkyldiyl)-(C₃-C₂₀ carbocyclyl), (C₁-C₆ alkyldiyl)-(C₂-C₂₀ heterocyclyl), (C₁-C₆ alkyldiyl)-(C₁-C₂₀ heteroaryl), C₆-C₂₀ aryl, C₃-C₂₀ carbocyclyl, C₂-C₂₀ heterocyclyl, C₁-C₂₀ heteroaryl, —C(═NH)NH(OH), —C(═NH)NH₂, —C(═O)NR^{a}R^{b}, —C(═O)NR^{a}—NR^{a}R^{b}, —C(═O)NH(C₁-C₆ alkyldiyl)-NR^{a}R^{b}, —C(═O)OR^{a}, —NR^{a}R^{b}, —ORₐ, —S(O)R^{a}, —S(O)₂R^{a}, —S(O)₂NR^{a}, and —S(O)₃H; or
(i) two geminal R¹ or two geminal R² form a 3-6 membered carbocyclyl or heterocyclyl spiro group, or
(ii) R¹ and R², R^{1a} and R², R¹ and R^{2a}, or R^{1a} and R^{2a} form a fused 5- or 6-membered aryl, carbocyclyl, heterocyclyl, or heteroaryl group, wherein
R^{a} and R^{b} are independently selected from H, OH, C₁-C₆ alkyl, phenyl, and benzyl, where phenyl and benzyl are optionally substituted with one or more groups independently selected from the group consisting of F, Cl, —CN, C₁-C₁₂ alkyl, C₂-C₁₂ alkenyl, and C₂-C₁₂ alkynyl;
wherein each alkyl, alkyldiyl, alkenyl, alkynyl, aryl, carbocyclyl, heterocyclyl, and heteroaryl is independently and optionally substituted with one or more groups selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H;
cDa is the remainder of a cereblon degrader moiety; and
p is an integer from 1 to 14.

38-41. (canceled)

42. The cereblon degrader antibody conjugate of claim 37, having the structure of Formula I-C′ 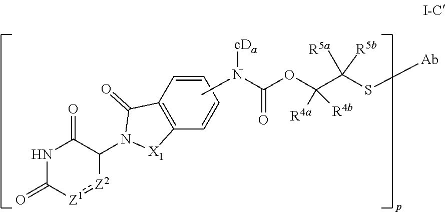
wherein X¹ is selected from CH₂ and C(═O).

43-55. (canceled)

56. A cereblon degrader antibody conjugate comprising a cereblon degrader moiety covalently attached to an antibody by an antibody linker,
wherein the cereblon degrader moiety is: (a) a target protein ligand covalently attached to a cereblon-binding, E3 ubiquitin ligase ligand by a degrader linker, or (b) a molecular glue, and
the antibody is a thiol-containing antibody.

57. (canceled)

58. The cereblon degrader antibody conjugate of claim 56, wherein the thiol-containing antibody binds to a tumor-associated antigen or cell-surface receptor selected from the group consisting of:
(1) BMPR1B (bone morphogenetic protein receptor-type IB);
(2) E16 (LAT1, SLC7A5);
(3) STEAP1 (six transmembrane epithelial antigen of prostate);
(4) MUC16 (0772P, CA125);
(5) MPF (MPF, MSLN, SMR, megakaryocyte potentiating factor, mesothelin);
(6) Napi2b (NAPI-3B, NPTIIb, SLC34A2, solute carrier family 34 (sodium phosphate), member 2, type II sodium-dependent phosphate transporter 3b);
(7) Sema 5b (FLJ10372, KIAA1445, Mm.42015, SEMA5B, SEMAG, Semaphorin 5b H log, sema domain, seven thrombospondin repeats (type 1 and type 1-like), transmembrane domain (TM) and short cytoplasmic domain, (semaphorin) 5B);
(8) PSCA hlg (2700050C12Rik, C530008016Rik, RIKEN cDNA 2700050C12, RIKEN cDNA 2700050C12 gene);
(9) ETBR (Endothelin type B receptor);
(10) MSG783 (RNF124, hypothetical protein FLJ20315);
(11) STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein);
(12) TrpM4 (BR22450, FLJ20041, TRPM4, TRPM4B, transient receptor potential cation channel, subfamily M, member 4);
(13) CRIPTO (CR, CR1, CRGF, CRIPTO, TDGF1, teratocarcinoma-derived growth factor);
(14) CD21 (CR2 (Complement receptor 2) or C3DR (C3d/Epstein Barr virus receptor) or Hs 73792);
(15) CD79b (CD79B, CD79p, IGb (immunoglobulin-associated beta), B29);
(16) FcRH2 (IFGP4, IRTA4, SPAP1A (SH2 domain containing phosphatase anchor protein 1a), SPAP1B, SPAP1C);
(17) HER2;
(18) NCA;
(19) MDP;
(20) IL20Rα;
(21) Brevican;
(22) EphB2R;
(23) ASLG659;
(24) PSCA;
(25) GEDA;
(26) BAFF-R (B cell-activating factor receptor, BLyS receptor 3, BR3);
(27) CD22 (B-cell receptor CD22-B isoform);
(28) CD79a (CD79A, CD79α, immunoglobulin-associated alpha);
(29) CXCR5 (Burkitt's lymphoma receptor 1);
(30) HLA-DOB (Beta subunit of MHC class II molecule (Ia antigen));
(31) P2X5 (Purinergic receptor P2X ligand-gated ion channel 5);
(32) CD72 (B-cell differentiation antigen CD72, Lyb-2);
(33) LY64 (Lymphocyte antigen 64 (RP105), type I membrane protein of the leucine rich repeat (LRR) family);
(34) FcRH1 (Fc receptor-like protein 1);
(35) FcRH5 (IRTA2, Immunoglobulin superfamily receptor translocation associated 2);
(36) TENB2 (putative transmembrane proteoglycan);
(37) PMEL17 (silver homolog; SILV; D12S53E; PMEL17; SI; SIL);
(38) TMEFF1 (transmembrane protein with EGF-like and two follistatin-like domains 1; Tomoregulin-1);
(39) GDNF-Ral (GDNF family receptor alpha 1; GFRA1; GDNFR; GDNFRA; RETL1; TRNR1; RET1L; GDNFR-alpha1; GFR-ALPHA-1);
(40) Ly6E (lymphocyte antigen 6 complex, locus E; Ly67, RIG-E, SCA-2, TSA-1);
(41) TMEM46 (shisa homolog 2 (Xenopus laevis); SHISA2);
(42) Ly6G6D (lymphocyte antigen 6 complex, locus G6D; Ly6-D, MEGT1);
(43) LGR5 (leucine-rich repeat-containing G protein-coupled receptor 5; GPR49, GPR67);
(44) RET (ret proto-oncogene; MEN2A; HSCR1; MEN2B; MTC1; PTC; CDHF12; Hs.168114; RET51; RET-ELE1);
(45) LY6K (lymphocyte antigen 6 complex, locus K; LY6K; HSJ001348; FLJ35226);
(46) GPR19 (G protein-coupled receptor 19; Mm.4787);
(47) GPR54 (KISS1 receptor; KISSIR; GPR54; HOT7T175; AXOR12);
(48) ASPHD1 (aspartate beta-hydroxylase domain containing 1; LOC253982);
(49) Tyrosinase (TYR; OCAIA; OCA1A; tyrosinase; SHEP3);
(50) TMEM118 (ring finger protein, transmembrane 2, RNFT2; FLJ14627);
(51) GPR172A (G protein-coupled receptor 172A; GPCR41; FLJ11856; D15Ertd747e);
(52) CD33;
(53) CLL-1;
(54) TROP2; and
(55) CD123.

59. The cereblon degrader antibody conjugate of claim 58, wherein the tumor-associated antigen or cell-surface receptor is HER2 or CD33.

60. (canceled)

61. (canceled)

62. The cereblon degrader antibody conjugate of claim 56, wherein the thiol-containing antibody is a cysteine-engineered antibody comprising one or more cysteine mutations selected from A118C, LC K149C, HC A140C, LC V205C, LC S121C, HC L174C, HC L177C, HC Y373C.

63-79. (canceled)

80. The cereblon degrader antibody conjugate of claim 56, wherein the cereblon degrader moiety has the formula:
TPL-L₂-E3UL
wherein:
TPL is a target protein ligand;
E3UL is the cereblon-binding, E3 ubiquitin ligase ligand;
L₂ is a degrader linker; and
one of TPL, E3UL and L₂ is attached to L₁; or
the cereblon degrader moiety is a molecular glue.

81. The cereblon degrader antibody conjugate of claim 80, wherein TPL has the formula: 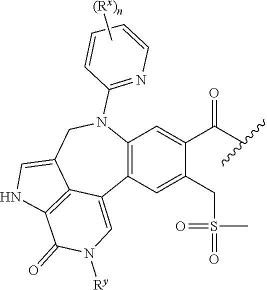
wherein
R^{x} is selected from F, Cl, and Br, and n is 0, 1, 2 or 3;
R^{y} is selected from H and C₁-C₆ alkyl; and
the wavy line indicates the point of attachment of L₂.

82-85. (canceled)

86. The cereblon degrader antibody conjugate of claim 80, wherein E3UL is selected from: 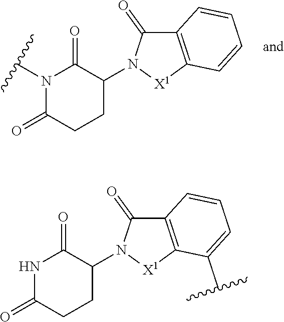
wherein X¹ is selected from CH₂ and C(═O); and the wavy line indicates the point of attachment of L₁ or L₂.

87. The cereblon degrader antibody conjugate of claim 80 having the formula: 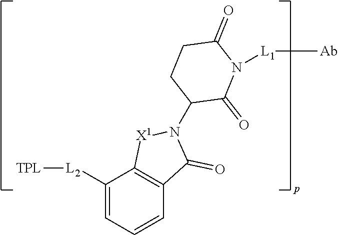
wherein L₁ is a protease-cleavable, non-peptide linker having the formula:
-Str-PM-IM-
wherein Str is a stretcher unit covalently attached to X,
PM is a peptidomimetic unit, and
IM is an immolator unit covalently attached to the glutarimide group of E3UL and has the formula: 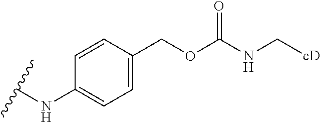
where the wavy line is the attachment to PM.

88. The cereblon degrader antibody conjugate of claim 80, having the formula: 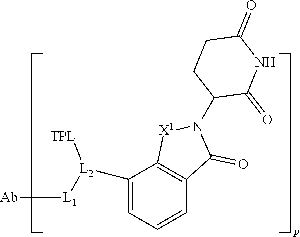
wherein L₁ is a protease-cleavable, non-peptide linker having the formula:
-Str-PM-IM-
wherein Str is a stretcher unit covalently attached to X,
PM is a peptidomimetic unit, and
IM is an immolator unit covalently attached to L₂ of cD and has the formula: 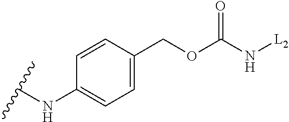
where the wavy line is the attachment to PM.

89. (canceled)

90. The cereblon degrader antibody conjugate of claim 88, selected from: 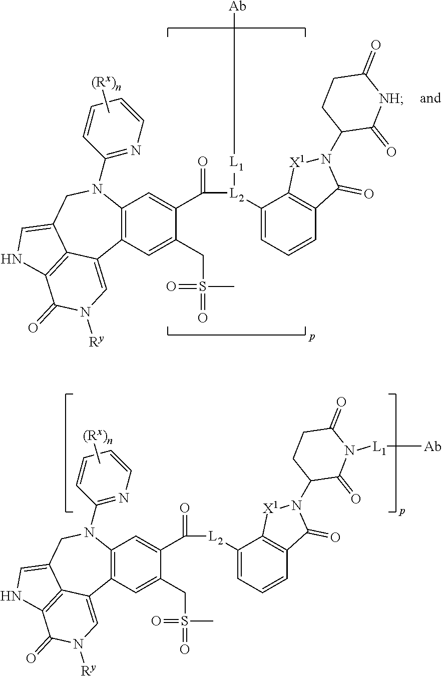
wherein R^{x} is selected from F, Cl, and Br, and n is 0, 1, 2 or 3; R^{y} is selected from H and C₁-C₆ alkyl, and X¹ is selected from CH₂ and C(═O).

91. (canceled)

92. The cereblon degrader antibody conjugate of claim 56, selected from: 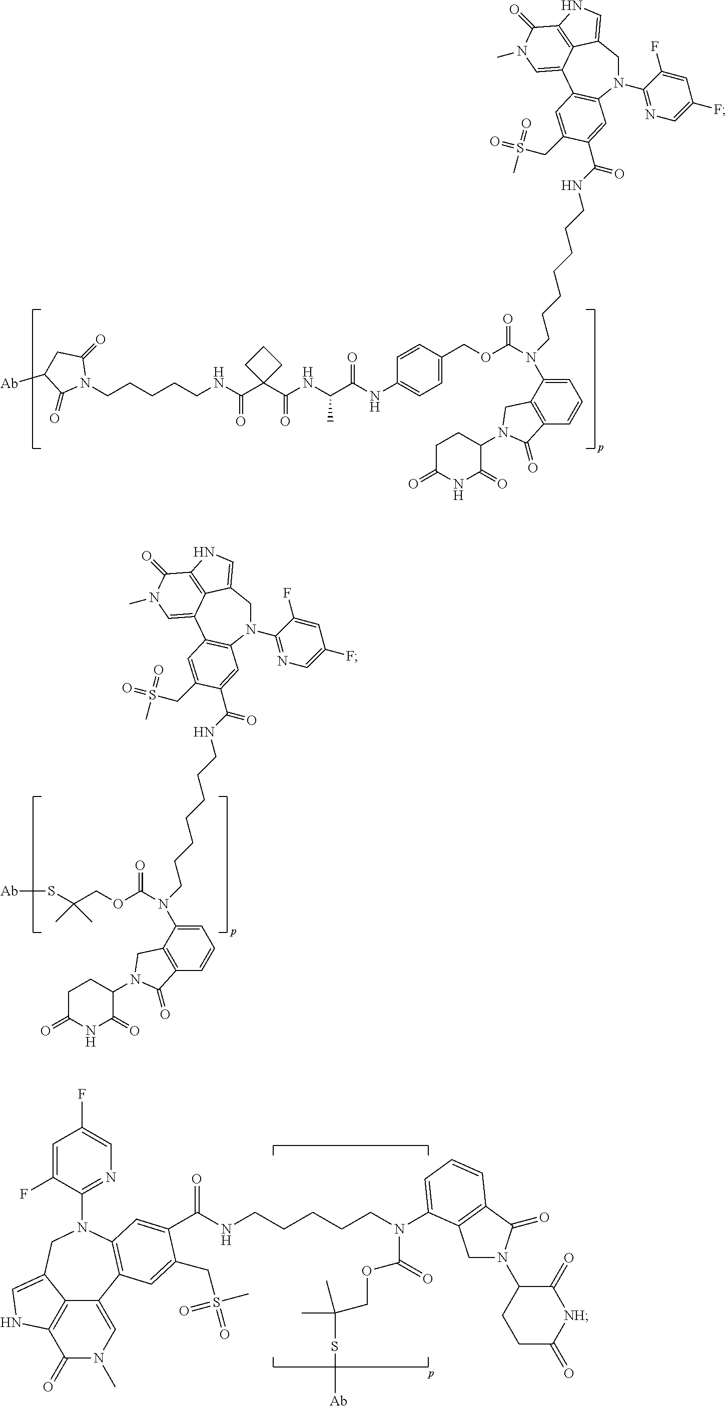 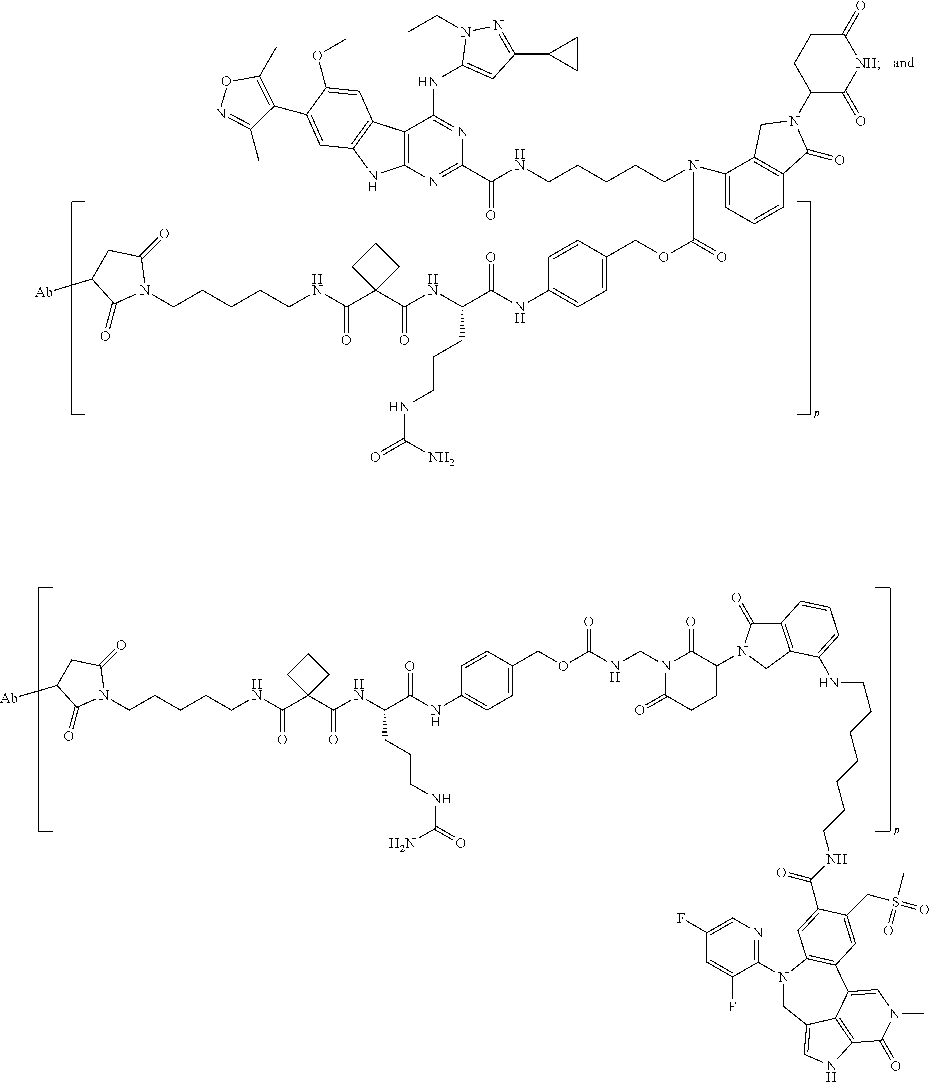
wherein Ab is the thiol-containing antibody.

93-98. (canceled)

99. A cereblon degrader-linker intermediate having Formula II:
X-L₃-cD II
wherein:
X is a thiol-reactive group;
L₃ is a linker selected from:
(i) a protease-cleavable, non-peptide linker having the formula:
-Str-PM-IM-
wherein Str is a stretcher unit covalently attached to X,
PM is a peptidomimetic unit, and
IM is an immolator unit covalently attached to cD;
(ii) a disulfide linker selected from: 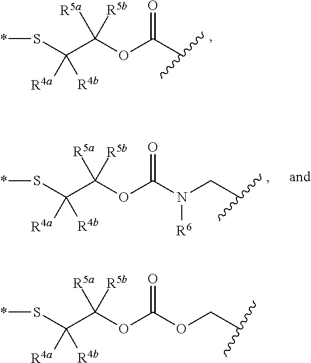
and
(iii) a linker having the formula: 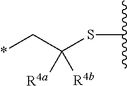
wherein * indicates the point of attachment to X,
R^{4a}, R^{4b}, R^{5a}, and R^{5a} are independently selected from H and C₁-C₆ alkyl, or R^{4a} and R^{4b} together with the carbon atom to which they are bound form a three-, four-, or five-membered cycloalkyl or heterocyclyl, optionally substituted with F, Cl, and C₁-C₆ alkyl;
R⁶ is selected from H and C₁-C₆ alkyl,
the wavy line indicates the attachment to cD,
C₁-C₆ alkyl is independently and optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H; and
cD is a cereblon degrader moiety having the formula:
TPL-L₂-E3UL
wherein:
TPL is a target protein ligand;
E3UL is a cereblon-binding, E3 ubiquitin ligase ligand; and
and L₂ is a degrader linker; or
cD is a molecular glue.

100-109. (canceled)

110. The cereblon degrader-linker intermediate of claim 99, selected from: 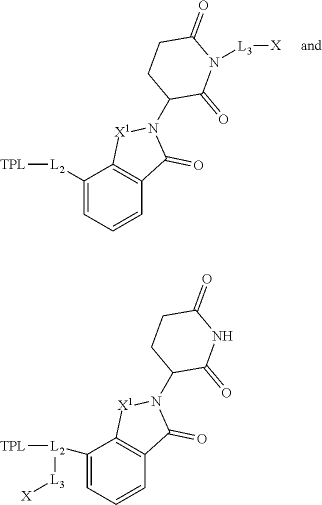
wherein
X¹ is selected from CH₂ and C(═O):
L₃ is a protease-cleavable, non-peptide linker having the formula:
-Str-PM-IM-
wherein Str is a stretcher unit covalently attached to X,
PM is a peptidomimetic unit, and
IM is an immolator unit covalently attached to cD and has the formula: 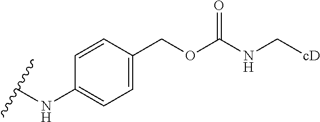
where the wavy line is the attachment to PM.

111-115. (canceled)

116. The cereblon degrader-linker intermediate of claim 99, selected from:

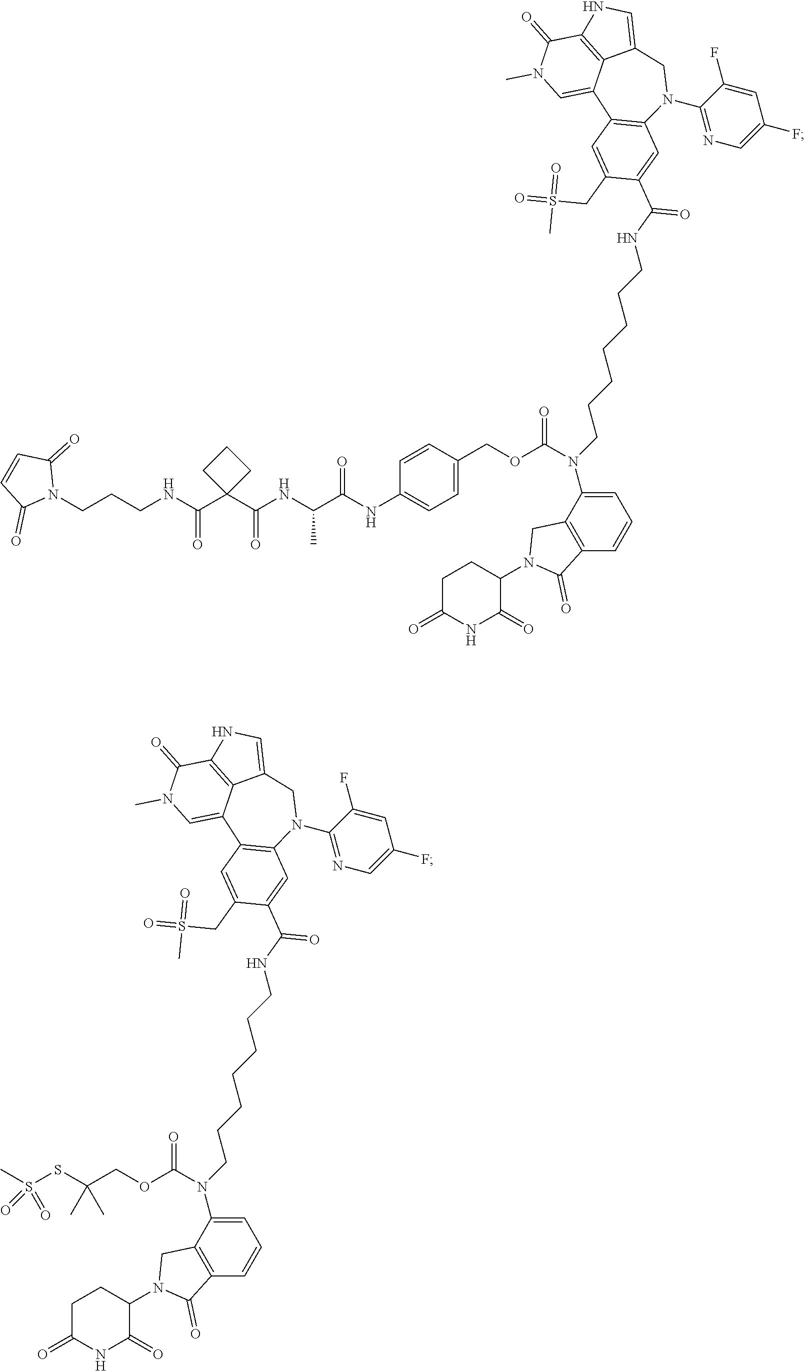

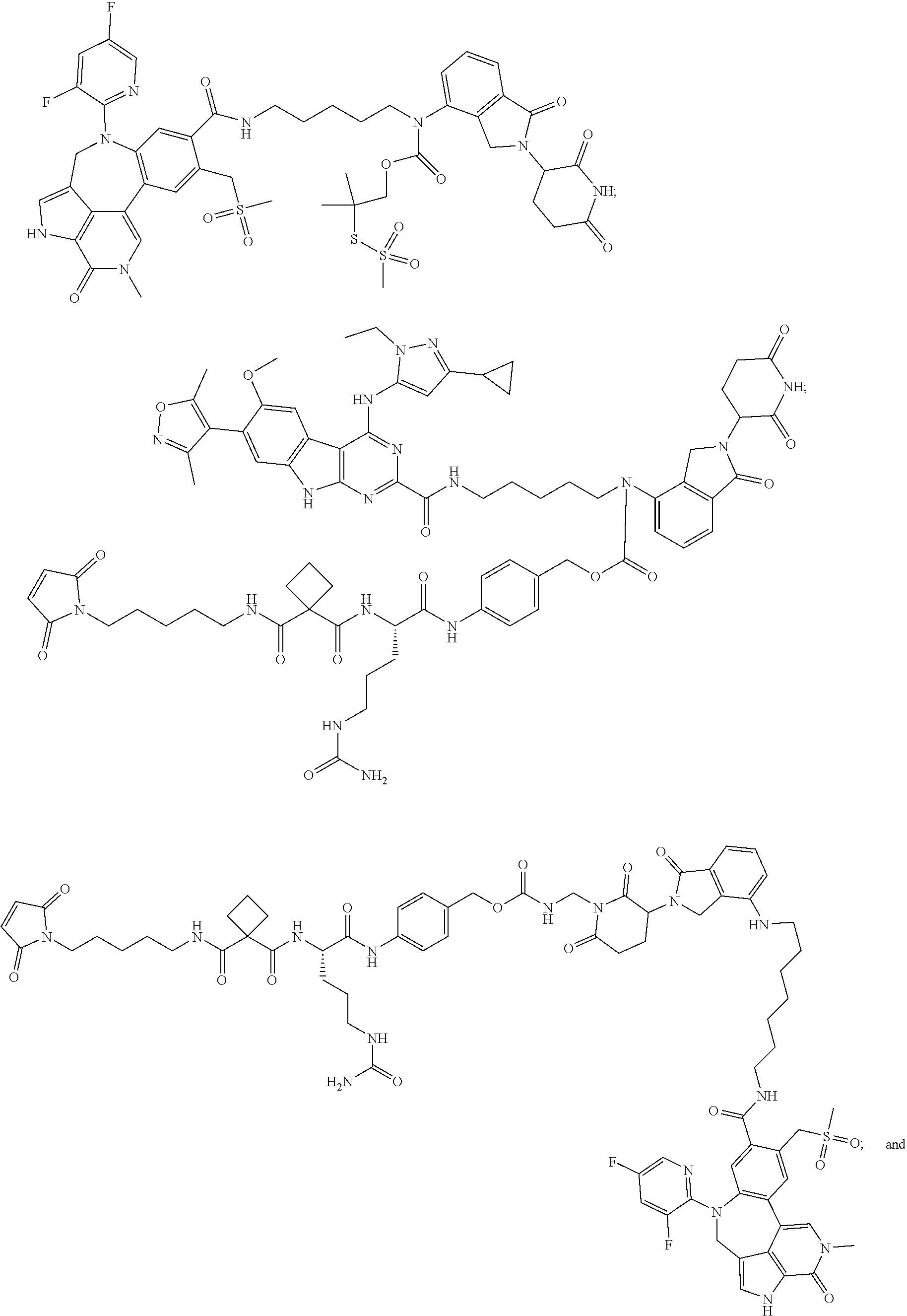

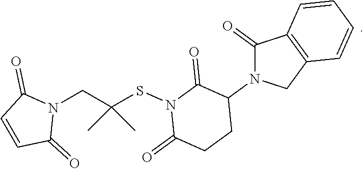

117. The cereblon degrader antibody conjugate of claim 56 prepared by conjugation of a thiol-containing antibody with a cereblon degrader intermediate selected from Table 3.

118. (canceled)

119. (canceled)

120. A pharmaceutical composition comprising the cereblon degrader antibody conjugate of claim 56 and one or more pharmaceutically acceptable diluent, vehicle, carrier or excipient.

121. A method for treating cancer in a patient in need thereof comprising administering to the patient a therapeutically effective amount of the pharmaceutical composition of claim 120, wherein the cancer is selected from carcinoma, lymphoma, blastoma, sarcoma, leukemia or lymphoid malignancies including acute myeloid leukemia, squamous cell cancer, epithelial squamous cell cancer, lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, and head and neck cancer.

122-126. (canceled)

127. The cereblon degrader antibody conjugate of claim 19, wherein L₁ₐ has the structure of Formula L₁-A 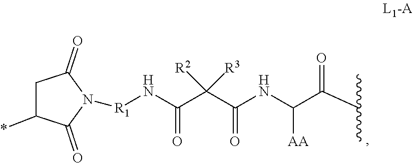
wherein
* indicates the point of attachment to a cysteine thiol of the Ab;
R¹ is selected from the group consisting of C₁-C₁₂ alkylene, C₁-C₁₂ alkylene-C(═O), C₁-C₁₂ alkylene-NH, (CH₂CH₂O)ᵣ, C₁-C₁₂ alkylene-NH, (CH₂CH₂O)ᵣ—C(═O), (CH₂CH₂O)ᵣ—C(═O), (CH₂CH₂O)ᵣ—CH₂, and C₁-C₁₂ alkylene-NHC(═O)CH₂CH(thiophen-3-yl), wherein
r is an integer ranging from 1 to 10, and
C₁-C₁₂ alkylene is optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H;
R² and R³ together form a C₃-C₇ cycloalkyl ring optionally substituted with one or more groups selected from F, Cl, —CN, —NH₂, —CH₂NH₂, —OH, —OCH₃, —OCH₂CH₃, —OCH₂CH₂OCH₃, —OCH₂CH₂OH, —OCH₂CH₂N(CH₃)₂, —OCH₂F, —OCHF₂, —OCF₃, —OP(O)(OH)₂, —S(O)₂N(CH₃)₂, —SCH₃, —S(O)₂CH₃, and —S(O)₃H; and
AA is a side chain of an amino acid selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, and citrulline.
